(19) United States
(12) Patent Application Publication
(10) Pub. No.: US 2006/0287378 A1
(43) Pub. Date: Dec. 21, 2006

(54) Novel pyrroles and imidazoles

(76) Inventors: Gary Louis Bolton, Ann Arbor, MI (US); Daniel Merritt Bowles, Howell, MI (US); David Christopher Boyles, Howell, MI (US); Walter Allen Howard JR., New Ipswich, NH (US); Richard Henry Hutchings, Ann Arbor, MI (US); Robert Michael Kennedy, Ann Arbor, MI (US); William Keun-Chan Park, Ann Arbor, MI (US); Yuntao Song, Ann Arbor, MI (US); Bharat K. Trivedi, Farmington Hills, MI (US)

Correspondence Address: WARNER-LAMBERT COMPANY, 2800 PLYMOUTH RD, ANN ARBOR, MI, 48105, US

(21) Appl. No.: 11/389,664

(22) Filed: Mar. 24, 2006

Related U.S. Application Data
(63) Continuation of application No. 11/105,288, filed on Apr. 13, 2005.
(63) Continuation of application No. PCT/IB04/02540, filed on Jul. 30, 2004.
(60) Provisional application No. 60/563,124, filed on Apr. 16, 2004.
(60) Provisional application No. 60/600,705, filed on Aug. 11, 2004.
(60) Provisional application No. 60/494,216, filed on Aug. 11, 2003.

(51) Int. Cl. A61K 31/4178; A61K 31/4172; C07D 405/02; C07D 233/54

(52) U.S. Cl. 514/397; 514/400; 548/341.5; 548/311.1

(57) ABSTRACT

Novel imidazoles and novel pyrroles are provided. Also provided are pharmaceutical compositions, methods of making and methods of using the compounds.

The present application claims priority under 35 U.S.C. Section 120, and 35 U.S.C. Section 365(c) to U.S. Ser. No. 11/105,288, filed Apr. 13, 2005, which claims priority to U.S. Provisional Application Nos. 60/563,124, filed Apr.16, 2004, and 60/600,705, filed Aug. 11, 2004; and to PCT/IB2004/002540, filed Jul. 30, 2004, which claims priority to U.S. Provisional Application No. 60/494,216, filed Aug. 11, 2003.

### BACKGROUND OF THE INVENTION

High levels of blood cholesterol and blood lipids are conditions involved in the onset of atherosclerosis. The conversion of HMG-CoA to mevalonate is an early and rate-limiting step in the cholesterol biosynthetic pathway. This step is catalyzed by the enzyme HMG-CoA reductase. It is known that inhibitors of HMG-CoA reductase are effective in lowering the blood plasma level of low density lipoprotein cholesterol (LDL-C), in man. (cf. M. S. Brown and J. L. Goldstein, New England Journal of Medicine, 305, No. 9, 515-517 (1981)). It has been established that lowering LDL-C levels affords protection from coronary heart disease (cf. Journal of the American Medical Association, 251, No. 3, 351-374 (1984)).

Statins are collectively lipid lowering agents. Representative statins include atorvastatin, lovastatin, pravastatin, simvastatin and rosuvastatin. Atorvastatin and pharmaceutically acceptable salts thereof are selective, competitive inhibitors of HMG-CoA reductase. A number of patents have issued disclosing atorvastatin. These include: U.S. Pat. Nos. 4,681,893; 5,273,995 and 5,969,156, which are incorporated herein by reference.

All statins interfere, to varying degrees, with the conversion of HMG-CoA to the cholesterol precursor mevalonate by HMG-CoA reductase. These drugs share many features, but also exhibit differences in pharmacalogic attributes that may contribute to differences in clinical utility and effectiveness in modifying lipid risk factors for coronary heart disease. (Clin. Cardiol. Bol. 26 (Suppl. III), III-32-III-38 (2003)). Some of the desirable pharmocologic features with statin therapy include potent reversible inhibition of HMG-CoA reductase, the ability to produce large reductions in LDL-C and non-high-density lipoprotein cholesterol (non-HDL-C), the ability to increase HDL cholesterol (HDL-C), tissue selectivity, optimal pharmacokinetics, availability of once a day dosing and a low potential for drug-drug interactions. Also desirable is the ability to lower circulating very-low-density-lipoprotein (VLDL) as well as the ability to lower triglyceride levels.

At the present time, the most potent statins display in vitro IC₅₀ values, using purified human HMG-CoA reductase catalytic domain preparations, of between about 5.4 and about 8.0 nM. (Am. J. Cardiol. 2001; 87(suppl): 28B-32B; Atheroscer Suppl. 2002;2:33-37). Generally, the most potent LDL-C-lowering statins are also the most potent non-HDL-C-lowering statins. Thus, maximum inhibitory activity is desirable. With respect to HDL-C, the known statins generally produce only modest increases in HDL-C. Therefore, the ability to effect greater increases in HDL-C would be advantageous as well.

With respect to tissue selectivity, differences among statins in relative lipophilicity or hydrophilicity may influence drug kinetics and tissue selectivity. Relatively hydrophilic drugs may exhibit reduced access to nonhepatic cells as a result of low passive diffusion and increased relative hepatic cell uptake through selective organic ion transport. In addition, the relative water solubility of a drug may reduce the need for extensive cytochrome P450 (CYP) enzyme metabolism. Many drugs, including the known statins, are metabolized by the CYP3A4 enzyme system. (Arch. Intern. Med. 2000; 160:2273-2280;J. Am. Pharm. Assoc. 2000; 40:637-644). Thus, relative hydrophilicity is desirable with statin therapy.

Two important pharmacokinetic variables for statins are bioavailability and elimination half-life. It would be advantageous to have a statin with limited systemic availability so as to minimize any potential risk of systemic adverse effects, while at the same time having enough systemic availability so that any pleiotropic effects can be observed in the vasculature with statin treatment. These pleiotropic effects include improving or restoring endothelial function, enhancing the stability of atherosclerotic plaques, reduction in blood plasma levels of certain markers of inflammation such as C-reactive protein, decreasing oxidative stress and reducing vascular inflammation. (Arterioscler. Thromb. Vasc. Biol. 2001; 21:1712-1719; Heart Dis. 5(1):2-7, 2003). Further, it would be advantageous to have a statin with a long enough elimination half-life to maximize effectiveness for lowering LDL-C.

Finally, it would be advantageous to have a statin that is either not metabolized or minimally metabolized by the CYP 3A4 systems so as to minimize any potential risk of drug-drug interactions when statins are given in combination with other drugs.

Accordingly, it would be most beneficial to provide a statin having a combination of desirable properties including high potency in inhibiting HMG-CoA reductase, the ability to produce large reductions in LDL-C and non-high density lipoprotein cholesterol, the ability to increase HDL cholesterol, selectivity of effect or uptake in hepatic cells, optimal systemic bioavailability, prolonged elimination half-life, and absence or minimal metabolism via the CYP3A4 system.

### SUMMARY OF THE INVENTION

This invention provides a novel series of imidazoles. Compounds of the invention are potent inhibitors of cholesterol biosynthesis. Accordingly, the compounds find utility as therapeutic agents to treat hyperlipidemia, hypercholesterolemia, hypertriglyceridemia and atherosclerosis. More specifically, the present invention provides a compound having a Formula I, 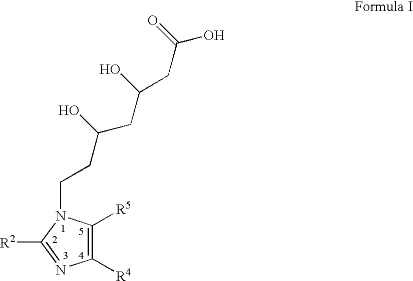
or a pharmaceutically acceptable salt, ester, amide, stereoisomer or prodrug thereof, or a pharmaceutically acceptable salt of the prodrug, wherein R² and R⁵ are each independently H; halogen; C₁-C₆ alkyl, C₃-C₈ cycloalkyl, aryl, aralkyl, heteroaryl or heteroaralkyl; optionally substituted; R⁴is halogen; H; C₁-C₆ alkyl, C₃-C₈ cycloalkyl, aryl, aralkyl, heteroaryl or heteroaralkyl; optionally substituted; —S(O)ₙNR⁶R⁷; R⁸S(O)ₙ; —(CH₂)ₙNR⁶R⁷; —(CH₂)ₙCOOR′; —(CH₂)ₙC(O)NR⁶R⁷; or —(CH₂)ₙCOR′; R⁶ and R⁷ are each independently H; C₁-C₁₀ alkyl, C₃-C₈ cycloalkyl, aryl, aralkyl, heteroaryl or heteroaralkyl; optionally substituted with aryl, heteroaryl, lower alkyl, halogen, OR′, —(CH₂)ₙCOOR′, —(CH₂)ₙCONR′R″, (CH₂)ₙSO₂R′, SO₂NR′R″ or CN; —(CH₂)ₙCOR′, —(CH₂)ₙCOOR′, —(CH₂)ₙCONR′R″ or —(CH₂)ₙSO₂R′; or N, R⁶ and R⁷ taken together form a 4-11 member ring optionally containing up to two heteroatoms selected from O, N and S, said ring being optionally substituted with aryl, aralkyl, heteroaryl, heteroaralkyl, C₁-C₁₀ alkyl, C₃-C₈ cycloalkyl, halogen, OR′, —(CH₂)ₙCOOR′, —(CH₂)ₙCONR′R″, —(CH₂)ₙSO₂R′, SO₂NR′R″ or CN; R⁸ is aryl, aralkyl, alkyl, heteroaryl, or heteroaralkyl; optionally substituted; R′ and R″ are each independently H; C₁-C₁₂ alkyl, aryl or aralkyl; optionally substituted; and n is 0-2.

Further provided is a compound having a Formula: 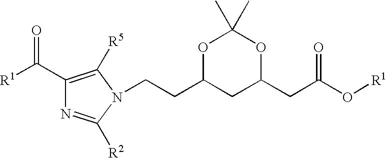
or a pharmaceutically acceptable salt, ester, amide, stereoisomer or prodrug thereof, or a pharmaceutically acceptable salt of the prodrug, wherein R² and R⁵ are each independently H; halogen; C₁-C₆ alkyl, C₃-C₈ cycloalkyl, aryl, aralkyl, heteroaryl or heteroaralkyl; optionally substituted; and R¹ is H; OH; C₁-C₁₂ alkyl, aryl or aralkyl; optionally substituted; or NR⁶R⁷ wherein R⁶ and R⁷ are each independently H; C₁-C₁₀ alkyl, C₃-C₈ cycloalkyl, aryl, aralkyl, heteroaryl or heteroaralkyl; optionally substituted, or N, R⁶ and R⁷ taken together form a 4-11 member ring optionally containing up to two heteroatoms selected from O, N and S, said ring being optionally substituted with aryl, aralkyl, heteroaryl, heteroaralkyl, C₁-C₁₀ alkyl, C₃-C₈ cycloalkyl, halogen, OR′, —(CH₂)ₙCOOR′, —(CH₂)ₙCONR′R″, —(CH₂)ₙSO₂R′, SO₂NR′R″ or CN.

Further provided is a compound having a Formula 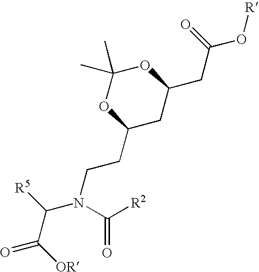
or a pharmaceutically acceptable salt, ester, amide, stereoisomer or prodrug thereof, or a pharmaceutically acceptable salt of the prodrug wherein R² and R⁵ are each independently H; halogen; C₁-C₆ alkyl, C₃-C₈ cycloalkyl, aryl, aralkyl, heteroaryl or heteroaralkyl, optionally substituted; and R′ is H; C₁-C₁₂ alkyl, aryl or aralkyl; optionally substituted.

Further provided is a compound having a formula: 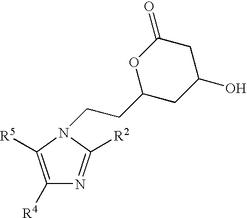
or a pharmaceutically acceptable salt, ester, amide, stereoisomer or prodrug thereof, or a pharmaceutically acceptable salt of the prodrug, wherein R², R⁴ and R⁵ are as defined above.

The invention further provides a novel series of N-alkyl pyrroles. Compounds of the invention are potent-inhibitors of cholesterol biosynthesis. Accordingly, the compounds find utility as therapeutic agents to treat hyperlipidemia, hypercholesterolemia, hypertriglyceridemia and atherosclerosis. More specifically, the present invention provides a compound having a Formula I, 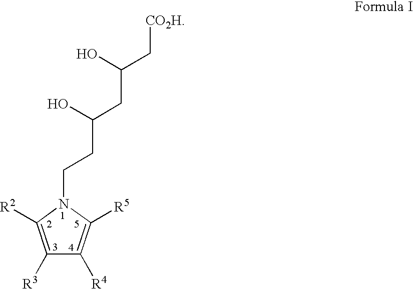
or a pharmaceutically acceptable salt, ester, amide, stereoisomer or prodrug thereof, or a pharmaceutically acceptable salt of the prodrug, wherein R² is benzyl, naphthyl or cyclohexyl, optionally substituted; phenyl or phenyl substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; one of R³ and R⁴ is H; aryl, aralkyl, heteroaryl, heteroaralkyl, optionally substituted; C₁-C₈ alkyl straight chain or branched; or C₃-C₈ cycloalkyl; and the other one of R³ and R⁴ is H, I, COOR′, R⁶R⁷NC(O)— or SO₂NR⁹R¹⁰; one of R⁶ and R⁷ is SO₂NHR⁸ or SO₂R⁸; and the other one of R⁶ and R⁷ is H or C₁-C₄ alkyl; R⁸ is aryl or heteroaryl, optionally substituted; R⁹ and R¹⁰ are each independently H; aryl, aralkyl, heteroaryl or heteroaralkyl optionally substituted with halogen, OR′, (CH₂)ₙCOOR′, (CH₂)ₙCONR′R″, (CH₂)ₙSO₂NR′R″, (CH₂)ₙSO₂R′ or CN; C₁-C₁₀ alkyl unsubstituted or substituted with OH, CO₂R′ or CONR′R″; or N, R⁹ and R¹⁰ taken together form a 4-11 member ring optionally containing up to 2 heteroatoms selected from O, N and S, said ring optionally substituted with ═O, OH, benzyl, phenyl, CO₂R′, R′OR″, (CH₂)ₙSO₂R′ or CONR′R″; R⁵ is alkyl of from one to four carbon atoms, optionally substituted with a halogen; R and R are each independently H, lower alkyl or taken together form a 4-7 member ring; and n is 0-2.

The present invention provides inter alia the following compounds: (3R,5R)-7-[2,3-Bis-(4-fluoro-phenyl)-5-isopropyl-4-methylsulfamoyl-pyrrol-1-yl]-3,5-dihydroxy-heptanoic acid; (3R,5R)-7-[2,3-Bis-(4-fluoro-phenyl)-5-isopropyl-pyrrol-1-yl]-3,5-dihydroxy-heptanoic acid (3R,5R)-7-[3-Benzylsulfamoyl-4,5-bis-(4-fluoro-phenyl)-2-isopropyl-pyrrol-1-yl]-3,5-dihydroxy-heptanoic acid; (3R,5R)-7-[2,3-Bis-(4-fluoro-phenyl)-4-(2-hydroxy-phenylsulfamoyl)-5-isopropyl-pyrrol-1-yl]-3,5-dihydroxy-heptanoic acid; (3R,5R)-7-[2,3-Bis-(4-fluoro-phenyl)-5-isopropyl-4-phenylsulfamoyl-pyrrol-1-yl]-3,5-dihydroxy-heptanoic acid; 4-[1-((3R,5R)-6-Carboxy-3,5-dihydroxy-hexyl)-4,5-bis-(4-fluoro-phenyl)-2-isopropyl-1H-pyrrole-3-sulfonylamino]-benzoic acid; 1-[1-((3R,5R)-6-Carboxy-3,5-dihydroxy-hexyl)-4,5-bis-(4-fluoro-phenyl)-2-isopropyl-1 H-pyrrole-3-sulfonyl]-piperidine-4-carboxylic acid; (3R,5R)-7-[2,3-Bis-(4-fluoro-phenyl)-5-isopropyl-4-(2-methoxycarbonyl-ethylsulfamoyl)-pyrrol-1-yl]-3,5-dihydroxy-heptanoic acid; (3R,5R)-7-[2,3-Bis-(4-fluoro-phenyl)-5-isopropyl-4-(3-methoxycarbonyl-propylsulfamoyl)-pyrrol-1-yl]-3,5-dihydroxy-heptanoic acid; (3R,5R)-7-[3-(2,4-Difluoro-phenylsulfamoyl)-4,5-bis-(4-fluoro-phenyl)-2-isopropyl-pyrrol-1-yl]-3,5-dihydroxy-heptanoic acid; (3R,5R)-7-[3-Carbamoyl-4,5-bis-(4-fluoro-phenyl)-2-isopropyl-pyrrol-1-yl]-3,5-dihydroxy-heptanoic acid; (3R,5R)-7-[2,3-Bis-(4-fluoro-phenyl)-5-isopropyl-4-(toluene-4-sulfonylaminocarbonyl)-pyrrol-1-yl]-3,5-dihydroxy-heptanoic acid; (3R,5R)-7-[2,3-Bis-(4-fluoro-phenyl)-4-(2-hydroxy-ethylsulfamoyl)-5-isopropyl-pyrrol-1-yl]-3,5-dihydroxy-heptanoic acid; 4-{[1-((3R,5R)-6-Carboxy-3,5-dihydroxy-hexyl)-5-(4-fluoro-phenyl)-2-isopropyl-4-phenyl-1H-pyrrole-3-carbonyl]-amino}-benzoic acid; (3R,5R)-7-[3-(4-Cyano-phenyl)-2-(4-fluoro-phenyl)-5-isopropyl-4-phenylcarbamoyl-pyrrol-1-yl]-3,5-dihydroxy-heptanoic acid; (3R ,5R)-7-[3-(4-Bromo-phenyl)-2-(4-fluoro-phenyl)-5-isopropyl-4-phenylcarbamoyl-pyrrol-1-yl]-3,5-dihydroxy-heptanoic acid; (3R,5R)-7-[3-(3,4-Difluoro-phenyl)-2-(4-fluoro-phenyl)-5-isopropyl-4-phenylcarbamoyl-pyrrol-1-yl]-3,5-dihydroxy-heptanoic acid; 4-{[1-((3R,5R)-6-Carboxy-3,5-dihydroxy-hexyl)-5-(4-fluoro-phenyl)-2-isopropyl-4-phenyl-1H-pyrrole-3-carbonyl]-amino}-benzoic acid; (3R,5R)-7-[2,3-Bis-(4-fluoro-phenyl)-4-(2-hydroxy-phenylsulfamoyl)-5-isopropyl-pyrrol-1-yl]-3,5-dihydroxy-heptanoic acid; (3R,5R)-7-[2-(4-Fluoro-phenyl)-5-isopropyl-3-naphthalen-2-yl-4-phenylcarbamoyl-pyrrol-1-yl]-3,5-dihydroxy-heptanoic acid; (3R,5R)-7-[3-Cyclopropyl-2-(4-fluoro-phenyl)-5-isopropyl-4-phenylcarbamoyl-pyrrol-1-yl]-3,5-dihydroxy-heptanoic acid; (3R,5R)-7-[3-(4-Dimethylcarbamoyl-phenylcarbamoyl)-5-(4-fluoro-phenyl)-2-isopropyl-4-phenyl-pyrrol-1-yl]-3,5-dihydroxy-heptanoic acid; (3R,5R)-7-[2-(4-Fluoro-phenyl)-4-iodo-5-isopropyl-3-phenyl-pyrrol-1-yl]-3,5-dihydroxy-heptanoic acid; (3R,5R)-7-[3-(4-Diethylcarbamoyl-phenylcarbamoyl)-5-(4-fluoro-phenyl)-2-isopropyl-4-phenyl-pyrrol-1-yl]-3,5-dihydroxy-heptanoic acid; (3R,5R)-7-[2-(4-Fluoro-phenyl)-5-isopropyl-4-(4-methylcarbamoyl-phenylcarbamoyl)-3-phenyl-pyrrol-1-yl]-3,5-dihydroxy-heptanoic acid; (3R,5R)-7-[2-(4-Fluoro-phenyl)-5-isopropyl-4-phenylcarbamoyl-3-pyridin-4-yl-pyrrol-1-yl]-3,5-dihydroxy-heptanoic acid; (3R,5R)-7-[2,3-Bis-(4-fluoro-phenyl)-4-(2-fluoro-phenylsulfamoyl)-5-isopropyl-pyrrol-1-yl]-3,5-dihydroxy-heptanoic acid; (3R,5R)-7-[2,3-Bis-(4-fluoro-phenyl)-4-(3-hydroxy-phenylsulfamoyl)-5-isopropyl-pyrrol-1-yl]-3,5-dihydroxy-heptanoic acid; (3R,5R)-7-[3-(4-Carbamoyl-phenylsulfamoyl)-4,5-bis-(4-fluoro-phenyl)-2-isopropyl-pyrrol-1-yl]-3,5-dihydroxy-heptanoic acid; (3R,5R)-7-[2-Ethyl-5-(4-fluoro-phenyl)-4-isopropyl-3-phenylcarbamoyl-pyrrol-1-yl]-3,5-dihydroxy-heptanoic acid; (3R,5R)-7-[2,3-Bis-(4-fluoro-phenyl)-5-isopropyl-4-(4-sulfamoyl-phenylsulfamoyl)-pyrrol-1-yl]-3,5-dihydroxy-heptanoic acid; (3R,5R)-7-[2-(4-Fluoro-phenyl)-3,5-diisopropyl-4-phenylcarbamoyl-pyrrol-1-yl]-3,5-dihydroxy-heptanoic acid; (3R,5R)-7-[2-Ethyl-5-(4-fluoro-phenyl)-4-phenethyl-3-phenylcarbamoyl-pyrrol-1-yl]-3,5-dihydroxy-heptanoic acid; (3R,5R)-7-[3-Benzylcarbamoyl-2-ethyl-5-(4-fluoro-phenyl)-4-isopropyl-pyrrol-1-yl]-3,5-dihydroxy-heptanoic acid; (3R,5R)-7-[2-(4-Fluoro-phenyl)-5-isopropyl-4-(morphoine-4-sulfonyl)-3-phenyl-pyrrol-1-yl]-3,5-dihydroxy-heptanoic acid; (3R,5R)-7-[3-(Benzyl-methyl-sulfamoyl)-4,5-bis-(4-fluoro-phenyl)-2-isopropyl-pyrrol-1-yl]-3,5-dihydroxy-heptanoic acid; (3R,5R)-7-[3-(Benzyl-methyl-sulfamoyl)-5-(4-fluoro-phenyl)-2-isopropyl-4-p-tolyl-pyrrol-1-yl]-3,5-dihydroxy-heptanoic acid; (3R,5R)-7-[3-(Benzyl-methyl-sulfamoyl)-5-(4-fluoro-phenyl)-2-isopropyl-4-naphthalen-2-yl-pyrrol-1-yl]-3,5-dihydroxy-heptanoic acid; (3R,5R)-7-[3-(4-Benzxyl-piperidine-1-sulfonyl)-5-(4-fluoro-phenyl)-2-isopropyl-4-phenyl-pyrrol-1-yl]-3,5-dihydroxy-heptanoic acid; (3R,5R)-7-[2-Ethyl-5-methyl-4-(5-methyl-pyridin-2-ylcarbamoyl)-3-p-toly;-pyrrol-1-yl]-3,5-dihydroxy-heptanoic acid; (3R,5R)-7-(2,5-Dimethyl-3-naphthalen-2-yl-4-(5-phenylcarbamoyl-pyrrol-1-yl)-3,5-dihydroxy-heptanoic acid; (3R,5R)-7-[2-Ethyl-5-methyl-4-(5-methyl-pyridin-2-ylcarbamoyl)-3-naphthalen-2-yl-pyrrol-1-yl]-3,5-dihydroxy-heptanoic acid; (3R,5R)-7-(2-Ethyl-5-methyl-3-phenyl-4-phenylcarbamoyl-pyrrol-1-yl)-3,5-dihydroxy-heptanoic acid; (3R,5R)-7-(3-Benzylcarbamoyl-2,5-dimethyl-4-phenyl-pyrrol-1-yl)-3,5-dihydroxy-heptanoic acid; (3R,5R)-7-(3-Benzylcarbamoyl-2,5-dimethyl-4-p-tolyl-pyrrol-1-yl)-3,5-dihydroxy-heptanoic acid; (3R,5R)-7-(3-benzylcarbamoyl-2,5-dimethyl-4-naphthalen-2-yl-pyrrol-1-yl)-3,5-dihydroxy-heptanoic acid; (3R,5R)-7-(3-Benzylcarbamoyl-5-ethyl-2-methyl-4-phenyl-pyrrol-1-yl)-3,5-dihydroxy-heptanoic acid; (3R,5R)-7-[2-ethyl-4-(2-methoxy-ethylcarbamoyl)-3-(4-methoxy-phenyl)-5-methyl-pyrrol-1-yl]-3,5-dihydroxy-heptanoic acid; (3R,5R)-7-(3-benzylcarbamoyl-5-ethyl-2-methyl-4-p-tolyl-pyrrol-1-yl)-3,5-dihydroxy-heptanoic acid; (3R,5R)-7-[2-Ethyl-4-(2-methoxy-ethylcarbamoyl)-5-methyl-3-naphthalen-2-yl-pyrrol-1-yl]-3,5-dihydroxy-heptanoic acid; (3R,5R)-7-[3-benzylcarbamoyl-5-ethyl-4-(4-methoxy-phenyl)-2-methyl-pyrrol-1-yl]-3,5-dihydroxy-heptanoic acid; (3R,5R)-7-(3-Benzylcarbamoyl-5-ethyl-2-methyl-4-naphthalen-2-yl-pyrrol-1-yl)-3,5-dihydroxy-heptanoic acid; (3R,5R)-7-(2,5-Dimethyl-3-phenethylcarbamoyl-4-phenyl-pyrrol-1-yl)-3,5-dihydroxy-heptanoic acid; (3R,5R)-3,5-Dihydroxy-7-(3-isobutylcarbamoyl-2,5-dimethyl-4-phenyl-pyrrol-1-yl)-heptanoic acid; (3R,5R)-3,5-Dihydroxy-7-(3-isobutylcarbamoyl-2,5-dimethyl-4-p-tolyl-pyrrol-1-yl)-heptanoic acid; (3R,5R)-7-(2-Ethyl-4-isobutylcarbamoyl-5-methyl-3-p-tolyl-pyrrol-1-yl)-3,5-dihydroxy-heptanoic acid; (3R,5R)-7-(2,5-Dimethyl-3-phenethylcarbamoyl-4-p-tolyl-pyrrol-1-yl)-3,5-dihyroxy-heptanoic acid; (3R,5R)-7-(2-benzyl-5-methyl-3-phenyl-4-phenylcarbamoyl-pyrrol-1-yl)-3,5-dihydroxy-heptanoic acid; (3R,5R)-7-[3-(4-Chloro-phenyl)-5-isopropyl-2-methyl-4-phenylcarbamoyl-pyrrol-1-yl]-3,5-dihydroxy-heptanoic acid; (3R,5R)-3,5-Dihydroxy-7-(2-methyl-4,5-diphenyl-3-phenylcarbamoyl-pyrrol-1-yl)-heptanoic acid; (3R,5R)-7-[2-(4-fluoro-phenyl)-4-iodo-5-isopropyl-3-phenyl-pyrrol-1-yl]-3,5-dihydroxy-heptanoic acid; (3R,5R)-7-[3-(4-Carbamoyl-phenylsulfamoyl)-4,5-bis-(4-fluoro-phenyl)-2-isopropyl-pyrrol-1-yl]-3,5-dihydroxy-heptanoic acid; (3R,5R)-7-[2,3-Bis-(4-fluoro-phenyl)-5-isopropyl-4-(4-sulfamoyl-phenylsulfamoyl)-pyrrol-1-yl]-3,5-dihydroxy-heptanoic acid; (3R ,5R)-7-[2-(4-Fluoro-phenyl)-5-isopropyl-4-(morpholine-4-sulfonyl)-3-phenyl-pyrrol-1-yl]-3,5-dihydroxy-heptanoic acid; (3R,5R)-7-[3-(Benzyl-methyl-sulfamoyl)-5-(4-fluoro-phenyl)-2-isopropyl-4-naphthalen-2-yl -pyrrol-1-yl]-3,5-dihydroxy-heptanoic acid; (3R,5R)-7-[3-(4-Benzyl-piperidine-1-sulfonyl)-5-(4-fluoro-phenyl)-2-isopropyl-4-phenyl-pyrrol-1-yl]-3,5-dihydroxy-heptanoic acid; 7-[3-(3-Aza-spiro[5.5]undecane-3-sulfonyl)-4,5-bis-(4-fluoro-phenyl)-2-isopropyl-pyrrol-1-yl]-3,5-dihydroxy-heptanoic acid; 7-[2,3-Bis-(4-fluoro-phenyl)-4-(4-hydroxy-piperidine-1-sulfonyl)-5-isopropyl-pyrrol-1-yl]-3,5-dihydroxy-heptanoic acid; 7-[2,3-Bis-(4-fluoro-phenyl)-5-isopropyl-4-(pyrrolidine-1-sulfonyl)-pyrrol-1-yl]-3,5-dihydroxy-heptanoic acid; 7-[2,3-Bis-(4-fluoro-phenyl)-4-(2-hydroxymethyl-pyrrolidine-1-sulfonyl)-5-isopropyl-pyrrol-1-yl]-3,5-dihydroxy-heptanoic acid; 7-[2,3-Bis-(4-fluoro-phenyl)-4-(3-hydroxy-pyrrolidine-1-sulfonyl)-5-isopropyl-pyrrol-1-yl]-3,5-dihydroxy-heptanoic acid; 7-[2,3-Bis-(4-fluoro-phenyl)-5-isopropyl-4-(3-phenyl-pyrrolidine-1-sulfonyl)-pyrrol-1-yl]-3,5-dihydroxy-heptanoic acid; 7-[2,3-Bis-(4-fluoro-phenyl)-5-isopropyl-4-(3-methanesulfonyl-pyrrolidine-1-sulfonyl)-pyrrol-1-yl]-3,5-dihydroxy-heptanoic acid; 7-[2,3-Bis-(4-fluoro-phenyl)-4-(3-hydroxy-pyrrolidine-1-sulfonyl)-5-isopropyl-pyrrol-1-yl]-3,5-dihydroxy-heptanoic acid; 7-[3-Diphenylsulfamoyl-4,5-bis-(4-fluoro-phenyl)-2-isopropyl-pyrrol-1-yl]-3,5-dihydroxy-heptanoic acid; 7-[2,3-Bis-(4-fluoro-phenyl)-5-isopropyl-4-(thiomorpholine-4-sulfonyl)-pyrrol-1-yl]-3,5-dihydroxy-heptanoic acid; 7-[3-(1,1-Dioxo-1 16-thiomorpholine-4-sulfonyl)-4,5-bis-(4-fluoro-phenyl)-2-isopropyl-pyrrol-1-yl]-3,5-dihydroxy-heptanoic acid; 7-[3-(2,6-Dimethyl-morpholine-4-sulfonyl)-4,5-bis-(4-fluoro-phenyl)-2-isopropyl-pyrrol-1-yl]-3,5-dihydroxy-heptanoic acid; 7-[2,3-Bis-(4-fluoro-phenyl)-5-isopropyl-4-(octahydro-isoquinoline-2-sulfonyl)-pyrrol-1-yl]-3,5-dihydroxy-heptanoic acid; and pharmaceutically acceptable salts, esters and amides thereof.

Further, the present invention provides a process for making a compound having a Formula 10 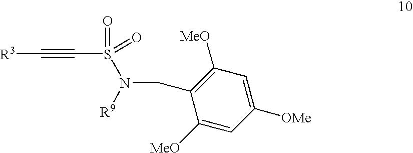
wherein R⁹ is aryl, aralkyl, heteroaryl or heteroaralkyl; optionally substituted; C₁-C₁₀ alkyl unsubstituted or substituted with OH, CO₂R′ or CONR′R″; and
R³ is aryl, aralkyl, heteroaryl, heteroaralkyl, optionally substituted; C₁-C₈ alkyl straight chain or branched; or C₃-C₈ cycloalkyl; comprising the following steps: 1.) reacting a compound having a Formula 1 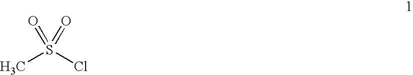
with R⁹-substituted 2,4,6-trimethoxy benzylaniline, wherein R⁹ is as defined above, to form a compound of Formula 8 wherein Me is methyl and R⁹ is as defined above, 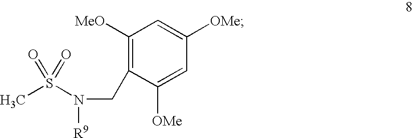
2.) reacting the compound of Formula 8 with a compound having a Formula R³COOMe wherein R³ and Me are as defined above, is in n-BuLi, to form a compound of Formula 9 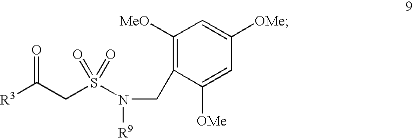
   wherein Me is methyl and R³ and R⁹ are as defined above; and 3.) contacting the compound 9 with 2-chloro N-methylpyridinium iodide and triethylamine to form the compound 10.

The present invention further provides a compound having a Formula 15 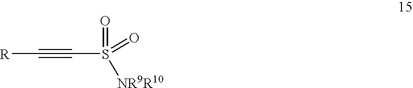
wherein R is C₁-C₈ alkyl straight chain or branched or C₃-C₈ cycloalkyl;
R⁹ and R¹⁰ are each independently H; aryl, aralkyl, heteroaryl or heteroaralkyl; optionally substituted; C₁-C₁₀alkyl unsubstituted or substituted with OH, CO₂R′ or CONR′R″; or N, R⁹ and R¹⁰ taken together form a 4-7 member ring, optionally containing up to 2 heteroatoms selected from O, N and S, said ring optionally substituted with OH, benzyl, phenyl, CO₂R′ or CON R′R″; and R and R are each independently H, lower alkyl or taken together form a 4-7 member ring.

The present invention also provides a compound having a formula C,

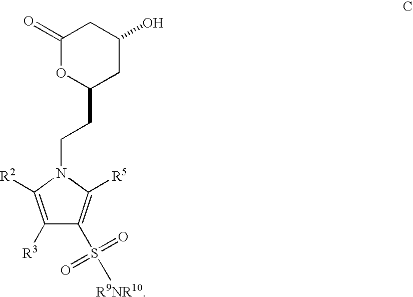

wherein R² is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; R³ is H; aryl, aralkyl, heteroaryl, heteroaralkyl, optionally substituted; C₁-C₈ alkyl straight chain or branched; or C₃-C₈ cycloalkyl;
R⁵ is alkyl of from one to four carbon atoms, optionally substituted with a halogen; and R⁹ and R¹⁰ are each independently H; aryl, aralkyl, heteroaryl or heteroaralkyl optionally substituted with halogen, OR′, (CH₂)ₙCOOR′R″, (CH₂)ₙCONR′R″, (CH₂)ₙSO₂NR′R″, (CH₂)ₙSO₂R′ or CN; C₁-C₁₀ alkyl unsubstituted or substituted with OH, CO₂R′ or CONR′R″;
or N, R⁹ and R¹⁰ taken together form a 4-7 member ring optionally containing up to 2 heteroatoms selected from O, N and S, said ring optionally substituted with OH, benzyl, phenyl, CO₂R′ or CONR′R″;
R⁵ is alkyl of from one to four carbon atoms, optionally substituted with a halogen; R and R are each independently H, lower alkyl or taken together form a 4-7 member ring; and n is 0-2.

The present invention also provides a compound having a Formula, 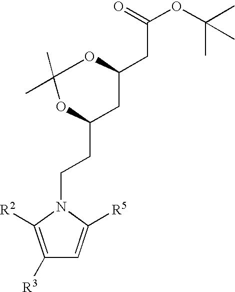
or a pharmaceutically acceptable salt, ester, amide, stereoisomer or prodrug thereof or a pharmaceutically acceptable salt of the prodrug
wherein R² is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; R³ is H; aryl, aralkyl, heteroaryl, heteroaralkyl, optionally substituted; C₁-C₈ alkyl straight chain or branched; or C₃-C₈ cycloalkyl; and R⁵ is alkyl of from one to four carbon atoms, optionally substituted with a halogen.

The present invention also provides a compound having a formula I. 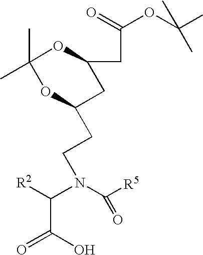
wherein R² is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; and R⁵ is alkyl of from one to four carbon atoms, optionally substituted with a halogen.

### DETAILED DESCRIPTION OF THE INVENTION

The present invention provides a compound having a Formula I, 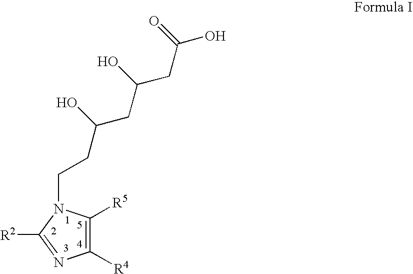
or a pharmaceutically acceptable salt, ester, amide, stereoisomer or prodrug thereof, or a pharmaceutically acceptable salt of the prodrug, wherein R², R⁴ and R⁵ are as defined above.

Further provided is the above-described compound, a pharmaceutically acceptable salt, ester, amide, stereoisomer or prodrug thereof, or a pharmaceutically acceptable salt of the prodrug wherein R⁵ is C₁-C₆ alkyl or C₃-C₈ cycloalkyl, optionally substituted. Further provided is the compound wherein R⁵ is isopropyl or cyclopropyl.

Further provided is the compound, a pharmaceutically acceptable salt, ester, amide, stereoisomer or prodrug thereof, or a pharmaceutically acceptable salt of the prodrug wherein R² is C₁-C₆ alkyl or C₃-C₈ cycloalkyl, optionally substituted. Further provided is the compound, a pharmaceutically acceptable salt, ester, amide, stereoisomer or prodrug thereof, or a pharmaceutically acceptable salt of the prodrug wherein R² is isopropyl.

Further provided is the compound, a pharmaceutically acceptable salt, ester, amide, stereoisomer or prodrug thereof, or a pharmaceutically acceptable salt of the prodrug wherein R² is aryl, aralkyl, heteroaryl or heteroaralkyl; optionally substituted.

Further provided is the compound, a pharmaceutically acceptable salt, ester, amide, stereoisomer or prodrug thereof, or a pharmaceutically acceptable salt of the prodrug wherein R⁵ is aryl, aralkyl, heteroaryl or heteroaralkyl; optionally substituted.

Further provided is the compound, a pharmaceutically acceptable salt, ester, amide, stereoisomer or prodrug thereof, or a pharmaceutically acceptable salt of the prodrug wherein R⁴ is —(CH₂)ₙC(O)NR⁶R⁷.

Further provided is the compound, a pharmaceutically acceptable salt, ester, amide, stereoisomer or prodrug thereof, or a pharmaceutically acceptable salt of the prodrug wherein R⁶ and R⁷ are each independently H; aryl, aralkyl, heteroaryl or heteroaralkyl; optionally substituted with lower alkyl, halogen, OR′, (CH₂)ₙCOOR′, —(CH₂)ₙCONR′R″, —(CH₂)ₙSO₂R′ or CN.

Further provided is the above-described compound, a pharmaceutically acceptable salt, ester, amide, stereoisomer or prodrug thereof, or a pharmaceutically acceptable salt of the prodrug wherein R² is phenyl, optionally substituted with one or more halogen.

Further provided is the compound, a pharmaceutically acceptable salt, ester, amide, stereoisomer or prodrug thereof, or a pharmaceutically acceptable salt of the prodrug wherein one of R⁶ and R⁷ is aryl, optionally substituted; and the other one of R⁶ and R⁷ is H.

Further provided is the compound, a pharmaceutically acceptable salt, ester, amide, stereoisomer or prodrug thereof, or a pharmaceutically acceptable salt of the prodrug wherein one of R⁶ and R⁷ is phenyl, optionally substituted.

Further provided is the compound, a pharmaceutically acceptable salt, ester, amide, stereoisomer or prodrug thereof, or a pharmaceutically acceptable salt of the prodrug wherein R⁶ and R⁷ are each independently H; C₁-C₁₀ alkyl, optionally substituted; or N R⁶ and R⁷ taken together form a 4-11 member ring optionally containing up to two heteroatoms selected from O, N and S, said ring being optionally substituted.

Further provided is the above-described compound, a pharmaceutically acceptable salt, ester, amide, stereoisomer or prodrug thereof, or a pharmaceutically acceptable salt of the prodrug wherein R⁴ is R⁸ S(O)ₙ.

Further provided is the compound, a pharmaceutically acceptable salt, ester, amide, stereoisomer or prodrug thereof, or a pharmaceutically acceptable salt of the prodrug wherein R⁸ is phenyl optionally substituted; and n is 2.

Further provided is the above-described compound, a pharmaceutically acceptable salt, ester, amide, stereoisomer or prodrug thereof, or a pharmaceutically acceptable salt of the prodrug wherein R⁴ is —(CH₂)ₙNR⁶R⁷.

Further provided is the compound, a pharmaceutically acceptable salt, ester, amide, stereoisomer or prodrug thereof, or a pharmaceutically acceptable salt of the prodrug wherein R⁴ is —(CH₂)ₙCOOR′ or —(CH₂)ₙCOR′.

Further provided is the compound, a pharmaceutically acceptable salt, ester, amide, stereoisomer or prodrug thereof, or a pharmaceutically acceptable salt of the prodrug wherein R⁴ is halogen; H; C₁-C₆ alkyl or C₃-C₈ cycloalkyl; optionally substituted.

Further provided is the compound, a pharmaceutically acceptable salt, ester, amide, stereoisomer or prodrug thereof, or a pharmaceutically acceptable salt of the prodrug wherein R⁴ is aryl, aralkyl, heteroaryl or heteroaralkyl; optionally substituted.

Further provided is a pharmaceutically acceptable salt of the above-described compound wherein the salt is a sodium salt.

Further provided is the above-described compound a pharmaceutically acceptable salt, ester, amide, stereoisomer or prodrug thereof, or a pharmaceutically acceptable salt of the prodrug wherein R⁶ and R⁷ are each independently H; —(CH₂)ₙCOR′; —(CH₂)ₙCOOR′; —(CH₂)ₙCONR′R″ or —(CH₂)ₘSO₂R′.

Further provided is the compound, a pharmaceutically acceptable salt, ester, amide, stereoisomer or prodrug thereof, or a pharmaceutically acceptable salt of the prodrug wherein one of R⁶ and R⁷ is phenyl, optionally substituted with one or more halogen.

Further provided is the compound, a pharmaceutically acceptable salt, ester, amide, stereoisomer or prodrug thereof, or a pharmaceutically acceptable salt of the prodrug wherein one of R⁶ and R⁷ is 4-fluorophenyl.

Further provided is the compound, a pharmaceutically acceptable salt, ester, amide, stereoisomer or prodrug thereof, or a pharmaceutically acceptable salt of the prodrug wherein one of R⁶ and R⁷ is benzyl, optionally substituted with lower alkyl, halogen, OR′, —(CH₂)ₙCOOR′, —(CH₂)ₙCONR′R″, (CH₂)ₙSO₂R′, SO₂NR′R″ or CN. Further provided is a pharmaceutically acceptable ester of the above-described compound.

Further provided is a pharmaceutical composition comprising the above-described compound, the pharmaceutically acceptable salt, ester, amide or prodrug thereof, or the pharmaceutically acceptable salt of the prodrug; or a mixture thereof; and a pharmaceutically acceptable carrier, diluent, or vehicle.

Further provided is a method of inhibiting cholesterol biosynthesis in a mammal requiring inhibition comprising administering to the mammal a therapeutically effective amount of the above-described compound or the pharmaceutically acceptable salt, ester, amide or prodrug thereof, or the pharmaceutically acceptable salt of the prodrug.

Further provided is a method of lowering LDL cholesterol in a mammal.

Further provided is a method of raising HDL cholesterol in a mammal.

Further provided is a method of treating, preventing or controlling hyperlipidemia in a mammal.

Further provided is a method of treating, preventing or controlling hypercholesterolemia in a mammal.

Further provided is a method of treating, preventing or controlling hypertriglyceridemia in a mammal.

Further provided is a method of treating, preventing or controlling Alzheimer's disease, BPH, diabetes or osteoporosis in a mammal.

Further provided is a compound having a Formula:

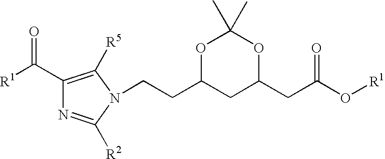

or a pharmaceutically acceptable salt, ester, amide, stereoisomer or prodrug thereof, or a pharmaceutically acceptable salt of the prodrug, wherein R¹, R² and R⁵ are as defined above.

Further provided is a compound having a Formula 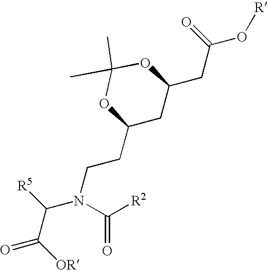
or a pharmaceutically acceptable salt, ester, amide, stereoisomer or prodrug thereof, or a pharmaceutically acceptable salt of the prodrug wherein R², R⁵ and R′ are as defined above.
Further provided is a compound having a Formula: 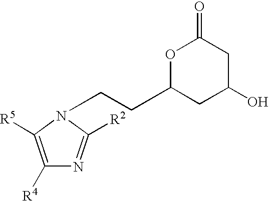
or a pharmaceutically acceptable salt, ester, amide, stereoisomer or prodrug thereof, or a pharmaceutically acceptable salt of the prodrug, wherein R², R⁴ and R⁵ are as defined above.

Further provided is the lactone form of a compound as described above, wherein R² is phenyl optionally substituted with one or more halogen, R⁴ is —(CH₂)ₙC(O)NR⁶R⁷, one of R⁶ and R⁷ is aralkyl, optionally substituted, and the other one of R⁶ and R⁷ is H; and R⁵ is C₁-C₆ alkyl or C₃-C₈ cycloalkyl.

Further provided are racemic mixtures of all compounds described herein.

Further provided is a process for preparing a compound having a Formula b. 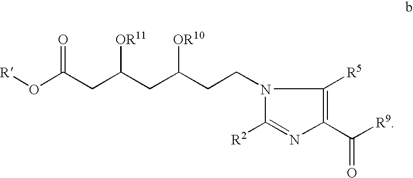
from a compound having a Formula a. 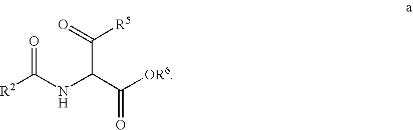
comprising the following steps:

1.) Reacting the compound a. with a compound having a formula c., 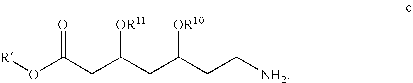
in a solvent; and
optionally reacting the compound a. with a compound NHR⁶R⁷, in a solvent, prior to the first step;
wherein R² and R⁵ are each independently H; halogen; C₁-C₆ alkyl, C₃-C₈ cycloalkyl, aryl, aralkyl, heteroaryl or heteroaralkyl; optionally substituted;
R⁹ is —OR⁶or —NR⁶R⁷;
R⁶ is H; C₁-C₁₀ alkyl, C₃-C₈ cycloalkyl, aryl, aralkyl, heteroaryl or heteroaralkyl; optionally substituted with aryl, heteroaryl, lower alkyl, halogen, OR′, —(CH₂)ₙCOOR′, —(CH₂)ₙCONR′R″, (CH₂)ₙSO₂R′, SO₂NR′R″ or CN;
R⁷ is H; C₁-C₁₀ alkyl, C₃-C₈ cycloalkyl, aryl, aralkyl, heteroaryl or heteroaralkyl; optionally substituted with aryl, heteroaryl, lower alkyl, halogen, OR′, —(CH₂)ₙCOOR′, —(CH₂)ₙCONR′R″, (CH₂)ₙSO₂R′, SO₂NR′R″ or CN; —(CH₂)ₙCOR′, —(CH₂)ₙCOOR′, —(CH₂)ₙCONR′R″ or —(CH₂)ₙSO₂R′; or N, R⁶ and R⁷taken together form a 4-11 member ring optionally containing up to two heteroatoms selected from O, N and S, said ring being optionally substituted with aryl, aralkyl, heteroaryl, heteroaralkyl, C₁-C₁₀ alkyl, C₃-C₈ cycloalkyl, halogen, OR′, —(CH₂)ₙCOOR′, —(CH₂)ₙCONR′R″, —(CH₂)ₙSO₂R′, SO₂NR′R″ or CN;
R′ and R″ are each independently H; C₁-C₁₂ alkyl, aryl or aralkyl; optionally substituted; n is 0-2;
R¹⁰ and R¹¹ are each independently C₁-C₁₀ alkyl, C(O)R⁷, —SiR¹²R¹³R¹⁴or R¹⁰ and R¹¹ taken together from isopropyl; and R¹², R¹³ and R¹⁴ are each independently C₁-C₆ alkyl.

Further provided is a process for preparing a compound having a Formula: 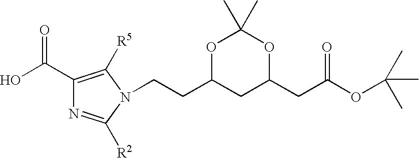
wherein R¹, R² and R⁵ are as defined above comprising the following steps:
1.) reacting a compound having a formula, 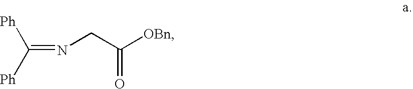
   wherein Ph is phenyl and Bn is benzyl, with a compound having a formula, 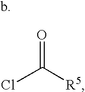
   wherein R⁵ is as defined above, under basic conditions, to form a compound having a formula; 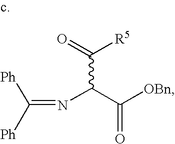
   wherein R⁵ and Bn are as defined above;
2.) hydrolyzing the compound c and subsequently reacting the hydrolyzed compound c with a compound 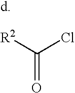
   wherein R² is as defined above, under basic conditions, to form a compound 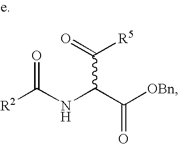
   wherein R², R⁵ and Bn are as defined above;
   3.) reacting the compound e with a compound having the formula 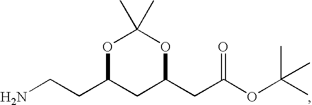
   to form a compound 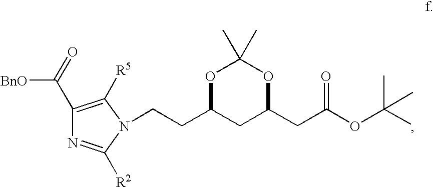
   wherein Bn, R² and R⁵ are as defined above; and hydrogenolysing the compound f to form the compound.

Further, the present invention provides a compound having a formula: 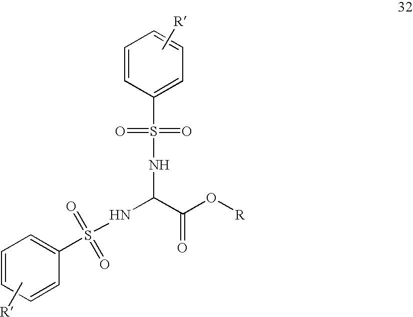
wherein R and R are as defined above.

Further provided is a compound having a formula: 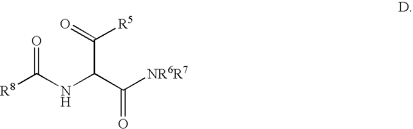
wherein R⁵, R⁶, R⁷ and R⁸ are as defined above.

The present invention provides inter alia the following compounds: (3R,5R)-7-[4-Benzylcarbamoyl-2-(4-f luoro-phenyl)-5-isopropyl-imidazol-1-yl]-3,5-dihydroxy-heptanoic acid;
(3R,5R)-7-[2-(4-Fluoro-phenyl)-5-isopropyl-4-(2-methoxy-ethylcarbamoyl)-imidazol-1-yl]-3,5-dihydroxy-heptanoic acid;
(3R,5R)-7-[2-(4-Fluoro-phenyl)-5-isopropyl-4-phenylcarbamoyi-imidazol-1-yl]-3,5-dihydroxy-heptanoic acid;
(3R,5R)-7-[4-(1,3-Dihydro-isoindole-2-carbonyl)-2-(4-fluoro-phenyl)-5-isopropyl-imidazol-1yl]-3,5-dihydroxy-heptanoic acid;
(3R,5R)-7-[4-(Benzyl-ethyl-carbamoyl)-2-(4-fluoro-phenyl)-5-isopropyl-imidazol-1-yl]-3,5-dihydroxy-heptanoic acid;
(3R,5R)-7-2-(4-Fluoro-phenyl)-5-isopropyl-4-[(pyridin-3-ylmethyl)-carbamoyl]-imidazol-1-yl}-3,5-dihydroxy-heptanoic acid;
(3R,5R)-7-[2-(4-Fluoro-phenyl)-5-isopropyl-4-(2-pyridin-3-yl-ethylcarbamoyl)-imidazol-1-yl]-3,5-dihydroxy-heptanoic acid;
(3R,5R)-7-[2-(4-Fluoro-phenyl)-5-isopropyl-4-((R)-2-phenyl-propylcarbamoyl)-imidazol-1-yl]-3,5-dihydroxy-heptanoic acid
(3R,5R)-7-[4-[2-(4-Chloro-phenyl)-3-hydroxy-propylcarbamoyl]-2-(4-fluoro-phenyl)-5-isopropyl-imidazol-1-yl]-3,5-dihydroxy-heptanoic acid;
(3R,5R)-7-{2-(4-Fluoro-phenyl)-5-isopropyl-4-[2-(4-sulfamoyl-phenyl)-ethylcarbamoyl]-ethylcarbamoyl]-imidazol-1-yl}-3,5-dihydroxy-heptanoic acid;
(3R,5R)-7-[2-(4-fluoro-phenyl)-5-isopropyl-4-((S)-1-methyl-3-phenyl-propylcarbamoyl)-imidazol-1-yl]-3,5-dihydroxy-heptanoic acid;
(3R,5R)-7-{2-(4-fluoro-phenyl)-4-[2-(3-fluoro-phenyl)-ethylcarbamoyl]-5-isopropyl-imadzol-1-yl}-3,5-dihydroxy-heptanoic acid;
(3R,5R)-7-[2-(4-fluoro-phenyl)-4-((1S,2S)-2-hydroxy-1-methoxymethyl-2-phenyl-ethylcarbamoyl)-5-isopropyl-imidazol-1-yl]-3,5-dihydroxy-heptanoic acid;
(3R,5R)-7-{2-(4-fluoro-phenyl)-5-isopropyl-4-[2-(4-methoxy-phenyl)-ethylcarbamoyl]-imidazol-1-yl}-3,5-dihydroxy-heptanoic acid; (3R,5R)-7-[2-(4-fluoro-phenyl)-4-((S)-1-hydroxymethyl-2-phenyl-ethylcarbamoyl)-5isopropyl-imidazol-1-yl]-3,5-dihydroxy-heptanoic acid;
(3R,5R)-7-{2-(4-fluoro-phenyl)-4-[(1 S,2S)-2-hydroxy-1-hydroxymethyl-2-(4-methylsulfanyl-phenyl)-ethylcarbamoyl]-5-isopropyl-imidazol-1-yl}-3,5-dihydroxy-heptanoic acid;
(3R,5R)-7-[4-[2-(4-chloro-phenyl)-ethylcarbamoyl]-2-(4-fluoro-phenyl)-5-isopropyl-imidazol-1-yl]-3,5-dihydroxy-heptanoic acid;
(3R,5R)-7-[2-(4-fluoro-phenyl)-5-isopropyl-4-((S)-2-phenyl-propylcarbamoyl)-imidazol-1-yl]-3,5-dihydroxy-heptanoic acid
(3R,5R)-7-[2-(4-fluoro-phenyl)-5-isopropyl-4-[2-(3-methoxy-phenyl)-ethylcarbamoyl]-imidazol-1-yl}-3,5-dihydroxy-heptanoic acid;
(3R,5R)-7-{2-(4-fluoro-phenyl)-4-[2-(4-fluoro-phenyl)-ethylcarbamoyl]-5-isopropyl-imidazol-1-yl}-3,5-dihydroxy-heptanoic acid;
(3R,5R)-7-[4-[2-(3-chloro-phenyl)-ethylcarbamoyl]-2-(4-fluoro-phenyl)-5-isopropyl-imidazol-1-yl]-3,5-dihydroxy-heptanoic acid;
(3R,5R)-7-[2-(4-fluoro-phenyl)-5-isopropyl-4-(2-pyridin-4-yl-ethylcarbamoyl)-imidazol-1-yl]-3,5-dihydroxy-heptanoic acid;
(3R,5R)-7-[2-(4-fluoro-phenyl)-4-((1R,2R)-2-hydroxy-1-hydroxymethyl-2-phenyl-ethylcarbamoyl)-5-isopropyl-imidazol-1-yl]-3,5-dihydroxy-heptanoic acid;
(3R,5R)-7-[2-(4-Fluoro-phenyl)-5-isopropyl-4-benzylcarbamoyl-imidazol-1-yl]-3,5-dihydroxy-heptanoic acid;
(3R,5R)-7-[2-(4-Fluoro-phenyl)-5-isopropyl-4-phenylcarbamoyi-imidazol-1-yl]-3,5-dihydroxy-heptanoic acid;
(3S,5R)-7-[2-(4-fluoro-phenyl)-5-isopropyl-4-(toluene-4-sulfonyl)-imidazol-1-yl]-3,5-dihydroxy-heptanoic acid; (3R,5R)-7-[2-(4-Fluoro-phenyl)-5-ethyl-4-(4-fluorophenylcarbamoyl)-imidazol-1-yl]-3,5-dihydroxy-heptanoic acid;
(3R,5R)-7-[2-(4-Fluoro-phenyl)-5-propyl-4-phenylcarbamoyl-imidazol-1-yl]-3,5-dihydroxy-heptanoic acid;
(3R,5R)-7-[2-(4-Fluoro-phenyl)-5-propyl-4-benzylcarbamoyl-imidazol-1-yl]-3,5-dihydroxy-heptanoic acid;
(3R,5R)-7-[2-(4-Fluoro-phenyl)-5-propyl-4-phenethylcarbamoyl-imidazol-1-yl]-3,5-dihydoxy-heptanoic acid;
(3R,5R)-7-[2-(4-Fluoro-phenyl)-5-propyl-4-(4-fluorophenylcarbamoyl)-imidazol-l -yl]-3,5-dihydroxy-heptanoic acid;
(3R,5R)-7-[2-(4-Fluoro-phenyl)-5-methyl-4-phenylcarbamoyl-imidazol-1-yl]-3,5-dihydrxy-heptanoic acid;
(3R,5R)-7-[2-(4-Fluoro-phenyl)-5-methyl-4-benzylcarbamoyl-imidazol-1-yl]-3,5-dihydroxy-heptanoic acid;
(3R,5R)-7-[2-(4-Fluoro-phenyl)-5-methyl-4-phenethylcarbambyl-imidazol-1-yl]-3,5-dihdroxy-heptanoic acid;
(3R,5R)-7-[4-[(Biphenyl-3-ylmethyl)-carbamoyl]-2-(4-fluoro-phenyl)-5-isopropyl-imidazol-1-yl]-3,5-dihydroxy-heptanoic acid;
(3R,5R)-7-[2-(4-Fluoro-phenyl)-5-isopropyl-4-phenethylcarbamoyl-imidazol-1-yl]-3,5-dihydroxy-heptanoic acid;
(3R,5R)-7-[2-(4-Fluoro-phenyl)-5-methyl-4-(4-sulfamoyl-benzylcarbamoyl)-imidazol-1-yl]-3,5-dihydroxy-heptanoic acid;
(3R,5R)-7-[4-benzylcarbamoyl-2-phenyl-5-isopropyl-imidazol-1-yl]-3,5-dihydroxy-heptanoic acid; (3R,5R)-7-[4-(3-Chloro-benzylcarbamoyl)-2-(4-fluoro-phenyl)-5-isopropyl-imidazol-1yl]-3,5-dihydroxy-heptanoic acid; (3R,5R)-7-[2-(4-Fluoro-phenyl)-4-(indan-1-ylcarbamoyl)-5-isopropyl-imidazol-1-yl]-3,5-dihydroxy-heptanoic acid;
(3R,5R)-7-[4-Benzylcarbamoyl-5-cyclopropyl-2-(4-fluoro-phenyl)-imidazol-1-yl]-3,5-dihydroxy-heptanoic acid; (3R,5R)-7-[5-Cyclopropyl-2-(4-fluoro-phenyl)-4-(4-methoxy-benzylcarbamoyl)-imidazol-1-yl]-3,5-dihydroxy-heptanoic acid; and pharmaceutically acceptable salts, amides, esters and lactone forms thereof.

The present invention further provides a compound of the Formula I, as described above, selected from the group consisting of (3R,5R)-7-[4-Benzylcarbamoyl-2-(4-fluoro-phenyl)-5-isopropyl-imidazol-1-yl]-3,5-dihydroxy-heptanoic acid; pharmaceutically acceptable salts, amides, esters and lactone forms thereof.

The present invention further provides a combination of a compound of the Formula I as defined above, or a pharmaceutically acceptable salt, amide, ester or lactone thereof, and one or more additional pharmaceutically active agent.

The present invention further provides a pharmaceutical composition comprising a compound of Formula I as defined above or a combination as defined above, and a pharmaceutically acceptable carrier, diluent or vehicle.

Further, the present invention provides inter alia the following compounds: (3R, 5R)-7-[2-(4-Fluoro-phenyl)-5-isopropyl-4-(3-phenyl-pyrrolidine-1-carbonyl)-imidazol-1-yl]-3,5-dihydroxy-heptanbic acid; (3R,5R)-7-[4-(3-Benzenesulfonyl-pyrrolidine-1-carbonyl)-2-(4-fluoro-phenyl)-5-isopropyl-imidazol-1-yl]-3,5-dihydroxy-heptanoic acid; (3R,5R)-7-[2-(4-Fluoro-phenyl)-5-isopropyl-4-(4-sulfamoyl-benzylcarbamoyl)-imidazol-1-yl]-3,5-dihydroxy-heptanoic acid; and pharmaceutically acceptable salts, and lactone forms thereof.

Still further, the present invention provides inter alia the following compounds:
(3R,5R)-7-[5-cyclopropyl-4-{[(3-fluorobenzyl)amino]carbonyl}-2-(4-fluorophenyl)-1H-imidazol-1-yl]-3,5-dihydroxyheptanoic acid;
(3R,5R)-7-[5-cyclopropyl-4-{[(3,4-difluorobenzyl)amino]carbonyl}-2-(4-fluorophenyl)-1H-imidazol-1-yl]-3,5-dihydroxyheptanoic acid;
(3R,5R)-7-(5-cyclopropyl-2-(4-fluorophenyl)-4-{[(3-methoxybenzyl)amino]carbonyl}-1H-imidazol-1-yl)-3,5-dihydroxyheptanoic acid;
(3R,5R)-7-[5-cyclopropyl-4-H[(3,4-dimethoxybenzyl)amino]carbonyl}-2-(4-fluorophenyl)-1H-imidazol-1-yl]-3,5-dihydroxyheptanoic acid;
(3R,5R)-7-[5-cyclopropyl-4-{[(3-ethoxybenzyl)amino]carbonyl}-2-(4-fluorophenyl)-1H-imidazol-1-yl]-3,5-dihydroxyheptanoic acid;
(3R,5R)-7-(5-cyclopropyl-2-(4-fluorophenyl)-4-{[(2-methoxybenzyl)amino]carbonyl}-1H-imidazol-1-yl)-3,5-dihydroxyheptanoic acid;
(3R,5R)-7-(5-cyclopropyl-2-(4-fluorophenyl)-4-{[(2-methylbenzyl)amino]carbonyl}-1H-imidazol-1-yl)-3,5-dihydroxyheptanoic acid;
(3R,5R)-7-(5-cyclopropyl-2-(4-fluorophenyl)-4-{[(3-methylbenzyl)amino]carbonyl}-1H-imidazol-1-yl)-3,5-dihydroxyheptanoic acid;
(3R,5R)-7-(5-cyclopropyl-2-(4-fluorophenyl)-4-{[(4-methylbenzyl)amino]carbonyl}-1H-imidazol-1-yl]-3,5-dihydroxyheptanoic acid;
(3R,5R)-7-[4-{[(4-cyanobenzyl)amino]carbonyl}-5-cyclopropyl-2-(4-fluorophenyl)-1H-imidazol-1-yl]-3,5-dihydroxyheptanoic acid;
(3R,5R)-7-[4-{[(4-chlorobenzyl)amino]carbonyl}-5-cyclopropyl-2-(4-fluorophenyl)-1H-imidazol-1-yl]-3,5-dihydroxyheptanoic acid;
(3R,5R)-7-[4-{[(3-cyanobenzyl)amino]carbonyl}-5-cyclopropyl-2-(4-fluorophenyl)-1H-imidazol-1-yl]-3,5-dihydroxyheptanoic acid;
(3R,5R)-7-[5-cyclopropyl-4-[({4-[(dimethylamino)carbonyl]benzyl}amino) carbonyl]-2-(4-fluorophenyl)-1H-imidazol-1-yl]-3,5-dihydroxyheptanoic acid;
(3R,5R)-7-[5-cyclopropyl-4-{[(3-fluorobenzyl)(methyl)amino]carbonyl}-2-(4-fluorophenyl)-1H-imidazol-1-yl]-3,5-dihydroxyheptanoic acid;
(3R,5R)-7-[5-cyclopropyl-4-{[(3,4-difluorobenzyl)(methyl)amino]carbonyl}-2-(4-fluorophenyl)-1H-imidazol-1-yl]-3,5-dihydroxyheptanoic acid;
(3R,5R)-7-[5-cyclopropyl-2-(4-fluorophenyl)-4-({methyl[(1R)-1-phenylethyl]amino)carbonyl)-1H-imidazol-1-yl]-3,5-dihydroxyheptanoic acid;
(3R,5R)-7-[4-{[(cyclohexylmethyl)amino]carbonyl)-5-cyclopropyl-2-(4-fluorophenyl)-1H-imidazol-1-yl]-3,5-dihydroxyheptanoic acid;
(3R,5R)-7-[5-cyclopropyl-2-(4-fluorophenyl)-4-({[2-(4-methoxyphenyl)ethyl]amino}carbonyl)-1H-imidazol-1-yl]-3,5-dihydroxyheptanoic acid;
(3R,5R)-7-[5-cyclopropyl-2-(4-fluorophenyl)-4-({[2-(3-fluorophenyl)ethyl]amino}carbonyl)-1H-imidazol-1-yl]-3,5-dihydroxyheptanoic acid;
(3R,5R)-7-(5-cyclopropyl-2-(4-fluorophenyl)-4-[(2-naphthylmethyl)amino]carbonyl}-1H-imidazol-1-yl)-3,5-dihydroxyheptanoic acid
(3R,5R)-7-[5-cyclopropyl-2-(4-fluorophenyl)-4-({[(6-phenylpyridin-3-yl)methyl]amino}carbonyl)-1H-imidazol-1-yl]-3,5-dihydroxyheptanoic acid;
(3R,5R)-7-[4-[(benzylamino)carbonyl]-2-(4-chlorophenyl)-5-cyclopropyl-1H-imidazol-1-yl]-3,5-dihydroxyheptanoic acid;
(3R,5R)-7-[4-[(benzylamino)carbonyl]-5-cyclopropyl-2-(6-methylpyridin-3-yl)-1H-imidazol-1-yl]-3,5-dihydroxyheptanoic acid;
and pharmaceutically acceptable salts and lactone forms thereof.

The present invention further encompasses each of the title compounds set forth in the Examples herein.

The present invention provides a compound having a Formula I, 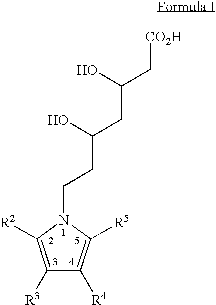
or a pharmaceutically acceptable salt, ester, amide, stereoisomer or prodrug thereof, or a pharmaceutically acceptable salt of the prodrug, wherein R² is benzyl, naphthyl or cyclohexyl, optionally substituted; phenyl or phenyl substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; one of R³ and R⁴ is H; aryl, aralkyl, heteroaryl, heteroaralkyl, optionally substituted; C₁-C₈ alkyl straight chain or branched; or C₃-C₈ cycloalkyl; and the other one of R³ and R⁴ is H, I, COOR′, R⁶R⁷NC(O)— or SO₂NR⁹R¹⁰; one of R⁶ and R⁷ is SO₂NHR⁸ or SO₂R⁸; and the other one of R⁶ and R⁷ is H or C₁-C₄ alkyl; R⁸ is aryl or heteroaryl, optionally substituted; R⁹ and R¹⁰ are each independently H; aryl, aralkyl, heteroaryl or heteroaralkyl optionally substituted with halogen, OR′, (CH₂)ₙCOOR′, (CH₂)ₙCONR′R″, (CH₂)ₙSO₂NR′R″, (CH₂)ₙSO₂R′ or CN; C₁-C₁₀ alkyl unsubstituted or substituted with OH, CO₂R′ or CONR′R″; or N, R⁹ and R¹⁰ taken together form a 4-11 member ring optionally containing up to 2 heteroatoms selected from O, N and S, said ring optionally substituted with ═O, OH, benzyl, phenyl, CO₂R′, R′OR″, (CH₂)ₙSO₂R′ or CONR′R″; R⁵ is alkyl of from one to four carbon atoms, optionally substituted with a halogen;
R′ and R″ are each independently H, lower alkyl or taken together form a 4-7 member ring;
and n is 0-2.

Further provided is the above-described compound wherein R² is phenyl or substituted phenyl. Further provided is the compound wherein R² is phenyl substituted with a halogen. Further provided is the compound wherein R² is para-fluorophenyl.

Further provided is the above-described compound wherein R³ is indolyl, phenyl, biphenyl or substituted phenyl, pyridyl or substituted pyridyl, lower alkyl, or naphthyl.

Further provided is the above compound wherein R³is cyclohexyl-, clyclopentyl-, cyclobutyl-, cyclopropyl-, methyl-, ethyl-, isopropyl-, difluoromethyl, trifluoro-methyl or phenyl substituted with one or more halogen.

Further provided is the compound wherein R³ is para-fluorophenyl, 3,4-difluorophenyl, para-cyanophenyl or para-methylphenyl.

Further provided is the above described compound wherein R⁵ is C₁-C₄ alkyl. Further provided is the compound wherein R⁵ is C₁-₃ alkyl.

Further provided is the above compound wherein R⁴is SO₂NR⁹R¹⁰.

Further provided is the above compound wherein R⁹ and R¹⁰ are each independently H, methyl, phenyl or phenyl substituted with OH, F, CO₂R′, CONR′R″, SO₂NR′R″ or one or more halogen; or benzyl or benzyl substituted with OH, CO₂R or CONR′R″.

Further provided is the above compound wherein R⁵ is isopropyl, ethyl, trifluoromethyl or difluoromethyl.

Further provided is the compound wherein R⁵ is isopropyl and R² is parafluorophenyl.

Further provided is a pharmaceutically acceptable salt of the above compound wherein the salt is a sodium salt or a calcium salt.

Further provided is a sterioisomer of the above compound comprising a (3R, 5R)-isomer.

Further provided is a sterioisomer of the above compound comprising a (3S, 5R)-isomer.

Further provided is a sterioisomer of the above compound comprising a (3R, 5S)-isomer.

Further provided is a sterioisomer of the above compound comprising a (3S, 5S)-isomer.

Further provided is a pharmaceutically acceptable ester of the above compound wherein the ester is a methyl ester.

Further provided is the above-described compound wherein R⁵ is isopropyl.

Further provided is the above compound wherein R² and R³ are each independently phenyl or substituted phenyl and R⁵ is C₁-C₄ alkyl.

Further provided is the compound wherein R⁵ is C₁-C₄ alkyl and R⁴ is SO₂NR⁹R¹⁰. Further provided is the compound wherein R⁵ is C₁-C₄ alkyl, R⁴ is SO₂NR⁹R¹⁰ and R⁹ and R¹⁰ are each independently H, Me, phenyl substituted with OH, F, CO₂R, SO₂NR′R″ or CONR′R″, benzyl or benzyl substituted with OH, F, CO₂R′ or CONR.

Further provided is the above compound wherein R⁸ is phenyl or substituted phenyl.

Further provided is the above compound wherein N, R⁹ and R¹⁰ taken together form a 4-7 member ring, optionally containing up to 2 heteroatoms selected form O, N, and S, said ring optionally substituted with OH, benzyl, phenyl, CO₂R′ or CONR′R″; and R′ and R″ are each independently H, lower alkyl or taken together form a 4-7 member ring.

Further provided are pharmaceutical compositions of compounds of the present invention.

Further, the present invention provides a process for making a compound having a Formula 10 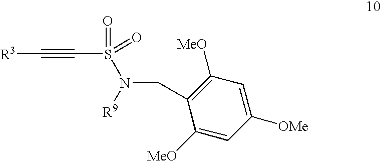
wherein R⁹ is aryl, aralkyl, heteroaryl or heteroaralkyl; optionally substituted; C₁-C₁₀ alkyl unsubstituted or substituted with OH, CO₂R′ or CONR′R″; and
R³ is aryl, aralkyl, heteroaryl, heteroaralkyl, optionally substituted; C₁-C₈ alkyl straight chain or branched; or C₃-C₈ cycloalkyl;
comprising the following steps: 1.) reacting a compound having a Formula 1 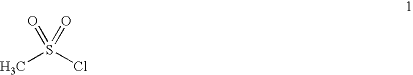
   with R⁹-substituted 2,4,6-trimethoxy benzylaniline, wherein R⁹ is as defined above, to form a compound of Formula 8 wherein Me is methyl and R⁹ is as defined above, 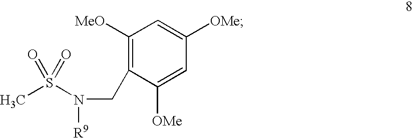
   2.) reacting the compound of Formula 8 with a compound having a Formula R³COOMe wherein R³ and Me are as defined above, is in n-BuLi, to form a compound of Formula 9

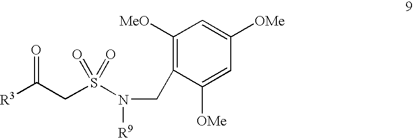

wherein Me is methyl and R³ and R⁹ are as defined above; and

3.) contacting the compound 9 with 2-chloro N-methylpyridinium iodide and triethylamine to form the compound 10.

The present invention further provides a compound having a Formula 15 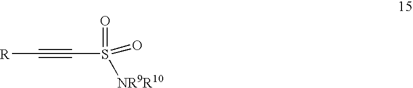
wherein R is C₁-C₈ alkyl straight chain or branched or C₃-C₈ cycloalkyl;
R⁹ and R¹⁰ are each independently H, aryl, aralkyl, heteroaryl or heteroaralkyl; optionally substituted; C₁-C₁₀alkyl unsubstituted or substituted with OH, CO₂R′ or CONR′R″; or N, R⁹ and R¹⁰ taken together form a 4-7 member ring, optionally containing up to 2 heteroatoms selected from O, N and S, said ring optionally substituted with OH, benzyl, phenyl, CO₂R′ or CONR′R″; and R and R are each independently H, lower alkyl or taken together form a 4-7 member ring. 45.

Further provided is a process for making a compound having a Formula 15 wherein R, R⁹ and R¹⁰ are as defined above 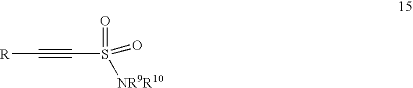
comprising the following steps: 1.) reacting a compound of Formula 1 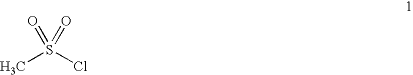
with NR⁹R¹⁰ wherein R⁹ and R¹⁰ are as defined above, to form a compound of Formula 13, wherein R⁹ and R¹⁰ are as defined above, 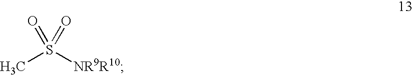
2.) reacting the compound 13 with RCOOMe wherein R is as defined above and Me is methyl, in n-BuLi, to form a corresponding, β-ketosulfonamide; 3.) reacting said corresponding β-ketosulfonamide with a Hunig's base to form the compound 15.

Further provided is a process for making a compound having a Formula cc 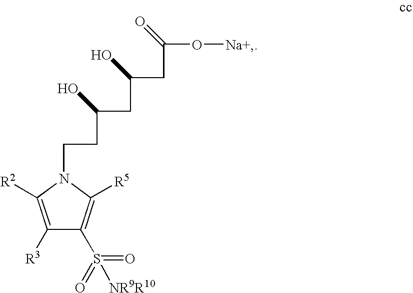
wherein R², R³, R⁵, R⁹ and R¹⁰ are as defined above, comprising the following steps:1.) reacting a compound of Formula a, wherein R³, R⁹ and R¹⁰ are as defined above, in a solvent,

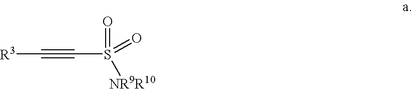

with a compound of Formula 5, wherein R² and R⁵ are as defined above 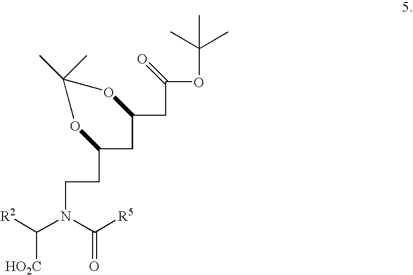
to form a compound of Formula b wherein R², R³, R⁵, R⁹ and R¹⁰ are as defined above; 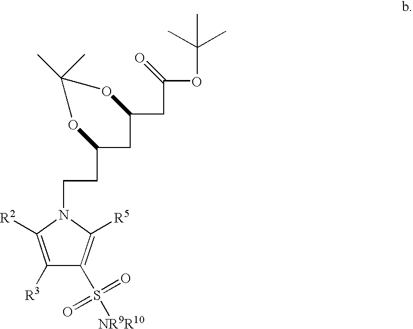
2.) forming a lactone corresponding to the compound b; and 3) hydrolyzing the lactone to form the compound cc.

Further provided is a process for making a compound having a Formula 12a 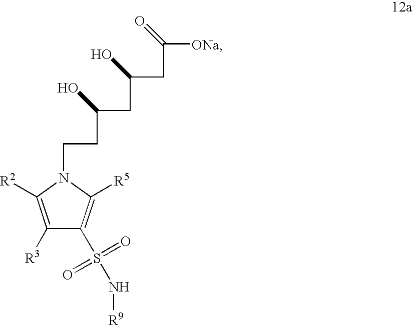
wherein R², R³ and R⁵ are as defined in claim 1 and R⁹ is aryl, aralkyl, heteroaryl or heteroaralkyl; optionally substituted; or C₁₋₁₀ alkyl, optionally sukbstituted, comprising the following steps: 1.) reacting a compound of Formula 5 wherein R² and R⁵ are as defined above, 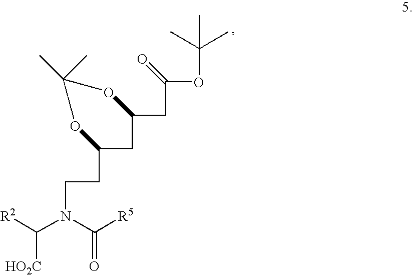
with a compound of Formula 10 wherein Me is methyl, R³ and R⁹ are as defined above, 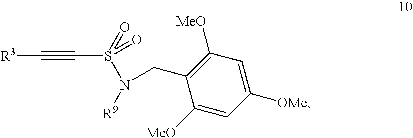
to form a compound of Formula 11 comprising a 2, 4, 6 trimethoxybenzyl protecting group, wherein R², R³, R⁵ and R⁹ are as defined above, 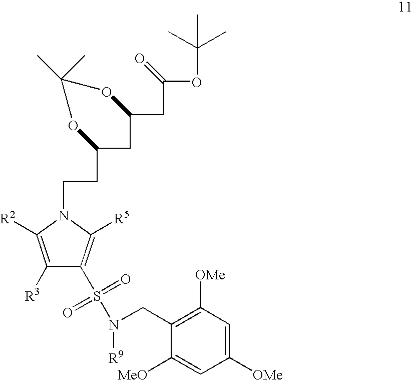
2.) forming a lactone corresponding to the compound 11; 3) removing the protecting group; and 4.) hydrolyzing the lactone to produce the compound 12.

The present invention also provides a compound having a formula C,

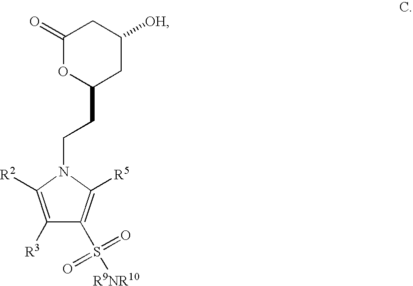

wherein R²is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; R³ is H; aryl, aralkyl, heteroaryl, heteroaralkyl, optionally substituted; C₁-C₈ alkyl straight chain or branched; or C₃-C₈ cycloalkyl;
R⁵ is alkyl of from one to four carbon atoms, optionally substituted with a halogen; and R⁹ and R¹⁰ are each independently H; aryl, aralkyl, heteroaryl or heteroaralkyl optionally substituted with halogen, OR′, (CH₂)ₙCOOR′, (CH₂)ₙCONR′R″, (CH₂)ₙSO₂NR′R″, (CH₂)ₙSO₂R′ or CN; C₁-C₁₀ alkyl unsubstituted or substituted with OH, CO₂R′ or CONR′R″;
or N, R⁹ and R¹⁰ taken together form a 4-7 member ring optionally containing up to 2 heteroatoms selected from O, N and S, said ring optionally substituted with OH, benzyl, phenyl, CO₂R′ or CONR′R″;
R⁵ is alkyl of from one to four carbon atoms, optionally substituted with a halogen; R′ and R″ are each independently H, lower alkyl or taken together form a 4-7 member ring; and n is 0-2.

The present invention further provides a process for making a compound having a formula C

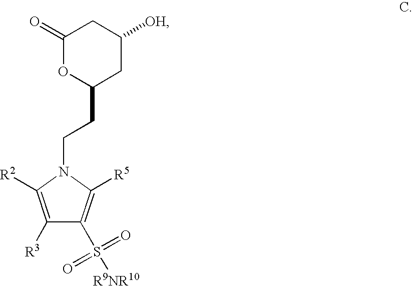

wherein R², R³, R⁵, R⁹ and R¹⁰ are as defined above, comprising the following steps:
1). Reacting a compound A,

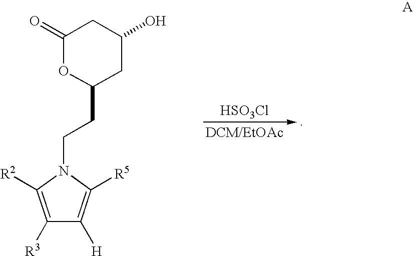

wherein R²; R³ and R⁵ are as defined above with HSO₃Cl in DCM/EtOA_{c} to form a compound B,

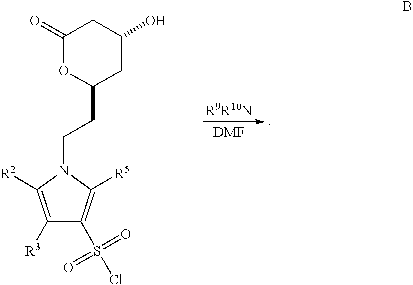

wherein R², R³ and R⁵ are as defined above; and 2.) reacting the compound B with R⁹R¹⁰N wherein R⁹ and R¹⁰ are as defined above in DMF to form the compound C.

The present invention also provides a compound having a Formula, 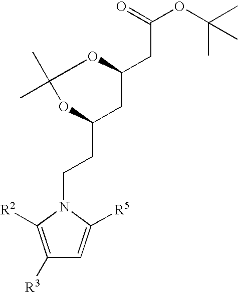
or a pharmaceutically acceptable salt, ester, amide, stereoisomer or prodrug thereof or a pharmaceutically acceptable salt of the prodrug

wherein R² is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; R³ is H; aryl, aralkyl, heteroaryl, heteroaralkyl, optionally substituted; C₁-C₈ alkyl straight chain or branched; or C₃-C₈ cycloalkyl; and R⁵ is alkyl of from one to four carbon atoms, optionally substituted with a halogen.

The present invention also provides a compound having a formula 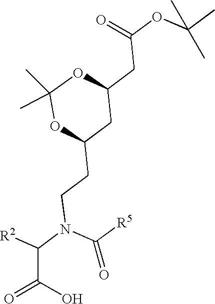
wherein R² is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; and R⁵ is alkyl of from one to four carbon atoms, optionally substituted with a halogen. Also provided is the above compound wherein R⁵ is isopropyl. Also provided is the above compound wherein R⁵ is isopropyl and R² is para-fluorophenyl.

Also provided is a racemic mixture comprising a compound of Formula I.

The term “alkyl” as used herein refers to a straight or branched hydrocarbon of from 1 to 11 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower alkoxy, lower thioalkoxy, —O(CH₂)₀₋₂CF₃, —Oaryl, halogen, nitro, cyano, ═O, ═S, —OH, —SH, —CF₃, —CO₂H, —CO₂C₁-C₆ alkyl, —NR′R″, NR′SO₂R″, NR′CONR′R″, or —CONR′R″ where R′ and R″ are independently H, alkyl, cycloalkyl, akenyl, alkynyl, aryl, aralkyl, heteroaryl, heteroaralkyl, or joined together to form a 4 to 7 member ring; or N, R′ and R″ taken together form a 4-7 member ring. Useful alkyl groups have from 1 to 6 carbon atoms (C₁-C₆ alkyl).

The term “lower alkyl” as used herein refers to a subset of alkyl which means a straight or branched hydrocarbon radical having from 1 to 6 carbon atoms and includes, for example, methyl, ethyl, n-propyl, isopropyl, n-butyl, sec-butyl, isobutyl, tert-butyl, n-pentyl, n-hexyl, and the like. Optionally, lower alkyl is referred to as “C₁-C₆alkyl.”

The term “haloalkyl” as used herein refers to a lower alkyl radical, as defined above, bearing at least one halogen substituent, for example, chloromethyl, fluoroethyl, trifluoromethyl, or 1,1,1-trifluoroethyl and the like. Haloalkyl can also include perfluoroalkyl wherein all hydrogens of a lower alkyl group are replaced with fluorine atoms.

The term “alkenyl” means a straight or branched unsaturated hydrocarbon radical from 2 to 12 carbon atoms and includes, for example, ethenyl, 1-propenyl, 2-propenyl, 1-butenyl, 2-butenyl, 1-pentenyl, 2-pentenyl, 3-methyl-3-butenyl, 1-hexenyl, 2-hexenyl, 3-hexenyl, 3-heptenyl, 1-octenyl, 1-nonenyl, 1-decenyl, 1-undecenyl, 1-dodecenyl, and the like.

The term “alkynyl” means a straight or branched hydrocarbon radical of 2 to 12 carbon atoms having at least one triple bond and includes, for example, 3-propynyl, 1-butynyl, 3-butynyl, 1-pentynyl, 3-pentynyl, 3-methyl-3-butynyl, 1-hexynyl, 3-hexynyl, 3-hexynyl, 3heptynyl, 1-octynyl, 1-nonynyl, 1-decynyl, 1-undecynyl, 1-dodecynyl, and the like.

The term “alkylene” as used herein refers to a divalent group derived from a straight or branched chain saturated hydrocarbon having from 1 to 10 carbon atoms by the removal of two hydrogen atoms, for example methylene, 1,2-ethylene, 1,1-ethylene, 1,3-propylene, 2,2-dimethylpropylene, and the like. The alkylene groups of this invention can be optionally substituted with one or more of the substituents selected from lower alkyl, lower alkoxy, lower thioalkoxy, —O (CH₂)₀₋₂CF₃, halogen, nitro, cyano, ═O, ═S, —OH, —SH, —CF₃, —CO₂H, —CO₂C₁-C₆ alkyl, NR′R″, or —CONR′R″, where R′ and R″ are independently H, alkyl, cycloalkyl, akenyl, alkynyl, aryl, aralkyl, heteroaryl, heteroaralkyl, or joined together to form a 4 to 7 member ring; or N, R′ and R″ taken together form a 4-7 member ring. Useful alkylene groups have from 1 to 6 carbon atoms (C₁-C₆ alkylene).

The term “heteroatom” as used herein represents oxygen, nitrogen, or sulfur (O, N, or S) as well as sulfoxyl or sulfonyl (SO or SO₂) unless otherwise indicated.

The term “hydrocarbon chain” as used herein refers to a straight hydrocarbon of from 2 to 6 carbon atoms. The hydrocarbon chain is optionally substituted with one or more substituents selected from lower alkyl, lower alkoxy, lower thioalkoxy, —O (CH₂)₀₋₂CF₃, halogen, nitro, cyano, ═O, ═S, —OH, —SH, —CF₃, —CO₂H, —CO₂C₁-C₆ alkyl, NR′R″ or —CONR′R″, where R′ and R″ are independently H, alkyl, cycloalkyl, akenyl, alkynyl, aryl, aralkyl, heteroaryl, heteroaralkyl or joined together to form a 4 to 7 member ring; or N, R′ and R″ taken together form a 4-7 member ring.

The term “hydrocarbon-heteroatom chain” as used herein refers to a hydrocarbon chain wherein one or more carbon atoms are replaced with a heteroatom. The hydrocarbon-heteroatom chain is optionally substituted with one or more substituents selected from lower alkyl, lower alkoxy, lower thioalkoxy, —O (CH₂)₀₋₂CF₃, halogen, nitro, cyano, ═O, ═S, —OH, —SH, —CF₃, —CO₂H, —CO₂C₁-C₆ alkyl, NR′R″ or —CONR′R″, where R′ and R″ are independently H, alkyl, cycloalkyl, akenyl, alkynyl, aryl, aralkyl, heteroaryl, heteroaralkyl or joined together to form a 4 to 7 member ring; or N, R′ and R″ taken together form a 4-7 member ring.

The term “heteroalkylene” as used herein, refers to an alkylene radical as defined above that includes one or more heteroatoms such as oxygen, sulfur, or nitrogen (with valence completed by hydrogen or oxygen) in the carbon chain or terminating the carbon chain.

The terms “lower alkoxy” and “lower thioalkoxy” as used herein refers to O-alkyl or S-alkyl of from 1 to 6 carbon atoms as defined above for “lower alkyl.”

The term “aryl” as used herein refers to an aromatic ring which is unsubstituted or optionally substituted by 1 to 4 substituents selected from lower alkyl, lower alkoxy, lower thioalkoxy, —O(CH₂)₀₋₂CF₃, —Oaryl, —OSO₂R′, nitro, cyano —OH, —SH, —CF₃, —CO₂H, —CO₂C₁-C₆ alkyl, —NR′R″, NR′SO₂R″, NR′CONR′R″, —SO₁₋₂alkyl, SO₁₋₂aryl, SO₂NR′R″, or —CONR′R″, where R′ and R″ are independently H, alkyl, cycloalkyl, akenyl, alkynyl, aryl, aralkyl, heteroaryl, heteroaralkyl or joined together to form a 4 to 7 member ring; or N, R′ and R″ taken together form a 4-7 member ring. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, or the like. Further, the term “aryl” means a cyclic or polycyclic aromatic ring having from 5 to 12 carbon atoms, and being unsubstituted or substituted with up to 4 of the substituent groups recited above for alkyl, alkenyl, and alkynyl.

The term aralkyl as used herein means aryl, as defined above, attached to an alkyl group, as defined above.

The term “heteroaryl” means an aromatic ring containing one or more heteroatom. The heteroaryl is optionally substituted with one or more groups enumerated for aryl. Examples of heteroaryl include, but are not limited to thienyl, furanyl, pyrrolyl, pyridyl, pyrimidyl, imidazoyl, pyrazinyl, oxazolyl, thiazolyl, benzothienyl, benzofuranyl, indolyl, quinolinyl, isoquinolinyl, and quinazolinyl, and the like. Further, the term “heteroaryl” means an aromatic mono-, bi-, or polycyclic ring incorporating one or more (i.e. 1-4) heteroatoms selected from N, O, and S, which mono-, bi-, or polycyclic ring is optionally substituted with lower alkyl, lower alkoxy, lower thioalkoxy, —O(CH₂)₀₋₂CF₃, halogen, nitro, cyano —OH, —SH, —CF₃, —CO₂H, —CO₂C₁-C₆ alkyl, —NR′R″, —SO₂alkyl, SO₂aryl, SO₂NR′R″, or —CONR′R″, where R′ and R″ are independently H, alkyl, cycloalkyl, akenyl, alkynyl, aryl, aralkyl, heteroaryl, heteroaralkyl or joined together to form a 4 to 7 member ring; or N, R′ and R″ taken together form a 4-7 member ring. Examples further include I-, 2-, 4-, or 5-imidazolyl, I-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5- isoxazolyl, 1,3-, or 5-triazolyl, I-, 2-, or 3-tetrazolyl, 2-pyrazinyl, 2-, 4-, or 5-pyrimidin piperazinyl, 2-, 3-, or 4-morpholinyl. Examples of suitable bicyclic heteroaryl compounds include, but are not limited to indolizinyl, isoindolyl, benzofuranyl, benzothienyl, benzoxazolyl, benzimidazolyl, quinolinyl, isoquinolinyl, quinazolinyl, I-, 2-, 3-, 4-, 5-, 6-, or 7-indolyl, I-, 2-, 3-, 5-, 6-, 7-, or 8-indolizinyl, I-, 2-, 3-, 4-, 5-, 6-, or 7-isoindolyl, 2-, 3-, 4-, 5-, 6-, or 7-benzothienyl, 2-, 4-, 5-, 6-, or 7-benzoxazolyl, I-, 2-, 4-, 5-, 6-, or 7-benzimidazolyl, 2-, 3-, 4-, 5-, 6-, 7-, or 8-quinolinyl, and I-, 3-, 4-, 5-, 6-, 7-, or 8-isoquinolinyl.

The term heteroaralkyl, as used herein, means heteroaryl, as defined above, attached to an alkyl group as defined above.

The term “heterocycle” means a saturated mono- or polycyclic (i.e. bicyclic) ring incorporating one or more (i.e. 1-4) heteroatoms selected from N, O, and S. It is understood that a heterocycle is optionally substituted with one or more of the substituents selected from lower alkoxy, lower thioalkoxy, —O(CH₂)₀₋₂CF₃, halogen, nitro, cyano, ═O, ═S, —OH, —SH, —CF₃, —CO₂H, —CO₂C₁-C₆ alkyl, —NR′R″ or —CONR′R″ where R′ and R″ are independently H, alkyl, cycloalkyl, akenyl, alkynyl, aryl, aralkyl, heteroaryl, heteroaralkyl, or joined together to form a 4 to 7 member ring; or N, R′ and R″ taken together form a 4-7 member ring. Useful alkyl groups have from 1 to 6 carbon atoms (C₁-C₆ alkyl). Examples of suitable monocyclic heterocycles include, but are not limited to piperidinyl, pyrrolidinyl, piperazinyl, azetidinyl, aziridinyl, morpholinyl, thietanyl, oxetaryl.

The term “ring” as used herein includes heteroaryl, cycloalkyl or aryl and further includes fused, monocyclic and polycyclic permutations thereof.

The term “cycloalkyl” means a saturated hydrocarbon ring. Further, the term “cycloalkyl” means a hydrocarbon ring containing from 3 to 12 carbon atoms, for example, cyclopropyl, cyclobutyl, cyclopentyl, cyclohexyl, cycloheptyl, cycloctyl, decalinyl, norpinanyl, or adamantyl. The cycloalkyl ring may be unsubstituted or substituted by 1 to 3 substituents selected from one or more of the substituents selected from lower alkoxy, lower thioalkoxy,

—(CH₂)₀₋₂CF₃, halogen, nitro, cyano, ═O, ═S, —OH, —SH, —CF₃, —CO₂H, —CO₂C₁-C₆ alkyl, —NR′R″ or —CONR′R″ where R′ and R″ are independently H, alkyl, cycloalkyl, akenyl, alkynyl, aryl, aralkyl, heteroaryl, heteroaralkyl, or joined together to form a 4 to 7 member ring; or N, R′ and R″ taken together form a 4-7 member ring. Useful alkyl groups have from 1 to 6 carbon atoms (C₁-C₆ alkyl),

wherein alkyl, aryl, and heteroaryl are as defined herein. Examples of substituted cycloalkyl groups include fluorocyclopropyl, 2-iodocyclobutyl, 2,3-dimethylcyclopentyl, 2,2-dimethoxycyclohexyl, and 3-phenylcyclopentyl.

The term “cycloalkenyl” means a cycloalkyl group having one or more carbon-carbon double bond. Example includes cyclobutene, cyclopentene, cyclohexene, cycloheptene, cyclobutadiene, cyclopentadiene, and the like.

The term “isomer” means “stereoisomer” and “geometric isomer” as defined below.

The term “stereoisomer” means compounds that possess one or more chiral centers and each center may exist in the R or S configuration. Stereoisomers includes all diastereomeric, enantiomeric and epimeric forms as well as racemates and mixtures thereof.

The term “geometric isomer” means compounds that may exist in cis, trans syn, anti, entgegen (E), and zusammen (Z) forms as well as mixtures thereof.

The symbol “=” means a double bond.

The symbol “n” means a bond to a group wherein a 4 to 8 membered ring is formed. Typically this symbol will appear in pairs.

When a bond to a substituent is shown to cross the bond connecting 2 atoms in a ring, then such substituent may be bonded to any atom in the ring, provided the atom will accept the substituent without violating its valency. When there appears to be several atoms of the substituent that may bond to the ring atom, then it is the first atom of the listed substituent that is attached to the ring.

When a bond from a substituent is shown to cross the bond connecting 2 atoms in a ring of the substituent, then such substituent may be bonded from any atom in the ring which is available.

When a bond is represented by a line such as “---” this is meant to represent that the bond may be absent or present provided that the resultant compound is stable and of satisfactory valency. If an asymmetric carbon is created by such a bond, a particular stereochemistry is not to be implied.

As used herein, the following terms have the meanings given: RT or rt means room temperature. MP means melting point. MS means mass spectroscopy. TLC means thin layer chromatography. [S]at. means saturated. [C]onc. means concentrated. TBIA means [(4R,6R)-6-(2-Amino-ethyl)-2,2-dimethyl-[1,3]dioxan-4-yl]-acetic acid tert-butyl ester. DCM means dichloromethane, which is used interchangeably with methylene chloride. NBS means N-Bromosuccinimide. “h” means hour. “v/v” means volume ratio or “volume per volume”. “Rₜ38 means retention factor. “Tf₂O” or “TfO”means triflic anhydride or C (F)₃S(O)₂O S(O)₂C(F)₃. Ac₂O means acetic anhydride. “[T]rifluorotol.” Or “TFT” means trifluoro methyl-benzene. “DMF” means dimethylformamide. “DCE” means dichloroethane. “Bu” means butyl. “Me” means methyl. “Ef” means ethyl. “DBU” means 1,8-Diazabicyclo-[5.4.0]undec-7-ene. “TBS” means “TBDMS” or tert-Butyidimethylsilyl. “DMSO” means dimethyl sulfoxide. “TBAF” means tetrabutylammonium fluoride. THF means tetrahydrofuran. n-BuLi or Buli means n-butyl lithium. TFA means trifluoroacetic acid. i-Pr means isopropyl. [M]in means minutes. ml or mL means milliliter. “M” or “m” means molar. “Bn” means benzyl. “PyBOP” means bromo-tris-pyrrolidino-phosphonium hexafluorophosphate. “OtBu” means t-butoxy. “Ts” or “Tosyl” means p-toluenesulfonyl. “PS-DIEA” means polystyrene-bound diisopropylethylamine. “PS-NCO” means polystyrene-bound isocyanate resin. “Ph” means phenyl. As used herein, “hydrogenolysis” means the cleaving of a chemical bond by hydrogen. “EDCI” or “EDC” means 1-(3-dimethylaminopropyl)-3-ethylcarbondiimide hydrochloride. “NMP” means 1-methyl-2-pyrrolidinone. “DPP” or “DPPA” means diphenyl phosphoryl azide. “HOBt” 1-hydroxybenzptriazole.

The term “patient” means all mammals including humans. Examples of patients include humans, cows, dogs, cats, goats, sheep, pigs, and rabbits.

A “therapeutically effective amount” is an amount of a compound of the present invention that when administered to a patient ameliorates a symptom of hyperlipidemia, hypercholesterolemia, hypertriglyceridemia or atheroscelerois.

The terms pharmaceutically acceptable salt, ester, amide, lactone forms or prodrug as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “lactone form(s) thereof” means a six-membered ring lactone form of the compounds of the invention disclosed herein, as illustrated throughout the specification and claims. The term “a pharmaceutically acceptable salt” refers to the relatively non-toxic, inorganic and organic acid or base addition salts of compounds of the present invention. These salts can be prepared in situ during the final isolation and purification of the compounds or by separately reacting the purified compound in its free form with a suitable organic or inorganic acid or base and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977;66:1-19, which is incorporated herein by reference.) The free base form may be regenerated by contacting the salt form with a base. While the free base may differ from the salt form in terms of physical properties, such as solubility, the salts are equivalent to their respective free bases for the purposes of the present invention.

Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C6 dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

The use of prodrugs is contemplated by the present invention. “Prodrugs” are intended to include any covalently bonded carrier which releases the active parent drug according to Formula I in vivo. Further, the term “prodrug” refers to compounds that are transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include acetates, formates, benzoate derivatives of alcohols, and amines present in compounds of Formula I.

In some situations, compounds may exist as tautomers. All tautomers are included within Formula I and are provided by this invention.

Certain compounds of the present invention can exist in unsolvated form as well as solvated form including hydrated form. In general, the solvated form including hydrated form is equivalent to unsolvated form and is intended to be encompassed within the scope of the present invention.

Certain of the compounds of the present invention possess one or more chiral centers and each center may exist in the R or S configuration. The present invention includes all diastereomeric, enantiomeric, and epimeric forms as well as the appropriate mixtures thereof. Stereoisomers may be obtained, if desired, by methods known in the art as, for example, the separation of stereoisomers by chiral chromatographic columns and by chiral synthesis. Additionally, the compounds of the present invention may exist as geometric isomers. The present invention includes all cis, trans, syn, anti, entgegen (E), and zusammen (Z) isomers as well as the appropriate mixtures thereof.

The compounds of the present invention are suitable to be administered to a patient for the treatment, control, or prevention of, hypercholesteremia, hyperlipidemia, atherosclerosis and hypertriglyceridemia. The terms “Treatment”, “treating”, “controlling”, “preventing” and the like, refers to reversing, alleviating, or inhibiting the progress of the disease or condition to which such term applies, or one or more symptoms of such disease or condition. As used herein, these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of the compound of the invention to a subject that is not at the time of administration afflicted with the disease or condition. “Preventing” also encompasses preventing the recurrence of a disease or condition or of symptoms associated therewith. Accordingly, the compounds of the present invention can be administered to a patient alone or as part of a composition that contains other components such as excipients, diluents, and carriers, all of which are well-known in the art. The compositions can be administered to humans and animals either orally, rectally, parenterally (intravenously, intramuscularly, or subcutaneously), intracisternally, intravaginally, intraperitoneally, intravesically, locally (powders, ointments, or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol, or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

The compounds of the present invention can be administered to a patient at dosage levels in the range of about 0.1 to about 2,000 mg per day. For a normal human adult having a body weight of about 70 kilograms, a dosage in the range of about 0.01 to about 100 mg per kilogram of body weight per day is preferable. The specific dosage used, however, can vary. For example, the dosage can depend on a numbers of factors including the requirements of the patient, the severity of the condition being treated, and the pharmacological activity of the compound being used. The determination of optimum dosages for a particular patient is well-known to those skilled in the art.

### Combination Aspect of the Invention

The compounds of this invention may be used, either alone or in combination with the other pharmaceutical agents described herein, in the treatment of the following diseases/conditions: dyslipidemia, hypercholesterolemia, hypertriglyceridemia, atherosclerosis, peripheral vascular disease, cardiovascular disorders, angina, ischemia, cardiac ischemia, stroke, myocardial infarction, reperfusion injury, angioplastic restenosis, hypertension, diabetes and vascular complications of diabetes, obesity, unstable angina pectoris, Alzheimer's Disease, BPH, osteoporosis, cerebrovascular disease, coronary artery disease, ventricular dysfunction, cardiac arrhythmia, pulmonary vascular disease, renal-vascular disease, renal disease, vascular hemostatic disease, autoimmune disorders, pulmonary disease, anti-oxidant disease, sexual dysfunction, cognitive dysfunction, cancer, organ transplant rejection, psoriasis, endometriosis, and macular degeneration.

The compounds of this invention may also be used in conjunction with other pharmaceutical agents (e.g., HDL-cholesterol raising agents, triglyceride lowering agents) for the treatment of the disease/conditions described herein. A combination aspect of this invention includes a pharmaceutical composition comprising a compound of this invention or its pharmaceutically acceptable salt and at least one other compound. For example, the compounds of this invention may be used in combination with cholesterol absorption inhibitors, MTP/Apo B secretion inhibitors, or other cholesterol modulating agents such as fibrates, niacin, ion-exchange resins, antioxidants, ACAT inhibitors, PPAR-activators, CETP inhibitors or bile acid sequestrants. In combination therapy treatment, both the compounds of this invention and the other drug therapies are administered to mammals by conventional methods. The following discussion more specifically describes the various combination aspects of this invention.

Any cholesterol absorption inhibitor can be used in a combination aspect of this invention. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a recently approved cholesterol absorption inhibitor is ZETIA™.

Any cholesterol ester transfer protein (“CETP”) inhibitor may be used in a combination aspect of this invention. The effect of a CETP inhibitor on lipoprotein profile is believed to be anti-atherogenic. Such inhibition is readily determined by those skilled in the art by determining the amount of agent required to alter plasma lipid levels, for example HDL cholesterol levels, LDL cholesterol levels, VLDL cholesterol levels or triglycerides, in the plasma of certain mammals, (e.g., Crook et al. Arteriosclerosis 10, 625, 1990; U.S. Pat. No. 6,140,343). A variety of these compounds are described and referenced below, however other CETP inhibitors will be known to those skilled in the art. For example, U.S. Pat. Nos. 6,197,786, 6,723,752 and 6,723,753 (the disclosures of each of which is incorporated herein by reference) disclose cholesteryl ester transfer protein inhibitors, pharmaceutical compositions containing such inhibitors and the use of such inhibitors to elevate certain plasma lipid levels, including high density lipoprotein-cholesterol and to lower certain other plasma lipid levels, such as LDL-cholesterol and triglycerides and accordingly to treat diseases which are exacerbated by low levels of HDL cholesterol and/or high levels of LDL-cholesterol and triglycerides, such as atherosclerosis and cardiovascualar diseases in some mammals, including humans. Examples of useful CETP inhibitors include the following compounds: [2R, 4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as Torcetrapib™, and 3-{[3-(4-Chloro-3-ethyl-phenoxy)-phenyl]-[3-(1,1,2,2-tetrafluoro-ethoxy)-benzyl]-amino}-1,1,1-trifluoro-propan-2-ol. Many of the CETP inhibitors of this invention are poorly soluble and a dosage form that increases solubility facilitates the administration of such compounds. One such dosage form is a dosage form comprising (1) a solid amorphous dispersion comprising a cholesteryl ester transfer protein (CETP) inhibitor and an acidic concentration-enhancing polymer; and (2) an acid-sensitive HMG-CoA reductase inhibitor. This dosage form is more fully described in U.S. Ser. No. 10/739,567 and entitled “Dosage Forms Comprising a CETP Inhibitor and an HMG-CoA Reductase Inhibitor”, the specification of which is incorporated herein by reference.

Any compound that activates or otherwise interacts with a human peroxisome proliferator activated receptor (“PPAR”) may be used in a combination aspect of this invention. Three mammalian peroxisome proliferator-activated receptors have been isolated and termed PPAR-alpha, PPAR-gamma, and PPAR-beta (also known as NUC1 or PPAR-delta). PPAR-gamma receptors are associated with regulation of insulin sensitivity and blood glucose levels. PPAR-α activators are associated with lowering plasma triglycerides and LDL cholesterol. PPAR-β activators have been reported to both increase HDL-C levels and to decrease LDL-C levels. Thus, activation of PPAR-β alone, or in combination with the simultaneous activation of PPAR-α and/or PPAR-gamma may be desirable in formulating a treatment for dyslipidemia in which HDL is increased and LDL lowered. PPAR-activation is readily determined by those skilled in the art by the standard assays (e.g. US 2003/0225158 and US 2004/0157885). A variety of these compounds are described and referenced below, however other PPAR-activator compounds will be known to those skilled in the art. The following patents and published patent applications, the disclosure of each of which is incorporated herein by reference, provides a sampling. US 2003/0225158 discloses compounds that alter PPAR activity and methods of using them as therapeutic agents for treating or preventing dyslipidemia, hypercholesterolemia, obesity, hyperglycemia, atherosclerosis and hypertriglyceridemia. U.S. Pat. No. 6,710,063 discloses selective activators of PPAR delta. US 2003/0171377 discloses certain PPAR-activator compounds that are useful as anti-diabetic agents. US 2004/0157885 relates to PPAR agonists, in particular, certain PPARα agonists, pharmaceutical compositions containing such agonists and the use of such agonists to treat atherosclerosis, hypercholesterolemia, hypertriglyceridemia, diabetes, obesity, osteoporosis and Syndrome X or metabolic syndrome.

Examples of useful PPAR-activator compounds include the following compounds: [5-Methoxy-2-methly-4-(4′-trifluoromethly-biphenyl-4ylmethylsulfanyl)-phenoxy]-acetic acid; [5-Methoxy-2-methyl-4-(3′-trifloromethly-biphenyl-4-ylmethylsulfanyl)-phenoxy]-acetic acid; [4-(4′Fluoro-biphenyl-4-ylmethylsulfanyl)-5-methoxy-2methyl-phenoxy]-acetic acid; {5-Methoxy-2methyl-4-[4-(4-trifluoromethyl-benzyloxy)-benzylsulfanyl]-phenoxy-acetic acid; {{5-Methoxy-2-methyl-4-[4-(5-trifluoromethyl-pryidin-2-yl)-benzylsulfanyl]-phenoxy-acetic acid; (4-14-[2-(3-Fluoro-phenyl)-vinyl]-benzylsulfanyl]-5-methoxy-2-methyl-phenoxy)-acetic acid; [5-Methoxy-2-methyl-4-(3-methyl-4′-trifluoromethyl-biphenyl-4-ylmethylsulfanyl)-phenoxy]-acetic acid; [5-Methoxy-2-methyl-4-(4′-trifuoromethyl-biphenyl-3-ylmethylsulfanyl)-phenoxy]-acetic acid;
{5-Methoxy-2-methyl-4-[2-(4-trifluoromethyl-benzyloxy)-benzylsulfanyl]-phenoxyactic acid;
3-{5-[2-(-5-Methyl-2 phenyl-oxazol-4-yl-ethoxy]-indol-1-yl}-propionic acid; 3-{4[2-(5-methyl-2- phenyl-1,3-oxazol-4-yl)ethoxy-1H-indazol-1yl}propanoic acid; 2-Methyl-2-{3-[({2-(5methyl-2-phenyl-1,3-oxazol-4-yl)ethoxy]carbonyl}amino)methyl]phenoxy}propionic acid; 1-{3′-[2-5-Methyl-2-phenyl-1,3-oxazol-4-y]-1,1′-biphenyl-3-yl}oxy)cyclobutanecarboxylic acid;
3-[3-(1-Carboxy-1-methyl-ethoxy)-phenyl]-piperidine-1-carboxylic acid 3-trifluoromethyl-benzyl ester;
2-{2-methyl-4-[({4-methyl-2-[4-(trifluoromethyl)phenyl]-1,3-thiazol-5-yl}methyl)sulfanyl]phenoxy}acetic acid;
2-{2-methyl-4-[({4-methyl-2-[4-(trifluoromethyl )phenyl]-1,3-oxazol-5-yl}methyl)sulfanyl]phenoxy}acetic acid;
methyl 2-{4-[({4-methyl-2-[4-(trifluoromethyl)phenyl]-1,3-thiazol-5-yl}methyl)sulfanyl]phenoxy}acetate;
2-{4-[({4-methyl-2-[4-(trifluoromethyl)phenyl]-1,3-thiazol-5-yl}methyl)sulfanyl]phenoxy}acetic acid;
(E)-3-[2-methyl-4-({4-methyl-2-[4-(trifluoromethyl) phenyl]-1,3-th iazol-5-yl }methoxy)phenyl]-2-propenoic acid;
2-{3-chloro-4-[({4-methyl-2-[4-(trifluoromethyl)phenyl]-1,3-thiazol-5-yl}methyl)sulfanyl]phenyl}acetic acid;
2-{2-methyl-4-[({4-methyl-2-[3-fluoro-4-(trifluoromethyl)phenyl]-1,3-thiazo I-5-yl}methyl)sulfanyl]phenoxy}acetic acid; and pharmaceutically acceptable salts thereof.

Any MTP/Apo B secretion (microsomal triglyceride transfer protein and/or apolipoprotein B secretion) inhibitor can be used in the combination aspect of the present invention. Such inhibition is readily determined by those skilled in the art according to standard assays (e.g., Wetterau, J. R. 1992; Science 258:999). A variety of these compounds are known to those skilled in the art, including imputapride (Bayer) and additional compounds such as those disclosed in WO 96/40640 and WO 98/23593.

Any ACAT inhibitor can serve in the combination therapy aspect of the present invention. Such inhibition may be determined readily by one of skill in the art according to standard assays, such as the method of Heider et al. described in Journal of Lipid Research., 24:1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor can serve in the combination therapy aspect of the present invention. Such lipase inhibition activity is readily determined by those skilled in the art according to standard assays (e.g., Methods Enzymol. 286: 190-231). Pancreatic lipase mediates the metabolic cleavage of fatty acids from triglycerides at the 1- and 3-carbon positions. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231). Gastric lipase is an immunologically distinct lipase that is responsible for approximately 10 to 40% of the digestion of dietary fats. Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl)-bis-(iminoc-arbonyl)]dioxime, and the various bis(iminocarbonyl)dioximes related thereto may be prepared as described in Petersen et al., Liebig's Annalen, 562, 205-229 (1949). Lipstatin, (2S,3S,5S,7Z, 10Z)-5-[(S)-2-formamido-4-methyl-valeryloxy]-2-hexyl-3-hydro-xy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexa- decanoic 1,3 acid lactone, and the variously substituted N-formylleucine derivatives and stereoisomers thereof, are disclosed in U.S. Pat. No. 4,598,089. Tetrahydrolipstatin may be prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[-(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpipe- ridin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF2, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

Bile acid sequestrants, such as Welchol®, Colestid®, LoCholest®, Questran® and fibric acid derivatives, such as Atromid®, Lopid® and Tricor® may be used in a combination aspect of the invention.

Compounds of the present invention can be used with anti-diabetic compounds. Diabetes can be treated by administering to a patient having diabetes (especially Type II), insulin resistance, impaired glucose tolerance, or the like, or any of the diabetic complications such as neuropathy, nephropathy, retinopathy or cataracts, a therapeutically effective amount of a Formula I compound in combination with other agents (e.g., insulin) that can be used to treat diabetes. This includes the classes of anti-diabetic agents (and specific agents) described herein.

Any glycogen phosphorylase inhibitor can be used in combination with a Formula I compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385. Any aldose reductase inhibitor can be used in combination with a Formula I compound of the present invention. Aldose reductase inhibition is readily determined by those skilled in the art according to standard assays (e.g., J. Malone, Diabetes, 29:861-864 (1980). “Red Cell Sorbitol, an Indicator of Diabetic Control”). A variety of aldose reductase inhibitors are known to those skilled in the art.

Any sorbitol dehydrogenase inhibitor can be used in combination with a Formula I compound of the present invention. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a Formula I compound of the present invention. Such glucosidase inhibition activity is readily determined by those skilled in the art according to standard assays (e.g., Biochemistry (1969) 8: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethy-I)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydr-oxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihyd- roxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-.beta.-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxym-ethyl)piperidino]-.alpha.-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor Al-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, may be used in combination with a Formula I compound of the present invention, includes, for example, the following: biguanides (e.g., metformin), insulin secretagogues (e.g., sulfonylureas and glinides), glitazones, non-glitazone PPAR gamma agonists, PPAR.beta. agonists, inhibitors of DPP-IV, inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products). Other examples would include PKC-.beta. inhibitors and AGE breakers.

Compounds of the present invention can be used in combination with anti-obesity agents. Any anti-obesity agent can be used in such combinations and examples are provided herein. Such anti-obesity activity is readily determined by those skilled in the art according to standard assays known in the art. Suitable anti-obesity agents include phenylpropanolamine, ephedrine, pseudoephedrine, phentermine, beta sub.3 adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor antagonists (e.g., rimonabant (SR-141,716A)), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine.TM.), human agouti-related proteins (AGRP), ghrelin receptor antagonists, histamine 3 receptor antagonists or inverse agonists, neuromedin U receptor agonists, and the like.

Any thyromimetic can be used in combination with compounds of the present invention. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121; 4,826,876; 4,910,305; 5,061,798; 5,284,971; 5,401,772; 5,654,468; and 5,569,674. Other antiobesity agents include sibutramine which can be prepared as described in U.S. Pat. No. 4,929,629. and bromocriptine which can be prepared as described in U.S. Pat. Nos. 3,752,814 and 3,752,888.

Anti-resorptive agents (for example progestins, polyphosphonates, bisphosphonate(s), estrogen agonists/antagonists, estrogen, estrogen/progestin combinations, Premarin.RTM., estrone, estriol or 17.alpha.- or 17.beta.-ethynyl estradiol) may be used in conjunction with the compounds of Formula I of the present invention. Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol. Preferred progestins are medroxyprogestrone, norethindrone and norethynodrel. Exemplary bone resorption inhibiting polyphosphonates include polyphosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate and resindronate are especially preferred polyphosphonates. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl) amino methane diphosphonic acid, butane-4-amino-1-hydroxy-1,1-diphosphonic acid, pentane-5-amino-1-hydroxy- -1,1-diphosphonic acid, hexane-6-amino-1-hydroxy-1,1-diphosphonic acid and pharmaceutically acceptable esters and salts thereof.

The compounds of this invention may be combined with a mammalian estrogen agonist/antagonist. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods (Eriksen E. F. et al., Bone Histomorphometry, Raven Press, New York, 1994, pages 1-74; Grier S. J. et. al., The Use of Dual-Energy X-Ray Absorptiometry In Animals, “Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below.

Another preferred estrogen agonist/antagonist is 3-(4-(1,2-diphenyl-but-1-enyl)-phenyl)-acrylic acid, which is disclosed in Willson et al., Endocrinology, 1997, 138, 3901-3911. Another preferred estrogen agonist/antagonist is tamoxifen: (ethanamine,2-(-4-(1,2-diphenyl-1-butenyl)phenoxy)-N,N-dimethyl, (Z)-2-, 2-hydroxy-1,2,3-propanetricarboxylate (1:1)) and related compounds which are disclosed in U.S. Pat. No. 4,536,516, the disclosure of which is incorporated herein by reference. Another related compound is 4-hydroxy tamoxifen, which is disclosed in U.S. Pat. No. 4,623,660, the disclosure of which is incorporated herein by reference.

A preferred estrogen agonist/antagonist is raloxifene: (methanone, (6-hydroxy-2-(4-hydroxyphenyl)benzo[b]thien-3-yl)(4-(2-(1-piperidinyl)eth-oxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl) phenoxy)-N,N-dimethyl-, (Z)-, 2-hydroxy-1,2,3-propanetricarboxylate (1:1) which is disclosed in U.S. Pat. No. 4,996,225, the disclosure of which is incorporated herein by reference. Another preferred estrogen agonist/antagonist is centchroman: 1-(2-((4-(-methoxy-2,2, dimethyl-3-phenyl-chroman-4-yl)-phenoxy)-ethyl)-p-yrrolidine, which is disclosed in U.S. Pat. No. 3,822,287, the disclosure of which is incorporated herein by reference. Also preferred is levormeloxifene. Another preferred estrogen agonist/antagonist is idoxifene: (E)-1-(2-(4-(1-(4-iodo-phenyl)-2-phenyl-but-1-enyl)-phenoxy)-ethyl)-pyrro-lidinone, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thio-phen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)-benzyl)-naphthalen-2-ol, which is disclosed in U.S. Pat. No. 5,484,795, the disclosure of which is incorporated herein by reference.

Another preferred estrogen agonist/antagonist is (4-(2-(2-aza-bicyclo[2.2.1]hept-2-yl)-ethoxy)-phenyl)-(6-hydroxy-2-(4-hyd-roxy-phenyl)-benzo[b]thiophen-3-yl)-methanone which is disclosed, along with methods of preparation, in PCT publication no. WO 95/10513 assigned to Pfizer Inc., the disclosure of which is incorporated herein by reference.

Other preferred estrogen agonist/antagonists include the compounds, TSE-424 (Wyeth-Ayerst Laboratories) and arazoxifene.

Other preferred estrogen agonist/antagonists include compounds as described in commonly assigned U.S. Pat. No. 5,552,412, the disclosure of which is incorporated herein by reference. Especially preferred compounds described therein are:

cis-6- (4-fluoro-phenyl)-5-(4-(2-piperidin-1-yl-ethoxy)-phenyl)-5,6,-7,8-tetrahydro-naphthalene-2-ol;
(−)-cis-6-phenyl-5-(4-(2-pyrrolidin-1-yl-ethoxy)-phenyl)-5,6,7,8-te-trahydro-naphthalene-2-ol (also known as lasofoxifene);
cis-6-phenyl-5-(4-(2-pyrrolidin-1-yl-ethoxy)-phenyl)-5,6,7,8-tetrah-ydro-naphthalene-2-ol;
cis-1-(6′-pyrrolodinoethoxy-3′-pyridyl)-2-phenyl-6-hydroxy-1,2,3,4-tetrahydronaphthalene;
1-(4′-pyrrolidinoethoxyphenyl)-2-(4″-fluorophenyl)-6-hydroxy-1,2,3,-4-tetrahydroisoquinoline;
is-6-(4-hydroxyphenyl)-5-(4-(2-piperidin-1-yl-ethoxy)-phenyl)-5,6,-7,8-tetrahydro-naphthalene-2-ol; and
1-(4′-pyrrolidinolethoxyphenyl)-2-phenyl-6-hydroxy-1,2,3,4-tetrahyd- roisoquinoline.

Other estrogen agonist/antagonists are described in U.S. Pat. No. 4,133,814 (the disclosure of which is incorporated herein by reference). U.S. Pat. No. 4,133,814 discloses derivatives of 2-phenyl-3-aroyl-benzoth-iophene and 2-phenyl-3-aroylbenzothiophene-1-oxide.

Other anti-osteoporosis agents, which can be used in combination with a Formula I compound of the present invention, include, for example, the following: parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs.

Any compound that is an antihypertensive agent may be used in a combination aspect of this invention. Such compounds include amlodipine and related dihydropyridine compounds, calcium channel blockers, angiotensin converting enzyme inhibitors (“ACE-Inhibitors”), angiotensin-II receptor antagonists, beta-adrenergic receptor blockers and alpha-adrenergic receptor blockers. Such antihypertensive activity is determined by thoseskilled in the art according to standard tests (e.g. blood pressure measurements).

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate and other pharmaceutically acceptable acid addition salts of amlodipine have utility as antihypertensive agents and as antiischemic agents. Amlodipine and its pharmaceutically acceptable acid addition salts are also disclosed in U.S. Pat. No. 5,155,120 as having utility in the treatment of congestive heart failure. Amlodipine besylate is currently sold as Norvasc®.

Calcium channel blockers which are within the scope of a combination aspect of this invention include, but are not limited to: bepridil, which may be prepared as disclosed in U.S. Pat. No. 3,962, 238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, prenylamine, semotiadil, terodiline, verapamil, aranipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexiline The disclosures of all such U.S. Patents are incorporated herein by reference.

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, ceronapril, delapril, enalapril, fosinopril, imadapril, lisinopril, moveltopril, perindopril, quinapril, ramipril, spirapril, temocapril, and trandolapril. The disclosures of all such U.S. patents are incorporated herein by reference.

Angiotensin-ll receptor antagonists (A-Il antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, losartan, and valsartan. The disclosures of all such U.S. patents are incorporated herein by reference.

Beta-adrenergic receptor blockers (beta- or. beta. -blockers) which are within the scope of this invention include, but are not limited to: acebutolol, which may be prepared as disclosed in U.S. Pat. No. 3,857,952; alprenolol, amosulalol, which may be prepared as disclosed in U.S. Pat. No. 4,217,305; arotinolol, atenolol, befunolol, betaxolol; The disclosures of all such U.S. patents are incorporated herein by reference.

Alpha-adrenergic receptor blockers (alpha- or .alpha.-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, doxazosin, fenspiride, indoramin, labetolol, naftopidil, nicergoline, prazosin, tamsulosin, tolazoline, trimazosin, and yohimbine, which may be isolated from natural sources according to methods well known to those skilled in the art. The disclosures of all such U.S. patents are incorporated herein by reference.

Any compound that is known to be useful in the treatment of Alzheimer's Disease may be used in a combination aspect of this invention. Such compounds include acetylcholine esterase inhibitors. Examples of known acetylcholine esterase inhibitors include donepezil (Aricept®), tacrine (Cognex®), rivastigmine (Exelon®) and galantamine (Reminyl). Aricept® is disclosed in the following U.S. patents, all of which are fully incorporated herein by reference: U.S. Pat. Nos. 4,895,841, 5,985,864, 6,140,321, 6,245,911 and 6,372,760. Exelone is disclosed in U.S. Patent Nos. 4,948,807 and 5,602,176 which are fully incorporated herein by reference. Cognex® is disclosed in U.S. Pat. Nos. 4,631,286 and 4,816,456 (fully incorporated herein by reference). Remynile is disclosed in U.S. Pat. Nos. 4,663,318 and 6,099,863 which are fully incorporated herein by reference.

### Preparation of the Compounds of the Invention

The present invention contains compounds that can be synthesized in a number of ways familiar to one skilled in organic synthesis. The compounds outlined herein can be synthesized according to the methods described below, along with methods typically used by a synthetic organic chemist, and combinations or variations of those methods, which are generally known to one skilled in the art of synthetic chemistry. The synthetic route of compounds in the present invention is not limited to the methods outlined below. One skilled in the art will be able to use the schemes below to synthesize compounds claimed in this invention. Individual compounds may require manipulation of the conditions in order to accommodate various functional groups. A variety of protecting groups known to one skilled in the art may be required. Purification, if necessary, may be accomplished on a silica gel column eluted with the appropriate organic solvent system. Also, reverse phase HPLC or recrystallization may be employed. The following non-limiting descriptions also demonstrate methods for the synthesis of compounds of the invention.

Schemes 1-13 relate to the preparation of imidizoles.

Schemes 14 et sea relate to the prepration of pyrrole compounds.

### Preparation of Imidazole Compounds

Schemes 1-3 relate to the preparation of compounds of the invention having a Formula I wherein R² is, for example, 4-fluorophenyl, R⁴ is, for example, benzyl amide, and R⁵ is, for example, isopropyl.

A general procedure for the preparation of the cycloaddition precursor 4 is illustrated in Scheme 1. The synthesis of 4 begins with a selective bromination of commercially available 4-fluorophenylacetic acid methyl ester via the method of Kikuchi, D. et al (J. Org. Chem., 1998, 63, 6023) to give racemic Bromo- (4-fluoro-phenyl)-acetic acid methyl ester 1. Reaction of 1 with [(4R, 6R)-6-(2-Amino-ethyl)-2,2-dimethyl-[1,3]dioxan-4-yl]-acetic acid tert-butyl ester (Baumann, Kelvin L.; Butler, Donald E.; Deering, Carl F.; Mennen, Kenneth E.; Millar, Alan; Nanninga, Thomas N.; Palmer, Charles W.; Roth, Bruce D.; Tetrahedron Letters (1992), 33(17), 2283) provides the amino ester 2 as a mixture of diastereomers. Acylation of 2 and saponification of the intermediate methyl ester 3 yields {[2-((4R,6R)-6-tert-Butoxycarbonylmethyl-2,2-dimethyl-[1 ,3]dioxan-4-yl)-ethyl]-isobutyryl-amino}-(4-fluoro-phenyl)-acetic acid, 4, which is isolated as a mixture of diastereomers.

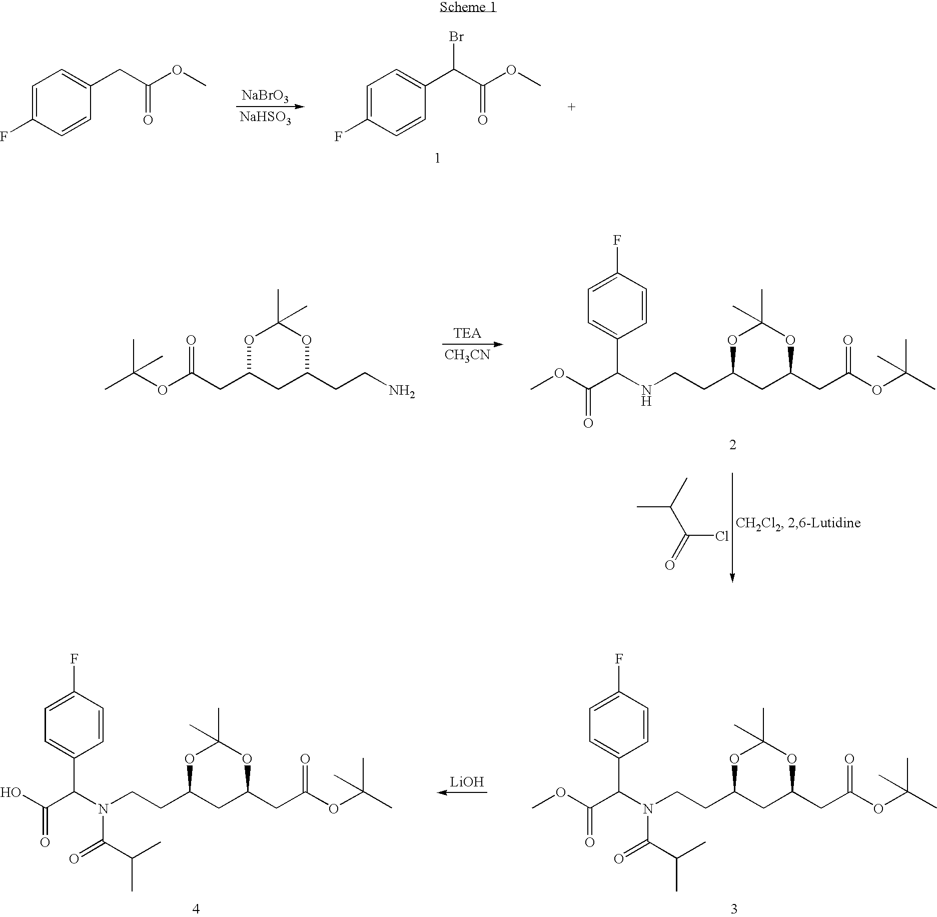

The following cycloaddition precursor compounds for example, may be prepared in a similar manner:

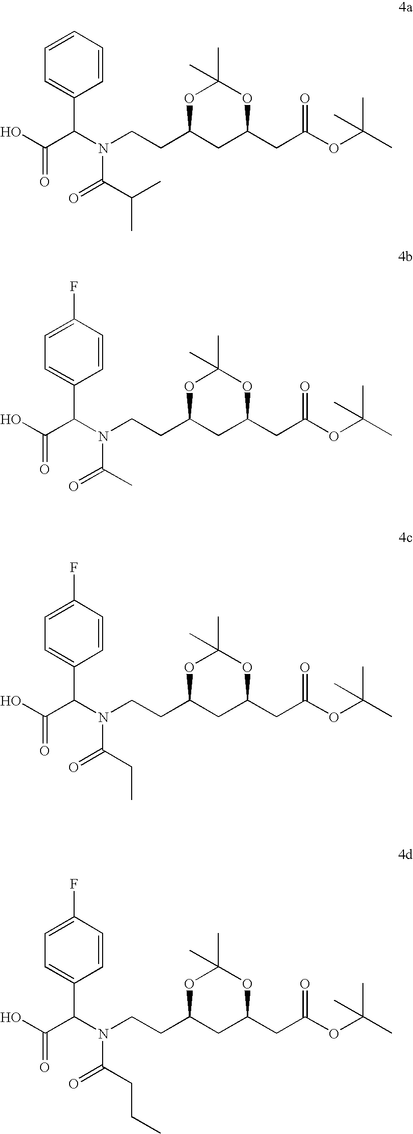

Scheme 2 illustrates the preparation of imidazole 5 and the imidazole-4-carboxylic acid 6. Thus, in a manner similar to that described by R. Huisgen et al (Chem. Ber. 1971, 104, 1562), treatment of compound 4 with acetic anhydride in the presence benzyl cyanoformate gives the desired 1-[2-((4R,6R)-6-tert-butoxycarbonylmethyl-2,2-dimethyl [1,3]dioxan-4-yl)-ethyl]-2-(4-fluoro-phenyl)-5-isopropyl-1 H-imidazole-4-carboxylic acid benzy ester 5. Hydrogenolysis of 5 gives the free acid 6.

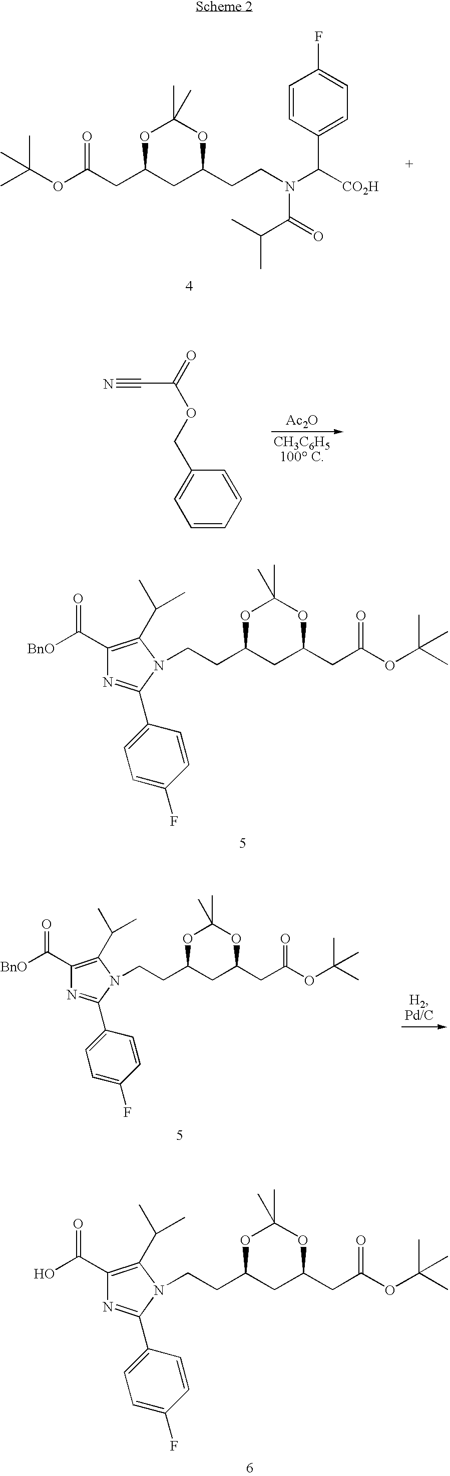

The following compounds may be prepared as shown in Scheme 2, from which the corresponding free acids may also be prepared.

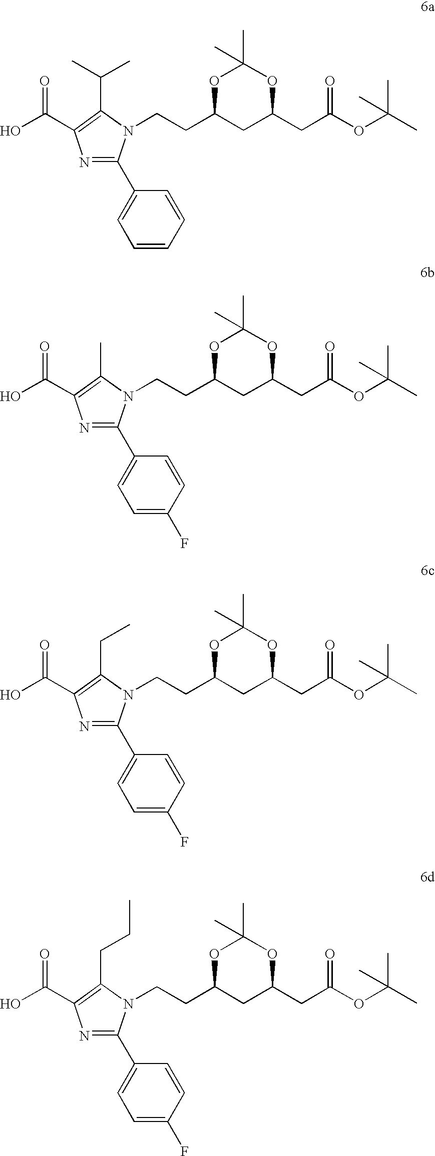

Scheme 3 illustrates the preparation of imidazole compound 9 from compound 6. Thus, the free acid 6 is transformed to the pentafluorophenyl ester 7. Reaction of compound 7 with benzyl amine and subsequent deprotection yields the lactone compound 8. The lactone 8 is converted to 9 on treatment with sodium hydroxide.

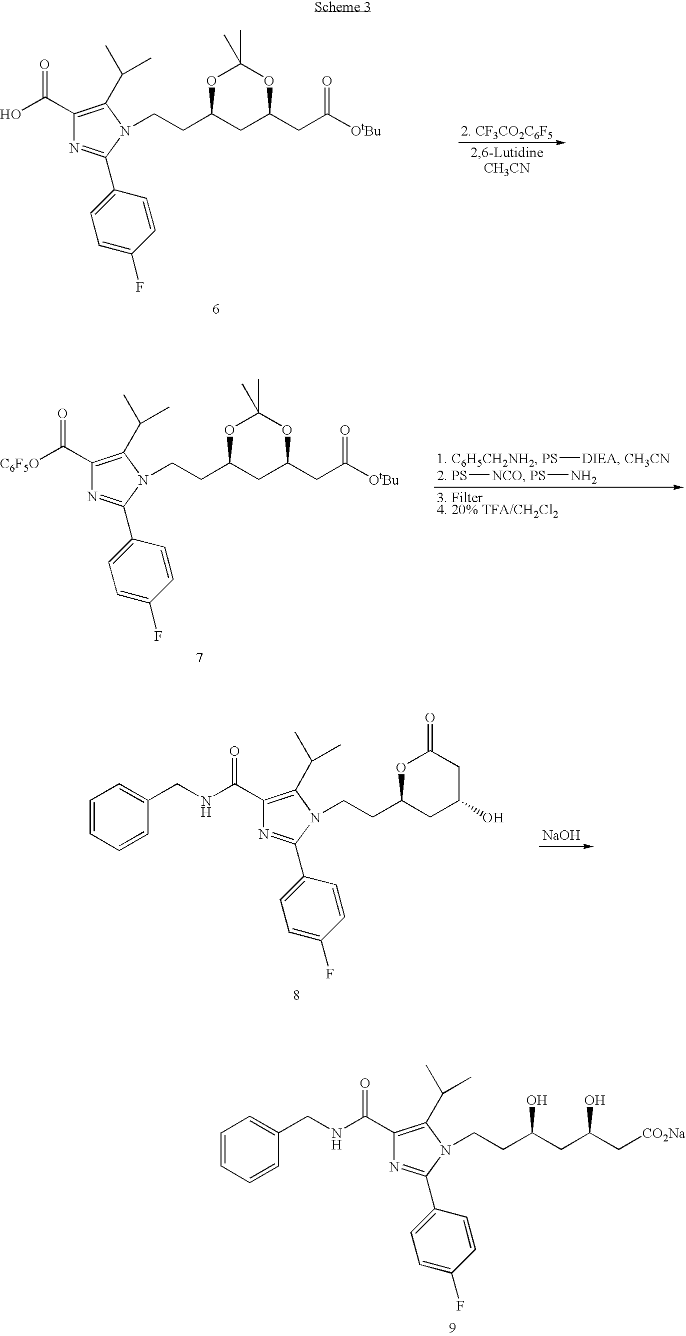

Scheme 4 illustrates an alternate preparation of compounds of the invention from the carboxylic acid 6. Thus, in situ activation of 6 with PyBOP or EDCI/HOBt, or a similar activating agent, and treatment with 3-aminomethyl pyridine gives the amide 10. Exposure of 10 to TFA provides the lactone 11 which is converted to 12 on treatment with base. Alternatively, the crude coupling product 10 may be converted to the Lactone 11 without isolation.

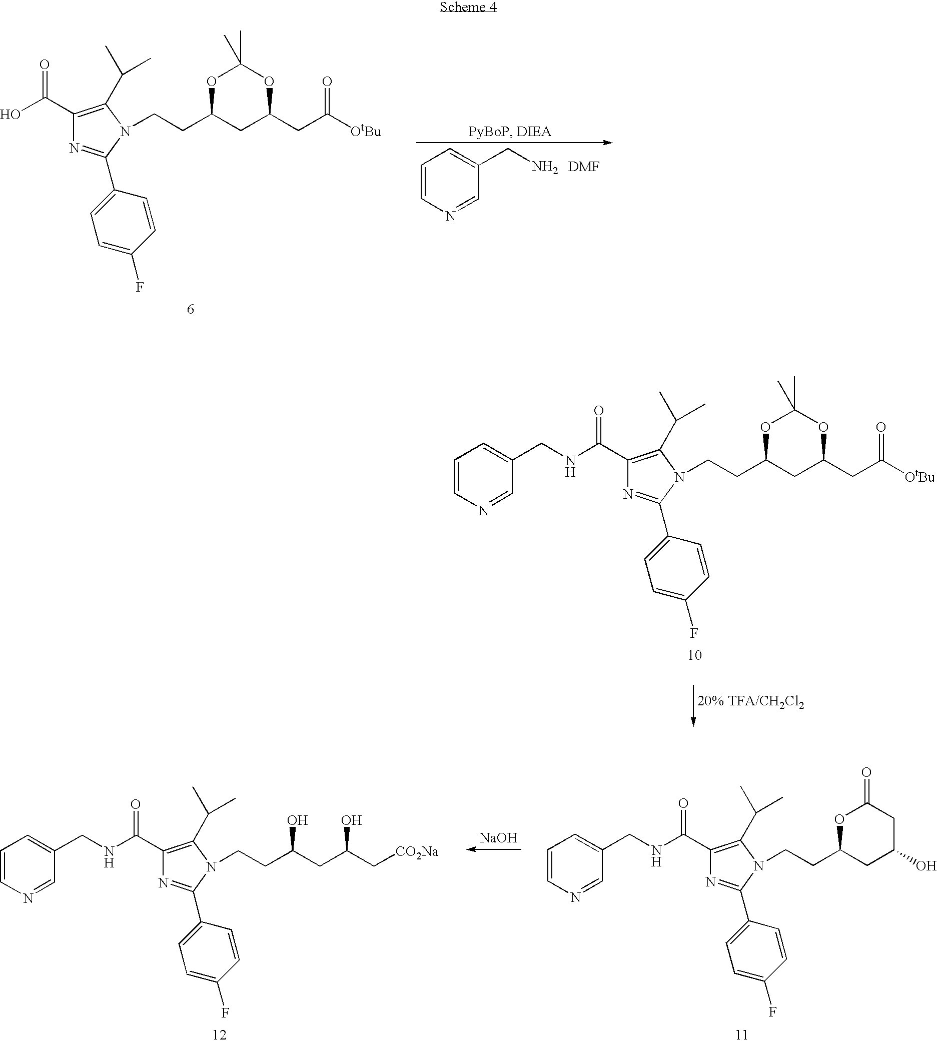

Scheme 5 illustrates the preparation of compounds of the invention having a Formula I wherein R² is, for example, 4-fluorophenyl, R⁴ is a sulfone and R5 is, for example, isopropyl.

Scheme 5 exemplifies the preparation of the sulfone 15 from the carboxylic acid 4. Thus, reaction of compound 4 with commercially available tosyl cyanide yields the imidazole 13. Exposure of 13 to TFA provides the lactone 14 which is converted to 15 on treatment with base.

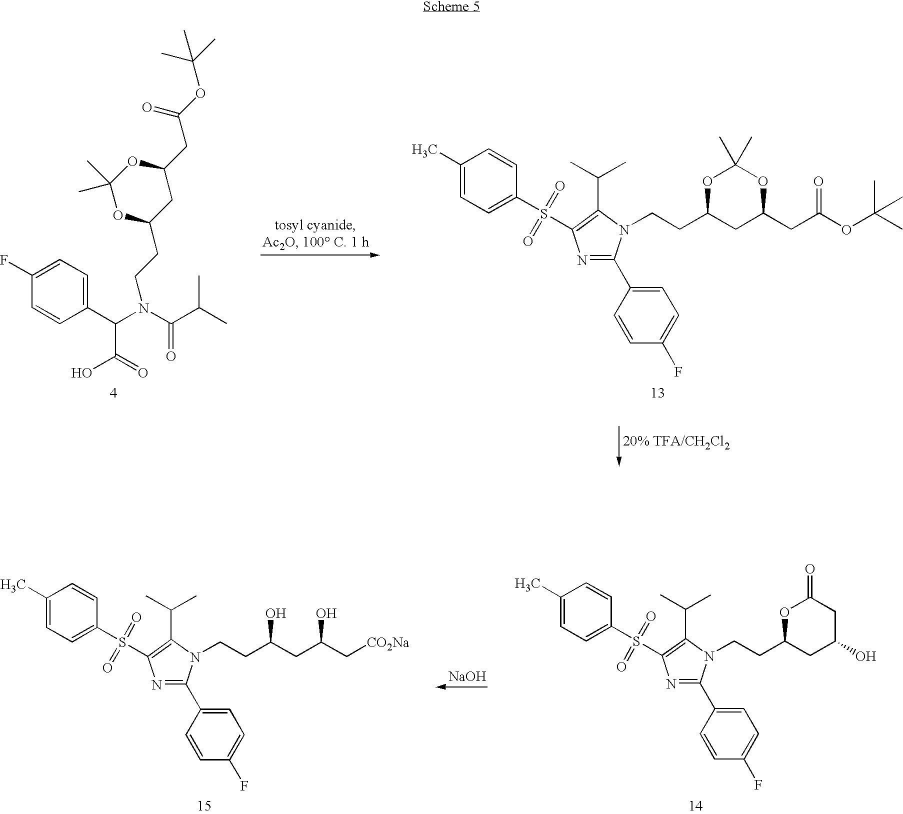

Scheme 6 illustrates a preparation of 4-aminoimidazoles 21 from the acid 16, wherein R², R⁵ and R⁶ are as defined supra. Thus, reaction of the acid 16 with diphenyl phosphoryl azide, (DPPA), in the presence of benzyl alcohol provides 17. This compound is transformed to the aminoimidazole 18 by catalytic hydrogenation. Acylation or sulfonylation of 18 yields 19. Exposure of 19 to TFA provides the lactone 20 which is converted to 21 on treatment with base.

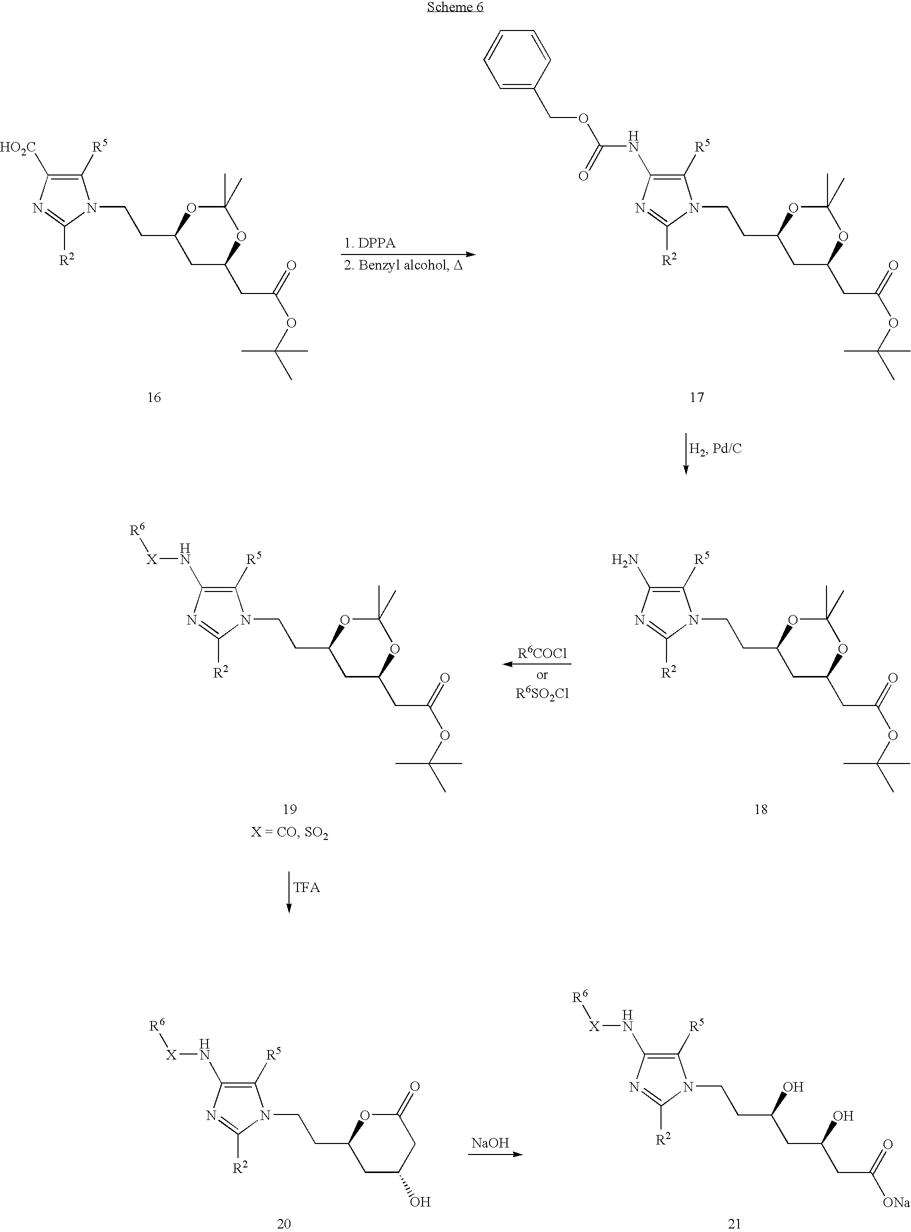

An alternate synthesis of 1-[2-((4R, 6R)-6-tert-Butoxycarbonylmethyl-2,2-dimethyl [1,3]dioxan-4-yl)-ethyl]-2-(4-fluoro-phenyl)-5-isopropyl-1 H-imidazole-4-carboxylic acid 6 is illustrated in Scheme 7. Thus, (benzhydrylidene-amino)-acetic acid benzyl ester 22, prepared by the condensation of benzhydrylideneamine with glycine benzyl ester, is acylated with isobutyryl chloride according to the method of J. Singh et al (Tetrahedron Left. 1993, 34, 21 1). Subsequent hydrolysis gives 23. A second acylation is accomplished by reacting the 23 with p-fluorobenzoyl chloride under basic condition to give 24. Cyclodehydration of 24 with [(4R, 6R)-6-(2-Amino-ethyl)-2,2-dimethyl-[1,3]dioxan-4-yl]-acetic acid tert-butyl ester yields the benzyl ester 25. Hydrogenolysis of 25 yields the free acid 6.

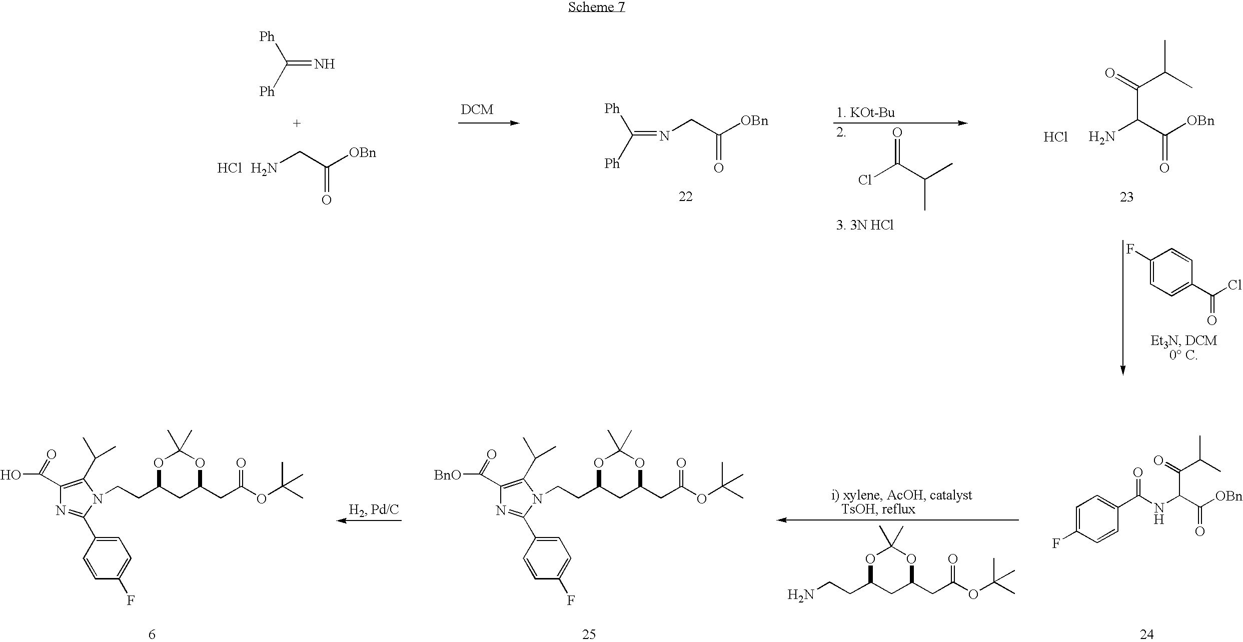

Scheme 8 exemplifies the preparation of 2-[[2-((4R, 6R)-6-tert-Butoxycarbonylmethyl-2,2-dimethyl [1,3] dioxan-4-yl)-ethyl]-(4-fluoro-benzoyl)-amino]-3-methyl-butyric acid 31. Thus, selective reduction of the benzyl ester 26, prepared from commercially available sodium 3-methyl-2-oxo-butyrate according to the method of Manfred Hesse et al (Helvetica Chim. Acta, 2001, 84, 3766), with sodium triacetoxyborohydride in ethanol at 0° C. yields racemic 2-Hydroxy-3-methyl-butyric acid benzyl ester 27. Compound 27 is converted to the corresponding triflate 28 on treatment with triflic anhydride in the presence of 2,6-lutidine (Michael Walker, Tetrahedron, 1997, 53, 14591). Reaction of 28 with [(4R, 6R)-6-(2-Amino-ethyl)-2,2-dimethyl-[1,3]dioxan-4-yl]-acetic acid tert-butyl ester provides the amino ester 29 as a mixture of diastereomers which are not seperated. Acylation of 29 and hydrogenolysis of the resulting benzyl ester 30 yields 2-[[2-((4R, 6R)-6-tert-Butoxycarbonylmethyl-2, 2-dimethyl [1,3] dioxan-4-yl)-ethyl]-(4-fluoro-benzoyl)-amino]-3-methyl-butyric acid, 31, as a mixture of diastereomers.

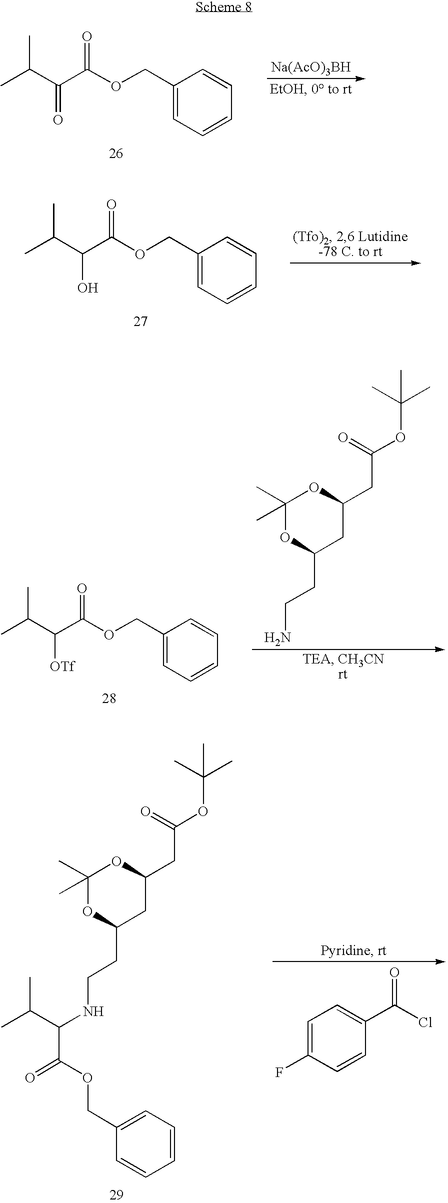

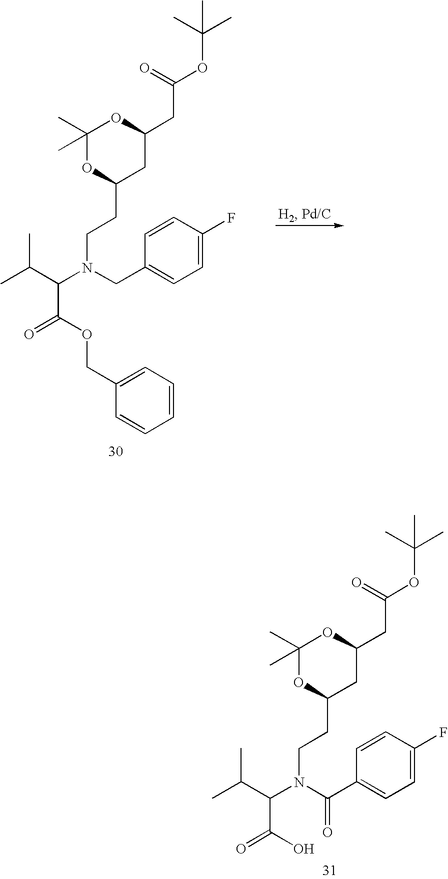

Scheme 9 illustrates an alternate method for the preparation of 1-[2-((4R, 6R)-6-tert-Butoxycarbonylmethyl-2,2-dimethyl[1,3]dioxan-4-yl)-ethyl]-2-(4-fluoro-phenyl)-5-isopropyl-1H-imidazole-4-carboxylic acid 6. Thus, reaction of 31 with Bis(toluene-4-sulfonyl amino) acetic acid benzyl ester 32, prepared by condensation of benzyl glyoxalate hydrate with p-toluene sulfonamide, in the presence of EDCI yields 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1 H-imidazole-4-carboxylic acid benzyl ester 5. Hydrogenolysis of 5 gives the free acid 6.

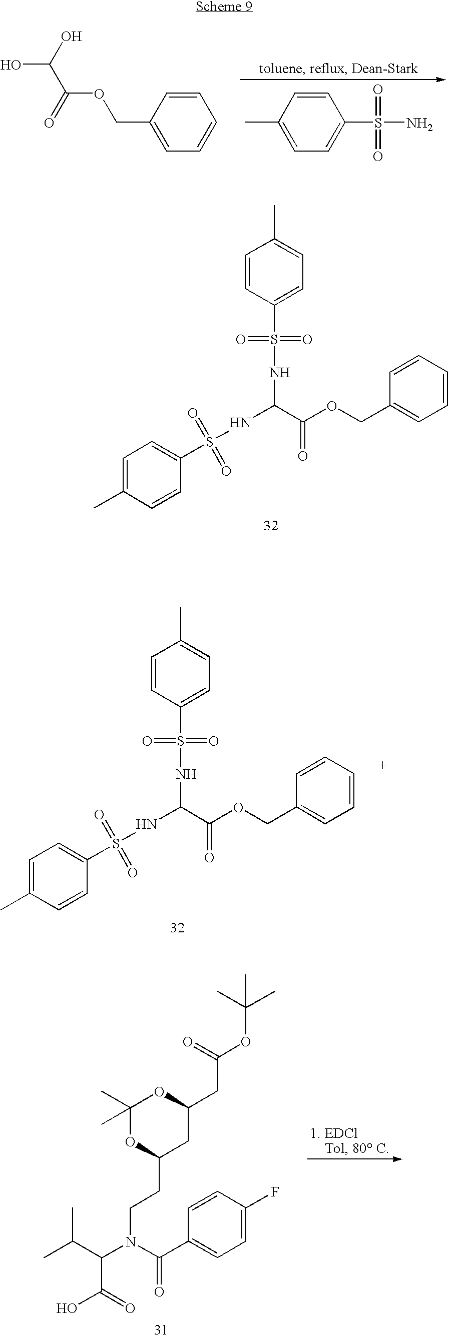

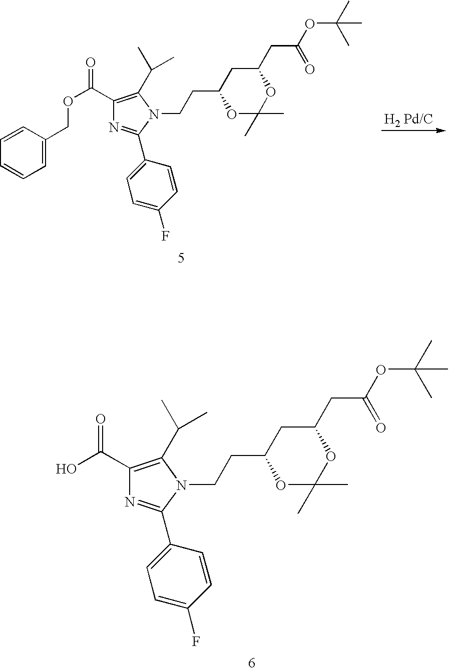

Scheme 10 illustrates an alternate method for the preparation of the sodium salt of (3R,5R)-7-[4-Benzylcarbamoyl-2-(4-fluoro-phenyl)-5-isopropyl-imidazol-1-yl]-3,5-dihdroxy-heptanoic acid 9. Thus a reaction of 31 with Bis-(toluene-4-sulfonylamino)-acetic acid benzyl amide 33 in the presence of EDCl yields 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1 H-imidazole-4-carboxylic acid benzyl amide 34. Exposure of 34 to TFA provides the lactone 8 which is converted to 9 on treatment with base. The novel Bis-(toluene-4-sulfonylamino)-acetic acid benzyl amide 33 is prepared in two steps from commercially available N,N′-dibenzyl-oxalamide.

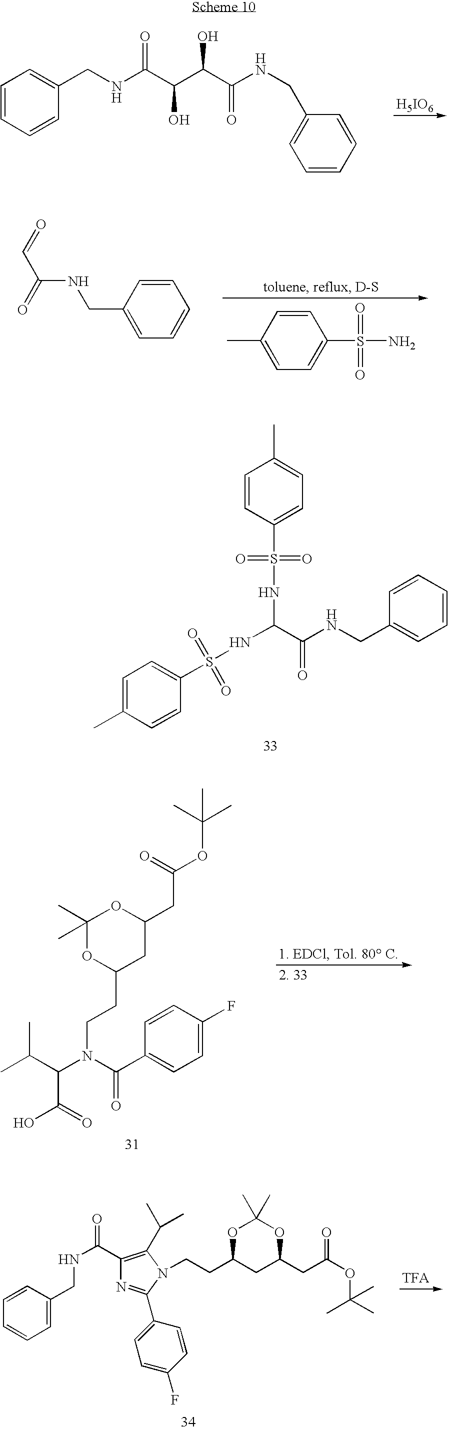

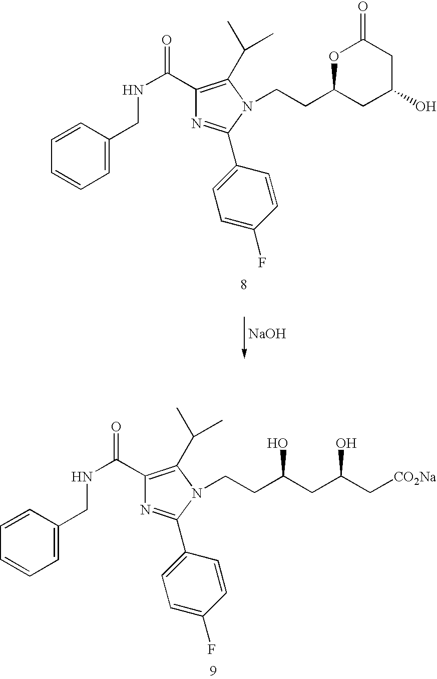

Scheme 1 1 illustrates an alternate method for the preparation of imidazole sodium salt 9 from ketoamide 24. Trans-amidation of ketoamide 24 with benzylamine yields ketobenzamide 35. Treatment of 35 with TBIA and benzoic acid or phenylacetic acid in refluxing heptane affords imidazole 34. Acid-catalyzed removal of the acetal yields diol 36, and subsequent hydroxide saponification, followed by acid-catalyzed condensation affords lactone 8. Lactone 8 is converted to imidazole sodium salt 9 by treatment with aqueous sodium hydroxide. Alternatively, treatment of diol 36 with NaOH will give 9 directly. Recrystallization of crude sodium salt 9 affords material of high purity.

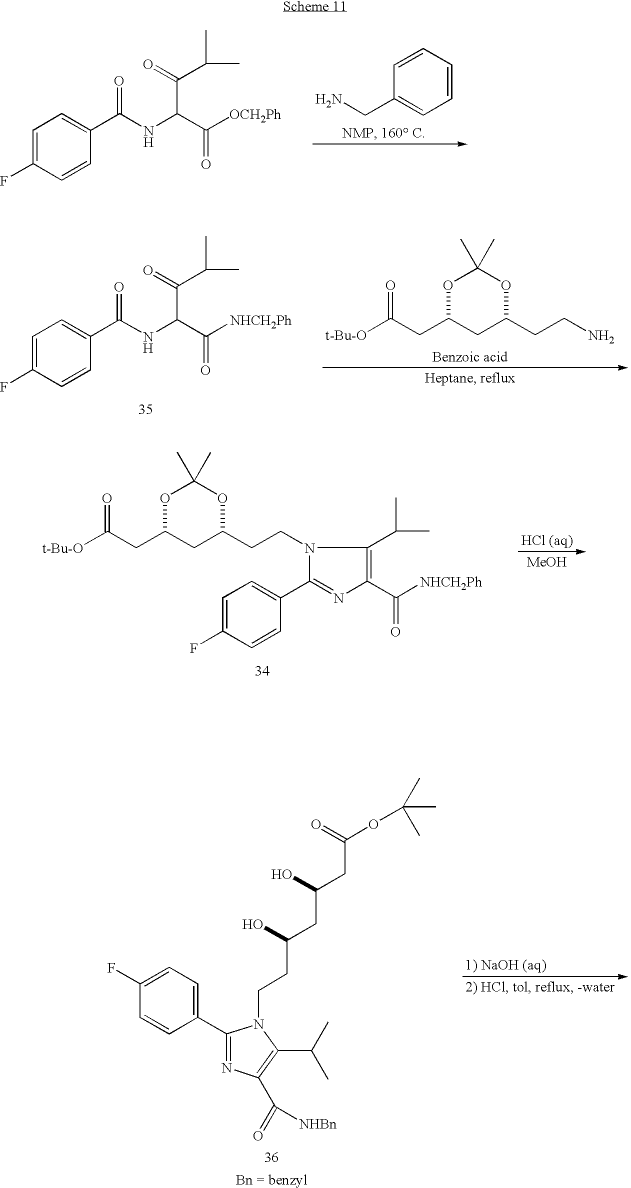

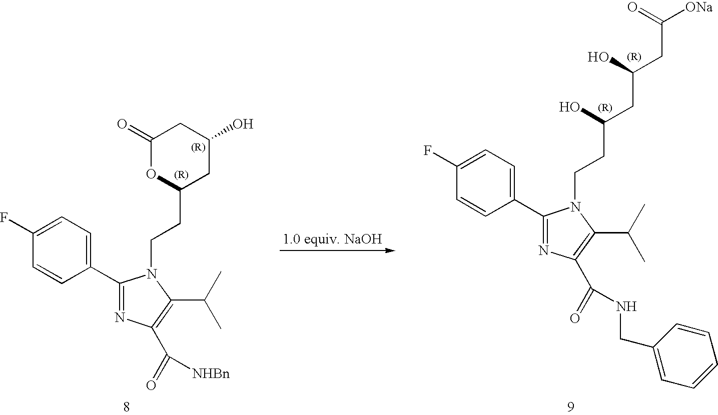

Scheme 12 illustrates an alternate method for the preparation of Imidazole 34. As shown in Scheme 12, Compound 38 reacts with compound 39 to give compound 40 that is converted to acid 41. The acid 41 is coupled with an amine of choice under standard peptide bond formation reaction conditions to afford amide 42 that is subsequently converted to compound 43 in a salt form under acidic conditions. Compound 44 is derived from TBIA and an acid chloride of choice. Compound 44 is treated with oxalyl chloride in presence of a organic base such as 2,6-lutidine to form iminochloride in situ that reacts with compound 43 to give midazole 34. 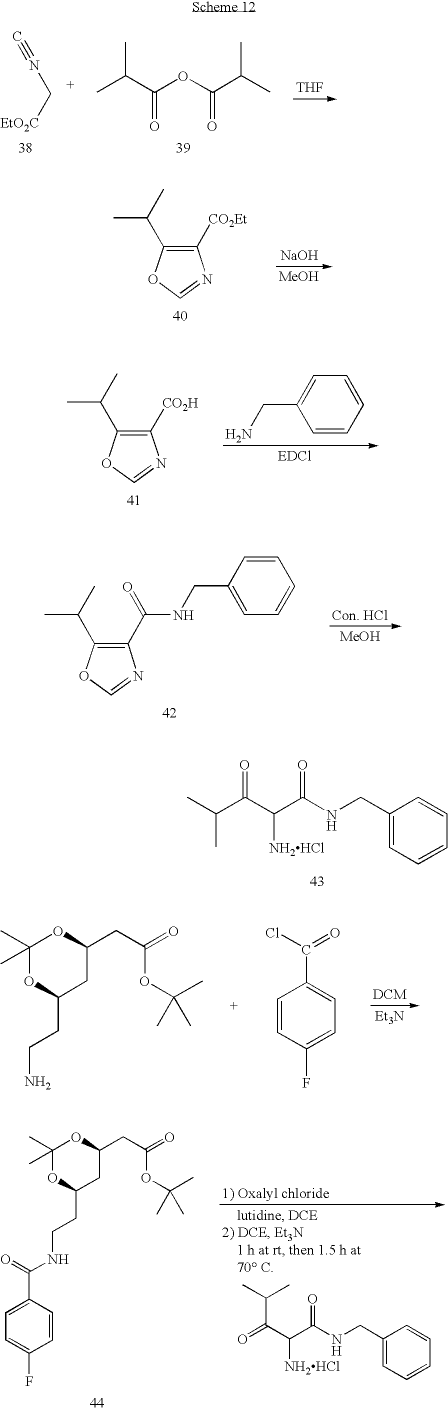 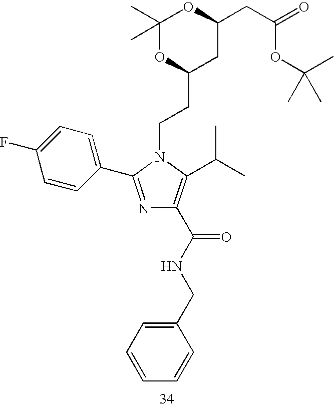
Scheme 13 shows the preparation of compound 48 from compound 7. Compound 7 is selectively reduced to the alcohol 45 on treatment with sodium borohydride. Manganese (IV) oxide oxidation of 45 gives the aldehyde 46. Reductive amination of 46 followed by sulfonylation and global provides the lactone 47, which is converted to 48 on treatment with sodium hydroxide.

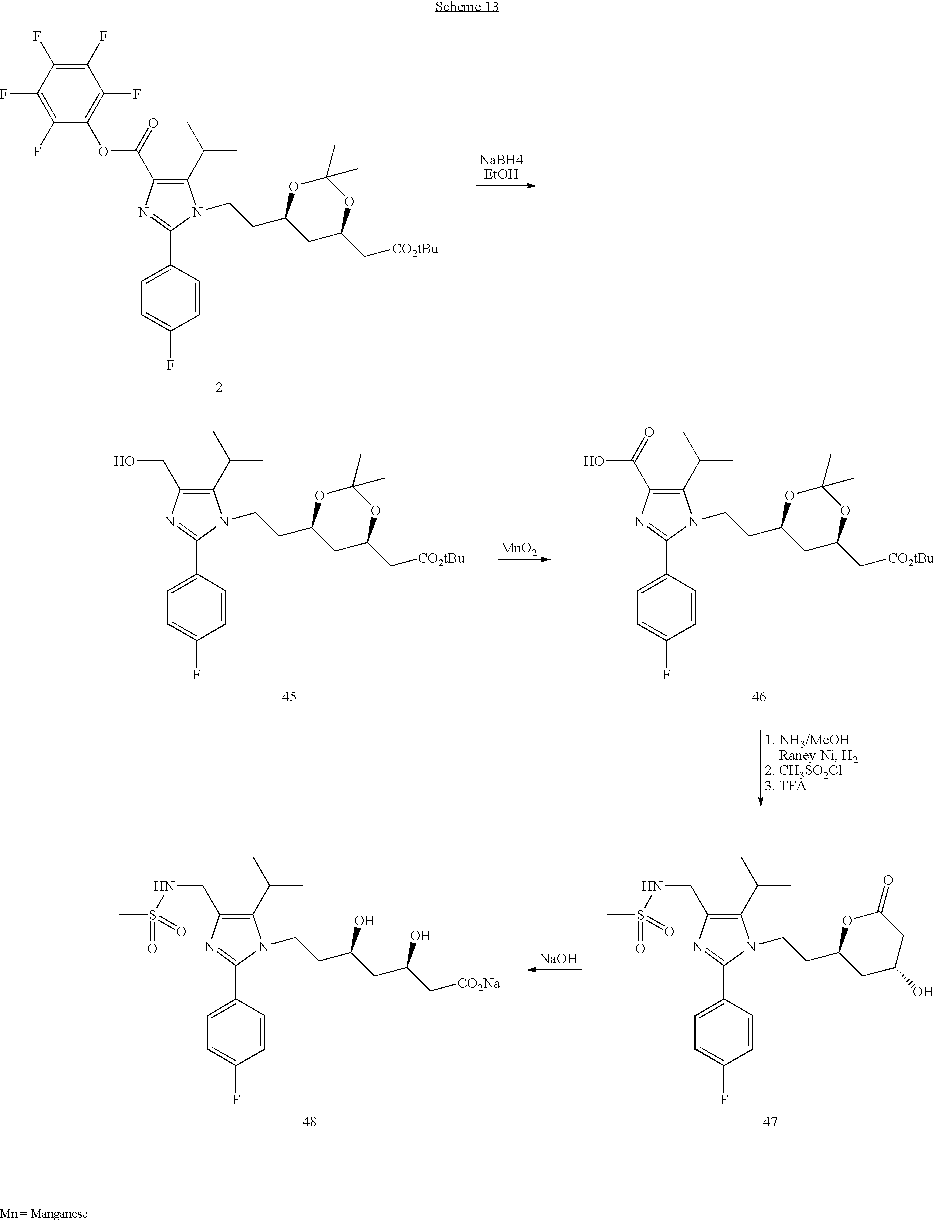

### Preparation of Pyrrole Compounds

Scheme 14 shows the preparation of compounds of Formula I wherein R² and R³ are each parafluorophenyl, R⁴ is SO₂NR⁹R¹⁰ and R⁵ is isopropyl.

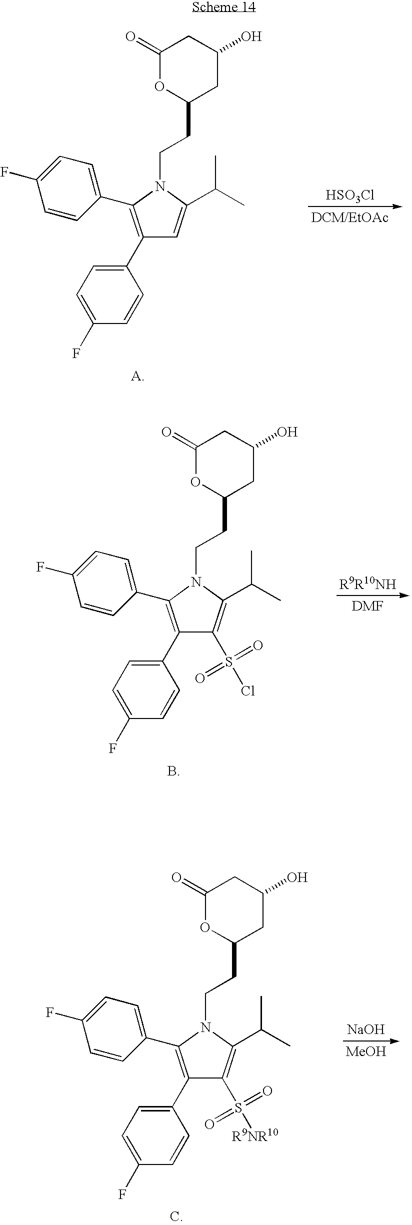

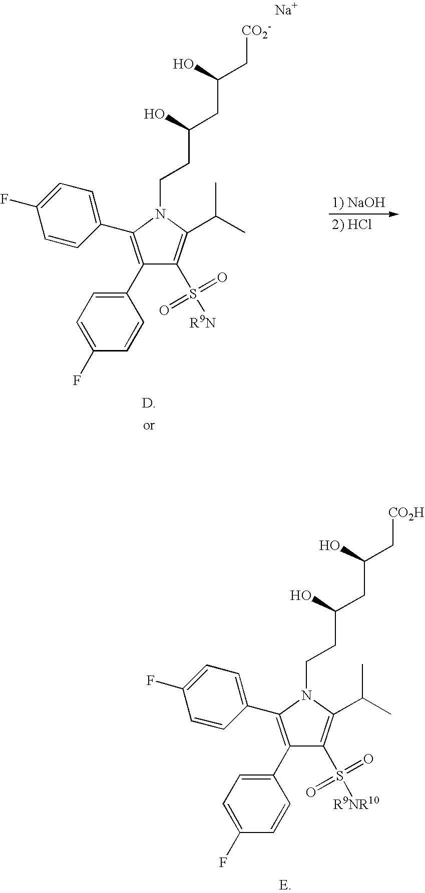

Compound A is treated with chlorosulfonic acid in dichloromethane to give compound B which reacts with an amine of interest in DMF to afford the sulfonamide C. Hydrolysis of the lactone gives the desired compound D, a sodium salt or di-sodium salt depending on the chemical nature of R⁹ and R¹⁰ groups. Alternatively, one could work up the reaction under acidic conditions to isolate the corresponding free acid. Preparation of starting material A is shown in Scheme 14a: 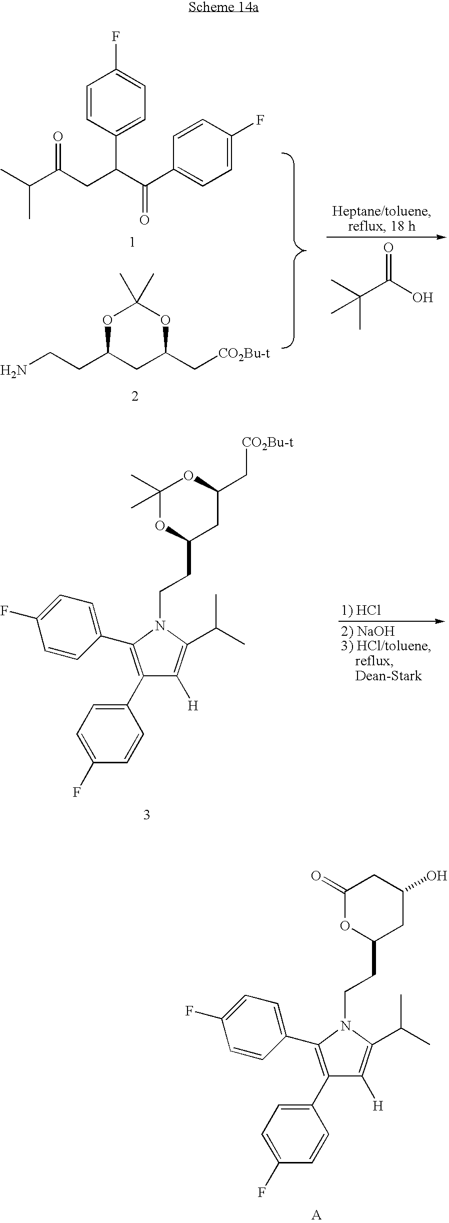
The di-ketone 1, which was prepared in a similar manner as described by Bruce et al (J. Med. Chem. 1991, 34, 357-366), reacts with amine 2 (see US0051 49837A for the synthesis) under acidic conditions to give compound 3. Acid catalyzed hydrolysis of the acetonide 3 followed by saponification of the ester using aqueous NaOH solution affords the di-hydroxy acid. Acid catalyzed lactonization of the di-hydroxy acid gives the compound A.

Alternate methods for preparing compounds of the invention are shown in Scheme 15 and in Scheme 15i.

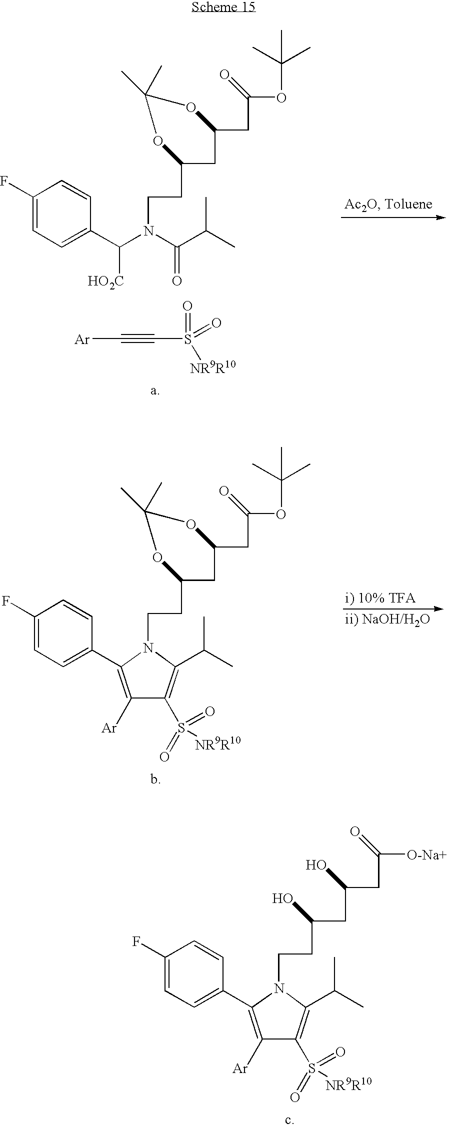

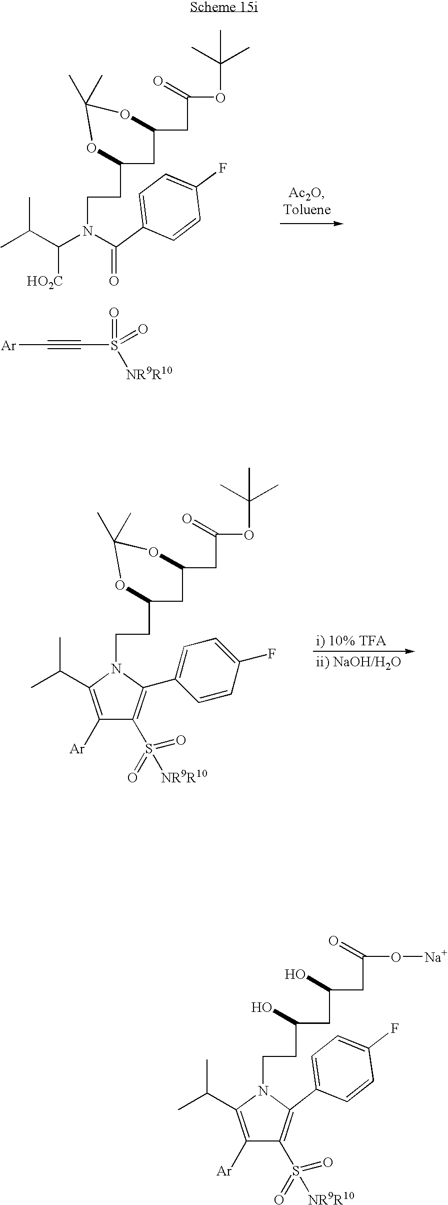

Similar compounds may be made using Scheme 15 and 15i, for example, as shown in Scheme 15a and Scheme 15ai:

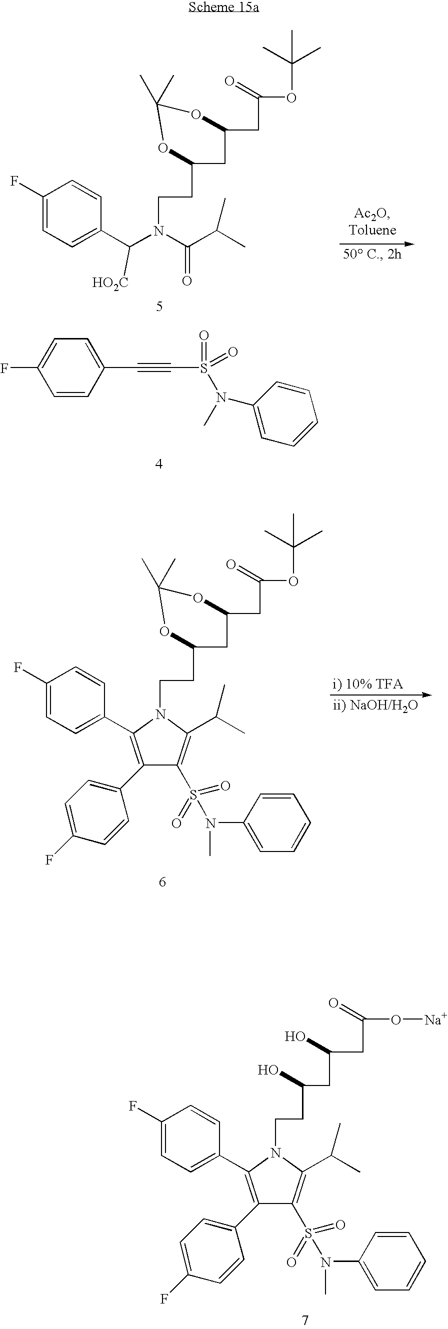

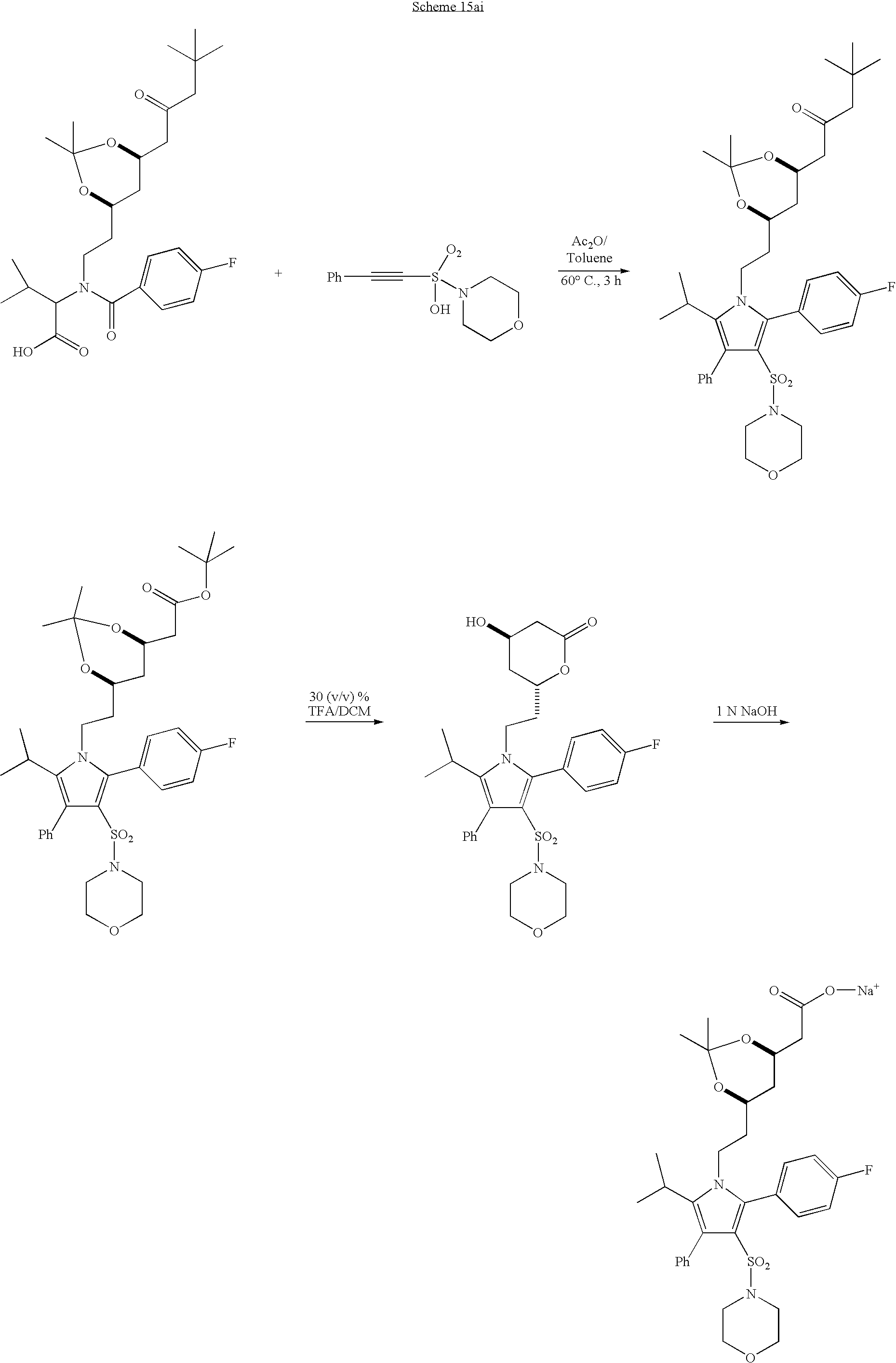

In Scheme 1 5a, a Munchnone intermediate is formed in situ from acetic anhydride and compound 5, and subsequent [3+2] cycloaddition of this intermediate with the alkynylsulfonamide 4 gives the desired regioisomer 6 in high yield. Treatment of 6 with TFA yields a lactone which in turn is hydrolyzed by NaOH to provide the final compound 7.

The starting materials for reaction Scheme 15a and 15ai may be made as shown in the following Schemes 15b, 15c and 15ci.

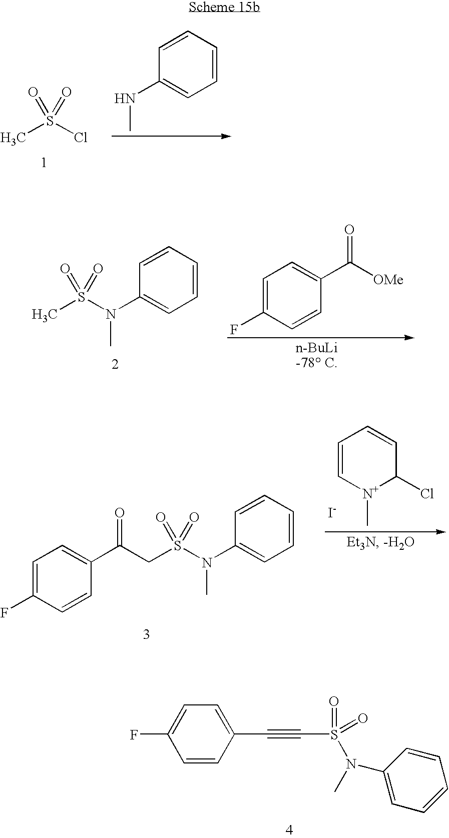

The main advantages of synthetic Schemes 15, 15a, 15b and 15c are two-fold: first, an effective route to compound 4. Second, achieving the right regioisomer in high yield (for example, compound 6). In Scheme 1 5b, the reaction of methane sulfonyl chloride (1) with N-methylaniline provides the corresponding sulfonamide (2) with high yield. The subsequent alkylation of 2 with 4-fluorobenzoic acid methyl ester yields the desired β-ketosulfonamide 3. Mukiyama reaction conditions ( for example, 2-chloro N-methylpyridinium iodide, triethylamine) were employed for dehydration of 3 to give the corresponding alkynyl sulfonamide 4. As used herein, “Mukiyama reaction conditions” means reaction conditions that effect dehydration of β-ketosulfonamide.

Scheme 15c describes the formation of the Munchnone precursor 5. Starting with 4-fluorobenzoic acid esterification to make methyl ester, subsequent bromination with NBS (N-bromosuccinimide) with a catalytic amount of HBr results in a bromo methyl ester 5b. A simple nucleophilic substitution reaction with the amine (TBIA) in the presence of base (triethylamine) provides the 5c with high yield. This secondary amine is acylated with for example, isobutyryl chloride (I-PrC(O)Cl) to give the desired methyl ester product. The methyl ester is hydrolyzed with a base LiOH to give the Munchnone precursor 5.

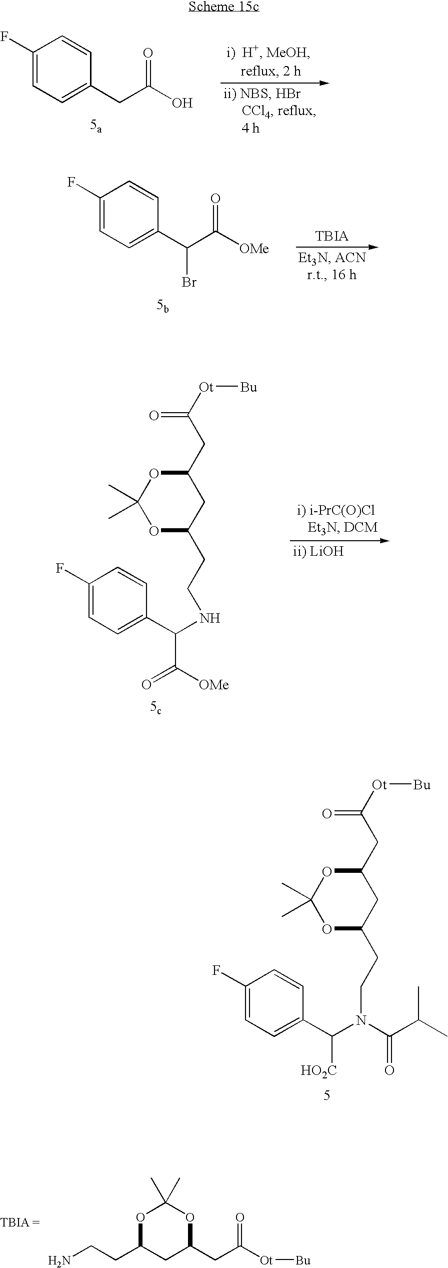

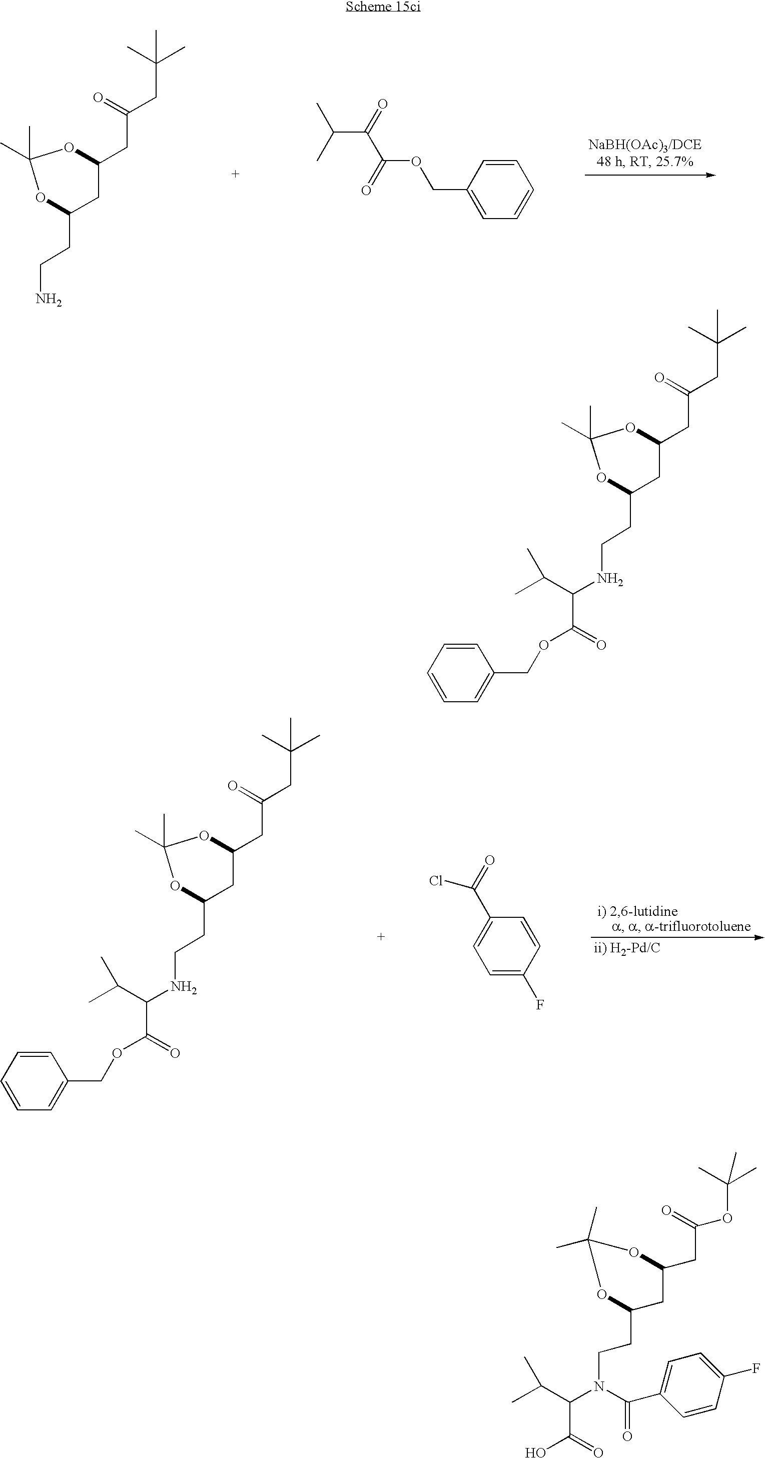

Scheme 16 describes preparation of compounds of the invention wherein one of R⁹ or R¹⁰ is H and the other one is denoted simply as “R”. Scheme 16 is similar to scheme 14 with the following advantage. Secondary or primary sulfonamides may give undesired amides instead of pyrrole formation in the Munchnone reaction. As used herein, “secondary sulfonamide” means when one of R⁹ and R¹⁰ is hydrogen. As used herein, “primary sulfonamide” means when both of R⁹ and R¹⁰ are hydrogen. In order to circumvent this, a protecting group (2,4,6-trimethoxybenzaldehyde) is temporarily added through sulfonamide 8. Scheme 16 provides an alternative synthetic method for the preparation of compounds of the invention with the advantages of fewer reaction steps and higher overall yield than previous methods.

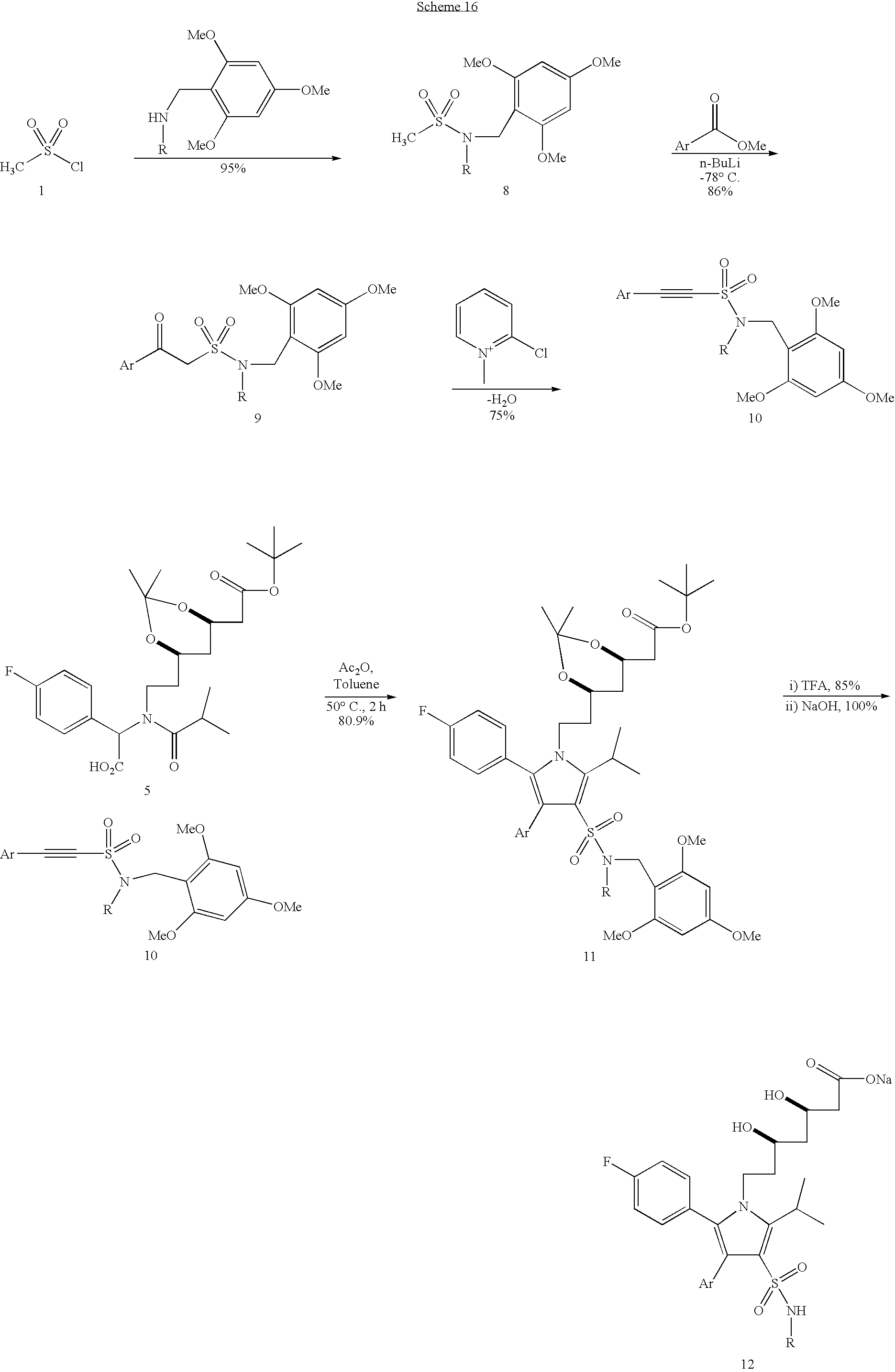

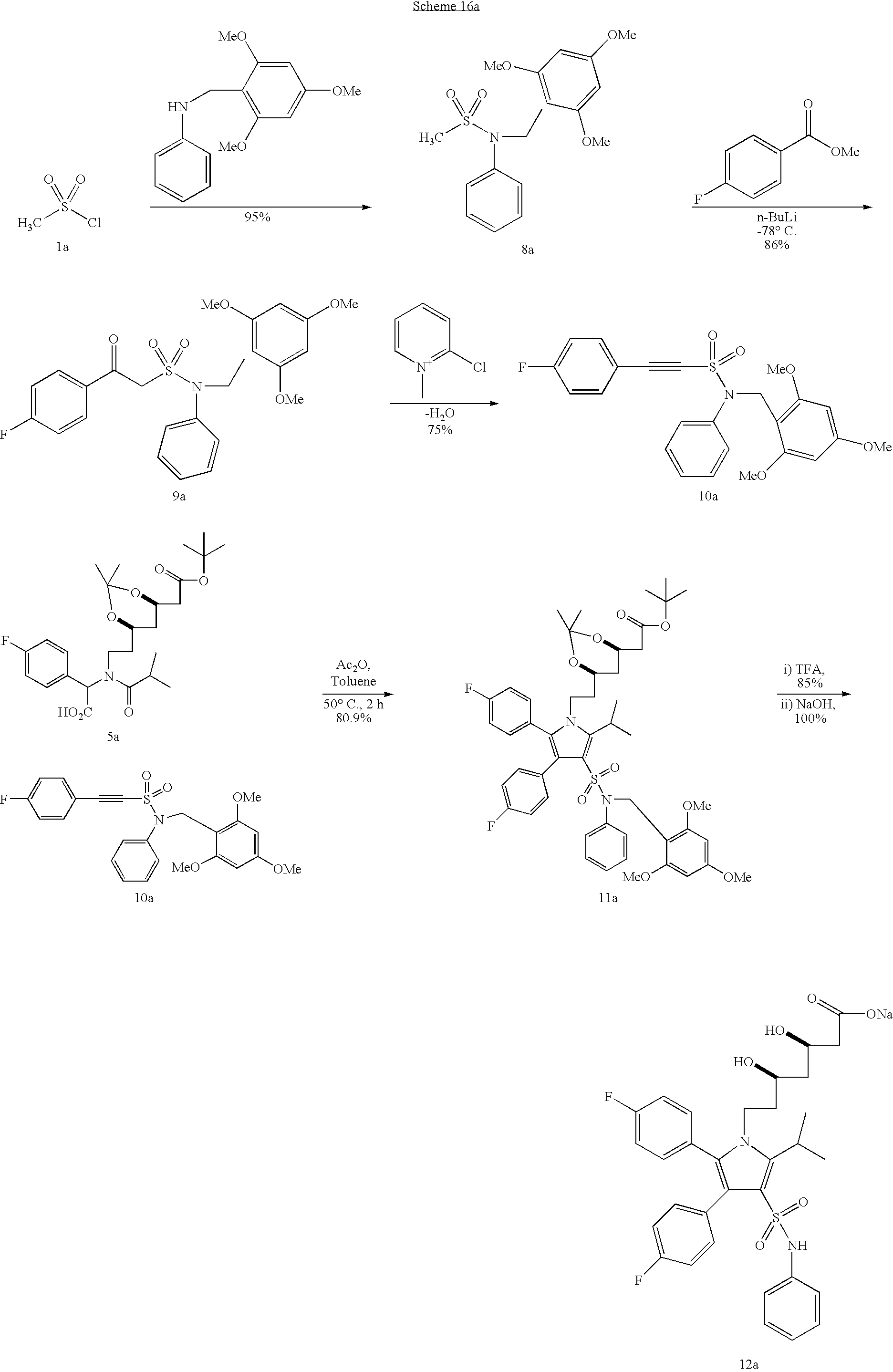

Both synthetic routes shown in Schemes 14 and 16 include aryl-substituted alkyne sulfonamides (compound 4 and 10). The conversion of β-ketosulfonamides with a non-aryl, for example an alkyl group, may be difficult with Mukiyama conditions (2-chloro N-methylpyridinium iodide and triethylamine). For example, in Scheme 17, the non-aryl group is isopropyl and NR⁹R¹⁰ taken together are morpholino-. The use of Tf₂O-Hunig's base (for example, diisopropylethylamine, DIEA) conditions for dehydration of β-ketosulfonamides overcomes this problem. As used herein, “Hunig's base” can include any strong organic base capable of deprotonation of β-ketosulfonamide, preferably, diisopropylethylamine.

Scheme 17 shows the dehydration of a non-aryl or an alkyl-substituted ,β-ketosulfonamides to the corresponding alkyne. Scheme 17a shows one example.

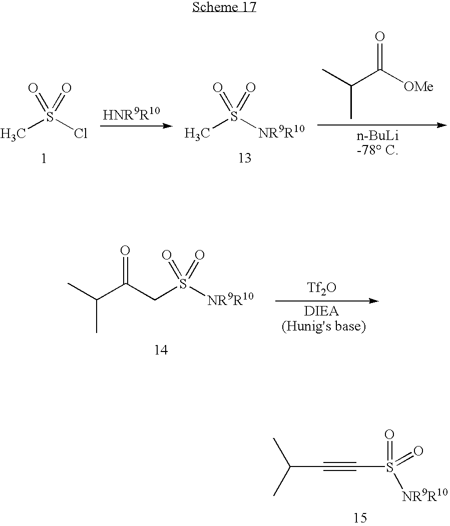

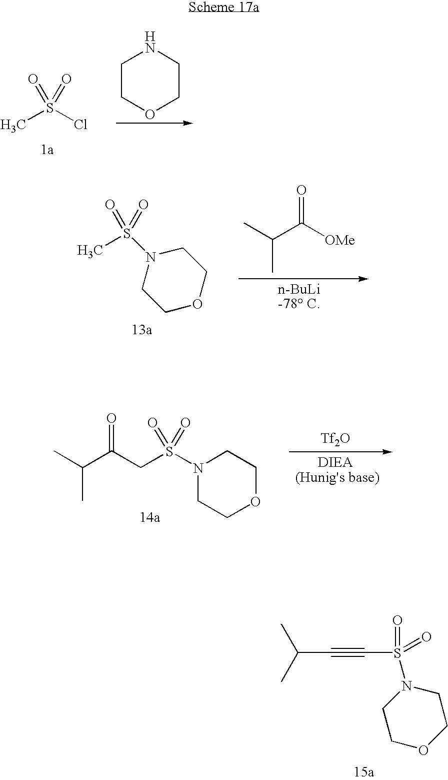

In Scheme 17, a variety of methyl esters including but not limited to those listed below may be used. An appropriate methyl ester is one that will result in the desired end product as would be known to one skilled in the art. Likewise, a variety of secondary amines including those listed below may be used.

Scheme 17, Examples of Methyl esters;

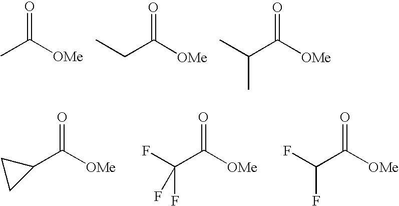

Scheme 17, Examples of secondary amines;

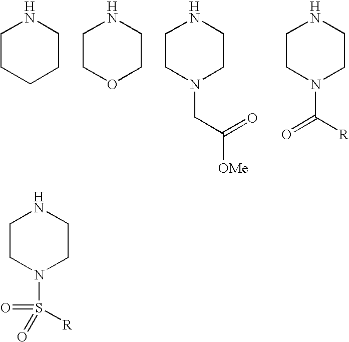

Scheme 18 shows the cycloaddition of a Munchnone precursor, 5′* and compound 15 via Munchnone reaction. The advantage of the reaction scheme shown in Scheme 18 is that the specific desired isomer e.g., 17, is obtained in high yield.

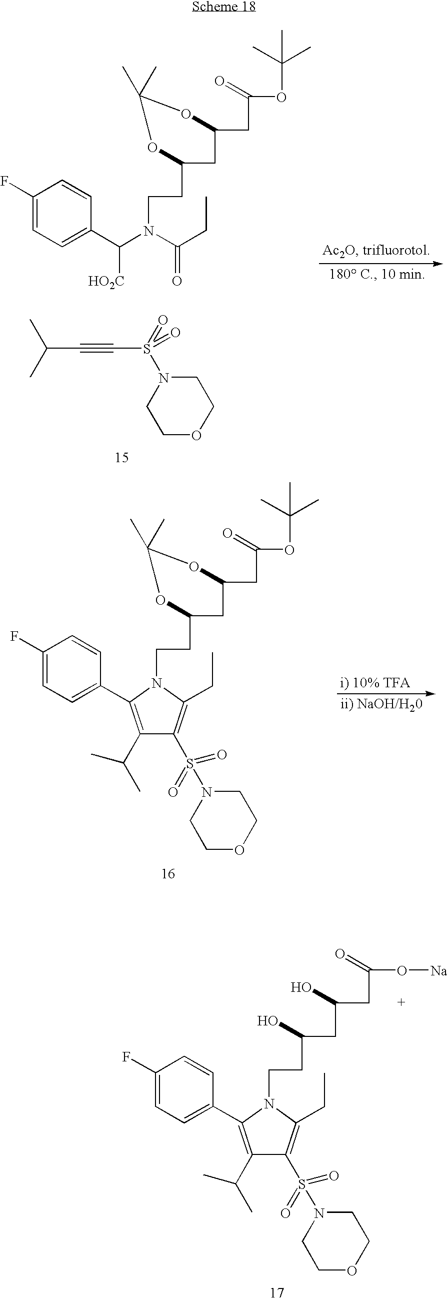

Scheme 19 shows, for example, the preparation of a compound of Formula I wherein R² and R³ are each parafluorophenyl, R⁴ is R⁶R⁷NC(O)— wherein one of R and R⁷ is H and the other one of R⁶ and R⁷ is SO₂R⁶, and R⁵ is isopropyl (compound 6). Scheme 19 also shows the preparation of a compound wherein R⁴ is R⁶R⁷N(C)O— and one of RC and R⁷ is H and the other one of R and R⁷ is SO₂NHR⁸(compound 4). Further, Scheme 19 shows the preparation of a compound wherein R⁴ is R⁶R⁷N(C)O— and R⁶ and R⁷ are each H(compound 3).

In scheme 19, condensation reaction of compound 1 (see Scheme 14a for preparation of acetonide) with sulfonyl isocyanate 5 gives compound 6; condensation of compound 1 with chlorosufonyl isocyanate gives compound 2. Treatment of compound 2 with an aryl amine gives compound 4. When compound 2 is reacted with benzylamine, compound 3 may be isolated.

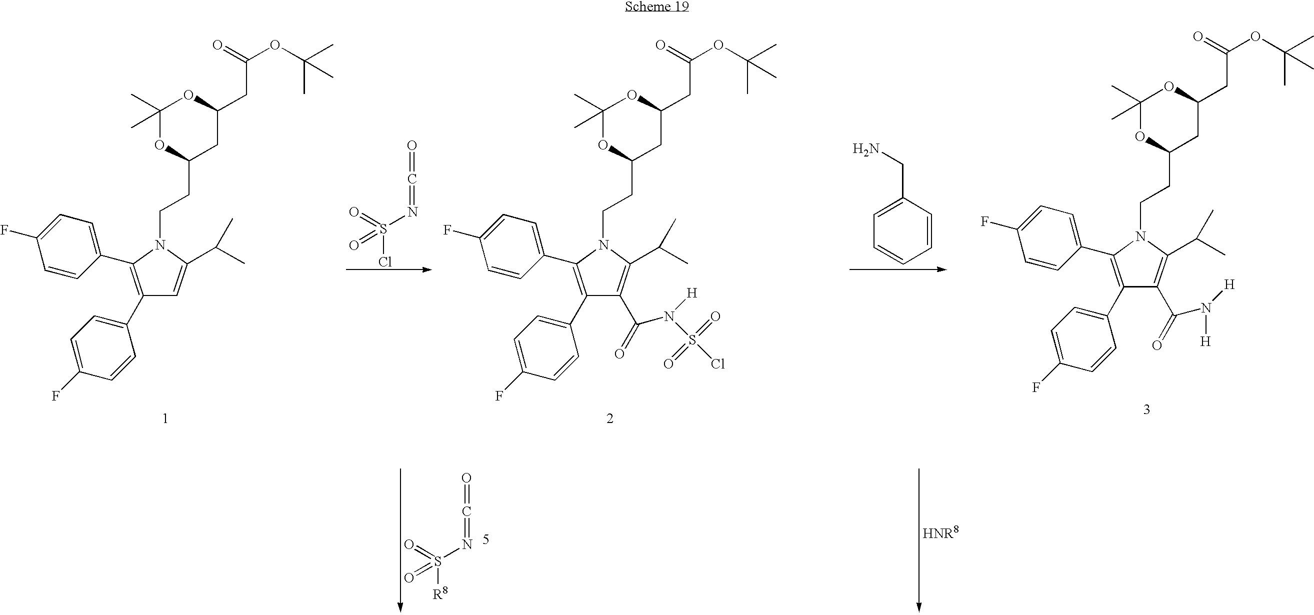

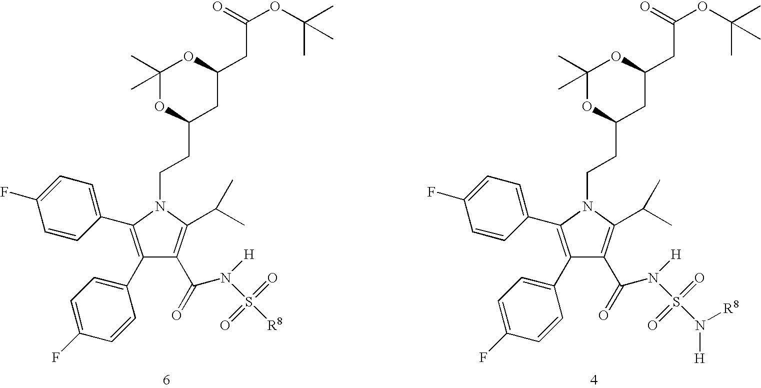

In Scheme 20, the acetonide functional group is hydrolyzed using HC1 (1N) in methanol, and the hydrolysis of the ester affords the desired product a sodium salt or di-sodium salt depending on the chemical nature of the R ⁴group.

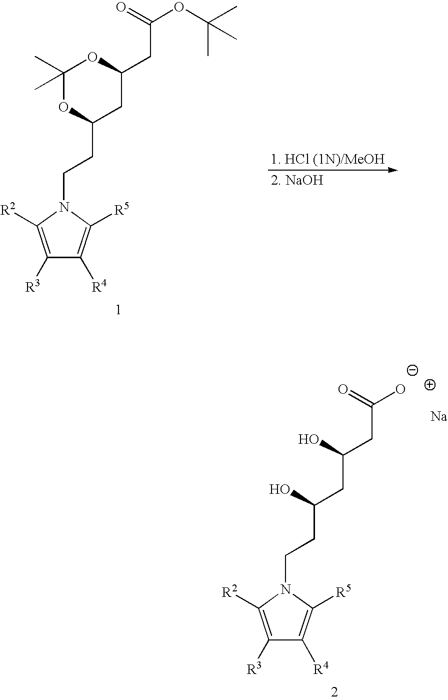

### EXAMPLES

Examples 1-424 relate to the preparation of imidazoles. Examples 1P-43P relate to the preparation of pyrroles.

### EXAMPLES

### Imidazoles

### EXAMPLES

The following non-limiting Examples show how to carry out the present invention. The synthetic route of compounds of the present invention is not limited to the methods outlined below. One skilled in the art will be able to use the schemes outlined below to synthesize various compounds claimed in this invention. Examples 1-3 illustrate preparations of useful intermediate compounds of the invention.

### Example 1

### 2-[[2-((4R,6R)-6-tert-Butoxycarbonylmethyl-2,2-dimethyl-[1,3]dioxan-4-yl)-ethyl]-(4-fluoro-benzoyl)-amino]-3-methyl-butyric acid

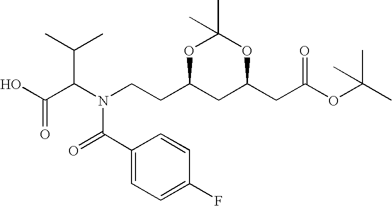

Step A

### 2-Hydroxy-3-methyl-butyric acid benzyl ester

A rt solution of 3-Methyl-2-oxo-butyric acid benzyl ester (20.0g, 97 mmol), prepared according to the method of M. Hesse et al (Helvetica Chimica Acta 2001, 84, 3766), in abs. EtOH (400 mL) was treated with sodium triacetoxyborohydride (25.0 g, 116 mmol) in portions over a period of 5 minutes. The reaction mixture became warm and the evolution of gas was noted. After stirring at rt for 12 h. The reaction mixture was concentrated to a slurry, diluted with water (300 mL), treated with sat. NaHCO₃ (pH ˜9), and extracted (2×) with hexanes/EtOAc (150 mL, 3:1). The combined extracts were dried (Na₂SO₄) and concentrated to a colorless oil. Purification by flash chromatography [SiO₂, EtOAc/hexanes 5-65%] provided the above named compound as a colorless liquid; yield: 17.7 g (87%); ¹H NMR (400 MHz, CD₃CN): δ 0.83 (d, J=6.8 Hz, 3H), 0.95 (d, J=7.0 Hz, 3H), 2.03 (m, 1H), 3.22 (d, J=6.1 Hz, 1H), 4.00 (dd, J=6.2, 4.2 Hz, 1H) 5.15 (d, J=12.2 Hz, 1H), 5.21 (d, J=12.2 Hz, 1 H), 7.38 (m, 5 H).

Step B

### 3-Methyl-2-trifluoromethanesulfonyloxy-butyric acid benzyl ester

According to the method of M. Walker (Tetrahedron 1997, 53,14591), a solution of 2-Hydroxy-3-methyl-butyric acid benzyl ester (16.0 g, 76.8 mmol) and 2,6-lutidine (10.74 mL, 92 mmol) in anhydrous CH₂CI₂ (300 mL) was cooled to −78° C. and treated with triflic anhydride, dropwise over a period of 5 minutes. The golden yellow reaction mixture was stirred at −78° C. for 30 min, then allowed to warm to rt. After stirring at rt for 1.5 h, the reaction mixture was poured into water (150 mL) and treated with 1 M HCl (150 mL). The organic layer was separated, dried (Na₂SO₄) and concentrated to a yellow-brown oil. Purification by flash chromatography [SiO₂, EtOAc/hexanes 5-15%] provides the above named compound as a colorless liquid; yield: 25.3 g (96%); 1H NMR (400 MHz, CD₃CN): δ 0.93 (d, J=8 Hz, 3 H), 1.05 (d, J=7.0 Hz, 3 H), 2.41 (m, 1 H), 5.22 (d, J=3.9 Hz, 1 H), 5.26 (d, J=12.2 Hz, 1 H), 5.29 (d, J=12.2 Hz, 1 H), 7.41 (m, 5 H).

Step C

### 2-[2-((4R,6R)-6-tert-Butoxycarbonylmethyl-2,2-dimethyl-[1,3]dioxan-4-yl)-ethylamino]-3-methyl-butyric acid benzyl ester

A solution of [(4R,6R)-6-(2-Amino-ethyl)-2,2-dimethyl-[1,3]dioxan-4-yl]-acetic acid tert-butyl ester (21.1 g, 77.1 mmol) and 3-Methyl-2-trifluoromethanesulfonyloxy-butyric acid benzyl ester (25 g, 73.5 mmol) in anhydrous acetonitrile was treated with TEA (12.3 mL, 88 mmol). The resulting mixture was allowed to stir at rt over the weekend (60 h). The reaction mixture was concentrated to a brown oil, poured into water (200 mL), made basic (pH >10) with 1 M NaOH, and extracted (2×) with hexane/EtOAc (1:1). The extracts were combined, washed with sat. NH₄Cl, dried (Na₂SO₄), and concentrated to a crude oil. Purification by flash chromatography [SiO₂, EtOAc/hexanes 5-60%] provided the above named compound as a mixture of diastereomers; yield: 30.6 g (89%); Low resolution mass spectroscopy (APCl) m/z 464 [M+H]⁺.

Step D

### 2-[[2-((4R,6R)-6-tert-Butoxycarbonylmethyl-2,2-dimethyl-[1,3]dioxan-4-yl)-ethyl]-(4-fluoro-benzoyl)-amino]-3-methyl-butyric acid benzyl ester

A solution of 2-[2-((4R,6R)-6-tert-Butoxycarbonylmethyl-2,2-dimethyl-[1,3]dioxan-4-yl)-ethylamino]-3-methyl-butyric acid benzyl ester (30 g, 64.7 mmol) in anhydrous pyridine was treated with and 4-fluorobenzoyl chloride (8 mL, 67.9 mmol). The mixture becomes warm (36° C.). The reaction was allowed to stir at rt overnight then concentrated to a brown slurry, poured into water (250 mL), made basic (pH >10) with 1 M NaOH, and extracted (2×) with hexane/EtOAc (1:1). The extracts were combined, washed with sat. NH₄Cl, dried (Na₂SO₄), and concentrated to a crude oil. Purification by flash chromatography [SiO2, EtOAc/hexanes 5-45%] provided the above named compound as a mixture of diastereomers; yield: 34.7 g (94%); Low resolution mass spectroscopy (APCI) m/z 496 [M+H]⁺ Analysis calculated for C₂₆H₃₈F₁N₁O7: C, 63.01; H, 7.73; N, 2.83. Found: C, 62.81; H, 7.82; N, 2.78.

Step E

A solution of 2-[[2-((4R,6R)-6-tert-Butoxycarbonylmethyl-2,2-dimethyl-[1,3]dioxan-4-yl)-ethyl]-(4-fluoro-benzoyl)-amino]-3-methyl-butyric acid benzyl ester (34.0 g, 58.0 mmol) in THF (200 mL) was hydrogenated over 20% Pd/C (2.0 g) until the uptake of hydrogen ceased (10 h). The solution was filtered through celite and concentrated to give the title compound as a colorless foam; yield: 24.4 g (84%); Low resolution mass spectroscopy (APCI) m/z 586 [M+H]⁺.

### Example 2

### 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid

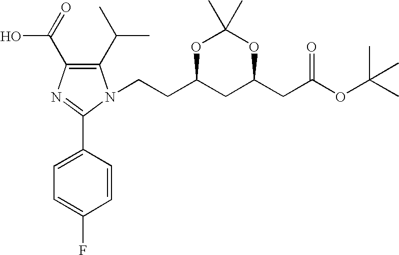

Step A

### Bromo-(4-fluoro-phenyl)-acetic acid methyl ester

According to the method of Y. Ishii et al (J. Org. Chem. 1998, 63, 6023), a solution of (4-Fluoro-phenyl)-acetic acid methyl ester (25 g, 0.15 mol) in ethyl acetate (300 mL) was added to an aqueous sodium bromate solution (67 g ; 0.45 mol in 225 mL water). The biphasic mixture was treated with 1M NaHSO₃ (450 mL) and the reaction was allowed to stir at ambient temperature for 6 h. The phases were separated, the organic layer was washed with NaOH and Sat. NH₄Cl, dried (Na₂SO4), and concentrated to give a yellow oil. Residual starting material was removed by vacuum distillation (75° C., <0.1 mm Hg); yield: 22.6 g (62%); Low resolution mass spectroscopy (APCI) m/z 2471249 [M+H]⁺; ¹H NMR (400 MHz, CDCl₃): δ 3.8 (s, 3 H), 5.3 (s, 1 H), 7.0 (t, J=8.7 Hz, 2 H), 7.5 (m, 2 H).

Step B

### [2-((4R,6R)-6-tert-Butoxycarbonylmethyl-2,2-dimethyl-[1,3]dioxan-4-yl)-ethylamino]-4-fluoro-phenyl)-acetic acid methyl ester

A solution of [(4R, 6R)-6-(2-Amino-ethyl)-2,2-dimethyl-[1,3]dioxan-4-yl]-acetic acid tert-but ester (26.3 g; 96 mmol) and bromo-(4-fluoro-phenyl)-acetic acid methyl ester ( 22.6 g; 92 mmol) in acetonitrile (200 mL) was treated with triethylamine (18.5 g; 182 mmol). After 30 minutes a considerable precipitate was noted. The reaction was allowed to stir at rt overnight then filtered to remove the precipitate. The filtrate was concentrated to dryness. The residue was dissolved in EtOAc, washed with H₂O and brine, dried (MgSO₄), and concentrated to give a crude oil. The oil was triturated with hexanes to give a white solid which was collected by vacuum filtration and air dried; yield: 38.1 g (95%); Low resolution mass spectroscopy (APCl) m/z 440 [M+H]⁺.

Step C

### {[2-((4R,6R)-6-tert-Butoxycarbonylmethyl-2,2-dimethyl-[1,3]dioxan-4-yl)-ethyl]-isobutyryl-amino}-(4-fluoro-phenyl)-acetic acid methyl ester

A solution of [2-((4R,6R)-6-tert-Butoxycarbonylmethyl-2,2-dimethyl-[1,3]dioxan-4-yl)-ethylamino]-(4-fluoro-phenyl)-acetic acid methyl ester (10 g; 23 mmol) and 2,6 lutidine (3.7 g; 34 mmol) in CH₂Cl₂ (100 mL) was cooled to −78° C. and treated with isobutyryl chloride (2.46 g 23.1 mmol). The reaction mixture was allowed to warm to rt and stirred overnight. The reaction was treated with 100 mL sat. aq. NaHCO₃ and the organic layer was separated, washed with 1 M HCl, and brine, dried (MgSO₄), and concentrated to a crude glass. Purification by flash chromatography (EtOAc/hexanes 0-60%) gave the above named compound as a yellow oil: yield 9.71 g (96%); Low resolution mass spectroscopy (APCI) m/z 510 [M+H]⁺.

Step D

{[2-((4R,6R)-6-tert-Butoxycarbonylmethyl-2.2-dimethyl-[1,3]dioxan-4-yl)-ethyl]-isobutyryl-amino}-4-fluoro-phenyl)-acetic acid

A solution of {[2-((4R,6R)-6-tert-Butoxycarbonylmethyl-2,2-dimethyl-[1,3]dioxan-4-yl)-ethyl]-isobutyryl-amino}-4-fluoro-phenyl)-acetic acid methyl ester (9.71 g; 19.1 mmol) in THF: H₂O ( 150 mL, 2:1) was treated with solid LiOH (2 g; 95 mmol) and the resulting mixture was stirred at rt overnight. The reaction mixture was diluted with H₂O and extracted with Hexanes-EtOAc (1:1). The aqueous layer was made acidic with 1 M HCl (pH ˜4) and extracted with CH₂Cl₂. The organic layers were combined, dried (MgSO₄) and concentrated to dryness. The residue was concentrated from diethyl ether until a white solid is obtained. Yield: 9.0 g (95%); Low resolution mass spectroscopy (APCl) m/z 494 [M−H]⁻.

Step E

### 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid benzyl ester

A solution of {[2-((4R,6R)-6-tert-Butoxycarbonylmethyl-2,2-dimethyl-[1,3]dioxan-4-yl)-ethyl]-isobutyryl-amino}-(4-fluoro-phenyl)-acetic acid (800 mg, 1.6 mmol) and benzyl cyanoformate (520 mg, 3.2 mmol) in α,α,α-trifluorotoluene (5 mL) was treated with acetic anhydride (0.228 mL, 2.4 mmol). The resulting mixture was heated to reflux until TLC analysis indicated the absence of starting material (4 h). The reaction mixture was cooled to rt, concentrated to a light yellow oil, and partitioned between EtOAc and 1 M NaHCO₃. The organic layer was separated, dried (Na₂SO₄), and concentrated to an oil. Purification by flash chromatography (SiO₂, EtOAc/hexanes 10-75%) provided the desired product as an oil; yield: 293 mg (16%); Low resolution mass spectroscopy (APCl) m/z 595 [M+H]⁺.

Step F

A solution of 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid benzyl ester (14.84 g, 24.95 mmol) in THF (200 mL) was hydrogenated over 20% Pd/C until the uptake of hydrogen ceased. The solution was filtered through celite and concentrated to give the title compound as a white foam; yield: 12.2 g (97%); Low resolution mass spectroscopy (APCl) m/z 505 [M+H]⁺; Anal. Calcd for C₂₇H₃₇FN₂O₆: C, 64.27; H, 7.39; N, 5.55. Found: C, 64.52; H, 7.53; N, 5.15.

### Example 3

### 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid

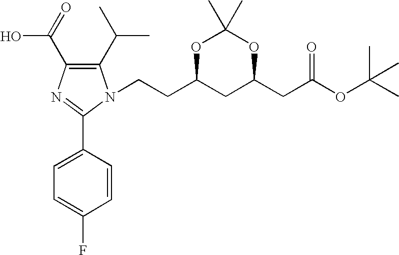

Step A

(Benzhydrylidene-amino)-acetic acid benzyl ester

Benzophenone imine (100.0 g, 496 mmol) and glycine benzylester hydrochloride (89.9 g, 496 mmol) were combined in CH₂Cl₂ (250 mL) and the resulting mixture was stirred at ambient temperature for 24 h. The reaction mixture was filtered to remove precipitated NH₄Cl and the filtrate was concentrated under reduced pressure. The residue was taken up in EtOAc, washed with 1 M NaHCO₃, dried with (Na₂SO₄), and concentrated to give off-white solid. Recrystallization from hot EtOAc-hexane gives the desired product as colorless plates; Yield: 123.6 g (76%); Low resolution mass spectroscopy (APCl) m/z 330 [M+H]⁺; Anal. Calcd for C₂₂H₁₉N₁O₂: C, 80.22.; H, 5.81; N, 4.25. Found: C, 80.16.; H, 5.77; N, 4.22.

Step B

### 2-amino-4-methyl-3-oxo-pentenoic acid benzyl ester hydrochloride

A cooled (dry ice-acetone bath) solution of KOtBu (6.81 g, 60.7 mmole, 60.7 mL THF solution) in anhydrous THF (100 mL) was treated with (Benzhydrylidene-amino)-acetic acid benzyl ester (20.0 g, 60.7 mmole) as a solution in THF (10 mL). After 30 min., this mixture was added via cannula to a cooled (dry ice-acetone bath) solution of isobutyryl chloride (60.7 mmole, 6.41 mL) in THF (50 mL). The resulting mixture was allowed to stir for 30 min, then quenched with 3N HCl solution (30 mL). The reaction mixture was warmed to rt and concentrated to dryness under reduced pressure. The residue was dissolved in water (20 mL) and extracted with ether (2×50 mL). The aqueous solution was concentrated to dryness under reduced pressure, concentrated twice from methanol, and re-dissolved residue in methanol. The insoluble salts were removed by filtration and the filtrate was concentrated to dryness. The residue was dissolved in THF (20 mL) and the above named compound was precipitated out upon addition of ether (50 mL); yield: ¹H NMR spectrum (400 MHz, CD₃OD) δ 7.30-7.37 (m, 5H), 5.18-5.29 (dd, J=23.8, 12.2 Hz, 2H), 3.00-3.06 (m, 1H), 1.13 (d, J=7.1 Hz, 3H), 1.00 (d, J=6.9 Hz 3H); Low resolution mass spectroscopy (APCl) m/z 236 [M+H]⁺.

Step C

### 2-(4-Fluoro-benzoylamino)-4-methyl-3-oxo-pentenoic acid benzyl ester

A solution of 2-amino-4-methyl-3-oxo-pentenoic acid benzyl ester hydrochloride (6.00 g, 22.1 mmole) in CH₂Cl₂ (50 mL), cooled in an ice-water bath, was treated sequentially with p-fluorobenzoyl chloride (1.1 equiv.), and TEA (2.2 equiv). After 2h the reaction mixture was diluted with EtOAc (25 mL) and washed sequentially with 1M HCl, 1 M NaHCO₃, and water. The organic layer was dried (Na₂SO₄) and concentrated under reduced pressure to give a crude yellow liquid that solidifies on standing. Recrystallization from hot ether-hexanes gave the above named compound as a colorless solid; yield 5.8 g (72%); Low resolution mass spectroscopy (APCl) m/z 358 [M+H]⁺; ¹H NMR (400 MHz, CDCl₃) δ 7.81 (dd, J=7.0, 4.8 Hz, 2H), 7.38-7.29 (m, 5H), 7.09 dd, J=8.5, 8.6 Hz, 2H), 5.60 (d, J=6.5 Hz, 1H), 5.22 (dd, J=21.2, 12.2 Hz, 2H), 3.00-3.07 (m, 1H), 1.20 (d, J=7.0 Hz, 3H), 1.00 (d, J=7.0 Hz, 3H).

Step D

### 1-[2-(6-tert-Butoxymethyl-2,2-dimethyl-[1,3]dioxan-4-yl)-ethyl]-2-(4-fluorophenyl)-5-isopropyl-1H-imidazole-4-carboxylic acid benzyl ester

A solution of 2-(4-Fluoro-benzoylamino)-4-methyl-3-oxo-pentenoic acid benzyl ester (1.50 g, 4.5 mmole), TBIA (1.5 equiv.), and acetic acid (glacial, 1.20 mL) acid in xylenes was warmed to 50° C. and treated with catalytic p-toluenesulfonic acid. The reaction mixture was heated to reflux for 24 h using a Dean-Stark trap charged with Na₂SO₄. The reaction mixture was cooled and concentrated under reduced pressure to give a light-brown amorphous residue.

This material was taken up in EtOAc (25 mL), washed with 1M HCl, NaHCO₃, water, and brine, dried (MgSO₄), and concentrated under reduced pressure to give an amorphous material. Purification by flash chromatography (SiO2, EtOAc/hexanes 0-20%) gave the above named compound as a tan glass; Yield: 1.39 g (55.69%); Low resolution mass spectroscopy (APCl) m/z 595 [M+H]⁺.

Step E

The title compound was prepared by following a process analogous to the one described in example 2, Step F

Example 4 and Example 4A exemplify the preparation of compounds of the invention wherein, for example, R² is 4-fluorophenyl, R⁴ is —(CH₂)ₙC(O)NR⁶R⁷, R⁵ is isopropyl, one of R⁶ and R⁷ is H, the other one of R⁶ and R⁷ is aralkyl or heteroaryl, and n is 0.

### Example 4

### Sodium, (3R,5R)-7-[2-(4-Fluoro-3phenyl)-5-isopropyl-4-benzylcarbamoyl-imidazol-1-yl-3,5-dihydroxy-heptanoate

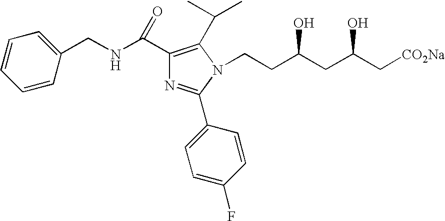

Step A

### 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid pentafluorophenyl ester

An ice cold solution of 1-[2-((4R, 6R)-6-tert-Butoxycarbonylmethyl-2,2-dimethyl-[1,3]dioxan-4-yl)-ethyl]-2-(4fluoro-phenyl)-5-isopropyl-1H-imidazole-4-carboxylic acid pentafluorophenyl ester

An ice cold solution of 1-[2-((4R, 6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid (9.33 g, 18.5 mmol) and 2,6-lutidine (3.96 g, 37 mmol) in acetonitrile (50 mL) was treated with pentafluorophenyl trifluoroacetate (7.77 g, 27.7 mmol). The resulting solution was stirred at rt for 2 h then treated with 1 M HCl. The reaction mixture was diluted with water and EtOAc. The organic layer was separated, washed with sat. NaHCO₃, dried (Na₂SO₄), and concentrated to a crude oil. Purification by flash chromatography (EtOAc/hexanes 5-40%) provided the above named product as a yellow glass; yield: 4.5 g (36%); Low resolution mass spectroscopy (APCl) m/z 671 [M+H]⁺; ¹H NMR (400 MHz, CDCl₃): δ 1.19 (dd, J=11.5, 24.2 Hz, 1 H), 1.30 (s, 3 H), 1.39(s, 3 H), 1.41 (s, 9 H), 1.46 (d, J=6.8 Hz, 3 H), 1.46 (d, J=6.8 Hz, 3 H), 1.48 (partially obscured m, 1 H), 1.76 (m, 2 H), 2.25 (dd, J=15.4, 6.3 Hz, 1 H), 2.40 (dd, J=15.4, 6.8 Hz, 1 H), 3.38 (septet, J=6.8 Hz, 1 H), 3.79 (m, 1 H), 3.95 (m, 1 H), 4.19 (m, 2H), 7.13 (m, 2 H), 7.56 (m, 2 H).

Step B

### 2-(4-Fluoro-Phenyl)-1-[2-((2R,4R)-4-hydroxy-6-oxo-tetrahydro-pyran-2-yl)-ethyl]-5-isopropyl-1H-imidazole-4-carboxylic acid benzylamide

A solution of 1-[2-((4R, 6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid pentafluorophenyl ester (1.0 mL, 0.298 mmol, 0.298 M in acetonitrile) was added to a screw-capped tube containing benzylamine (95 mg, 0.89 mmol) and resin bound DIEA (156 mg, loading 3.83 mmol/g) in acetonitrile (5 mL) . The mixture was allowed to stir at rt overnight, then treated with polystyrene bound isocyanate (600 mg, loading 1.49 mmol/g) and allowed to stir at rt for 6 hr. The spent resins were removed by filtration, rinsing with MeOH and acetonitrile, and the filtrate was concentrated to a crude oil. LC-MS is consistent with the desired amide (APCl) m/z 594 [M+H]⁺. The crude amide was dissolved in CH₂Cl₂ (4 mL), treated with neat TFA (1.0 mL) and allowed to stir at rt for 30 min. The reaction mixture was concentrated to an oil, then partitioned between CH₂Cl₂ and water and carefully neutralized with 1 M NaHCO₃ (pH ˜8). The organic layer was separated, dried (Na₂SO₄), and concentrated to a crude glass. Purification by flash chromatography (SiO₂, EtOAc/hexanes 60-100% gave a yellow glass; yield: 75 mg (52%); Low resolution mass spectroscopy (APCl) m/z 480 [M+H]⁺; ¹H NMR (400 MHz, CD₃CN) δ 7.96 (br t, 1H), 7.57-7.60 (m, 2H), 7.29-7.33 (m, 4H), 7.19-7.25 (m, 3H), 4.49-4.58 (m, 1H), 4.49 (d, J=6.6 Hz, 2H), 4.02-4.23 (m, 3H), 3.36 (septet, J=7.1 Hz, 1H), 3.29 (br s, 1H), 2.57 (dd, J=4.8, 17.5 Hz, 1H), 2.38 (ddd, J=1.7, 3.6, 17.5 Hz, 1H), 1.86-1.94 (m, 2H), 1.75-1.78 (m, 1H), 1.63 (ddd, J=3.1, 11.3, 17.3 Hz, 1H), 1.46 (d, J=7.1 Hz, 3H), 1.46 (d, J=7.1 Hz, 3H).

Step C

A solution of the 2-(4-Fluoro-phenyl)-1-[2-((2R, 4R)-4-hydroxy-6-oxo-tetrahydro-pyran-2yl)-ethyl]-5-isopropyl-1H-imidazole-4-carboxylic acid benzylamide (75 mg, 0.15 mmol) in THF (4 mL) was treated with aqueous NaOH (1.53 mL, 1.02 eq) The reaction mixture was allowed to stir @ rt for 30 min at which time analysis by loop LC-MS indicated that the starting material was consumed. The sample was concentrated to ca. 0.5 mL, diluted with water (30 mL) and lyophilized to give a colorless powder; yield: 79 mg (97%); Low resolution mass spectroscopy (APCl) m/z 498 [M+H]⁺; Anal. Calcd for C₂₇H₃₁F₁N₃O₅Na₁/1.7H2O: C, 58.94; H, 6.30; N, 7.64. Found: C, 58.84; H, 6.07; N, 7.34. ¹H NMR (400 MHz, DMSO-D6) □ 1.23 (m, 1 H); 1.40 (m, 7 H); 1.57 (m, 1 H); 1.69 (m, 1 H); 1.78 (dd, J=15.14, 8.30 Hz, 1 H); 1.97 (dd, J=15.14, 4.15 Hz, 1 H); 3.35 (m partially obscured, 1 H); 3.67 (m, 2 H); 3.94 (m, 1 H); 4.08 (m, 1 H); 4.40 (d, J=6.35 Hz, 2 H); 4.94 (br s, 1 H); 7.21 (m, 1 H); 7.30 (m, 6 H); 7.50 (br s, 1 H); 7.64 (m, 2 H); 8.38 (br t, J=6.35 Hz, 1 H).

### Example 4A

### Sodium: (3R, 5R)-7-[2-(4-Fluoro-phenyl)-5-isopropyl-4-phenylcarbamoyl-imidazol-1-yl]-3,5-dihydroxy-heptanoate

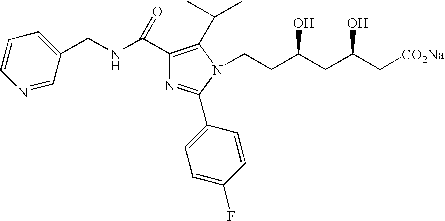

Step A

### [(4R,6R)-6-(2-{2-(4-Fluoro-phenyl)-5-isopropyl-4-[(pyridin-3-ylmethyl)-carbamoyl]-imidazol-1-yl}-ethyl)-2,2-dimethyl-[1,3]dioxan-4-yl]-acetic acid tert-butyl ester

A solution of 1-{2-[(4R,6R)-6-tert-butoxycarbonylmethyl-2,2-dimethyl-[1,3]dioxan-4-yl]-ethyl]-2-(4-fluoro-phenyl)-5-isopropyl-1H-imidazole-4-carboxylic acid (1.4 g; 2.8 mmol) in CH₂Cl₂ was treated with PyBOP (1.44 g, 2.8 mmol ), diisopropylethylamine (0.72 g, 5.5 mmol ), and 3-aminomethylpyridine (0.6 g, 5.5 mmol). The reaction was allowed to stir at rt for 2 hours. The reaction mixture was washed with H₂O, dried (MgSO₄), and concentrated to dryness. The residue was purified by flash chromatography (SiO₂; MeOH/EtOAc 0-10%) to give a white solid; yield: 500 mg (30%); Low resolution mass spectroscopy (APCl) m/z 595 [M+H]⁺.

Step B

### 2-(4-Fluoro-phenyl)-1-[2-((2R,4R)-4-hydroxy-6-oxo-tetrahydro-Pyran-2-yl)-ethyl]-5-isopropyl-1H-imidazole-4-carboxylic acid (pyridin-3-ylmethyl)-amide

A solution of [(4R,6R)-6-(2-(2-(4-Fluoro-phenyl)-5-isopropyl-4-[(pyridin-3-ylmethyl)-carbamoyl]-imidazol-1-yl}-ethyl)-2,2-dimethyl-[1,3]dioxan-4-yl]-acetic acid tert-butyl ester (500 mg, 0.8 mmol) in CH₂Cl₂ (4 mL) was treated with neat TFA (1 mL) and stirring was continued for 30 min. The reaction mixture was concentrated to dryness, then partitioned between CH₂Cl₂ and water and carefully neutralized with 1 M NaHCO₃ (pH ˜8). The organic layer was separated, dried (Na₂SO₄), and concentrated to a crude glass. Purification by flash chromatography (SiO₂, MeOH/EtOAc 0-10%) gives the lactone as a colorless solid; yield: 116 mg (29%); Low resolution mass spectroscopy (APCl) m/z 481 [M+H]⁺; ¹H NMR (400 MHz, CDCl₃): δ 1.43 (m, 6 H), 1.58 (m, 1 H), 1.76 (d, J=13.0 Hz, 2 H), 1.88 (m, 1 H), 2.56 (m, 1 H), 3.37 (m, 1 H), 4.06 (dq, J=7.3, 7.2 Hz, 2 H), 4.24 (m, 2 H), 4.56 (m, 3 H), 7.12 (t, J=8.4 Hz, 2 H), 7.21 (dd, J=7.6, 5.0 Hz, 1 H), 7.46 (dd, J=8.4, 5.3 Hz, 2 H), 7.66 (d, J=7.8 Hz, 1 H), 7.79 (t, J=6.1 Hz, 1 H), 8.40 (d, J=5.3 Hz, 1 H), 8.52 (s, 1 H).

Step C

The title compound was prepared by following a process analogous to the one described in example 4, Step C to give a colorless powder; yield: 102 mg (81%); Low resolution mass spectroscopy (APCl) m/z 499 [M+H]⁺; Anal. Calculated for C₂₆H₃F₁N₄O₅Na₁·2.65H₂O: C, 54.95; H, 6.26; N, 9.86. Found C, 55.03; H, 6.20; N, 9.46.

### Example 5

### Sodium; (3S,5R)-7-[2-(4-fluoro-phenyl)-5-isopropyl-4-(toluene-4-sulfonyl)-imidazol-1-yl]-3,5-dihydroxy-heptanoate

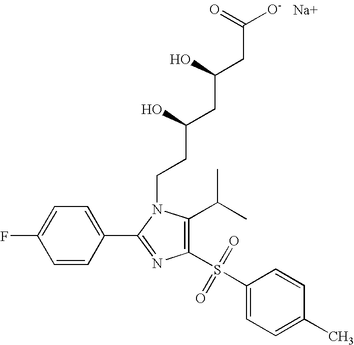

Step A

### ((4R,6R)-6-{2-[2-(4-Fluoro-phenyl)-5-isopropyl-4-(toluene-4-sulfonyl)-imidazol-1-yl]-ethyl}-2,2-dimethyl-[1,3]dioxan-4-yl)-acetic acid tert-butyl ester

A solution of {[2-((4R,6R)-6-tert-Butoxycarbonylmethyl-2,2-dimethyl-[1,3]dioxan-4-yl)-ethyl]-isobutyryl-amino}-(4-fluoro-phenyl)-acetic acid (250 mg; 0.5 mmol) and acetic anhydride ( 155 mg; 1.5 mmol) in toluene (10 mL) was combined with ptoluenesulfonyl cyanide (90 mg; 0.5 mmol) and heated to reflux for 1 hour. After cooling to rt the reaction mixture was washed with sat. aq. NaHCO₃, dried (MgSO₄), and concentrated to dryness. Purification of the residue by MPLC (SiO₂; EtOAc/hexanes 0-60%) gave the above named compound as a yellow film; yield:113 mg (36%); Low resolution mass spectroscopy (APCl) m/z 615 [M+H]⁺; ¹H NMR (400 MHz, CDCl₃) δ 1.23 (d, J=23.44 Hz, 6 H), 1.30 (m, 6 H), 1.37 (s, 9 H), 1.44 (m, 2 H), 2.24 (m, 5 H), 2.98 (septet, J=6.8 Hz, 1 H), 3.42 (q, J=7.1 Hz, 2 H), 3.57 (m, 1 H), 3.80 (m, 1 H), 4.07 (m, 1 H), 7.10 (m, 4 H), 7.21 (m, Hz, 2 H), 7.60 (dt, J=8.36, 1.80 Hz, 2 H).

Step B

### (4R,6R)-6-{2-[2-(4-Fluoro-phenyl)-5-isopropyl-4-(toluene-4-sulfonyl)-imidazol-1-yl]-ethyl}-4-hydroxy-tetrahydro-pyran-2-one

Prepared in a manner analogous to Example 4A, step B to give a white solid; yield: 77 mg (87%); Low resolution mass spectroscopy (APCl) m/z 501 [M+H]⁺; ¹H NMR (400 MHz, CDCl₃) δ 1.29 (m, 6 H), 1.49 (m, 1 H), 1.60 (m, 1 H), 1.69 (m, 1 H), 1.76 (m, 1 H), 2.34 (s, 3 H), 2.55 (d, J=3.78 Hz, 2 H), 3.01 (septet, J=6.7 Hz, 1 H), 3.67 (d, J=2.93 Hz, 1 H), 3.80 (m, 1 H), 3.94 (m, 1 H), 4.08 (q, J=7.1 Hz, 1 H), 4.30 (m, 1 H), 4.48 (m, 1 H), 7.10 (m, 2 H), 7.18 (m, 4 H), 7.54 (m, 2 H).

Step C

The title compound was prepared by following a process analogous to the one described in Examples 4, Step C, to give a colorless powder; yield: 66 mg (79%); Low resolution mass spectroscopy (APCl) m/Z 519 [M+H]⁺; Anal. Calculated for C₂₆H₂₉FN₂O₆SNa 1.55 H₂O; Theory: C, 54.93; H, 5.87; N, 4.93. Found C, 54.54; H, 5.52; N, 4.77.

### Example 6

### Sodium: (3R,5R)-7-[4-benzyloxycarbonyl-2-(4-fluoro-phenyl)-5-isopropyl-imidazol-1-yl -3,5-dihydroxy-heptanoate

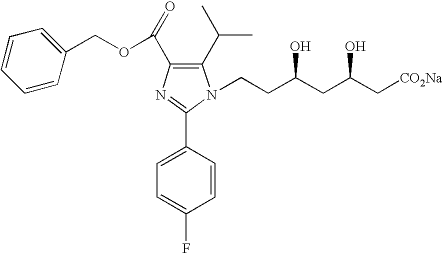

Step A

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid benzyl ester

A solution of 1-[2-((4R, 6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid benzyl ester (40 mg, 0.067 mmol) was dissolved in CH₂Cl₂ (5 mL), treated with neat TFA (1.0 mL) and allowed to stir at rt for 30 min. The reaction mixture was concentrated to an oil, then partitioned between CH₂Cl₂ and water and carefully neutralized with 1 M NaHCO₃ (pH ˜8). The organic layer was separated, dried (Na₂SO₄), and concentrated to a crude glass. Purification by flash chromatography (SiO₂, EtOAc/hexanes 60-100%) gives a colorless glass; yield: 30 mg (92%); Low resolution mass spectroscopy (APCl) m/z 481 [M+H]⁺; ¹H NMR (400 MHz, CD₃CN) δ 7.54-7.58 (m, 2H), 7.43-7.46 (m, 2H), 7.31-7.41 (m, 3H), 7.20-7.24 (m, 2H), 5.29 (s, 2H), 4.51 (ddd, J=3.6, 7.8, 15.6 Hz 1H), 4.05-4.22 (m, 3H), 3.40 (septet, J=7.1 Hz, 1H), 3.35 (br, s, 1 H), 2.57 (dd, J=4.7, 17.4 Hz, 1H), 2.38 (ddd, J=1.7, 3.4, 17.4 Hz, 1H), 1.86-193 (m, 2H), 1.69-1.75 (m, 1H), 1.61 (ddd, J=2.9, 11.2, 14.1 Hz, 1H), 1.43 (d, J=7.1 Hz, 3H), 1.43 (d, J=7.1 Hz, 3H).

Step B

The title compound was prepared following a process analogous to the one described in Examples 4, Step C, to give a colorless powder; yield: 28 mg (90%); Low resolution mass spectroscopy (APCl) m/z 49 [M+H]⁺; Anal. Calcd for C₂₇H₃₀F₁N₂Na₁O₆/1.3 H2O: C, 59.6.; H, 6.04; N, 5.15. Found: C, 59.28; H, 5.65; N, 4.89.

### Example 7

### Sodium, (3R,5R)-7-[2-(4-Fluoro-phenyl)-5-isopropyl-4-benzylcarbamoyl-imidazol-1-yl]-3,5-dihydroxy-heptanoate

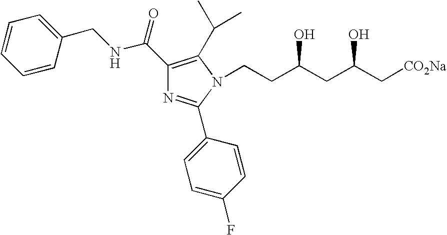

Step A

### N-Benzyl-2-oxo-acetamide

To a suspension of N,N′-dibenzyl-L-tartramide (3.07 g, 9.35 mmol) in THF (30 mL) was added periodic acid (2.13 g, 9.35 mmol) in two portions over 15 min. The mixture became slightly exothermic and slowly became homogeneous. After 1 hr, the solution was concentrated to give 5.0 g of a light orange foam, which was taken up in EtOAc, washed with saturated NaHCO₃ (2×), brine, dried over MgSO₄, and concentrated to give of a yellow foam which is a mixture of aldehyde and hydrate; yield: 2.90 g (95%); IH NMR (aldehyde) 8 9.34 (s, 1H), 7.40-7.20 (m, 5H), 4.51 (d, J=6 Hz); %); low resolution mass spectroscopy (APCl) m/z 162 [M−H]⁻.

Step B

### N-Benzyl-2,2-bis-(toluene-4-sulfonylamino)-acetamide

To a solution of crude N-benzyl-2-oxo-acetamide (2.80 g, 17.2 mmol) in toluene (40 mL) was added p-toluenesulfonamide (2.94 g, 17.2 mmol). The mixture was heated in an oil bath and initially became homogeneous, then a large amount of white precipitate formed before oil bath temp reached 100° C. The mixture was heated at reflux for 1 hr with a Dean Stark trap. The mixture was cooled and filtered to afford N-benzyl-2,2-bis-(toluene-4-sulfonylamino)-acetamide as an off-white solid; yield:3.68 g (88%); low resolution mass spectroscopy (APCl) m/z 486 [M−H]⁻; Anal. Calcd. for C₂₃H₂₅N₃O₅S₂: C, 56.66; H, 5.17; N, 8.62. Found: C, 56.85; H, 5.01; N, 8.58.

Step C

### (6-{2-[4-Benzylcarbamoyl-2-(4-fluoro-phenyl)-5-isopropyl-imidazol-1-yl]-ethyl}-2,2-dimethyl-[1,3]dioxan-4-yl)-acetic acid tert-butyl ester

To a solution of acid 2-[[2-((4R, 6R)-6-tert-Butoxycarbonylmethyl-2,2-dimethyl-[1,3]dioxan-4-yl)-ethyl]-(4-fluoro-benzoyl)-amino]-3-methyl-butyric acid (0.30 g, 0.605 mmol) in toluene (5 mL) was added EDC (0.128 g, 0.67 mmol) followed by N-benzyl-2,2-bis-(toluene-4-sulfonylamino)-acetamide 0.44 g, 0.91 mmol). The suspension was heated at 80-90° C. for 90 min. Additional EDC (45 mg, 0.4 eq) and bis-sulfonamide (0.15 g, 0.5 eq) were added and heating was continued for 3 hr. The mixture was cooled and filtered, washing with EtOAc. The filtrate was diluted with EtOAc, washed with sat. NaHCO₃, brine, dried over MgSO₄, and concentrated to give 0.49 g of a yellow foam. Flash chromatography (30-40% EtOAc/hexane) gave the title compound product as a white foam; yield: 0.13 g (36%); low resolution mass spectroscopy (APCl) m/z 594 [M+H]⁺.

Step D

The title compound is prepared by a process analogous to that described in Example 4A, Steps B and C.

Following a reaction scheme analogous to Examples 4 and 4A, a variety of esters, lactones and salts were prepared having the following variations on R², R⁴ and R⁵ (Examples 8-93). Such representative compounds follow along with characterizing data.

### Example 8

### 4-({[1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carbonyl]-amino}-methyl)-benzoic acid methyl ester

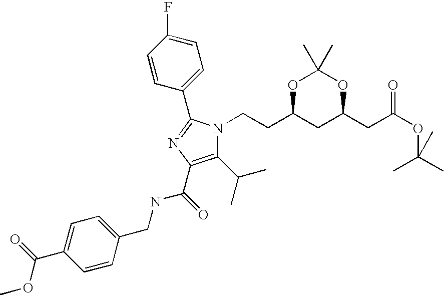

Obtain 255mg (39%) as a white solid.

Low resolution mass spectroscopy (APCl) m/z 652 [M+H]⁺. ¹H NMR (400 MHz, CDCl₃) δ 1.31 (s, 3 H) 1.35 (s, 3 H) 1.43 (s, 9 H) 1.46 (m, 2 H) 1.51 (dd, J=7.02, 3.36 Hz, 6 H) 1.76 (m, 2 H) 2.33 (m, 2 H) 3.42 (sept, J=21.4, 14.3, 7.3 Hz, 1 H) 3.79 (m, 1 H) 3.88 (s, 3 H) 3.94 (m, 1 H) 4.18 (m, 2 H) 4.63 (d, J=6.23 Hz, 2 H) 7.13 (t, J=8.67 Hz, 2 H) 7.40 (d, J=8.42 Hz, 2 H), 7.55 (dd, J=8.85, 531 Hz, 2 H) 7.80 (s, 1 H) 7.97 (m, 2 H).

### Example 9

### ((4R,6R)-6-{2-[4-(4-Dimethylsulfamoyl-benzylcarbamoyl)-2-(4-fluoro-phenyl)-5-isopropyl-imidazol-1-yl]-ethyl-2.2-dimethyl-[1,3]dioxan-4-yl)-acetic acid tert-butyl ester

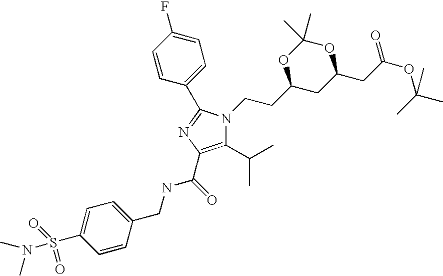

Obtain 220mg (44%) as a white solid.

Low resolution mass spectroscopy (APCl) m/z 701 [M−H]⁺. ¹H NMR (400 MHz, CDCl₃) δ 1.31 (s, 3 H) 1.35 (s, 3 H) 1.43 (s, 9 H) 1.47 (m, 2 H) 1.51 (dd, J=7.1, 3.3 Hz, 6 H) 1.76 (m, 2 H) 2.33 (m, 2 H) 2.67 (s, 6 H) 3.43 (sept, J=13.9, 6.9, 6.8 Hz, 1 H) 3.80 (m, 1 H) 3.94 (m, 1 H) 4.20 (m, 2 H) 4.66 (d, J=6.35 Hz, 2 H) 7.15 (t, J=8.6 Hz, 2 H), 7.51 (d, J=8.4 Hz, 2 H) 7.56 (dd, J=8.6, 5.4 Hz, 2 H) 7.71 (m, 2 H) 7.90 (s, 1 H).

### Example 10

### ((4R,6R)-6-{2-[4-(3-Dimethylcarbamoyl-benzylcarbamoyl)-2-(4-fluoro-phenyl)-5-isopropyl-imidazol-1-yl]-ethyl]-2,2-dimethyl-[1,3]dioxan-4-yl)-acetic acid tert-butyl ester

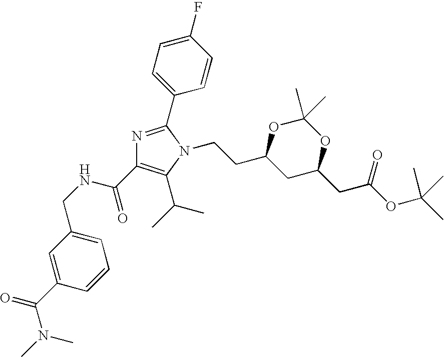

Obtain 143 mg (22%) as a white solid.

Low resolution mass spectroscopy (APCl) m/z 665 [M−H]⁺. ¹H NMR (400 MHz, CDCl₃) δ 1.13 (q, J=11.80 Hz, 1 H) 1.31 (s, 3 H) 1.34 (s, 3 H) 1.42 (s, 9 H) 1.50 (dd, J=7.0, 3.5 Hz, 6 H), 1.74 (m, 2 H) 2.25 (dd, J15.3, 6.3 Hz, 1 H) 2.40 (m, 1 H) 3.03 (m, 7 H) 3.42 (sept, J=20.8, 13.8, 6.8 Hz, 1 H) 3.79 (m, 1 H) 3.93 (m, 1 H) 4.18 (m, 2 H) 4.59 (d, J=6.2 Hz, 2 H) 7.12 (m, 2 H) 7.33 (m, 3 H) 7.54 (m, 2 H) 7.92 (m, 1 H) 10.03 (s, 1 H).

### Example 11

### [(4R,6R)-6-(2-{2-(4-Fluoro-phenyl)-5-isopropyl-4-[3-(piperidine-1-carbonyl)-benzylcarbamoyl]-imidazol-1-yl]-ethyl)-2,2-dimethyl-[1,3]dioxan-4-yl]-acetic acid tert-butyl ester

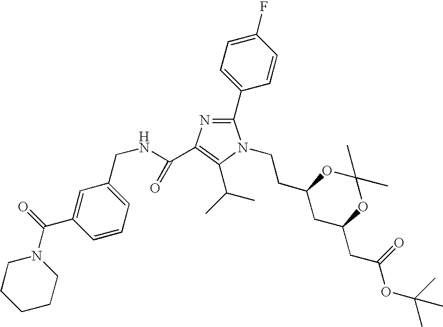

Obtain 124 mg (18%) as a white solid.

Low resolution mass spectroscopy (APCl) m/z 705 [M−H]⁺. ¹H NMR (400 MHz, CDCl₃) δ 1.15 (m, 2 H) 1.31 (s, 3 H) 1.35 (s, 3 H) 1.43 (s, 9 H) 1.51 (dd, J=7.1, 3.5 Hz, 6 H) 1.56 (s, 2 H) 1.64 (s, 4 H) 1.75 (m, 2 H) 2.25 (dd, J=15.4, 6.2 Hz, 1 H) 2.40 (m, 1 H) 3.31 (s, 2 H) 3.42 (sept, J=14.1, 6.9 Hz, 1 H) 3.67 (s, 2 H) 3.79 (m, 1 H) 3.93 (m, 1 H) 4.19 (m, 2 H) 4.60 (d, J=6.3 Hz, 2 H) 7.16 (m, 2 H) 7.35 (m, 4 H) 7.55 (m, 2 H) 7.75 (s, 1 H).

### Example 12

### [(4R,6R)-6-(2-{2-(4-Fluoro-phenyl)-5-isopropyl-4-[3-(morpholine-4-carbonyl)-benzylcarbamoyl]-imidazol-1-yl-ethyl)-2,2-dimethyl-[1,3]dioxan-4-yl]-acetic acid tert-butyl ester

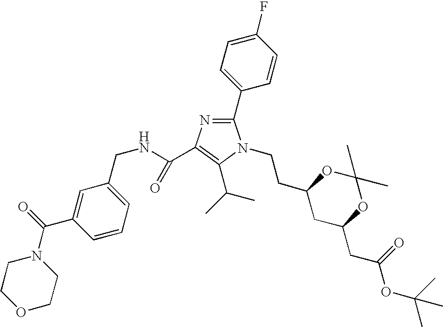

Obtain 116 mg (17%) as a white solid.

Low resolution mass spectroscopy (APCl) m/z 707 [M−H]⁺. ¹H MR (400 MHz, CDCl₃) δ ppm 1.16 (m, 2 H) 1.31 (s, 3 H) 1.35 (s, 3 H) 1.44 (m, 10 H) 1.50 (dd, J=7.1, 3.4 Hz, 6 H) 1.56 (s, 2 H) 1.76 (m, 2 H) 2.25 (m, J=15.4, 6.3 Hz, 1 H) 2.40 (m, J=15.4, 6.9 Hz, 1 H) 3.40 (m, J=20.9, 13.4, 6.9 Hz, 1 H) 3.60 (s, 2 H) 3.72 (s, 2 H) 3.79 (m, 2 H) 3.93 (m, 1 H) 4.18 (m, 2 H) 4.60 (d; J=6.2 Hz, 2 H) 7.14 (m, 2 H) 7.27 (t, J=1.5 Hz, 1 H) 7.34 (m, 1 H) 7.40 (m, 2 H) 7.54 (m, 2 H) 7.76 (s, 1 H)

### Example 13

### ((4R,6R)-6-{2-[2-(4-Fluoro-phenyl)-5-isopropyl-4-(4-methoxy-benzylcarbamoyl)-imidazol-1-yl]-ethyl]-2,2-dimethyl-[1,3]dioxan-4-yl)-acetic acid tert-butyl ester

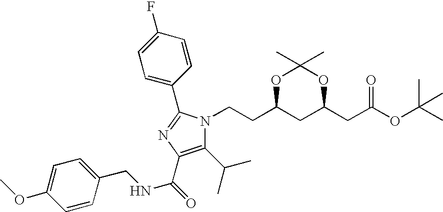

Obtain 472mg (76%) as a white solid.

Low resolution mass spectroscopy (APCl) m/z 624 [M−H]⁺. ¹H NMR (400 MHz, CDCl₃) δ ppm 1.12 (m, 1 H) 1.30 (s, 3 H) 1.34 (s, 3 H) 1.45 (m, 10 H) 1.51 (dd, J=7.1, 3.5 Hz, 6 H) 1.73 (m, 2 H) 2.24 (dd, J=15.4, 6.3 Hz, 1 H) 2.39 (m, J=15.3, 6.8 Hz, 1 H) 3.43 (m, J=21.1, 15.1, 7.0 Hz, 1 H) 3.76 (s, 3 H) 3.79 (m, 1 H) 3.92 (m, 1 H) 4.15 (m, 2 H) 4.50 (d, J=6.0 Hz, 2 H) 6.82 (m, 2 H) 7.11 (m, 2 H) 7.26 (m, 2 H) 7.52 (m, 2 H) 7.62 (t, J=5.9 Hz, 1 H).

### Example 14

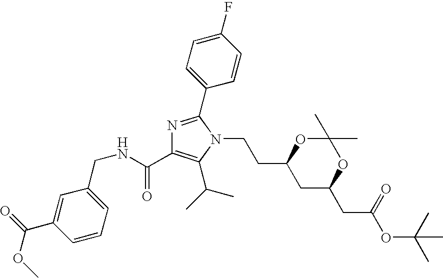

### 3-({[1-[2-((4R ,6R)-6-tert-Butoxycarbonylmethyl-2,2-dimethyl-[1,3]dioxan-4-yl)-ethyl]-2-(4-fluoro-phenyl)-5-isopronyl-1H-imidazole-4-carbonyl]-amino}-methyl)-benzoic acid methyl ester Obtain 107 mg (8%) as a white solid.

Low resolution mass spectroscopy (APCl) m/z 652 [M−H]⁺. ¹H NMR (400 MHz, CD₃OD) δ ppm 1.01 (m, 1 H) 1.19 (s, 3 H) 1.30 (s, 3 H) 1.36 (m, 10 H) 1.43 (dd, J=7.0, 1.4 Hz, 6 H) 1.64 (m, 2 H) 1.74 (m, 1 H) 2.20 (m, J=15.1, 7.8 Hz, 1 H) 3.40 (m, J=20.3, 13.3, 3.5 Hz, 1 H) 3.79 (m, 1 H) 3.82 (s, 3 H) 4.01 (m, 1 H) 4.17 (m, 2 H) 4.52 (s, 2 H) 7.18 (m, 2 H) 7.37 (t, J=7.7 Hz, 1 H) 7.57 (m, 3 H) 7.84 (m, 1 H) 7.96 (t, J=1.0 Hz, 1 H).

### Example 15

### 2-(4-Fluoro-phenyl)-1-[2-((2R ,4R)-4-hydroxy-6-oxo-tetrahydro-pyran-2-yl)-ethyl]-5-isopropyl-1H-imidazole-4-carboxylic acid (2-methoxy-ethyl)-amide

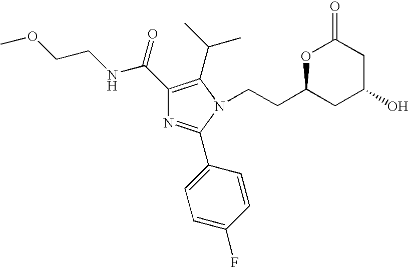

Low resolution mass spectroscopy (APCl) m/z 448 [M+H]⁺; ¹H NMR (400 MHz, CD₃CN): δ 7.57-7.62 (m, 3H), 7.21-7.27 (m, 2H), 4.53 (ddd, J=3.6, 8.0, 15.6 Hz 1H), 4.05-4.21 (m, 3H), 3.47 (m, 4H), 3.36 (septet, J=7.1 Hz, 1H), 3.32 (br s, 1H), 3.31 (s, 3H), 2.58 (dd, J=4.6, 17.3 Hz, 1H), 2.38 (ddd, J=1.8, 3.5, 17.3 Hz, 1H), 1.86-1.93 (m, 2H), 1.72-1.79 (m, 1H), 1.67(ddd, J=3.1, 11.3, 17.3 Hz, 1H), 1.47(d, J=7.1 Hz, 3H), 1.46(d, J=7.1 Hz, 3H), 1.46 (d, J=7.1 Hz, 3H).

### Example 16

### (4R,6R)-6-{2-[4-(1,3-Dihydro-isoindole-2-carbonyl)-2-(4-fluoro-phenyl)-5-isopropyl-1-yl]-ethyl}-4-hydroxy-tetrahydro-pyran-2-one

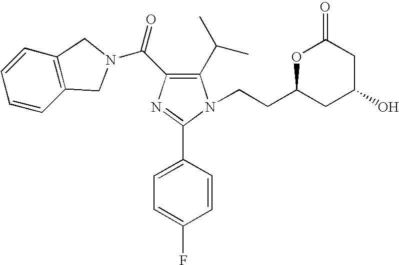

Low resolution mass spectroscopy (APCl) m/z 492 [M+H]+; ¹H NMR (400 MHz, CD₃CN): δ 7.64-7.68 (m, 2H), 7.21-7.38 (m, 6H), 5.05 (s, 2H), 4.87 (s, 2H), 4.53 (ddd, J=3.9, 7.8, 15.6 Hz 1H), 4.05-4.28 (m, 3H), 3.41 (br s, 1H), 3.24 (septet, J=7.1 Hz, 1H), 2.58 (dd, J=4.6, 17.6 Hz, 1H), 2.41 (ddd, J=1.4, 3.4, 17.3 Hz, 1H), 1.83-1.93 (m, 2H), 1.72-1.79 (m, 1H), 1.64 (ddd, J=3.2, 11.5, 14.4 Hz, 1H), 1.39 (apparent d, J=7.1 Hz, 6H).

### Example 17

### 2-(4-Fluoro-phenyl)-1-[2-((2R ,4R)-4-hydroxy-6-oxo-tetrahydro-pyran-2-yl)-ethyl]-5-isopropyl-1H-imidazole-4-carboxylic acid benzyl-ethyl-amide

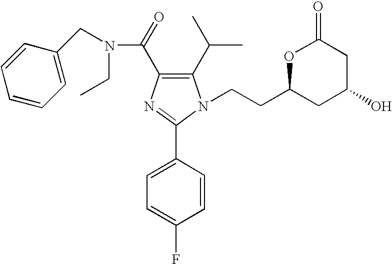

Low resolution mass spectroscopy (APCl) m/z 508 [M+H]⁺; ¹H NMR (400 MHz, CD₃CN) δ 7.53-7.63 (m, 2H), 7.16-7.59 (m, 7H), 4.69 (s, 2H), 4.50 (ddd, J=3.9, 7.8, 15.6 Hz 1H), 4.00-4.24 (m, 3H), 3.4 (m, 2H), 3.12 (m, 1H), 2.56 (apparent dt, J=4.4, 17.6 Hz, 1H), 2.38 (m,1H), 1.80-1.93 (m, 2H), 1.54-1.76 (m, 2H), 1.34 (apparent t, J=6.4 Hz, 6H) 1.13 (apparent dt, J=7.1, 13.9 Hz, 3H).

### Example 18

### 2-(4-Fluoro-phenyl)-1-[2-((2R ,4R)-4-hydroxy-6-oxo-tetrahydro-pyran-2-yl)-ethyl]-5-isopropyl-1H-imidazole-4-carboxylic acid phenylamide

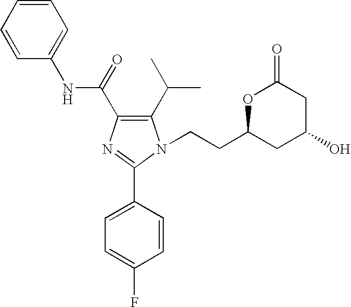

Low resolution mass spectroscopy (APCl) m/z 466 [M+H]⁺.

### Example 19

### 2-(4-Fluoro-phenyl)-1-[2-((2R ,4R)-(4-hydroxy-6-oxo-tetrahydro-pyran-2-yl))-ethyl]-5-isopropyl-1H-imidazole-4-carboxylic acid (biphenyl-4-ylmethyl)-amide

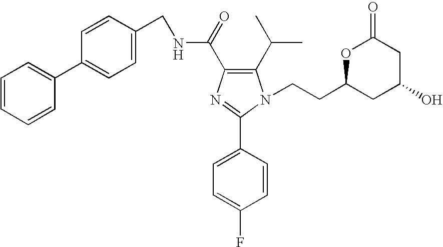

Low resolution mass spectroscopy (APCl) m/z 556 [M+H]⁺; ¹H NMR (400 MHz, CD₃CN) δ8.02 (br t, J=6.3 Hz, 1H), 7.56-7.63 (m, 6H), 7.31-7.45 (m, 5H), 7.24-7.19 (m, 2H), 4.50-4.54 (m, 3H), 4.05-7.25 (m, 3H), 3.36 (septet, J=7.1 Hz, 1H), 3.30 (obscured br s, 1H), 2.58 (dd, J=4.6, 173 Hz, 1H), 2.39 (ddd, J=1.7, 3.4, 17.3 Hz, 1H), 1.83-1.93 (m, 2H), 1.72-1.79 (m, 1H), 1.63 (ddd, J=3.2, 11.2, 14.4 Hz, 1 H), 1.47 (d, J=7.1 Hz, 3H), 1.47 (d, J=7.1 Hz, 3H).

### Example 20

### 2-(4-Fluoro-phenyl)-1-[2-((2R, 4R)-(4-hydroxy-6-oxo-tetrahydro-pyran-2-yl))-ethyl]-5-isopropyl-1H-imidazole-4-carboxylic acid 3-chloro-4-fluoro-benzylamide

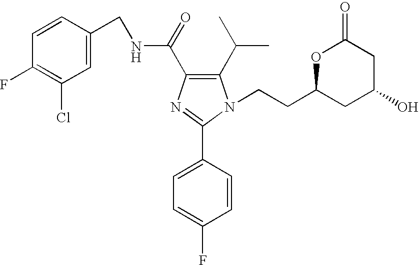

Low resolution mass spectroscopy (APCl) m/z 532 [M+H]⁺; ¹H NM R (400 MHz, CD₃CN) δ8.04 (br t, J=6.3 Hz, 1H), 7.56-7.61 (m, 2H), 7.41 (dd, J=2.2, 7.3 Hz, 1H), 7.15-7.29 (m, 4H), 4.53 (ddd, J=3.4, 7.8, 15.6 Hz 1H), 4.44 (d, J=6.4 Hz, 2H), 4.05-4.25 (m, 3H), 3.35 (septet, J=7.1 Hz, 1H), 3.28 (br s, 1H), 2.58 (dd, J=4.6, 17.3 Hz, 1H), 2.39 (ddd, J=1.7, 3.4, 17.3 Hz,1 H), 1.83-1.93 (m, 2H), 1.72-1.79 (m,1H), 1.63 (ddd, J=3.2, 11.2, 14.4 Hz,1H), 1.45 (d, J=7.1 Hz, 3H), 1.45 (d, J=7.1 Hz, 3H).

### Example 21

### 2-(4-Fluoro-phenyl)-1-[2-((2R ,4R)-(4-hydroxy-6-oxo-tetrahydro-pyran-2))-ethyl]-5-isopropyl-1H-imidazole-4-carboxylic acid 2,6-difluoro-benzylamide

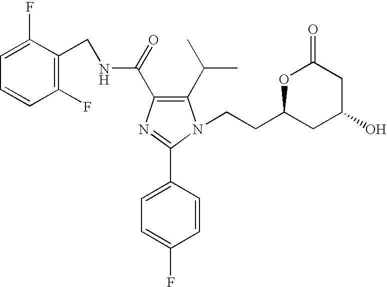

Low resolution mass spectroscopy (APCl) m/z 516 [M+H]⁺; ¹H NMR (400 MHz, CD₃CN) δ7.81 (br t, J=5.7 Hz, 1H), 7.54-7.61 (m, 2H), 7.41 (dd, J=2.2, 7.3 Hz, 1H), 7.15-7.29 (m, 4H). 4.59 (d, J=6.1 Hz, 2H), 4.51 (ddd, J=3.9, 7.6, 15.6 Hz 1H), 4.05-4.25 (m, 3H), 3.33 (septet, J=7.1 Hz, 1H), 3.28 ( br s, 1H), 2.56 (dd, J=4.6, 17.3 Hz, 1H), 2.38 (ddd, J=1.4, 3.4, 17.3 Hz, 1H), 1.83-1.93 (m, 2H), 1.71-1.79 (m, 1H), 1.62 (ddd, J=3.2, 11.2, 14.4 Hz,1H), 1.44 (d, J=7.1 Hz, 3H), 1.44 (d, J=7.1 Hz, 3H).

### Example 22

### 2-(4-Fluoro-phenyl)-1-[2-((2R ,4R)-(4-hydroxy-6-oxo-tetrahydro-pyran-2-yl))-ethyl]-5-isopropyl-1H-imidazole-4-carboxylic acid 3-fluoro-benzylamide

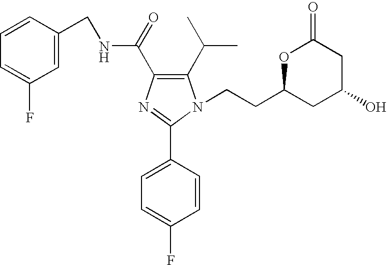

Low resolution mass spectroscopy (APCl) ml/z498 [M+H]⁺; ¹H NMR (400 MHz, CD₃CN) δ8.02 (br t, J=5.7 Hz, 1H), 7.58-7.61 (m, 2H), 7.33 (ddd, J=6.1, 7.8, 13.9 Hz, 1H), 7.20-7.24 (m, 2H), 7.12-7.18 (m, 1H), 7.04-7.09 (m, 1H), 6.97-7.05 (m, 1H), 4.53 (ddd, J=3.9, 7.6, 15.6 Hz 1H), 4.49 (d, J=6.6 Hz, 2H), 4.05-4.25 (m, 3H), 3.35 (septet, J=7.1 Hz, 1H), 3.28 (br s, 1H), 2.58 (dd, J=4.6, 17.3 Hz, 1H), 2.41 (ddd, J=1.4, 3.4, 17.3 Hz, 1H), 1.83-1.93 (m, 2H), 1.71-1.79 (m, 1H), 1.63 (ddd, J=3.2, 11.2, 14.4 Hz, 1H), 1.46 (d, J=7.1 Hz, 3H), 1.46 (d, J=7.1 Hz, 3H).

### Example 23

### 2-(4-Fluoro-phenyl)-1-[2-((2R,4R)-(4-hydroxy-6-oxo-tetrahydro-pyran-2-yl)-ethyl]-5-isopropyl-1H-imidazole-4-carboxylic acid (5-methyl-isoxazol-3-ylmethyl)-amide

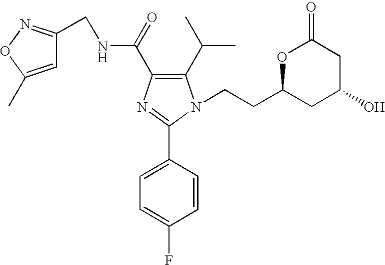

Low resolution mass spectroscopy (APCl) m/z 485 [M+H]⁺; ¹H NMR (400 MHz, CD₃CN) δ7.95 (br t, J=5.9 Hz,1H), 7.56-7.61 (m, 2H), 7.19-7.25 (m, 2H), 6.03 (m, 1H) 4.53 (ddd, J=2.9, 8.0, 15.8 Hz 1H), 4.48 (d, J=6.1 Hz, 2H), 4.05-4.25 (m, 3H), 3.35 (septet, J=7.1 Hz, 1H), 3.28 ( br s,1H), 2.58 (dd, J=4.7, 17.4 Hz, 1H), 2.41 (ddd, J=1.7, 3.7, 17.5 Hz, 1H), 2.35 (s, 3H), 1.83-1.93 (m, 2H), 1.71-1.79 (m, 1H), 1.63 (ddd, J=3.2, 11.5, 14.4 Hz, 1H), 1.46 (d, J=7.1 Hz, 3H), 1.46 (d, J=7.1 Hz, 3H).

### Example 24

### 2-(4-Fluoro-phenyl)-1-[2-((2R,4R)-(4-hydroxy-6-oxo-tetrahydro-pyran-2-yl))-ethyl]-5-isopropyl-1H-imidazole-4-carboxylic acid 4-fluoro-benzylamide

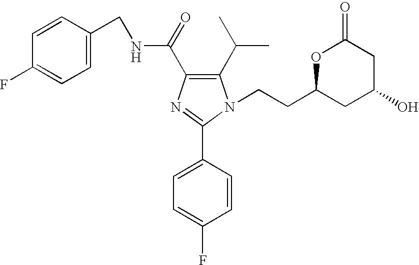

Low resolution mass spectroscopy (APCl) m/z 498 [M+H]⁺; ¹H NMR (400 MHz, CD₃CN) δ 7.97 (br t, J=6.4 Hz, 1H), 7.55-7.61 (m, 2H), 7.31-7.38 (m, 2H), 7.18-7.26 (m, 2H), 7.04-7.09 (m, 2H), 4.53 (ddd, J=4.2, 7.6, 15.6 Hz 1H), 4.46 (d, J=6.4 Hz, 2H), 4.05-4.25 (m, 3H), 3.35 (septet, J=7.1 Hz, 1H), 3.28 ( br s, 1H), 2.57 (dd, J=4.6, 17.6 Hz, 1H), 2.41 (ddd, J=1.7, 3.4, 17.3 Hz, 1H), 1.83-1.93 (m, 2H), 1.71-1.79 (m, 1H), 1.63 (ddd, J=3.2, 11.2, 17.3 Hz, 1H), 1.46 (d, J=7.1 Hz, 3H), 1.46 (d, J=7.1 Hz, 3H).

### Example 25

### 6-{2-[2-((2R,4R)-(4-Fluoro-phenyl)-5-isopropyl-4-(4-phenyl-piperazine-1-carbonyl)-imidazol-1-yl]-ethyl}-4-hydroxy-tetrahydro-pyran-2-one

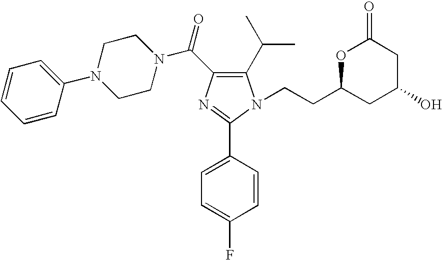

Low resolution mass spectroscopy (APCl) m/z 535 [M+H]⁺; ¹H NMR (400 MHz, CD₃CN): δ 1.36 (apparent d, J=6.8 Hz, 6 H), 1.63 (ddd, J=1 4.2, 11.2, 3.0 Hz, 1 H), 1.73 (m, 1 H), 1.91 (m, 2 H), 2.40 (ddd, J=17.4, 3.5, 1.7 Hz, 1 H), 2.58 (dd, J=17.3, 4.6 Hz, 1 H), 3.13 (m, 3 H), 3.19 (d, J=10.0 Hz, 2 H), 3.46 (s, 1 H), 3.72 (m, 2 H), 3.82 (m, 2 H), 4.10 (m, 1 H), 4.19 (m, 2 H), 4.51 (ddd, J=15.5, 7.8, 3.7 Hz, 1 H), 6.85 (m,1 H), 6.96 (m, 2 H), 7.24 (m, 4 H), 7.62 (m, 2 H).

### Example 26

### 6-{2-[2-((4R ,6R)-(4-Fluoro-phenyl)-5-isopropyl-4-(4-pyridin-2-yl-piperazine-1-carbonyl)- imidazol-1-yl-ethyl]-4-hydroxy-tetrahydro-pyran-2-one

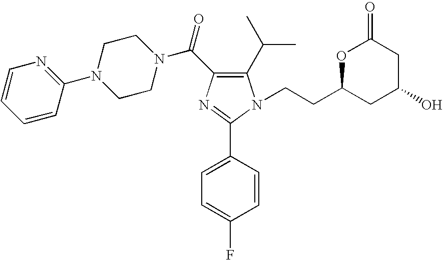

Low resolution mass spectroscopy (APCl) m/z 536 [M+H]⁺; ¹H NMR (400 MHz, CD₃CN): δ 1.36 (apparent d, J=7.0 Hz, 6 H), 1.63 (ddd, J=14.2, 11.2, 3.0 Hz, 1 H), 1.74 (m, 1 H), 1.90 (m, 2 H), 2.40 (ddd, J=17.5, 3.6, 1.5 Hz, 1 H), 2.58 (dd, J=17.3, 4.6 Hz, 1 H), 3.14 (septet, J=7.0 Hz, 1 H), 3.49 (m, 3 H), 3.58 (m, 2 H), 3.68 (m, 2 H), 3.78 (m, 2 H), 4.10 (m, 1 H), 4.19 (m, 2 H), 4.51 (ddd, J=15.3, 7.6, 3.7 Hz, 1 H), 6.65 (ddd, J=7.1, 4.9, 0.8 Hz, 1 H), 6.75 (m, 1 H), 7.23 (m, 2 H), 7.53 (ddd, J=8.7, 7.0, 2.0 Hz, 1 H), 7.63 (m, 2 H), 8.13 (ddd, J=4.8, 1.9, 0.7 Hz, 1 H).

### Example 27

### 2-(4-Fluoro-phenyl)-1-[2-((4R ,6R)(4-hydroxy-6-oxo-tetrahydro-pyran-2-yl)-ethyl]-5-isopropyl-1H-imidazole-4-carboxylic acid (2-phenoxy-ethyl)-amide

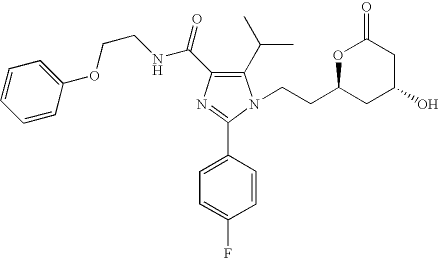

Low resolution mass spectroscopy (APCl) m/z 510 [M+H]⁺; ¹H NMR (400 MHz, CD₃CN): δ 1.48 (d, J=7.0 Hz, 3 H), 1.48 (d, J=7.0 Hz, 3 H), 1.64 (ddd, J=14.2, 11.2, 3.0 Hz, 1 H), 1.75 (m, 1 H), 1.89 (m, 2 H), 2.40 (ddd, J=17.5, 3.6, 1.7 Hz, 1 H), 2.59 (dd, J=17.5, 4.6 Hz, 1 H), 3.37 (septet, J=7.0 Hz, 1 H),, 3.37 (br s, 1 H), 3.70 (q, J=5.8 Hz, 2 H), 4.14 (m, 5 H), 4.53 (ddd, J=15.5, 7.8, 3.6 Hz, 1 H), 6.94 (m, 3 H), 7.26 (m, 4 H), 7.60 (m, 2 H), 7.75 (t, J=5.86 Hz, 1 H).

### Example 28

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid 3.4-dichloro-benzylamide

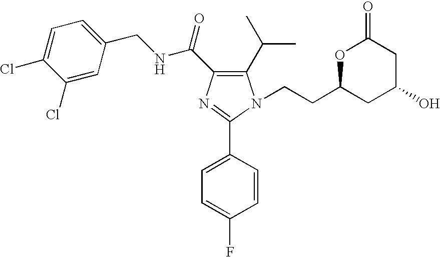

Low resolution mass spectroscopy (APCl) m/z 548/550/552 [M+H]⁺; ¹H NMR (400 MHz, CD₃CN): δ 1.45 (d, J=7.0 Hz, 3 H), 1.47 (d, J=7.0 Hz, 3 H), 1.65 (ddd, J=14.2, 11.2, 3.0 Hz, 1 H), 1.76 (m,1 H), 1.93 (m, 3 H), 2.40 (ddd, J=17.4, 3.5, 1.7 Hz, 1 H), 2.59 (dd, J=17.5, 4.64 Hz, 1 H), 3.36 (septet, J=7.0 Hz, 1 H), 4.11 (m, 1 H), 4.18 (m, 2 H), 4.45 (d, J=6.3 Hz, 2 H), 4.54 (ddd, J=15.5, 7.8, 3.6 Hz, 1 H), 7.23 (m, 3 H), 7.45 (m, 2 H), 7.60 (m, 2 H), 8.09 (t, J=6.3.Hz, 1 H).

### Example 29

### (4R,6R)-6-{2-[4-[4-(2,4-Difluoro-phenyl)-Piperazine-1-carbonyl]-2-(4-fluoro-phenyl)-5-isopropyl-imidazol-1-yl-ethyl]-4-hydroxy-tetrahydro-pyran-2-one

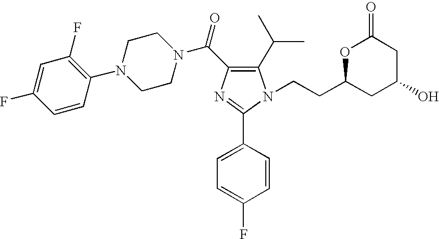

Low resolution mass spectroscopy (APCl) m/z 571 [M+H ¹H NMR (400 MHz, CD₃CN): δ 1.36 (d, J=7.0 Hz, 3 H), 1.36 (d, J=7.0 Hz, 3 H), 1.62 (ddd, J=1 4.2, 11.23, 3.0 Hz, 1 H), 1.73 (m, 1 H), 1.88 (m, 2 H), 2.39 (ddd, J=17.4, 3.5, 1.7 Hz, 1 H), 2.57 (dd, J=17.5, 4.6 Hz, 1 H), 2.95 (m, 2 H),, 3.02 (m, 2 H), 3.13 (septet, J=7.0 Hz, 1 H), 3.71 (m, 2 H), 3.82 (m, 2H), 4.12 (m, 3 H), 4.50 (ddd, J=15.3, 7.8, 3.6 Hz, 1 H) 6.90 (m, 2 H) 7.03 (td, J=9.2, 5.8 Hz, 1 H), 7.22 (m, 2 H) 7.61 (m, 2 H).

### Example 30

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid dibenzylamide

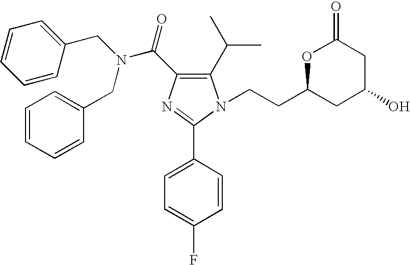

Low resolution mass spectroscopy (APCl) m/z 570 [M+H]⁺; ¹H NMR (400 MHz, CD₃CN): δ ppm 1.35 (d, J=7.0 Hz, 3 H), 1.35 (d, J=7.0 Hz, 3 H), 1.61 (ddd, J=14.2, 11.1, 3.1 Hz, 1 H), 1.72 (m, 1 H), 1.86 (m, 2 H), 2.38 (ddd, J=17.5, 3.6, 1.5 Hz, 1 H), 2.57 (dd, J=17.3, 4.6 Hz, 1 H), 3.14 (septet, J=7.0 Hz, 1 H), 3.35 (br s, 1 H), 4.13 (m, 3 H), 4.51 (m, J=7.8, 7.7, 7.7, 3.7 Hz, 1 H), 4.61 (s, 2 H), 4.74 (s, 2 H), 7.27 (m, 12 H), 7.59 (m, 2 H).

### Example 31

### 2-(4-Fluoro-phenyl)-1-[2-((2R ,4R)-4-hydroxy-6-oxo-tetrahydro-pyran-2-yl)-ethyl]-5-isopropyl-1H-imidazole-4-carboxylic acid ((R)-1-phenyl-ethyl)-amide

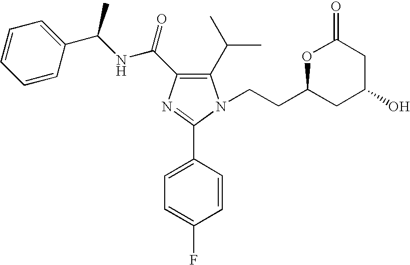

Low resolution mass spectroscopy (APCl) m/z 494 [M+H]⁺; ¹H NMR (400 MHz, CD₃CN) δ ppm 1.42 (d, J=6.8 Hz, 3 H), 1.46 (d, J=7.0 Hz, 3 H), 1.50 (d, J=7.0 Hz, 3 H), 1.64 (ddd, J=14.2, 11.3, 3.1 Hz, 1 H), 1.75 (m, J=14.2, 3.6, 3.6, 1.9 Hz, 1 H), 1.90 (m, 2 H), 2.40 (ddd, J=17.5, 3.6, 1.7 Hz, 1 H), 2.58 (dd, J=17.3, 4.6 Hz, 1 H), 3.34 (septet, J=7.0 Hz, 1 H), 3.34 (obscured brs, 1H), 4.14 (m, 3 H), 4.53 (ddd, J=15.6, 7.8, 3.6 Hz, 1 H), 5.15 (m, 1 H), 7.24 (m, 3 H), 7.35 (m, 4 H), 7.62 (m, 2 H), 7.80 (d, J=8.3 Hz, 1 H).

### Example 32

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid ((S)-1-Phenyl-ethyl)-amide

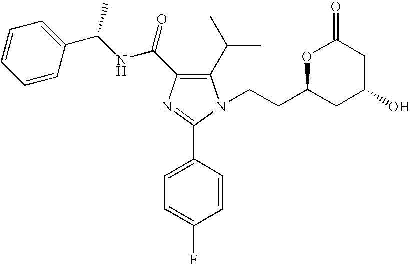

Low resolution mass spectroscopy (APCl) m/z 494 [M+H]⁺; ¹H NMR (400 MHz, CD₃CN): δ ppm 1.4 (d, J=7.1 Hz, 3 H), 1.5 (d, J=7.1 Hz, 3 H), 1.5 (d, J=17.1 Hz, 3 H), 1.6 (ddd, J=14.3, 11.4, 3.2 Hz, 1 H), 1.7 (m, J=14.3, 3.6, 3.6, 1.7 Hz, 1 H), 1.9 (m, 2 H), 2.4 (ddd, J=17.5, 3.5, 1.7 Hz, 1 H), 2.6 (dd, J=17.3, 4.6 Hz, 1 H), 3.3 (septet, J=7.0 Hz, 1 H), 4.1 (m, 3 H), 4.5 (ddd, J=15.6, 7.8, 3.7 Hz, 1 H), 5.1 (m, 1 H), 7.2 (m, 3 H), 7.3 (m, 4 H), 7.6 (m, 2 H), 7.8 (br d, J=8.3 Hz, 1 H).

### Example 33

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid 4-methanesulfonyl-benzylamide

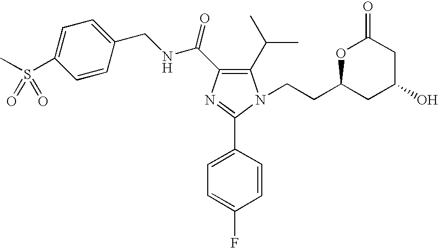

Low resolution mass spectroscopy (APCl) m/z 558 [M+H]⁺.

### Example 34

### 5-Ethyl-2-(4-fluoro-phenyl)-1-[2-((2R,4R)-4-hydroxy-6-oxo-tetrahydro-pyran-2-yl)-ethyl]-1H-imidazol-4-carboxylic acid phenylamide

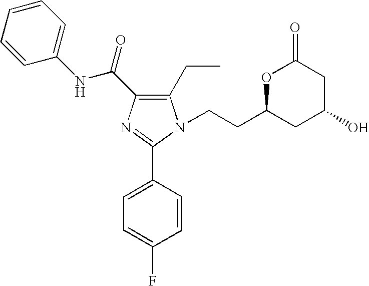

Low resolution mass spectroscopy (APCl) m/z 452 [M+H]⁺; ¹H NMR (400 MHz, CDCl₃) δ 1.32 (t, J=7.51 Hz, 3 H), 1.46 (m, 1 H), 1.64 (m, 1 H), 1.86 (m, 2 H), 2.39 (t, 1 H), 2.62 (m, 2 H), 3.15 (m, 1 H), 3.47 (q, J=6.9 Hz, 2 H), 4.13 (m, 1 H), 4.32 (m, 1 H), 4.58 (m, 1 H), 7.08 (m, 1 H), 7.19 (m, 2 H), 7.32 (m, 1 H), 7.39 (m, 1 H), 7.57 (m, 2 H), 7.68 (m, 2 H), 9.11 (s, 1 H).

### Example 35

### 5-Ethyl-2-(4-fluoro-phenyl)-1-[2-((2R,4R)-4-hydroxy-6-oxo-tetrahydro-pyran-2-yl)-ethyl]-1H-imidazole-4-carboxylic acid benzylamide

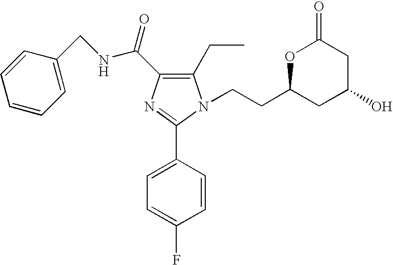

¹H NMR (400 MHz, CDCl₃) δ 1.16 (t, J=6.6 Hz, 4 H), 1.30 (t, J=7.5 Hz, 1 H), 1.44 (m, 1 H), 1.74 (m, 4 H), 2.60 (m, 2 H), 3.12 (m, 1 H), 3.45 (m, 1 H), 3.73 (s, 2 H), 4.22 (m, 1 H), 4.52 (m, 1 H), 7.06 (m, 2 H), 7.17 (m, 1 H), 7.28 (m, 2 H), 7.34 (m, 2 H), 7.56 (m, 2 H), 8.19 (t, J=7.93 Hz, 1 H).

### Example 36

### 5-Ethyl-2-(4-fluoro-phenyl)-1-[2-((2R ,4R)-4-hydroxy-6-oxo-tetrahydro-pyran-2-yl)-ethyl]-1-H-imidazole-4-carboxylic acid phenethyl-amide

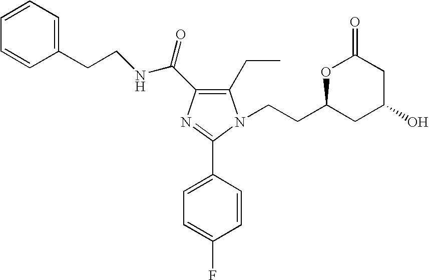

¹H NMR (400 MHz, CDCl₃) δ 1.41 (d, J=6.59 Hz, 3 H), 1.46 (m, 4 H), 1.63 (m, 1 H), 1.84 (m, 2 H), 2.60 (m, 2 H), 2.89 (m, 1 H), 3.13 (m, 2 H), 3.62 (m, 1 H), 3.70 (m, 1 H), 4.11 (m, 1 H), 4.29 (m, 1 H), 4.56 (m, 1 H), 7.20 (m, 4 H), 7.29 (m, 2 H), 7.38 (m, 1 H), 7.53 (m, 2 H).

### Example 37

### 5-Ethyl-2-(4-fluoro-phenyl)-1-[2-((2R,4R)-4-hydroxy-6-oxo-tetrahydro-pyran-2-yl)-ethyl]-1H-imidazole-4-carboxylic acid 4-fluoro-benzylamide

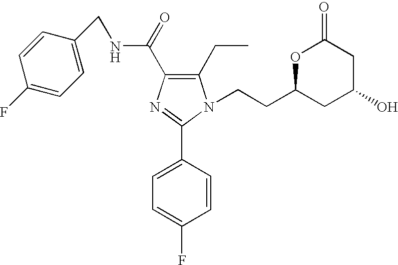

Low resolution mass spectroscopy (APCl) m/z 484 [M+H]⁺. ¹H NMR (400 MHz, CDCl₃) δ 1.30 (t, J=7.50 Hz, 2 H), 1.44 (m, 4 H), 1.62 (m, 1 H), 1.85 (m, 2 H), 2.60 (m, 1 H), 3.13 (m, 2 H), 3.69 (m, 1 H), 4.11 (m, 1 H), 4.26 (m, 1 H), 4.34 (m, 1 H), 4.53 (d, J=6.1 0 Hz, 2 H), 6.98 (m, 2 H), 7.17 (m, 2 H), 7.31 (m, 2 H), 7.53 (m, 2 H), 7.62 (t, J=4.70 Hz, 1 H).

### Example 38

### 2-(4-Fluoro-phenyl)-1-[2-((2R,4R)-4-hydroxy-6-oxo-tetrahydro-pyran-2-yl)-ethyl]-5-propyl-1H-imidazole-4-carboxylic acid phenylamide

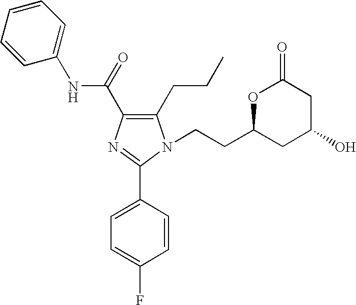

Low resolution mass spectroscopy (APCl) m/z 466 [M+H]⁺. ¹H NMR (400 MHz, CDCl₃) δ 1.06 (t, J=7.32 Hz, 3 H), 1.62 (m, 2 H), 1.77 (m, 3 H), 1.89 (m, J=14.29, 9.45, 9.45, 4.76 Hz, 1 H), 2.19 (s, 1 H), 2.59 (m, 2 H), 3.08 (dd, J=9.09, 6.65 Hz, 2 H), 4.11 (m, 1 H), 4.29 (m, 2 H), 4.56 (m, J=11.76, 9.29, 3.02, 3.02 Hz, 1 H), 7.07 (t, J=7.44 Hz, 1 H), 7.21 (t, J=8.66 Hz, 2 H), 7.32 (m, 2 H), 7.58 (dd, J=8.91, 5.25 Hz, 2 H), 7.66 (d, J=8.66 Hz, 2 H), 9.10 (s, 1 H).

### Example 39

### 2-(4-Fluoro-phenyl)-1-[2-((2R,4R)-4-hydroxy-6-oxo-tetrahydro-pyran-2-yl)-ethyl]-5-propyl-1H-imidazole-4-carboxylic acid benzylamide

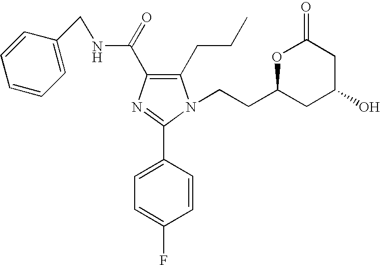

Low resolution mass spectroscopy (APCl) m/z 480 [M+H]+.¹H NMR (400 MHz, CDCl₃) δ 1.04 (t, J=7.32 Hz, 3 H), 1.59 (m, 1 H), 1.73 (m, 4 H), 1.86 (m, 1 H), 2.36 (s, 1 H), 2.57 (m, 2 H), 3.05 (m, 2 H), 4.06 (m, 1 H), 4.26 (m, 2 H), 4.52 (m, 1 H), 4.57 (d, J=6.1 Hz, 2 H), 7.15 (t, J=8.7 Hz, 2 H), 7.22 (m, 1 H), 7.29 (m, 2 H), 7.32 (m, 2 H), 7.53 (m, 2 H).

### Example 40

### 2-(4-Fluoro-phenyl)-1-[2-((2R,4R)-4-hydroxy-6-oxo-tetrahydro-pyran-2-yl)-ethyl]-5-propyl-1H-imidazole-4-carboxylic acid Phenethyl-amide

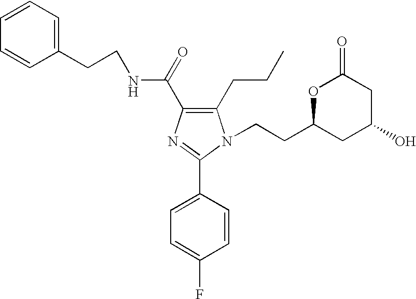

Low resolution mass spectroscopy (APCl) m/z 494 [M+H]⁺. ¹H NMR (400 MHz, CDCl₃) δ 1.04 (t, J=7.32 Hz, 3 H), 1.60 (m, 1 H), 1.69 (m, 3 H), 1.78 (m, 1 H), 1.87 (m, 1 H), 2.33 (s, 1 H), 2.60 (m, 2 H), 2.89 (m, 2 H), 3.04 (m, 2 H), 3.62 (m, 2 H), 4.09 (m, 1 H), 4.25 (m, 1 H), 4.32 (m, 1 H), 4.55 (m, 1 H), 7.17 (m, 2 H), 7.22 (m, 2 H), 7.29 (m, 2 H), 7.33 (t, J=6.16 Hz, 1 H), 7.53(m,2H).

### Example 41

### 2-(4-Fluoro-phenyl)-1-[2-((2R,4R)-4-hydroxy-6-oxo-tetrahydro-pyran-2-yl)-ethyl]-5-propyl-1H-imidazole-4-carboxylic acid 4-fluoro-benzylamide

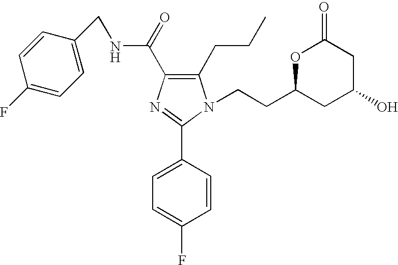

Low resolution mass spectroscopy (APCl) m/z 498 [M+H]⁺; ¹H NMR (400 MHz, CDCl₃) δ 1.05 (t, J=7.32 Hz, 2 H), 1.69 (m, 4 H), 1.87 (m, 1 H), 2.59 (m, 2 H), 3.06 (m, 2 H), 3.39 (s, 1 H), 4.11 (m, 1 H), 4.25 (m, 1 H), 4.31 (m, 2 H), 4.53 (d, J=5.98 Hz, 2 H), 6.98 (m, 2 H), 7.17 (m, 2 H), 7.30 (m, 2 H), 7.53 (m, 2 H), 7.71 (t, J=5.98 Hz, 2 H), 7.94 (s, 1 H).

### Example 42

### 2-(4-Fluoro-phenyl)-1-[2-((2R,4R)-4-hydroxy-6-oxo-tetrahydro-pyran-2-yl)-ethyl]-5-methyl-1H-imidazole-4-carboxylic acid phenylamide

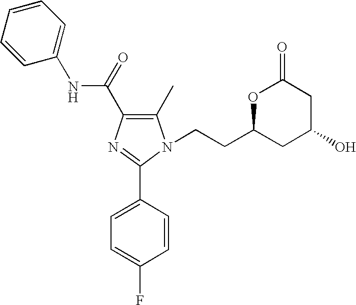

Low resolution mass spectroscopy (APCl) m/z 438 [M+H]⁺. ¹H NMR (400 MHz, CDCl₃) δ 1.39 (m, 1 H), 1.62 (m, 1 H), 1.87 (m, 2 H), 2.00 (s, 1 H), 2.58 (m, 1 H), 2.68 (s, 3 H), 3.32 (m, 1 H), 3.66 (m, 1 H), 4.08 (m, 1 H), 4.21 (m, 1 H), 4.31 (m, 1 H), 4.56 (m, 1 H), 7.05 (m, 1 H), 7.16 (m, 2 H), 7.30 (m, 2 H), 7.53 (m, 2 H), 7.63 (d, J=7.57 Hz, 2 H).

### Example 43

### 2-(4-Fluoro-phenyl)-1-[2-((2R,4R)-4-hydroxy-6-oxo-tetrahydro-pyran-2-yl)-ethyl]-5-methyl-1H-imidazole-4-carboxylic acid benzylamide

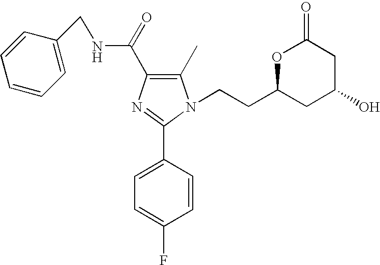

Low resolution mass spectroscopy (APCl) m/z 452 [M+H]⁺. ¹H NMR (400 MHz, CDCl₃) δ 1.63 (m, 1 H), 1.85 (m, 3 H), 2.59 (m, 2 H), 2.68 (s, 3 H), 3.11 (m, 1 H), 4.10 (m, 1 H), 4.23 (m, 1 H), 4.32 (m, 1 H), 4.55 (m, 2 H), 7.19 (m, 2 H), 7.31 (m, 3 H), 7.51 (m, 2 H), 7.65 (t, J=5.86 Hz, 2 H).

### Example 44

### 2-(4-Fluoro-phenyl)-1-[2-((2R,4R)-4-hydroxy-6-oxo-tetrahydro-pyran-2-yl)-ethyl]-5-methyl-1H-imidazole-4-carboxylic acid phenethyl-amide

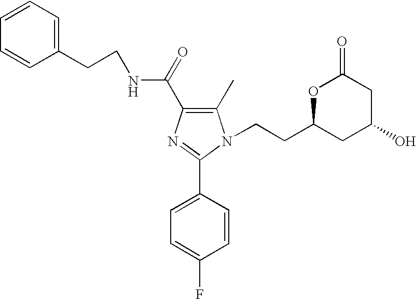

Low resolution mass spectroscopy (APCl) m/z 466 [M+H]⁺; ¹H NMR (400 MHz, CDCl₃) δ 1.41 (m, 2 H), 1.63 (m, 1 H), 1.82 (m, 2 H), 1.91 (m, 1 H), 2.61 (m, 4 H), 2.89 (m, 1 H), 3.11 (td, J=6.65, 3.66 Hz, 1 H), 3.61 (m, 1 H), 3.68 (m, 1 H), 4.10 (m, 1 H), 4.23 (m, 1 H), 4.33 (m, 1 H), 4.57 (m, 1 H), 7.19 (m, 4 H), 7.28 (m, 2 H), 7.43 (t, J=6.16 Hz, 1 H), 7.52 (m, 2 H), 10.04 (s, 1 H).

### Example 45

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid (biphenyl-3-ylmethyl)-amide

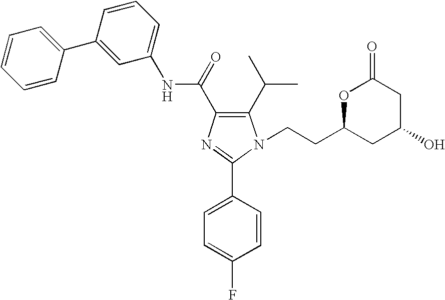

Low resolution mass spectroscopy (APCl) m/z 556 [M+H]⁺. ¹H NMR (400 MHz, CDC1₃) δ 1.53 (d, J=7.02, Hz, 3 H), 1.53 (d, J=7.02, Hz, 3 H), 1.65 (m, 1 H), 1.74 (s, 1 H), 1.80 (m, 2 H), 1.92 (m, 1 H), 2.61 (m, 2 H), 2.98 (m, 1 H), 4.11 (m, 1 H), 4.22 (m, 1 H), 4.33 (m, 1 H), 4.58 (m, 1 H), 4.64 (d, J=5.86 Hz, 2 H), 7.16 (m, 2 H), 7.37 (m, 4 H), 7.48 (m, 4 H), 7.57 (m, 1 H), 7.80 (s, 1 H).

### Example 46

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid phenethyl-amide

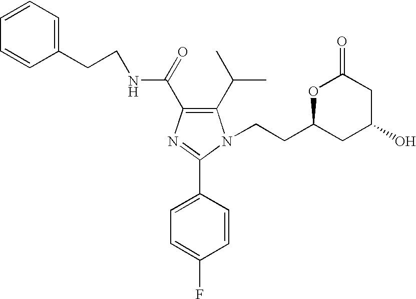

Low resolution mass spectroscopy (APCl) rrvz 494 [M+H]⁺.

### Example 47

### 2-(4-Fluoro-phenyl)-1-[2-((2R,4R)-4-hydroxy-6-oxo-tetrahydro-pyran-2-yl)-ethyl]-5-methyl-1H-imidazole-4-carboxylic acid 4-sulfamoyl-benzylamide

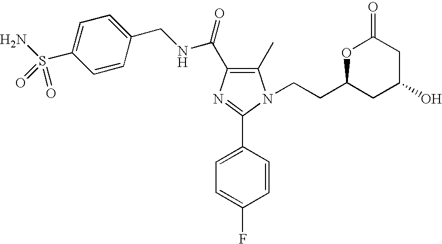

Low resolution mass spectroscopy (APCl) m/z 531 [M+H]⁺.

### Example 48

### 1-[2-((2R,4R)-4-Hydroxy-6-oxo-tetrahydro-pyran-2-yl)-ethyl-5-isopropyl-2-phenyl-1H-imidazole-4-carboxylic acid benzylamide

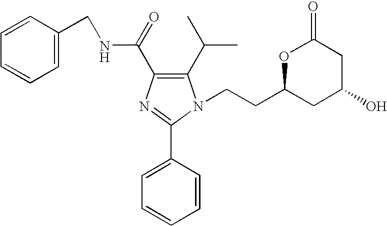

Low resolution mass spectroscopy (APCl) m/z 462 [M+H]⁺.

### Example 49

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid 3-chloro-benzylamide

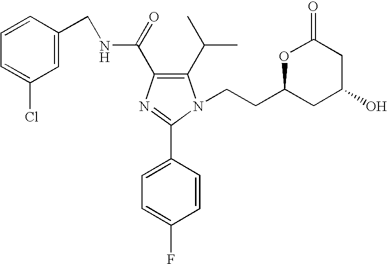

Low resolution mass spectroscopy (APCl) m/z 514 [M+H]⁺.

### Example 50

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid indan-1-ylamide

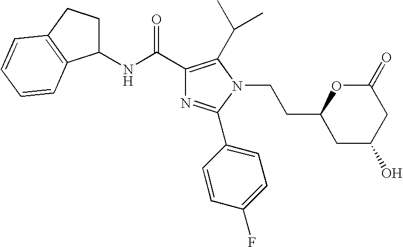

Low resolution mass spectroscopy (APCl) m/z 506 [M+H]⁺.

### Example 51

### (4R,6R)-6-{2-[2-(4-Fluoro-phenyl)-5-isopropyl-4-(3-phenyl-pyrrolidine-1-carbonyl)-imidazol-1-yl]-ethyl}-4-hydroxy-tetrahydro-pyran-2-one

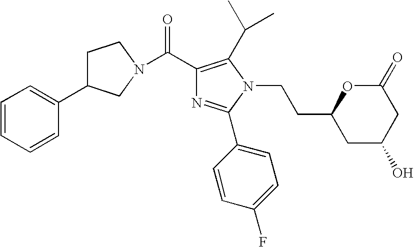

Low resolution mass spectroscopy (APCl) m/z 520 [M+H]⁺.

### Example 52

### (4R,6R)-6-{2-[4-(3-Benzenesulfonyl-pyrrolidine-1-carbonyl)-2-(4-fluoro-phenyl)-5-isopropyl-imidazol-1-yl]-ethyl]-4-hydroxy-tetrahydro-pyran-2-one

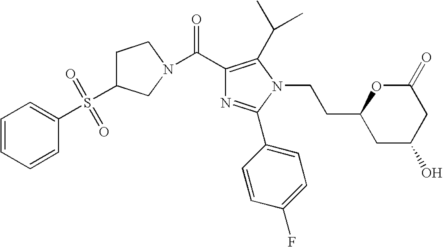

Low resolution mass spectroscopy (APCl) m/z 584 [M+H]⁺.

### Example 53

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid 4-sulfamoyl-benzylamide

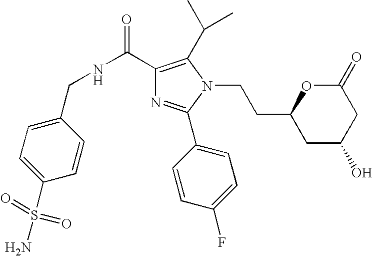

Low resolution mass spectroscopy (APCl) m/z 559 [M+H]⁺.

Following a scheme analogous to that described in Example 9, Step C a variety of sodium salts were prepared from the corresponding lactones having the following variations on R², R⁴ and R⁵

### Example 54

### Sodium: (3R,5R)-7-[5-(4-Fluoro-phenyl)-2-isopropyl-4-phenylcarbamoyl-imidazol-1-yl]-3,5-dihydroxy-heptanoate

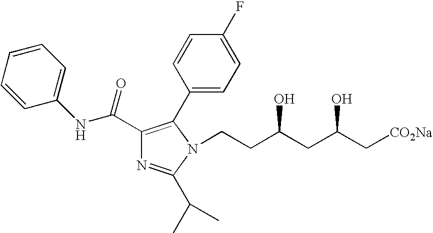

Low resolution mass spectroscopy (APCl) m/z 482 [M−H]⁻; Anal. Calcd for C₂₆H₂₉F₁N₃O₆/0.5 H₂O/1.0 NaOH: C, 56.32.; H, 5.63; N, 7.58. Found: C, 56.64; H, 5.38; N, 7.41.

### Example 55

### Sodium, (3R,5R)-7-[2-(4-Fluoro-phenyl)-5-isopropyl-4-(2-methoxy-ethylcarbamoyl)-imidazol-1-yl]-3,5-dihydroxy-heptanoate acid

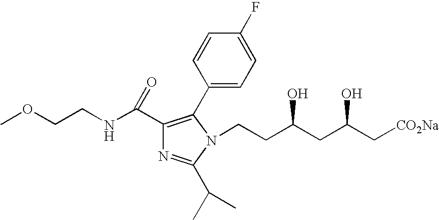

Low resolution mass spectroscopy (APCl) m/z 464 [M−H]⁻; Anal. Calcd for C₂₃H₃₁F₁N₃Na₁O₆/0.5 H₂O: C, 55.64.; H, 6.50; N, 8.46. Found: C, 55.86; H, 6.55; N, 8.33.

### Example 56

### (3R,5R)-7-[4-(1,3-Dihydro-isoindole-2-carbonyl)-2-(4-fluoro-phenyl)-5-isopropyl-imidazol-1-yl]-3,5-dihydroxy-heptanoate

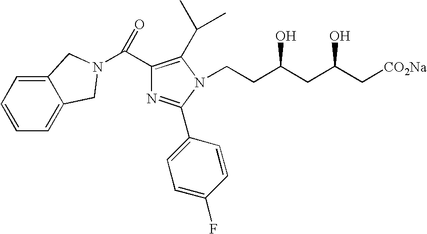

Low resolution mass spectroscopy (APCl) m/z 508 [M−H]⁻; Anal. Calcd for C₂₈H₃₁F₁N₃Na₁O₅/2.1 H₂O: C, 59.06.; H, 6.23; N, 7.38. Found: C, 58.81; H, 6.09; N, 7.18

### Example 57

### Sodium: (3R,5R)-7-[4-(Benzyl-ethyl-carbamoyl)-2-(4-fluoro-phenyl)-5-isopropyl-imidazol-1-yl]-3,5-dihydroxy-heptanoate

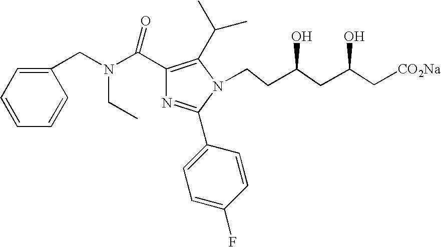

Low resolution mass spectroscopy (APCl) m/z524 [M−H]⁻; Anal. Calcd for C₂₉H₃₅F₁N₃Na₁O₅/1.0 H₂O: C, 61.58.; H, 6.59; N, 7.43. Found: C, 61.20.; H, 6.55; N, 7.23.

### Example 58

### Sodium; (3R,5R)-7-[4-[(Biphenyl-4-ylmethyl)-carbamoyl]-2-(4-fluoro-phenyl)-5-isopropyl-imidazol-1-yl-3,5-dihydroxy-heptanoate

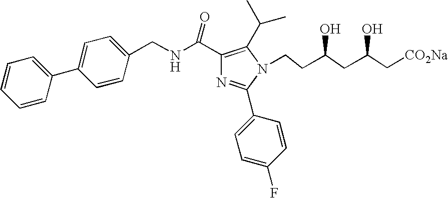

Low resolution mass spectroscopy (APCl) m/z 572 [M−H]⁻; Anal. Calcd for C₃₃H₃₅F₁N₃Na₁O₅/1.7 H₂O: C, 63.29.; H, 6.18; N, 6.71. Found: C, 63.16.; H, 6.11; N, 6.49.

### Example 59

### Sodium: (3R35R)-7-[4-(3-Chloro-4-fluoro-benzycaramoyl)-2-(4-fluoro-phenyl)-5-isopropyl-imidazol-1-yl]-3,5-dihydroxy-heptanoate

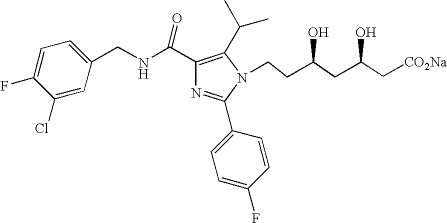

Low resolution mass spectroscopy (APCl) m/z 548 [M−H]⁻; Anal. Calcd for C₂₇H₂₉Cl₁F₂N₃Na₁O₅/1.3 H₂O: C, 54.47.; H, 5.35; N, 7.06. Found: C, 54.57.; H, 5.18; N, 6.85.

### Example 60

### Sodium: (3R,5R)-7-[4-(2,6-Difluoro-benzylcarbamoyl)-2-(4-fluoro-phenyl)-5-isopropyl-imidazol-1-yl]-3,5-dihydroxy-heptanoate

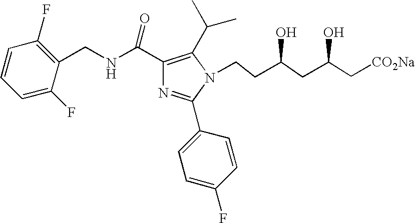

Low resolution mass spectroscopy (APCl) m/z 532 [M−H]⁻; Anal. Calcd for C₂₇H₂9F₃N₃Na₁O₅/1.0 H₂O: C, 56.54.; H, 5.45; N, 7.33. Found: C, 56.21.; H, 5.42; N, 7.10.

### Example 61

### Sodium: (3R,5R)-7-[4-(3-Fluoro-benzylcarbamoyl)-2-(4-fluoro-phenyl)-5-isopropyl-imidazol-1-yl]-3,5-dihydroxy-heptanoate

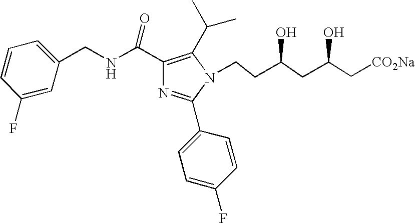

Low resolution mass spectroscopy (APCl) m/z 514 [M−H]⁻; Anal. Calcd for C₂₇H₃₀F₂N₃Na₁O₅/1.0 H₂O: C, 58.37.; H, 5.81; N, 7.56. Found: C, 58.47.; H, 5.76; N, 7.31 .

### Example 62

### Sodium: (3R,5R)-7-{2-(4-Fluoro-phenyl)-5-isopropyl-4-[5-methyl-isoxazol-3-ylmethyl)-carbamoyl]-imidazol-1-yl]-3,5-dihydroxy-heptanoate

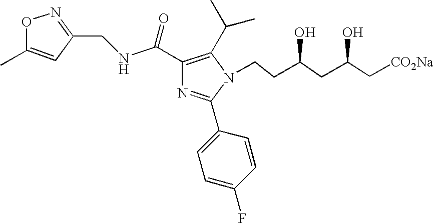

Low resolution mass spectroscopy (APCl) m/z 501 [M−H]⁻; Anal. Calcd for C₂₅H₃₀F₁N₄Na₁O₆/2.0 H₂O: C, 53.57.; H, 6.11; N, 10.00. Found: C, 53.17.; H, 5.82; N, 9.71.

### Example 63

### Sodium: (3R,5R)-7-[4-(4-Fluoro-benzylcarbamoyl)-2-(4-fluoro-phenyl)-5-isopropyl-imidazol-1-yl]-3,5-dihydroxy-heptanoate

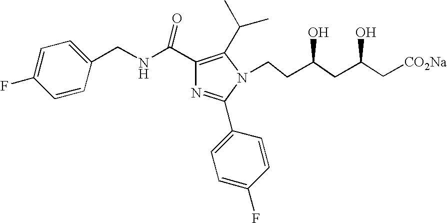

Low resolution mass spectroscopy (APCl) m/z 514 [M−H]⁻; Anal. Calcd for C₂₇H₃₀F₂N₃Na₁O₅/1.3 H₂O: C, 57.81.; H, 5.86; N, 7.49. Found: C, 57.81.; H, 5.70; N, 7.24.

### Example 64

### Sodium: (3R,5R)-7-[2-(4-Fluoro-phenyl)-5-isopropyl-4-(4-phenyl-piperazine-1-carbonyl)-imidazol-1-yl]-3,5-dihydroxy-heptanoate

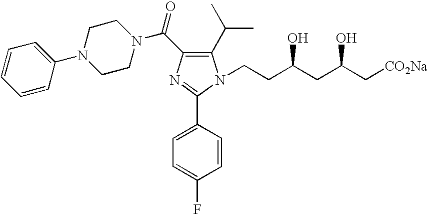

Low resolution mass spectroscopy (APCl) m/z 551 [M−H]⁻; Anal. Calcd for C₃₀H₃₆F₁N₄Na₁O₅/3.5 H₂O: C, 56.51.; H, 6.80; N, 8.79. Found: C, 56.54.; H, 6.66; N, 8.47.

### Example 65

There is no Example 65

### Example 66

### Sodium: (3R,5R)-7-[2-(4-Fluoro-phenyl)-5-isopropyl-4-(4-pyridin-2-yl-piperazine-1-carboyl)-imidazol-1-yl]-3,5-dihydroxy-heptanoate

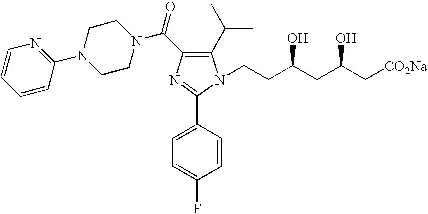

Low resolution mass spectroscopy (APCl) m/z 552 [M−H]⁻; Anal. Calcd for C₂₉H₃₅F₁N₅Na₁; O₅/3.0 H₂O/0.10 NaOH: C, 54.97.; H, 6.54; N, 11.05. Found: C, 54.81.; H, 6.53; N, 10.76.

### Example 67

### Sodium: (3R,5R)-7-[2-(4-Fluoro-phenyl)-5-isopropyl-4-(2-phenoxy-ethylcarbamoyl)-imidazol-1-yl]-3,5-dihydroxy-heptanoate

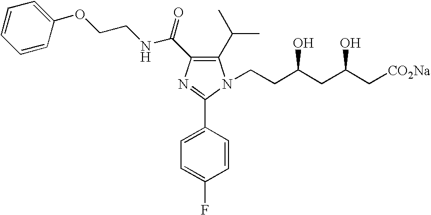

Low resolution mass spectroscopy (APCl) m/z 526 [M−H]⁻; Anal. Calcd for C₂₈H₃₃F₁N₃Na₁O₆/3.0 H₂O: C, 55.71.; H, 6.51; N, 6.96. Found: C, 55.41.; H, 6.39; N, 6.62.

### Example 68

### Sodium; (3R,5R)-7-[4-(3,4- Dichloro-benzylcarbamoyl)-2-(4-fluoro-phenyl)-5-isopropyl-imidazol-1-yl]-3.5-dihydroxy-heptanoate

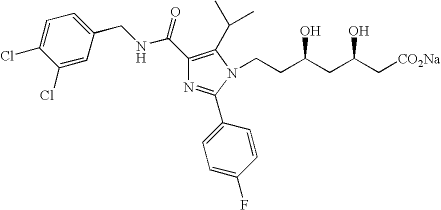

Low resolution mass spectroscopy (APCl) m/z 564/566 [M−H]⁻; Anal. Calcd for C₂₇H₂₉Cl₂F₁N₃Na₁O₅/3.0 H₂O/0.10 NaOH: C, 50.16.; H, 5.47; N, 6.50. Found: C, 50.11.; H, 5.07; N, 6.15.

### Example 69

### Sodium: (3R,5R)-7-[4-[4-(2,4-Difluoro-phenyl)-piperazine-1-carbonyl]-2-(4-fluoro-phenyl)-5-isopropyl-imidazol-1-yl1-3,5-dihydroxy-heptanoate

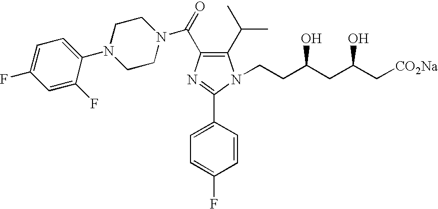

### Example 70

### Sodium; (3R ,5R)-7-[4-Dibenzylcarbamoyl-2-(4-fluoro-phenyl)-5-isopropyl-imidazol-1-yl]-3,5-dihydroxy-heptanoate

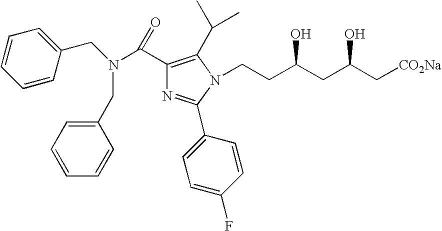

Low resolution mass spectroscopy (APCl) m/z 586 [M−H]⁻; Anal. Calcd for C₃₄H₃₇F₁N₃Na₁O₅/2.8 H₂O: C, 61.86.; H. 6.50; N. 6.37. Found: C, 61.91.; H. 6.14; N. 6.20.

### Example 71

### Sodium: (3R,5R)-7-[2-(4-Fluoro-phenyl)-5-isopropyl-4-((R)-1-phenyl-ethylcarbamoyl)-imidazol-1-yl]-3,5-dihydroxy-heptanoate

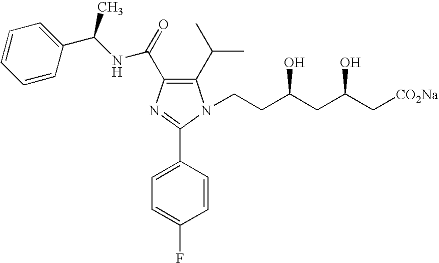

Low resolution mass spectroscopy (APCl) m/z 510 [M−H]⁻; C₂₈H₃₃F₁N₃Na₁O/2.8 H₂O/0.15 NaOH: C, 57.88; H, 6.55; N, 7.23. Found: C, 57.88.; H, 6.16; N, 6.92

### Example 72

### Sodium: (3R,5R)-7-[2-(4-Fluoro-phenyl)-5-isopropyl-4-((S)-1-phenyl-ethylcarbamoyl)-imidazol-1-yl]-3.5-dihydroxy-heptanoate

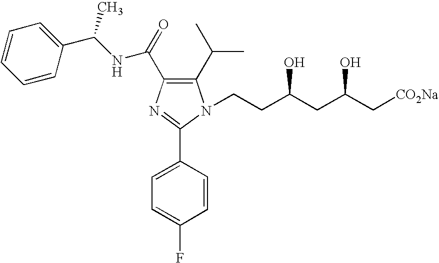

Low resolution mass spectroscopy (APCl) m/z 510 [M−H]⁻; C₂₈H₃₃F₁N₃Na₁O_{5/}2.7 H₂O/0.30 NaOH: C, 56.60; H, 6.56; N, 7.07. Found: C, 56.55.; H, 6.19; N, 6.68.

### Example 73

### Sodium: (3R,5R)-7-[2-(4-Fluoro-phenyl)-5-isopropyl-4-(4-methanesulfonyl-benzylcarbamoyl)-imidazol-1-yl-3,5-dihydroxy-heptanoate

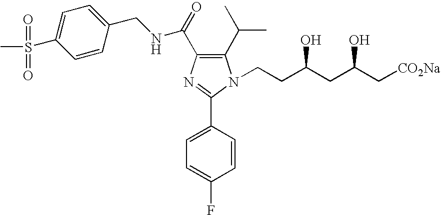

Low resolution mass spectroscopy (APCl) m/z 576 [M+H]⁺; C₂₈H₃₃F₁N₃Na₁O₇S₁/3.0 H₂O: C, 51.61; H, 6.03; N, 6.45. Found: C, 51.46.; H, 5.70; N, 6.27.

### Example 74

### Sodium: (3R,5R)-7-[2-(4-Fluoro-phenyl)-5-ethyl-4-phenylcarbamoyl-imidazol-1-yl]-3,5-dihydroxy-heptanoate

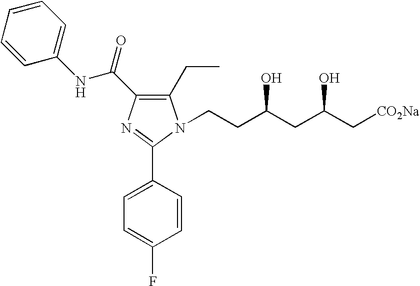

Low resolution mass spectroscopy (APCl) m/z 470 [M+H]⁺; ¹H NMR (400 MHz, DMSO-d6) □1.23 (t, J=7.3 Hz, 3 H) 1.41 (m, 2 H) 1.54 (m, 1 H) 1.67 (m, 1 H) 1.84 (dd, J=15.0, 8.3 Hz, 1 H) 2.02 (dd, J=15.0, 3.9 Hz, 1 H) 3.05 (m, 2 H), 3.59 (m, 1 H), 3.69 (m, 1 H), 4.00 (m, 1 H), 4.15 (m, 1 H), 4.91 (s, 1 H), 6.98 (m, 2 H), 7.37 (m, 4 H), 7.75 (m, 3 H), 9.64 (s, 1 H).

### Example 75

### Sodium: (3R,5R)-7-[2-(4-Fluoro-phenyl)-5-ethyl-4-benzylcarbamoyl-imidazol-1-yl]-3,5-dihydroxy-heptanoate

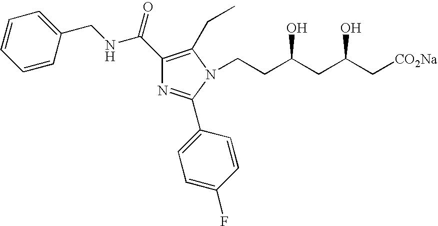

Low resolution mass spectroscopy (APCl) m/z 482 [M−H]⁻;

### Example 76

### Sodium: (3R,5R)-7-[2-(4-Fluoro-phenyl)-5-ethyl-4-phenethylcarbamoyl-imidazol-1-yl]-3,5-dihydroxy-heptanoate

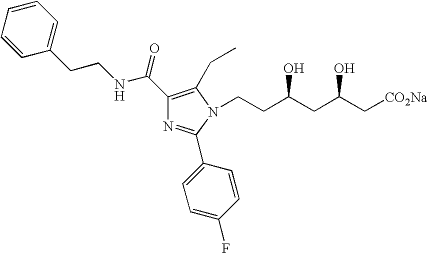

Low resolution mass spectroscopy (APCl) m/z 498 [M+H]⁺; ¹H NMR (400 MHz, DMSO-d6) δ 1.16 (t, J=7.3 Hz, 3 H), 1.23 (m, 1 H), 1.51 (m, 1 H), 1.63 (m, 1 H), 1.74 (m, 1 H), 1.81 (dd, J=15.0, 8.2 Hz, 1 H), 2.00 (dd, J=14.9, 4.2 Hz, 1 H), 2.80 (m, 2 H), 3.00 (m, 2 H), 3.43 (m, 2 H), 3.57 (m, 1 H), 3.67 (m, 1 H), 3.95 (m, 1 H), 4.09 (m, 1 H), 4.68 (s, 1 H), 7.20 (m, 3 H), 7.30 (m, 4 H), 7.66 (m, 2 H), 7.87 (t, J=5.9 Hz, 1 H).

### Example 77

### Sodium; (3R,5R)-7-[2-(4-Fluoro-phenyl)-5-ethyl-4-(4-fluorobenzylcarbamoyl)-imidazol-1-yl]-3,5-dihydroxy-heptanoate

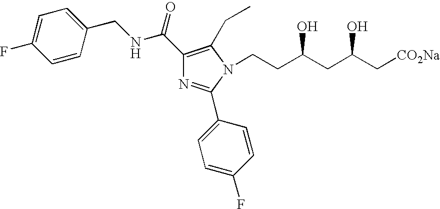

Low resolution mass spectroscopy (APCl) m/z 502 [M+H]⁺; ¹H NMR (400 MHz, DMSO-d6) δ 1.16 (t, J=7.3 Hz, 1 H), 1.23 (m, 1 H), 1.39 (m, 2 H), 1.51 (m, 1 H), 1.63 (m, 1 H), 1.75 (m, 1 H), 1.83 (dd, J=15.1, 8.3 Hz, 1 H), 2.01 (dd, J=15.0, 4.0 Hz, 1 H), 3.00 (m, 2 H), 3.58 (m, 1 H), 3.68 (m, 1 H), 3.95 (m, 1 H), 4.10 (m, 1 H), 4.37 (d, J=6.3 Hz, 2 H), 4.88 (s, 1 H), 7.11 (m, 2 H), 7.31 (m, 4 H), 7.68 (m, 2 H), 8.41 (t, J=6.4 Hz, 1 H).

### Example 78

### Sodium: (3R,5R)-7-[2-(4-Fluoro-phenyl)-5-propyl-4-phenylcarbamoyl-imidazol-1-yl]-3,5-dihydroxy-heptanoate

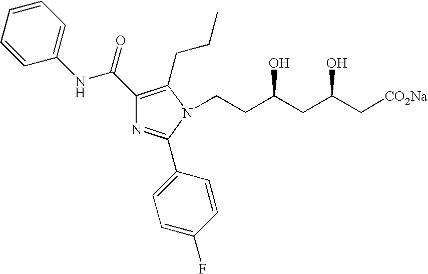

Low resolution mass spectroscopy (APCl) m/z 484 [M+H]⁺; Anal. Calculated for C₂₆H₂₉FN₃O₅Na/2.83 H₂O: C, 56.11; H, 6.28; N, 7.55. Found C, 56.50; H, 5.94; N, 7.15.

### Example 79

### Sodium: (3R,5R)-7-[2-(4-Fluoro-phenyl)-5-propyl-4-benzylcarbamoyl-imidazol-1-yl]-3,5-dihydroxy-heptanoate

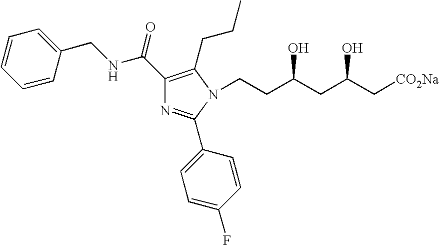

Low resolution mass spectroscopy (APCl) m/z498 [M+H]⁺; ¹H NMR (400 MHz, DMSO-d6) δ 0.93 (t, J=7.3 Hz, 2 H), 1.18 (m, 1 H), 1.36 (m, 1 H), 1.53 (m, 4 H), 1.73 (dd, J=14.8, 8.1 Hz, 1 H), 1.93 (dd, J=14.8, 4.1 Hz, 1 H), 2.96 (m, 2 H), 3.28 (s, 1 H), 3.56 (m, 1 H), 3.65 (m, 1 H), 3.95 (m, 1 H), 4.09 (m, 1 H), 4.39 (d, J=6.3 Hz, 2 H), 4.94 (s, 1 H), 7.20 (m, 1 H), 7.30 (m, 5 H), 7.68 (m, 3 H), 8.35 (t, J=6.3 Hz, 1 H).

### Example 80

### Sodium: (3R,5R)-7-[2-(4-Fluoro-phenyl)-5-propyl-4-phenethylcarbamoyl-imidazol-1-yl]-3,5-dihydroxy-heptanoate

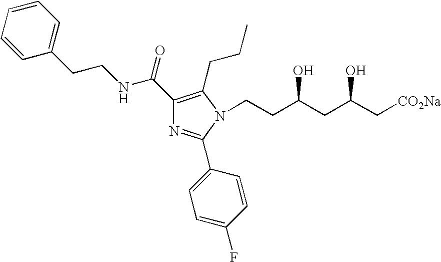

Low resolution mass spectroscopy (APCl) m/z 512 [M+H]⁺; ¹H NMR (400 MHz, DMSO-d6) δ

0.94 (t, J=7.3 Hz, 2 H), 1.18 (m, 1 H), 1.36 (m, 1 H), 1.53 (m, 4 H), 1.74 (dd, J=14.0, 8.2 Hz, 1 H), 1.94 (dd, J=14.8, 4.0 Hz, 1 H), 2.79 (m, 2 H), 2.96 (m, 2 H), 3.29 (s, 1 H), 3.43 (m, 2 H), 3.56 (m, 1 H), 3.64 (m, 1 H), 3.94 (m,1 H), 4.08 (m, 1 H), 4.93 (s, 1 H), 7.19 (m, 3 H), 7.29 (m, 4 H), 7.67 (m, 2 H), 7.87 (t, J=6.1 Hz, 1 H).

### Example 81

### Sodium; (3R, 5R)-7-[2-(4-Fluoro-phenyl)-5-methyl-4-(4-fluorophenylcarbamoyl)-imidazol-1-yl]-3,5-dihydroxy-heptanoate

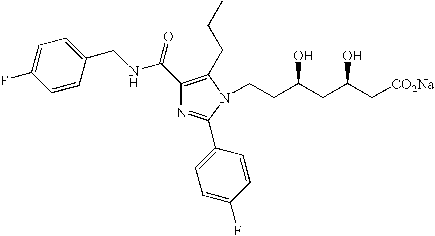

J=Low resolution mass spectroscopy (APCl) m/z 516 [M+H]⁺; ¹H NMR (400 MHz, DMSO-d6) δ 0.93 (t, 7.3 Hz, 3 H), 1.18 (m, 1 H), 1.36 (m, 1 H), 1.53 (m, 4 H), 1.73 (dd, J=15., 8.1 Hz, 1 H), 1.93 (dd, J=14.8, 4.0 Hz, 1 H), 2.9 (m, 2 H), 3.2 (s, 1 H), 3.56 (s, 1 H), 3.63 (m, 1 H), 3.95 (m, 1 H), 4.09 (m, 1 H), 4.36 (d, J=6.3 Hz, 2 H), 4.93 (s, 1 H), 7.11 (m, 2 H), 7.31 (m, 4 H), 7.67 (m, 2 H), 8.40 (t, J=6.4 Hz, 1 H)

### Example 82

### Sodium; (3R,5R)-7-[2-(4-Fluoro-phenyl)-5-methyl-4-phenylcarbamoyl-imidazol-1-yl]-1-3.5-dihydroxy-heptanoate

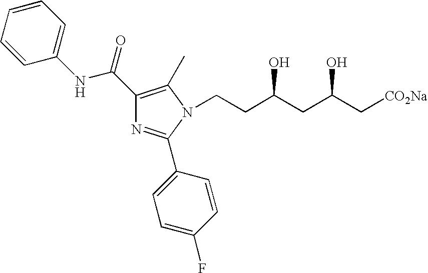

Low resolution mass spectroscopy (APCl) m/z 456 [M+H]⁺;

### Example 83

### Sodium; (3R,5R)-7-[2-(4-Fluoro-phenyl)-5-methyl-4-benzylcarbamoyl-imidazol-1-yl]-3,5-dihydroxy-heptanoate

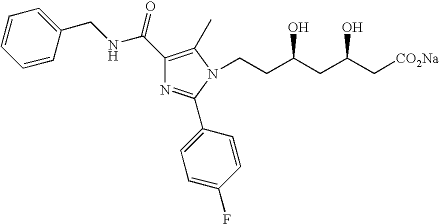

Low resolution mass spectroscopy (APCl) m/z 470 [M+H]⁺; ¹H NMR (400 MHz, DMSO-d6) δ 1.22 (m, 1 H), 1.39 (m, 1 H), 1.54 (m, 1 H), 1.73 (m, 1 H), 1.94 (dd, J=14.9, 3.9 Hz, 1 H), 2.56 (s, 3 H), 3.00 (m, 1 H), 3.28 (s, 1 H), 3.57 (m, 1 H), 3.66 (m, 1 H), 3.94 (m, 1 H), 4.06 (m, 1 H), 4.39 (d, J=6.2 Hz, 2 H), 4.94 (s, 1 H), 7.20 m, 1 H), 7.30 (m, 4 H), 7.67 (m, 2 H), 7.79 (s, 1 H), 8.36 (t, J=6.3 Hz, 1 H).

### Example 84

### Sodium; (3R,5R)-7-[2-(4-Fluoro-phenyl)-5-methyl-4-phenethylcarbamoyl-imidazol-1-yl]-3,5-dihydroxy-heptanoate

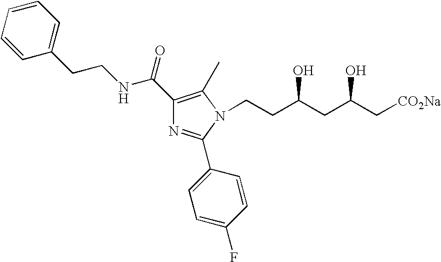

Low resolution mass spectroscopy (APCl) m/z 484 [M+H]⁺; ¹H NMR (400 MHz, DMSO-d6) δ 1.17 (m, 1 H), 1.34 (m, 1 H), 1.49 (m, 1 H), 1.68 (m, 1 H), 1.89 (dd, J=15.0, 4.0 Hz, 1 H), 2.51 (s, 3 H), 2.75 (m, 2 H), 2.96 (m, 2 H), 3.38 (m, 2 H), 3.52 (m, 1 H), 3.61 (m, 1 H), 3.89 (m, 1 H), 4.01 (m, 1 H), 4.89 (s, 1 H), 7.15 (m, 3 H), 7.26 (m, 4 H), 7.61 (m, 2 H), 7.83 (t, J=6.1 Hz, 1 H).

### Example 85

### Sodium: (3R,5R)-7-[4-[(Biphenyl-3-ylmethyl)-carbamoyl]-2-(4-fluoro-phenyl)-5-isopropyl-imidazol-1-yl]-3,5-dihydroxy-heptanoate

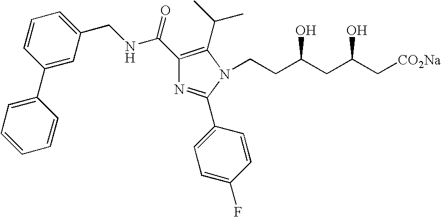

Anal. Calculated for C₃₃H₃₅FN₃O₅Na.8.48 H₂O: C, 52.96; H, 7.00; N, 5.61. Found C, 52.57; H, 7.06; N, 5.53.

### Example 86

### Sodium; (3R,5R)-7-[2-(4-Fluoro-phenyl)-5-isopropyl-4-phenethylcarbamoyl-imidazol-1-yl]-3,5-dihydroxy-heptanoate

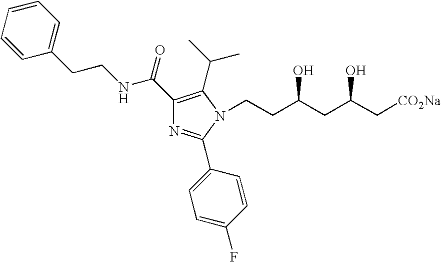

MS(C₂₈H₃₄FN₃O₅) sought 510; found 413,497 Anal. Calculated for C₂₈H₃₃FN₃O₅Na.23.5 H₂O: C, 35.14; H, 8.43; N, 4.39. Found C, 35.13; H, 3.65; N, 2.97.

### Example 87

### Sodium; (3R,5R)-7-[2-(4-Fluoro-phenyl)-5-methyl -4-(4-sulfamoyl-benzylcarbamoyl)-imidazol-1-yl1-3.5-dihydroxy-heptanoate

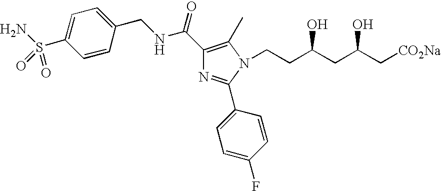

Low resolution mass spectroscopy (APCl) m/z 549 [M+H]⁺; ¹H NMR (400 MHz, DMSO-d6) δ 1.17 (m, 1 H), 1.34 (m, 3 H), 1.49 (m, 1 H), 1.63 (m, 1 H), 1.70 (dd, J=15.0, 8.3 Hz, 1 H). 1.90 (dd, J=14.7, 4.0 Hz, 1 H), 2.51 (s, 3 H), 3.24 (s, 1 H), 3.52 (m, 1 H), 3.62 (m, 1 H), 3.90 (m, 1 H), 4.02 (m, 1 H), 4.40 (d, J=6.4 Hz, 2 H), 4.89 (s, 1 H), 7.22 (s, 1 H), 7.27 (m, 1 H), 7.40 (m, 2 H), 7.63 (m, 2 H), 7.70 (m, 2 H), 8.49 (t, J=6.2 Hz, 1 H).

### Example 88

### Sodium: (3R,5R)-7-[4-benzylcarbamoyl-2-phenyl-5-isopropyl-imidazol-1-yl]-3,5-dihydroxy-heptanoate

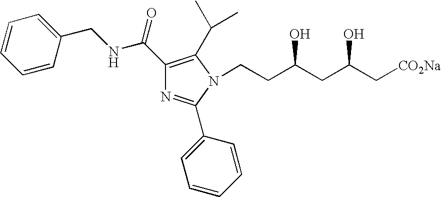

Low resolution mass spectroscopy (APCl) m/z 480 [M+H]⁺; ¹H NMR (400 MHz, DMSO-d6) δ 1.24 (m, 2 H), 1.40 (m, 6 H), 1.58 (m, 1 H), 1.70 (m, 1 H), 1.80 (dd, J=15.0, 8.3 Hz, 1 H), 1.98 (dd, J=15.1, 4.0 Hz, 1 H), 3.29 (s, 1 H), 3.37 (m, 1 H), 3.62 (m, 1 H), 3.69 (m, 1 H), 3.96 (m, 1 H), 4.12 (m, 1 H), 4.41 (d, J=6.3 Hz, 2 H), 4.92 (s, 1 H), 7.20 (m, 1 H), 7.29 (m, 4 H), 7.47 (m, 3 H), 7.59 (m, 2 H), 8.37 (t, J=6.4 Hz, 1 H).

### Example 89

### Sodium; (3R,5R)-7-[4-(3-Chloro-benzylcarbamoyl)-2-(4-fluoro-phenyl)-5-isopropyl-imidazol-1-yl]-3,5-dihydroxy-heptanoate

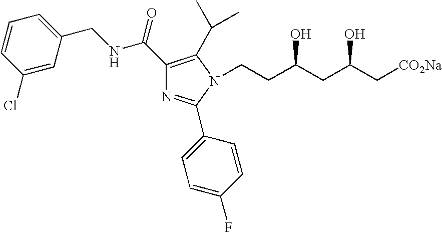

Low resolution mass spectroscopy (APCl) m/z 532 [M+H]⁺; ¹H NMR (400 MHz, DMSO-d6) δ 0.97 (t, J=7.5 Hz, 1 H), 1.18 (m, 1 H), 1.35 (m, 6 H), 1.63 (m, 1 H), 1.72 (dd, J=14.9, 8.1 Hz, 1 H), 1.92 (dd, J=14.8, 4.0 Hz, 1 H), 3.25 (m, 1 H), 3.33 (m, 2 H), 3.56 (m, 1 H), 3.63 (m, 1 H), 3.91 (m, 1 H), 4.06 (m, 1 H), 4.35 (d, J=6.4 Hz, 2 H), 4.91 (s, 1 H), 7.22 (m, 2 H), 7.28 (m, 4 H), 7.61 (m, 2 H), 8.49 (t, J=6.4 Hz, 1 H).

### Example 90

### Sodium: (3R,5R)-7-[2-(4-Fluoro-phenyl)-4-(indan-1-ylcarbamoyl)-5-isopropyl-imidazol-1-yl]-3,5-dihydroxy-heptanoate

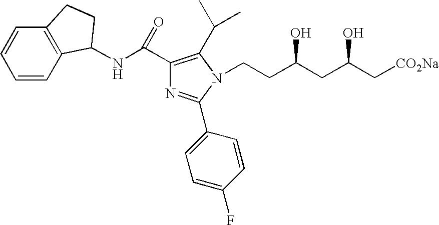

Low resolution mass spectroscopy (APCl) m/z 524 [M+H]⁺; ¹H NMR (400 MHz, DMSO-d6) δ 1.02 (m, 1 H), 1.12 (m, 1 H), 1.24 (m, 1 H), 1.37 (m, 1 H), 1.45 (m, 6 H), 1.69 (m, 1 H), 1.78 (dd, J=14.8, 8.1 Hz, 1 H), 1.97 (J=14.8, 3.9 Hz, 1 H), 2.41 (m, 1 H), 2.90 (m, 1 H), 3.39 (m, 2 H), 3.61 (m, 1 H), 3.68 (m, 1 H), 3.96 (m, 1 H), 4.09 (m, 1 H), 4.94 (s, 1 H), 5.41 (m, 2 H), 7.17 (m, 2 H), 7.29 (m, 3 H), 7.48 (s, 1 H), 7.64 (m, 2 H), 7.90 (d, J=8.9 Hz,

### Example 91

### Sodium: (3R,5R)-7-[2-(4-Fluoro-phenyl)-5-isopropyl-4-(3-phenyl-pyrrolidine-1-carbonyl)-imidazol-1-yl]-3,5-dihydroxy-heptanoate

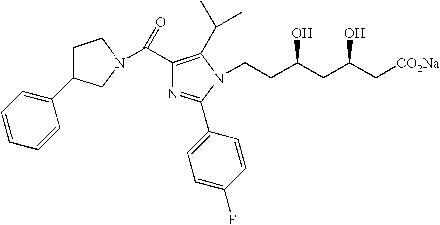

Low resolution mass spectroscopy (APCl) m/z 538 [M+H]⁺;

### Example 92

### Sodium: (3R,5R)-7-[4-(3-Benzenesulfonyl-pyrrolidine-1-carbonyl)-2-(4-fluoro-phenyl)-5-isopropyl-imidazol-1-yl]-3,5-dihydroxy-heptanoate

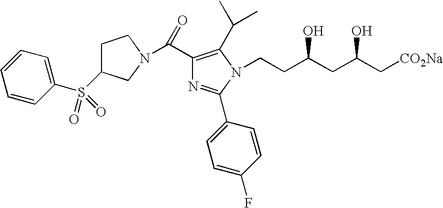

Low resolution mass spectroscopy (APCl) m/z 602 [M+H]⁺; Anal. Calculated for C₃₀H₃₅FN₃O₇S Na.0.85 H₂O: C, 56.39; H, 5.79; N, 6.58. Found C, 56.39; H, 5.65; N, 6.36.

### Example 93

### Sodium: (3R,5R)-7-[2-(4-Fluoro-phenyl)-5-isopropyl-4-(4-sulfamoyl-benzylcarbamoyl)-imidazol-1-yl]-3,5-dihydroxy-heptanoate

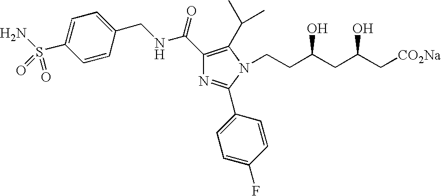

Low resolution mass spectroscopy (APCl) m/z 577 [M+H]⁺; ¹H NMR (400 MHz, DMSO-d6) δ 1.00 (m, 3 H), 1.23 (m, 2 H), 1.39 (m, 6 H), 1.55 (m, 1 H), 1.55 (m, 1 H), 1.68 (m, 1 H), 1.77 (dd, J=15.0, 8.2 Hz, 1 H), 1.97 (dd, J=14.8, 4.0 Hz, 1 H), 3.62 (m, 1 H), 3.68 (m, 1 H), 3.95 (m, 1 H), 4.10 (m, 1 H), 4.46 (d, J=6.4 Hz, 1 H), 4.94 (s, 1 H), 7.12 (s, 1 H), 7.31 (m, 1 H), 7.45 (m, 1 H), 7.53 (s, 1 H), 7.66 (m, 2 H), 7.74 (m, 2 H), 8.55 (t, J=6.2 Hz, 1 H).

### Example 94

### Sodium: (3R,5R)-7-[2-(4-Fluoro-phenyl)-5-isopropyl-4-(methanesulfonylamino-methyl)-imidazol-1-yl]-3,5-dihydroxy-heptanoate

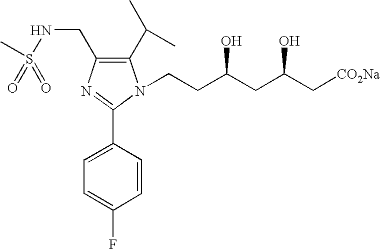

Step A

### ((4R,6R)-6-{2-[2-(4-Fluoro-phenyl)-4-hydroxymethyl-5-isopropyl-imidazol-1-yl]-ethyl]-2,2-dimethyl-[1,3]dioxan-4-yl)-acetic acid tert-butyl ester

A solution of 1-[2-((4R, 6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid pentafluorophenyl ester (4.0 g, 6.7 mmol) in absolute EtOH (120 mL) was carefully treated with excess NaBH₄ (2.5 g, 67 mmol) in portions over a period of 5 min. The reaction mixture was allowed to stir at ambient temperature for 48 h. The reaction mixture was carefully treated with neat HOAc (2 mL) and allowed to stir for 5 min. The mixture was concentrated to a crude oil and partitioned between EtOAc/1 M NaOH. The organic layer was separated, washed (sat. NH₄Cl), dried (Na₂SO4), and concentrated to a colorless oil. TLC analysis indicated one major component (R_{f}=0.17, (EtOAc, UV & KMnO4). Purification by flash chromatography (SiO₂, MeOH/EtOAc 5%) gave the desired product as a colorless foam; Yield: 2.03 g (61%); Low resolution mass spectroscopy (APCl) m/z 491 [M+H]⁺; Anal. Calcd. For C₂₇H₃₉F₁N₂O₅: C, 66.10; H, 8.01; N, 5.71. Found: C, 65.78; H, 8.01; N, 5.53.

Step B

### ((4R,6R)-6-{2-[2-(4-Fluoro-phenyl)-4-formyl-5-isopropyl-imidazol-1-yl]-ethyl}-2,2-dimethyl-[1,3]dioxan-4-yl)-acetic acid tert-butyl ester

A solution of ((4R,6R)-6-{2-[2-(4-Fluoro-phenyl)-4-hydroxymethyl-4-hydroxymethyl-5-isopropyl-imidazol-1-yl]-ethyl}-2,2-dimethyl-[1,3]dioxan-4-yl)-acetic acid tert-butyl ester (6.0 g, 12 mmol) in anhydrous CH₂Cl₂ (60 mL) was treated with excess Manganese (IV) oxide (11 g, 122 mmol). The heterogenous reaction mixture was vigorously stirred at rt under a nitrogen atmosphere overnight. TLC analysis (EtOAc, 100%) indicates complete consumption of the starting material (R_{f}=0.17) and a new non polar component (R_{f}=0.70). The reaction mixture was filtered through celite, concentrated to a colorless glass and dried under high vacuum to give the desired product; yield: 5.82 g (97%); Low resolution mass spectroscopy (APCl) m/z 490 [M+H]⁺; Anal. Calcd. For C₂₇H₃₇F₁N₂O₅: C, 66.37; H, 7.63; N, 5.73. Found: C, 66.42; H, 7.83; N, 5.73.

Step C

### N-{2-(4-Fluoro-phenyl)-1-[2-((2R,4R)-4-hydroxy-6-oxo-tetrahydro-pyran-2-yl)-ethyl]-5-isopropyl-1H-imidazol-4-ylmethyl-methanesulfonamide

A solution of ((4R, 6R)-6-{2-[2-(4-Fluoro-phenyl)-4-formyl-5-isopropyl-imidazol-1-yl]-ethyl}-2,2-dimethyl-[1,3]dioxan-4-yl)-acetic acid tert-butyl ester (1.5 g, mmol) in methanol (50 mL) saturated with ammonia was hydrogenated over Raney Nickel (0.5 g). The mixture was filtered through celite and concentrated to give crude ((4R, 6R)-6-{2-[4-Aminomethyl-2-(4-fluoro-phenyl)-5-isopropyl-imidazol-1-yl]-ethyl}-2,2-dimethyl-[1,3]dioxan-4-yl)-acetic acid tert-butyl ester as a glass; Low resolution mass spectroscopy (APCl) m/z 491 [M+H]⁺. A portion of this material (300 mg, 0.61 mmol) was dissolved in THF (5 mL) and treated sequentially with 2,6-lutidine (98 mg, 0.91 mmol) and neat methanesulfonyl chloride (77 mg, 0.67 mmol). The resulting mixture was allowed to stir at rt overnight. The reaction mixture was concentrated to an oil and partitioned between EtOAc and sat. NaHCO₃. The organic layer was separated, washed with sat. NH₄Cl, dried (Na₂SO₄), and concentrated to give ((4R,6R)-6-{2-[2-(4-Fluoro-phenyl)-5-isopropyl-4-(methanesulfonylamino-methyl)-imidazol-1-yl]-ethyl}-2,2-dimethyl-[1,3]dioxan-4-yl)-acetic acid tert-butyl ester as a crude solid; Low resolution mass spectroscopy (APCl) m/z 568 [M+H]⁺. The crude amide was taken up in CH₂Cl₂ (4 mL) and treated with neat TFA (1 mL). The reaction mixture was allowed to stir at rt for 120 min then diluted with trifluoromethylbenzene (5 mL) and concentrated to a crude oil. The oil was partitioned between EtOAc and water. The aqueous layer was carefully adjusted to pH ˜8 by the addition of sat. NaHCO₃ and the organic layer was separated, washed with sat. NH₄Cl, dried (Na₂SO₄), and concentrated to a crude solid. Purification by flash chromatography [SiO₂, MeOH/EtOAc 0-10%] provided the desired lactone a cream colored solid that was placed under high vacuum (overnight); yield: 63mg (22%); Low resolution mass spectroscopy (APCl) m/z 454 [M+H]⁺; Anal. Calcd. For C₂₁ H₂₈F₁N₃O₅S₁0.2 C₄H₈O₂:C, 55.57; H, 6.33; N, 8.92. Found: C, 55.76; H, 6.22; N, 8.77.

Step D

A solution of N-{2-(4-Fluoro-phenyl)-1-[2-((2R,4R)-4-hydroxy-6-oxo-tetrahydro-pyran-2-yl)-ethyl}-5-isopropyl-1H-imidazol-4-ylmethyl]-methanesulfonamide (58 mg, 0.12 mmol) in THF (5 mL) was treated with aqueous NaOH (1.12 mL, 0.12 mmol, 0.114M). The reaction was allowed to stir at rt and monitored by HPLC for the consumption of SM. The sample was concentrated to approximately 2 mL total volume, then diluted with water (5 mL) and lyophilized to give a colorless powder; Yield: 63 mg (100%); Low resolution mass spectroscopy (APCl) m/z 472 [M+H]⁺; Anal. Calcd. For C₂₁H₂₉F₁N₃Na₁O₆S₁ 1.5 H₂O: C, 48.45; H, 6.20; N, 8.07. Found: C, 48.44; H, 6.13; N, 7.92.

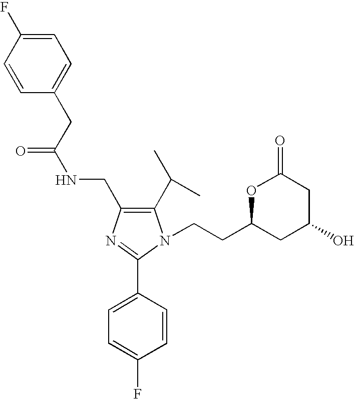

### Example 95

### 2-(4-Fluoro-phenyl)-N-{2-(4-fluoro-phenyl)-1-[2-((2R,4R)-4-hydroxy-6-oxo-tetrahydro-pyran-2-yl)-ethyl]-5-isopropyl-1H-imidazol-4-ylmethyl]-acetamide

Starting from ((4R, 6R)-6-{2-[2-(4-Fluoro-phenyl)-4-formyl-5-isopropyl-imidazol-1-yl]-ethyl]-2,2-dimethyl-[1,3]dioxan-4-yl)-acetic acid tert-butyl ester, this compound was prepared in a manner similar to that described for Example 94,

Step C.

Low resolution mass spectroscopy (APCl) m/z 512 [M+H]⁺;

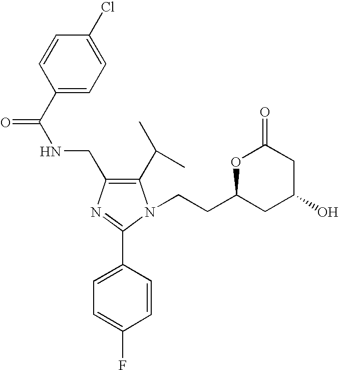

### Example 96

### 4-Chloro-N-{2-(4-fluoro-phenyl)-1-[2-((2R,4R)-4-hydroxy-6-oxo-tetrahydro-pyran-2-yl)-ethyl]-5-isopropyl-1H-imidazol-4-ylmethyl]-benzamide

Starting from ((4R, 6R)-6-{2-[2-(4-Fluoro-phenyl)-4-formyl-5-isopropyl-imidazol-1-yl]-ethyl}-2,2-dimethyl-[1,3]dioxan-4-yl)-acetic acid tert-butyl ester, this compound was prepared in a manner similar to that described for Example 94, Step C. Low resolution mass spectroscopy (APCl) m/z 514 [M+H]⁺; Anal. Calcd. For C₂₇H₂₉Cl₁F₁N₃O₄: C, 63.09; H, 5.69; N, 8.18. Found C, 62.96; H, 5.66; N, 8.17.

### Example 97

### 1-[2-((4R,6R)-6-tert-Butoxycarbonylmethyl-2,2-dimethyl-[1,3]dioxan-4-yl)-ethyl]-2-(3,4-difluoro-phenyl)-5-isopropyl-1H-imidazole-4-carboxylic acid

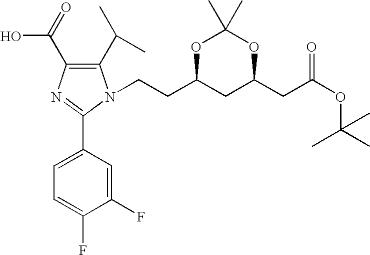

Step A

### 2-(3,4-Difluoro-benzoylamino)-4-methyl-3-oxo-pentanoic acid benzyl ester

Starting from 2-Amino-4-methyl-3-oxo-pentanoic acid benzyl ester hydrochloride, the above named compound was prepared by following a process analogous to the one described in Example 3, Step C. Recrystallization from hot MTBE-hexanes gives the desired product as a colorless solid. Yield (84%); Low resolution mass spectroscopy (APCl) m/z 376 [M+H]⁺; Anal. Calcd. For C₂₀H₁₉F₂N₁O₄: C, 64.00; H, 5.10; N, 3.73. Found: C, 64.01, H, 5.01; N, 3.75.

Step B

### 1-{2-((4R,6R)-6-tert-Butoxycarbonylmethyl-2,2-dimethyl-[1,3]dioxan-4-yl)-ethyl]-(3,4-difluoro-phenyl)-5-isopropyl-1H-imidazole-4-carboxylic acid benzyl ester

Starting from 2-(3,4-Difluoro-benzoylamino)-4-methyl-3-oxo-pentanoic acid benzyl ester (3.0 g, 8.0 mmol) the above named compound was prepared by following a process analogous to the one described in Example 3, Step D. Purification by flash chromatography (SiO₂, EtOAc/Hexanes 10-50%) gave the desired product as an amber glass. Yield: 2.2 g (44%); Low resolution mass spectroscopy (APCl) m/z 613 [M+H]⁺;]+; Anal. Calcd. For C₂₇H₃₇F₁N₂O₆; C, 66.65; H, 6.91; N, 4.57. Found: C, 66.41, H, 6.93; N, 4.23.

Step C

Starting from 1-[2-((4R,6R)-6-tert-Butoxycarbonylmethyl-2,2-dimethyl-[1,3]dioxan-4-yl)-ethyl]-2-(3,4-difluoro-phenyl)-5-isopropyl-1H-imidazole-4-carboxylic acid benzyl ester (2.1 g, 3.4 mmol), the title compound was prepared by following a process analogous to the one described in Example 2, Step F. Yield: 2.2 g (44%); Low resolution mass spectroscopy (APCl) m/z 523 [M+H]⁺; Anal. Calcd. For C₂₇H₃₆F₂N₂O₆: C, 62.06; H, 6.94; N, 5.36. Found: C, 62.44; H, 7.02; N, 5.09.

### Example 98

### 1-[2-((4R,6R)-6-tert-Butoxycarbonylmethyl-2,2-dimethyl-[1,3]dioxan-4-yl)-ethyl]-2-(4-fluoro-3-trifluoromethyl-phenyl)-5-isopropyl-1H-imidazole-4-carboxylic acid

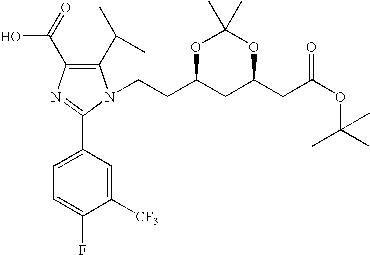

Step A

### 2-(4-fluoro-3-trifluoromethyl-benzoylamino)-4-methyl-3-oxo-pentanoic acid benzyl ester

Starting from 2-Amino-4-methyl-3-oxo-pentanoic acid benzyl ester hydrochloride the above named compound was prepared by following a process analogous to the one described in Example 3, Step C. Recrystallization from hot MTBE-hexanes gives the desired product as a colorless solid. Yield: (48%); Low resolution mass spectroscopy (APCI) m/z 426 [M+H]⁺; Anal. Calcd. For C₂₁H₁₉F₄N₁O₄: C, 59.30; H, 4.50; N, 3.29. Found: C, 59.00; H, 4.41; N, 3.36.

Step B

### 1-[2-((4R,6R)-6-tert-Butoxycarbonylmethyl-2,2-dimethyl-[1,3]dioxan-4-yl)-ethyl]-2-(4-fluoro-3-trifluoromethyl-phenyl)-5-isopropyl-1H-imidazole-4-carboxylic acid benzyl ester

Starting with 2-(4-fluoro-3-trifluoromethyl-benzoylamino)-4-methyl-3-oxo-pentanoic acid benzyl ester (3.5 g, 8.2 mmol) the above named compound was prepared by following a process analogous to the one described in Example 3, Step D. Purification by flash chromatography (SiO₂, EtOAc/Hexanes 25-40 %) gave the desired product as a colorless foam. Yield: 3.3 g (61 %); Low resolution mass spectroscopy (APCI) m/z 663 [M+H]⁺; Anal. Calcd. For C₃₅H₄₂F₄N₂O₆: C, 63.43; H, 6.39; N, 4.23. Found: C, 63.42; H, 6.39; N, 4.13.

Step C Starting with 1-[2-((4R,6R)-6-tert-Butoxycarbonylmethyl-2,2-dimethyl-[1,3]dioxan-4-yl)-ethyl]-2-(4-fluoro-3-trifluoromethyl-phenyl)-5-isopropyl-1H-imidazole-4-carboxylic acid benzyl ester (3.2 g, 4.8 mmol), the title compound was prepared by following a process analogous to the one described in Example 2, Step F. Yield: 2.6g (94%); Low resolution mass spectroscopy (APCI) m/z 573 [M+H]⁺; Anal. Calcd. For C₂₇H₃₆F₂N₂O₆: C, 58.73; H, 6.34; N, 4.89. Found: C, 58.82; H, 6.37; N, 4.69.

### Example 99

### 1-[2-((4R,6R)-6-tert-Butoxycarbonylmethyl-2,2-dimethyl-[1,3]dioxan-4-yl)-ethyl]-5-cyclopropyl-2-(4-fluoro-phenyl)-1H-imidazole-4-carboxylic acid

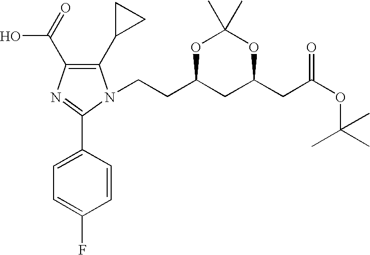

Step A

### 3-Cyclopropyl-2-(4-fluoro-benzoylamino)-3-oxo-propionic acid benzyl ester

A 500 mL round-bottomed flask was charged with potassium tert-butoxide (9.4 g, 83 mmol) and THF (150 mL). The solution was cooled, under nitrogen, in an ice-brine bath and treated with a solution of (Benzhydrylidene-amino)-acetic acid benzyl ester (25.0 g, 79.5 mmol) in THF (150 mL). The red-orange solution was stirred for 1 h at 0° C. and then cannulated into a −78° C. solution of cyclopropanecarbonyl chloride (8.33 g, 79.7 mmol) in THF (400 mL). The resulting mixture was stirred for 2 h at −78° C., then quenched with 3M HCl (75 mL, 225 mmol). The cold bath was removed and the reaction mixture was allowed to stand overnight. The reaction mixture was concentrated in vacuo to produce an oily yellow residue. The residue was dissolved in water (200 mL) and extracted with hexanes (2×100 mL). The aqueous layer was adjusted to pH>8 by the careful addition of solid NaHCO3. EtOAc was added (300 mL), the biphasic mixture was cooled in an ice-brine bath, and the cooled mixture was treated with 4-fluorobenzoyl chloride (12.6g, 79.7 mmol). The reaction mixture was allowed to warm to rt and left to stand overnight. The organic layer was separated, washed with 1 M HCl and sat. NH₄Cl, dried (Na2SO4), and concentrated to a crude oil that solidified on standing. The crude product was recrystallized from a minimum of hot 95% EtOH to give colorless needles that were collected by vacuum filtration. The purified material was dried in vacuo. Yield: 14.2g (52%); mp=94.5-96° C.; Low resolution mass spectroscopy (APCI) m/z 354[M+H]⁺; Anal. Calcd. For C₂₀H₁₈F₁N₁O₄. Theory: C, 67.67; H, 5.11; N, 3.94. Found: C, 67.48; H, 5.12; N, 3.90.

Step B

### 1-[2-((4R,6R)-6-tert-Butoxycarbonylmethyl-2,2-dimethyl-[1,3]dioxan-4-yl)-ethyl]-2-(4-fluoro-phenyl)-5-cyclopropyl-1H-imidazole-4-carboxylic acid benzyl ester

A mixture of 3-Cyclopropyl-2-(4-fluoro-benzoylamino)-3-oxo-propionic acid benzyl ester (6.0 g, 17 mmol), [(4R,6R)-6-(2-Amino-ethyl)-2,2-dimethyl-[1,3]dioxan-4-yl]-acetic acid tert-butyl ester (TBIA) (9.2 g, 33.8 mmol), benzoic acid (6.19 g, 50.7 mmol), and Ptoluenesulfonic acid (0.29 g, 1.7 mmol) in n-heptane (150 mL) was heated to reflux for 65 h with the removal of water (Dean-Stark trap). The reaction mixture was cooled, diluted with EtOAc (100 mL), and washed with 1 M NaOH (2×150 mL) and sat NH₄Cl, dried (Na₂SO₄) and concentrated to a yellow-brown oil. Purification by flash chromatography [SiO₂, Ethyl Acetate/hexanes 10-50%] provides the desired product as a yellow glass that was dried under high vacuum. Yield: 2.1 g (21 %); Low resolution mass spectroscopy (APCI) m/z 593 [M+H]⁺; Anal. Calcd. For C₃₄H₄₁F₁N₂O₆: C, 68.90; H, 6.97; N, 4.73. Found: C, 68.66; H, 7.01; N, 4.64.

Step C

Starting with 1-[2-((4R,6R)-6-tert-Butoxycarbonylmethyl-2,2-dimethyl-[1,3]dioxan-4-yl)-ethyl]-2-(4-fluoro-phenyl)-5-cyclopropyl-1H-imidazole-4-carboxylic acid benzyl ester (2.0 g, 3.4 mmol), the title compound was prepared by following a process analogous to the one described in Example 2, Step F. Yield: 1.69 g (99%); Low resolution mass spectroscopy (APCI) m/z503 [M+H]⁺; Anal. Calcd. For C₂₇H₃₅F₁N₂O₆: C, 64.53; H, 7.02; N, 5.57. Found: C, 63.99; H, 7.38; N, 5.25.

### Example 100

### 2-(3.4-Difluoro-phenyl)-1-[2-((2R,4R)-4-hydroxy-6-oxo-tetrahydro-pyran-2-yl)-ethyl]-5-isopropyl-1H-imidazole-4-carboxylic acid benzylamide

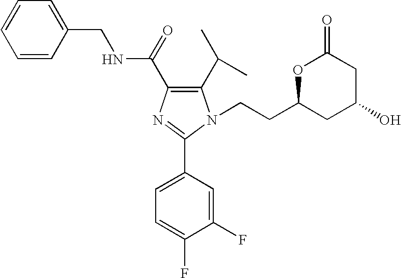

A rt solution 1-[2-((4R, 6R)-6-tert-Butoxycarbonylmethyl-2,2-dimethyl-[1,3]dioxan-4-yl)-ethyl]-2-(3,4-difluoro-phenyl)-5-isopropyl-1H-imidazole-4-carboxylic acid (522 mg, 1.0 mmol) in dry DMF (20 mL) was treated with EDCI (290 mg, 1.5 mmol) and HOBt (200 mg, 1.5 mmol). After stirring for 20 min, neat benzyl amine (128 mg, 1.2 mmol) was added and the reaction was allowed to stir at rt overnight. An LC-MS analysis of the crude reaction mixture indicates a mass corresponding to the expected product [M+H]⁺=612. The reaction mixture was poured into water (150 mL) and extracted with EtOAc (3×). The extracts were combined, washed with water (2×) and sat. NH₄Cl (2×), dried (Na₂SO₄) and concentrated to a colorless foam. The crude amide was taken up in CH₂Cl₂ (20 mL), treated with neat TFA (5 mL), and allowed to stir at rt for 30 min at which time an LC-MS analysis indicated no remaining SM and a new mass corresponding to the expected lactone [M+H]⁺=498. The reaction mixture was concentrated to dryness and residue was partitioned between EtOAc and 1 M NaHCO₃. (pH ˜8). The organic layer was separated, washed with sat. NH₄Cl, dried (Na₂SO₄), and concentrated to an oil. Purification by flash chromatography (silica, EtOAc/hexanes 50-100%) provides the lactone as a colorless glass. Yield: 302 mg (61%); Low resolution mass spectroscopy (APCI) m/z 498 [M+H]⁺; ¹H NMR (400 MHz, CD₃CN) δ 1.44 (d, J=1.46 Hz, 3 H), 1.46 (d, J=1.46 Hz, 3 H), 1.63 (ddd, J=14.40, 11.23, 3.17 Hz, 1 H), 1.74 (m, 1 H), 1.88 (m, 2 H), 2.38 (ddd, J=117.58, 3.66, 1.71 Hz, 1 H), 2.56 (dd, J=17.58, 4.64 Hz, 1 H), 3.27 (d, J=3.17 Hz, 1 H), 3.35 (m, 1 H), 4.16 (m, 3 H), 4.50 (m, 3 H), 7.30 (m, 7 H), 7.50 (m, 1 H), 7.95 (br t, J=6.35 Hz, 1 H).

### Example 101

### 4-[({2-(3,4-Difluoro-phenyl)-1-[2-((2R,4R)-4-hydroxy-6-oxo-tetrahydro-pyran-2-yl)-ethyl]-5-isopropyl-1H-imidazole-4-carbonyl]-amino)-methyl]-benzoic acid methyl ester

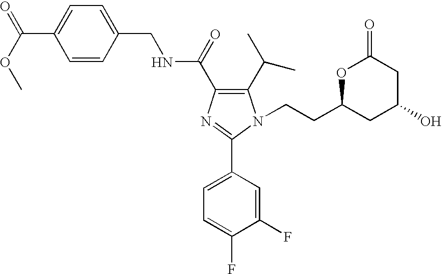

Starting from 1-[2-((4R,6R)-6-tert-Butoxycarbonylmethyl-2,2-dimethyl-[1,3]dioxan-4-yl)-ethyl]-2-(3,4-difluoro-phenyl)-5-isopropyl-1H-imidazole-4-carboxylic acid (522 mg, 1.0 mmol) the title compound was prepared in a manner similar to that described for Example 100. Yield: 332 mg (59%); Low resolution mass spectroscopy (APCI) m/z 556 [M+H]⁺; ¹H NMR (400 MHz, CD₃CN) δ 1.45 (d, J=1.71 Hz, 3 H), 1.46 (d, J=1.46 Hz, 3 H), 1.65 (ddd, J=14.40, 11.47, 3.17 Hz, 1 H), 1.76 (m, 1 H), 1.90 (m, 2 H), 2.39 (ddd, J=17.58, 3.42, 1.71 Hz, 1 H), 2.58 (dd, J=17.33, 4.39 Hz, 1 H), 3.26 (d, J=2.93 Hz, 1 H), 3.36 (m, 1 H), 3.85 (s, 3 H), 4.17 (m, 3 H), 4.51 (m, 1 H), 4.56 (d, J=6.35 Hz, 2 H), 7.39 (m, 4 H), 7.52 (m, 1 H), 7.94 (m, 2 H), 8.06 (br, t, 1 H).

### Example 102

### 2-(3,4-Difluoro-phenyl)-1-[2-((2R,4R)-4-hydroxy-6-oxo-tetrahydro-pyran-2-yl)-ethyl]-5-isopropyl-1H-imidazole-4-carboxylic acid 4-methoxy-benzylamide

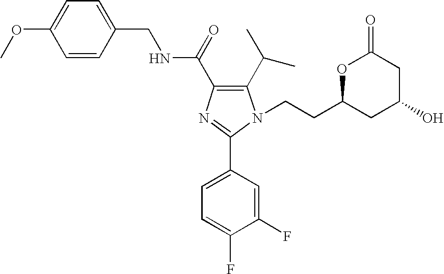

Starting from 1-[2-((4R,6R)-6-tert-Butoxycarbonylmethyl-2,2-dimethyl-[1,3]dioxan-4-yl)-ethyl]-2-(3,4-difluoro-phenyl)-5-isopropyl-1H-imidazole-4-carboxylic acid (522 mg, 1.0 mmol) the title compound was prepared in a manner similar to that described for Example 100. Yield: 335 mg (63%); Low resolution mass spectroscopy (APCI) m/z 528 [M+H]⁺; ¹H NMR (400 MHz, CD₃CN) δ 1.45 (d, J=1.46 Hz, 3 H), 1.47 (d, J=1.46 Hz, 3 H), 1.64 (ddd, J=14.40, 11.23, 2.93 Hz, 1 H), 1.75 (m, 1 H), 1.88 (m, 2 H), 2.39 (ddd, J=17.33, 3.42, 1.46 Hz, 1 H), 2.57 (dd, J=17.58, 4.64 Hz, 1 H), 3.28 (d, J=3.17 Hz, 1 H), 3.36 (m, 1 H), 3.75 (m, 3 H), 4.17 (m, 3 H), 4.41 (d, J=6.35 Hz, 2 H), 4.51 (ddd, J=15.87, 8.06, 3.91 Hz, 1 H), 6.87 (m, 2 H), 7.25 (m, 2 H), 7.37 (m, 2 H), 7.50 (m, 1 H), 7.89 (br t, J=6.35 Hz, 1 H).

### Example 103

### 5-Cyclopropyl-2-(4-fluoro-phenyl)-1-[2-((2R,4R)-4-hydroxy-6-oxo-tetrahydro-pyran-2-yl)-ethyl]-1H-imidazole-4-carboxylic acid benzylamide

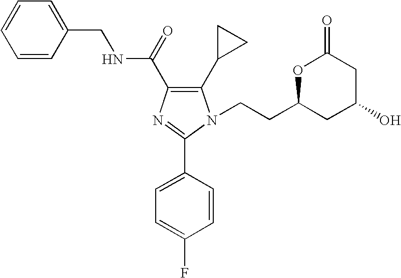

Starting from 1-[2-((4R,6R)-6-tert-Butoxycarbonylmethyl-2,2-dimethyl-[1,3]dioxan-4-yl)-ethyl]-5-cyclopropyl-2-(4-fluoro-phenyl)-1H-imidazole-4-carboxylic acid (4.85 g, 9.65 mmol) the title compound was prepared in a manner similar to that described for Example 100. Yield: 2.11 g (42%); Low resolution mass spectroscopy (APCI) m/z 478 [M+H]⁺; Anal. Calcd. For C₂₇H₂₈F₁N₃O₂/0.40 C₄H₈O₂: C, 66.99; H, 6.13; N, 8.19. Found: C, 66.63; H, 6.10; N, 8.22

### Example 104

### 5-Cyclopropyl-2-(4-fluoro-phenyl)-1-[2-((2R,4R)-4-hydroxy-6-oxo-tetrahydro-pyran-2-yl)-ethyl]-1H-imidazole-4-carboxylic acid 4-methoxybenzylamide

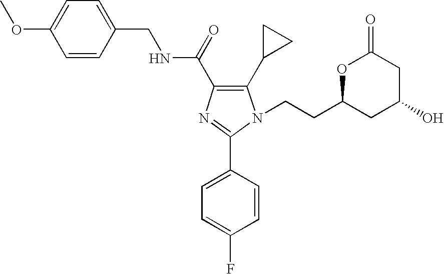

Starting from 1-[2-((4R,6R)-6-tert-Butoxycarbonylmethyl-2,2-dimethyl-[1,3]dioxan-4-yl)-ethyl]-5-cyclopropyl-2-(4-fluoro-phenyl)-1H-imidazole-4-carboxylic acid (500 mg, 1.0 mmol) the title compound was prepared in a manner similar to that described for Example 100. Yield: 243 mg (48%); Low resolution mass spectroscopy (APCI) m/z 508[M+H]⁺; ¹H NMR (400 MHz, CD₃CN) δ 0.97 (m, 2 H), 1.06 (m, 2 H), 1.63 (ddd, J=14.40, 11.23, 3.17 Hz, 1 H), 1.76 (m, 2 H), 1.94 (obscured m, 2 H), 2.39 (ddd, J=17.57, 3.66, 1.71 Hz, 1 H), 2.57 (dd, J=17.33, 4.64 Hz, 1 H), 3.26 (d, J=2.44 Hz, 1 H), 3.75 (s, 3 H), 4.16 (m, J=2.44 Hz, 1 H), 4.29 (m, 2 H), 4.40 (d, J6.34 Hz, 2 H), 4.50 (m, 1 H), 6.87 (m, 2 H), 7.23 (m, 4 H), 7.60 (m, 2 H), 7.76 (br t, J=5.86 Hz, 1 H).

### Example 105

### 5-Cyclopropyl-2-(4-fluoro-phenyl)- 1-[2-((2R,4R)-4-hydroxy-6-oxo-tetrahydro-pyran-2)-ethyl]-1H-imidazole-4-carboxylic acid benzyl-methyl-amide

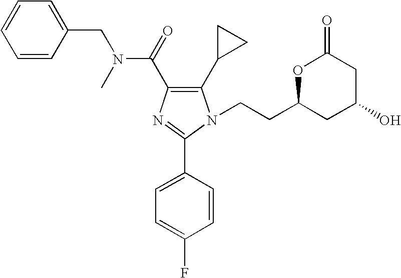

Starting from 1-[2-((4R,6R)-6-tert-Butoxycarbonylmethyl-2,2-dimethyl-[1,3]dioxan-4-yl)-ethyl]-5-cyclopropyl-2-(4-fluoro-phenyl)-1H-imidazole-4-carboxylic acid (700 mg, 1.39 mmol) the title compound was prepared in a manner similar to that described for Example 100. Yield: 298 mg (43%); Low resolution mass spectroscopy (APCI) m/z 492 [M+H]⁺; ¹H NMR (400 MHz, CD₃CN) δ 0.66 (m, 2 H), 0.94 (m, 2 H), 1.72 (m, 3 H), 1.97 (m, 2 H), 2.40 (m, 1 H), 2.58 (ddd, J=17.34, 4.64, 3.17 Hz, 1 H), 2.93 (d, J=5.37 Hz, 3 H), 3.32 (br t, J=3,42 Hz, 1 H), 4.24 (m, 3 H), 4.55 (m, 1 H), 4.68 (d, J=7.33 Hz, 2 H), 7.25 (m, 5 H), 7.39 (d, J=4.15 Hz, 2 H), 7.62 (m, 2 H).

### Example 106

### 2-(4-Fluoro-3-trifluoromethyl-phenyl)-1-[2-((2R,4R)-4-hydroxy-6-oxo-tetrahydro-pyran-2-yl)-ethyl]-5-isopropyl-1H-imidazole-4-carboxylic acid benzylamide

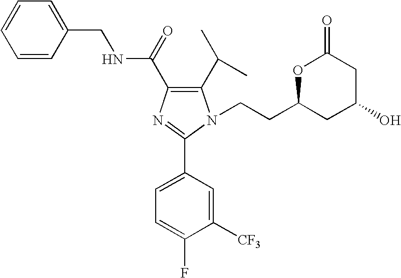

Starting from 1-[2-((4R,6R)-6-tert-Butoxycarbonylmethyl-2,2-dimethyl-[1,3]dioxan-4-yl)-ethyl]-2-(4-fluoro-3-trifluoromethyl-phenyl)-5-isopropyl-1H-imidazole-4-carboxylic acid (500 mg, 0.87 mmol) this compound was prepared in a manner similar to that described for Example 100. Yield: 167 mg (35%); Low resolution mass spectroscopy (APCI) m/z 548 [M+H]⁺; ¹H NMR (400 MHz, CD₃CN) δ 1.46 (d, J=1.46 Hz, 3 H), 1.48 (d, J=1.71 Hz, 3 H), 1.63 (ddd, J=14.40, 11.47, 2.93 Hz, 1 H), 1.75 (m, 2 H), 1.90 (m, 1 H), 2.38 (ddd, J=1 7.58, 3.42, 1.71 Hz, 1 H), 2.56 (dd, J=17.58, 4.64 Hz, 1 H), 3.32 (m, 1 H), 3.37 (m, 1 H), 4.18 (m, 3 H), 4.49 (m, 3 H), 7.23 (m, 1 H), 7.30 (m, 4 H), 7.42 (m, 1 H), 7.86 (m, 1 H), 7.91 (m, 1 H), 8.01 (t, J=6.35 Hz, 1 H).

### Example 107

### 4-[({2-(4-Fluoro-3-trifluoromethyl-phenyl)-1-[2-((2R,4R)-4-hydroxy-6-oxo-tetrahydro-pyran-2-yl)-ethyl]-5-isopropyl-1H-imidazole-4-carbonyl}-amino)-methyl]-benzoic acid methyl ester

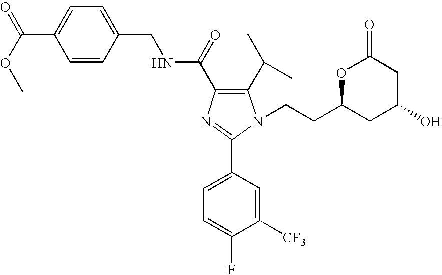

Starting from 1-[2-((4R,6R)-6-tert-Butoxycarbonylmethyl-2,2-dimethyl-[1,3]dioxan-4-yl)-ethyl]-2-(4-fluoro-3-trifluoromethyl-phenyl)-5-isopropyl-1H-imidazole-4-carboxylic acid (500 mg, 0.87 mmol) this compound was prepared in a manner similar to that described for Example 100. Yield: 186 mg (35%); Low resolution mass spectroscopy (APCI) m/z 606 [M+H]⁺; ¹H NMR (400 MHz, CD₃CN) δ 1.45 (d, J=2.20 Hz, 3 H), 1.47 (d, J=2.20 Hz, 3 H), 1.64 (ddd, J=14.16, 11.23, 2.93 Hz, 1 H), 1.75 (d, 1 H), 1.90 (m, 2 H), 2.38 (ddd, J=17.58, 3.42, 1.71 Hz, 1 H), 2.56 (dd, J=17.33, 4.39 Hz, 1 H), 3.36 (m, 2 H), 3.83 (s, 3 H), 4.18 (m, 3 H), 4.50 (m, 3 H), 7.39 (m, 3 H), 7.86 (ddd, J=7.32, 4.88, 1.95 Hz, 1 H), 7.90 (m, 3 H), 8.19 (t, J=6.35 Hz, 1 H),

### Example 108

### 2-(4-Fluoro-3-trifluoromethyl-phenyl)-1-[2-((2R,4R)-4-hydroxy-6-oxo-tetrahydro-pyran-2-yl)-ethyl]-5-isopropyl-1H-imidazole-4-carboxylic acid 4-methoxy-benzylamide

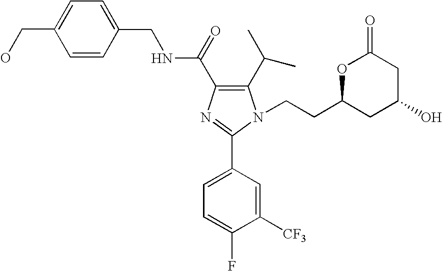

Starting from 1-[2-((4R,6R)-6-tert-Butoxycarbonylmethyl-2,2-dimethyl-[1,3]dioxan-4-yl)-ethyl]-2-(4-fluoro-3-trifluoromethyl-phenyl)-5-isopropyl-1H-imidazole-4-carboxylic acid (500 mg, 0.87 mmol) this compound was prepared in a manner similar to that described for Example 100. Yield: 239 mg (47%); Low resolution mass spectroscopy (APCI) m/z 578 [M+H]⁺; ¹H NMR (400 MHz, CD₃CN) δ 1.46 (d, J=1.71 Hz, 3 H), 1.48 (d, J=1.71 Hz, 3 H), 1.63 (ddd, J=14.28, 11.35, 2.93 Hz, 1 H), 1.74 (m,1 H), 1.89 (m, 2 H), 2.38 (ddd, J=17.58, 3.42, 1.71 Hz, 1 H), 3.35 (m,1 H), 3.40 (d, J=3.17 Hz, 1 H), 3.74 (s, 3 H), 4.16 (m, 3 H), 4.40 (d, J=6.35 Hz, 2 H), 4.49 (m,1 H), 6.84 (m, 2 H), 7.22 (m, 2 H), 7.41 (dd, J=10.25, 8.79 Hz, 1 H), 7.85 (m, 1 H), 7.90 (dd, J=6.84, 2.20 Hz, 1 H), 7.97 (t, J=6.23 Hz, 1 H).

### Example 109

### 2-(2,4-Difluoro-phenyl)-5-isopropyl-1-[2-((S)-6-oxo-3,6-dihydro-2H-pyran-2-yl)-ethyl]-1H-imidazole-4-carboxylic acid benzylamide

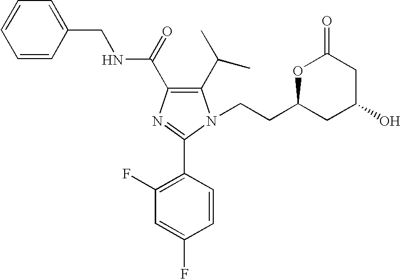

Starting from 1-[2-((4R,6R)-6-tert-Butoxycarbonylmethyl-2,2-dimethyl-[1,3]dioxan-4-yl)-ethyl]-2-(2,4-difluoro-phenyl)-5-isopropyl-1H-imidazole-4-carboxylic acid (234 mg, 0.44 mmol) this compound was prepared in a manner similar to that described for Example 100. Yield: 121 mg (54%); Low resolution mass spectroscopy (APCI) m/z 498 [M+H]⁺; ¹H NMR (400 MHz, CD₃CN) δ ppm (d, J=7.08 Hz, 6 H), 1.59 (ddd, J=14.28, 11.35, 3.17 Hz, 1 H), 1.70 (m, J=14.31, 3.59, 3.59, 1.95 Hz, 1 H), 1.82 (m, 2 H), 2.37 (ddd, J=17.46, 3.54, 1.46 Hz, 1 H), 2.55 (dd, J=17.33, 4.64 Hz, 1 H), 3.12 (s, 1 H), 3.39 (m, 1 H), 4.04 (m, 2 H), 4.14 (m, 1 H), 4.44 (m,1 H), 4.50 (d, J=6.35 Hz, 2 H), 7.10 (m, 2 H), 7.24 (m, 1 H), 7.32 (m, 4 H), 7.48 (m, 1 H), 7.90 (brt, J=6.10 Hz, 1 H).

### Example 110

### Sodium: (3R,5R)-7-[4-Benzylcarbamoyl-5-cyclopropyl-2-(4-fluoro-phenyl)-imidazol-1-yl]-3,5-dihydroxy-heptanoate

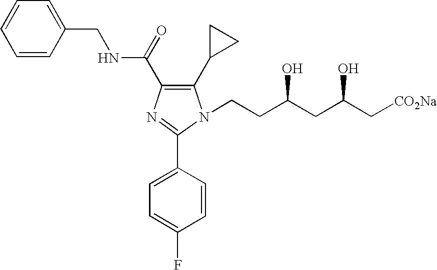

Starting from 5-Cyclopropyl-2-(4-fluoro-phenyl)-1-[2-((2R,4R)-4-hydroxy-6-oxo-tetrahydro-pyran-2-yl)-ethyl]-1H-imidazole-4-carboxylic acid benzylamide (1.52 g, 3.18 mmol) the title compound was prepared in a manner similar to that described for Example 4, step C. Yield: 1.69g (100%); Low resolution mass spectroscopy (APCI) ml,z 496 [M+H]⁺; Anal. Calcd. For C₂₇H₂₉F₁N₃Na₁O₅/1.4H₂O: C, 59.75; H, 5.91; N, 7.74. Found: C, 59.75; H, 5.75; N, 7.65.

### Example 111

### Sodium; (3R,5R)-7-[5-Cyclopropyl-2-(4-fluoro-phenyl)-4-(4-methoxy-benzylcarbamoyl)-imidazol-1-yl-3.5-dihydroxy-heptanoate

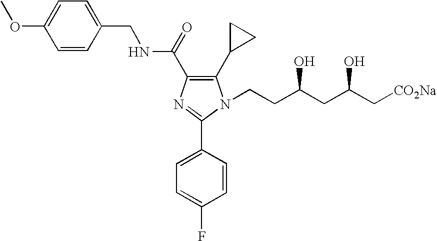

Starting from 5-Cyclopropyl-2-(4-fluoro-phenyl)-1-[2-((2R,4R)-4-hydroxy-6-oxo-tetrahydro-pyran-2-yl)-ethyl]-1H-imidazole-4-carboxylic acid 4-methoxybenzylamide (1.66 g, 3.28 mmol) the title compound was prepared in a manner similar to that described for Example 4, step C. Yield: 1.79 g (99%); Low resolution mass spectroscopy (APCI) m/z 526 [M+H]⁺; Anal Calcd. For C₂₈H₃₁F₁N₃Na₁O₆/0.9 H₂O: C, 59.65; H, 5.86; N, 7.45. Found: C, 59.69; H, 5.79; N, 7.40.

### Example 112

### Sodium: (3R,5R)-7-[4-(Benzyl-methyl-carbamoyl-5-cyclopropyl-2-(4-fluoro-phenyl)-imidazol-1-yl1-3,5-dihydroxy-heptanoate

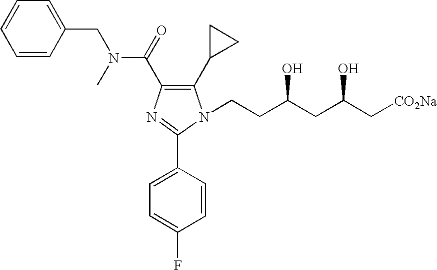

Starting from 5-Cyclopropyl-2-(4-fluoro-phenyl)-1-[2-((2R ,4R)-4-hydroxy-6-oxo-tetrahydro-pyran-2-yl)-ethyl]-1H-imidazole-4-carboxylic acid benzyl-methyl-amide (288 mg, 0.58 mmol) the title compound was prepared in a manner similar to that described for Example 4, step C. Yield: 305 mg (97%); Low resolution mass spectroscopy (APCI) m/z 510 [M+H]⁺; Anal. Calcd. For C₂₈H₃₁F₁N₃Na₁O₅/1.9H₂O: C, 59.44; H, 6.20; N, 7.43. Found: C, 59.43; H, 5.93; N, 7.39.

### Example 113

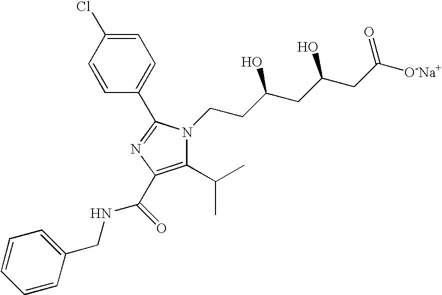

### Sodium: (3R,5R)-7-[4-benzylcarbamoyl-2-(4-chloro-phenyl)-5-isopropyl-imidazol- 1-yl]-3,5-dihydroxy-heptanoate

Step A

### (Benzhydrylidene-amino)-acetic acid methyl ester

Combined benzophenone imine (51 g, 273 mmol, Aldrich Chemical Co.), glycine methylester hydrochloride (35 g, 279 mmol, Aldrich Chemical Co.) and dichloromethane (340 ml) in a 500 ml round bottom flask under argon atmosphere. Stirred mixture 72 hours at rt. Removed solids by vacuum filtration, washing with diethyl ether. Concentrated solution to a pale yellow oil under reduced pressure. Diluted oil with diethyl ether (250 ml), washed twice with water, dried over sodium sulfate, filtered and concentrated to a pale yellow syrup. Product precipitates under vacuum drying to yield 64.9 g pale yellow prismatic crystals. MS (APCI) m/z 254 [M+H]⁺; ¹H NMR (400 MHz, CDCl₃) δ ppm 3.73 (s, 3H), 4.21 (s, 2H), 7.17 (m, 2H), 7.29-7.51 (m, 6H), 7.66 (m, 2H).

Step B

### 2-(4-Chloro-benzoylamino)-4-methyl-3-oxo-pentanoic acid methyl ester

To a 3-neck round bottom flask (equipped with overhead stirrer, N₂ line and thermocouple) charged with potassium tert-butoxide (124 ml,1.0 M in THF, Aldrich Chemical Co.) at −30° C. was added (Benzhydrylidene-amino)-acetic acid methyl ester (21 g, 82.9 mmol) The reaction mixture was stirred at −30° C. for 30 minutes under nitrogen positive pressure, then isobutyryl chloride (9.9 g, 91.2 mmol in 20 ml THF) was added via pressure equalizing addition funnel, dropwise, over 30 minutes. The reaction was stirred another 1 hour at the cold temperature than quenched with HCl (55 ml, 3.0 M). The precipitated yellow slurry was stirred 15 minutes, then concentrated under reduced pressure to a minimum volume. The residue was diluted with water (30 ml) and this mixture washed twice with diethyl ether (150 ml). The aqueous phase was returned to the 3-neck reaction flask, cooled to 2C and made basic (pH 9) by slow addition of neat sodium bicarbonate. Added ethyl acetate (150 ml), equilibrated mixture to 2C with stirring, then added 4-Chlorobenzoyl chloride (15.4 g, 87.1 mmol in 5 ml THF) via pressure equillizing funnel to maintain temperature below 5C. After 40 minutes stirring, warmed mixture to rt and transferred to a separation funnel. Removed aqueous phase and discarded. Washed organic phase with water, brine, dried over sodium sulfate, filtered and concentrated to a yellow powder. Purification by flash chromatography (SiO₂, 15%-60% ethyl acetate in hexanes) yielded 12.05 g fluffy white powder as desire product. MS (APCI) m/z 298 [M+H]⁺; ¹H NMR (400 MHz, CDCl₃) δ ppm 1.14 (d, J=6.8 Hz, 3H), 1.24 (d, J=7.1 Hz, 3H), 3.13 (septet, J=6.8 Hz, 1H), 3.83 (s, 3H), 5.58 (d, J=6.8 Hz, 1H), 7.42 (m, 2H), 7.78 (m, 2H), 8.01 (m, partially exchanged H).

Step C

### N-(1-Benzylcarbamoyl-3-methyl-2-oxo-butyl)-4-chloro-benzamide

To a solution of 2-(4-Chloro-benzoylamino)-4-methyl-3-oxo-pentanoic acid methyl ester (12.0 g, 40.3 mmol) in N-Methylpyrrolidinone (70 ml) was added benzylamine (4.8 g, 44.3 mmol) and a catalytic amount of p-Toluenesulfonic acid. The mixture was stirred and heated to 160C for 2 hours, then cooled and poured into chilled water (500 ml). The resultant slurry was extracted twice with ethyl acetate (150 ml). The organic phase was washed twice with 5% HCl solution, once with saturated sodium bicarbonate solution, once with brine, dried over sodium sulfate, filtered and concentrated to an off-white powder. The powder was dried overnight in vacuum oven at 40C to a stable weight of 10.3 g of desired product and ester. (APCI) m/z 371 [M−H]⁻.

Step D

### Sodium; (3R,5R)-7-[4-benzylcarbamoyl-2-(4-chloro-phenyl)-5-isopropyl-imidazol-1-yl]-3,5-dihydroxy-heptanoate

To a solution of N-(1-Benzylcarbamoyl-3-methyl-2-oxo-butyl)-4-chloro-benzamide (9.9 g, 26.7 mmol) in n-hepatne (80 ml), was added [(4R,6R)-6-(2-Amino-ethyl)-2,2-dimethyl-[1,3]dioxan-4-yl]-acetic acid tert-butyl ester (15 g, 53 mmol in 20 ml heptane), benzoic acid (9.8 g, 80 mmol) and a catalytic amount of p-toluenesulfonic acid. Attached Dean-Stark trap filled with heptane, condenser, nitrogen gas line and heated stirring mixture to reflux overnight. Cooled mixture to rt and concentrated under reduced pressure to a slurry. Dissolved mixture in ethyl acetate (100 ml), washed with saturated sodium bicarbonate solution (2×100 ml), water (3×100 ml), brine, dried over sodium sulfate, filtered and concentrated to a red-orange glass. Purified by flash chromatography (SiO₂, 10%-50% ethyl acetate in hexanes) to recover 4.8 g yellow glass as the protected imidazole amide. Dissolved glass in dichloromethane 25% trifluoroacetic acid (30 ml) and stirred at rt for 1.6 hours, then quenched and made basic with 1 M NaOH solution (pH 11). Concentrated product mixture to a minimum volume and purified by reverse phase (hemi-spherical C18, 100- 80% water/3% n-propanol in acetonitrile) and lyophilized to recover 1.92 g off-white powder as desired product. MS (APCI) m/z 514 [M+H]⁺; Anal. Calcd. for C₂₇H₃₁Cl₁N₃Na₁O₅/1.0 H₂O: C, 58.53; H, 6.00; N, 7.58. Found: C, 58.49; H, 6.17; N, 7.40.

### Example 114

### Sodium: (3R,5R)-7-[2-(4-chloro-phenyl)-5-isopropyl-4-(3-methoxy-benzylcarbamoyl)-imidazol-1-yl1-3,5-dihydroxy-heptanoate

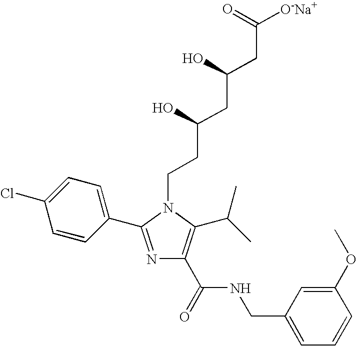

Starting from 2-(4-Chloro-benzoylamino)-4-methyl-3-oxo-pentanoic acid methyl ester this compound was made in a similar manner as described for example 113 (Steps C and D). MS (APCI) m/z 544 [M+H]⁺; Anal. Calcd. for C₂₈H₃₃Cl₁N₃Na₁O₆/1.15 H₂O: C, 57.32; H, 6.06; N, 7.16. Found: C, 57.22; H, 5.88; N, 7.01.

### Example 115

### Sodium: (3R,5R)-7-[4-benzylcarbamoyl-5-isopropyl-2-(4-methoxy-phenyl)-imidazol-1-yl]-3,5-dihydroxy-heptanoate

Starting from (Benzhydrylidene-amino)-acetic acid benzyl ester this compound was prepared in a similar manner as described for Example 113 (Steps B, C and D). MS (APCI) m/z 510

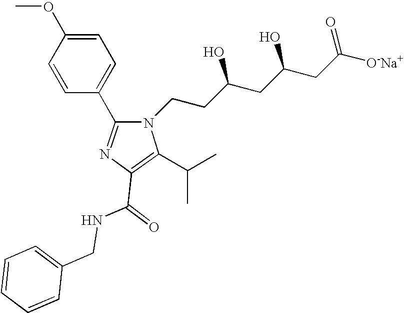

[M+H]⁺; Anal. Calcd. for C₂₈H₃₄N₃Na₁O₆/1.95 H₂O: C, 59.34; H, 6.74; N, 7.41. Found: C, 59.36; H, 6.62; N, 7.33.

### Example 116

### Sodium: (3R,5R)-3,5-dihydroxy-7-[5-isopropyl-4-(3-methoxy-benzylcarbamoyl)-2-(4-methoxy-phenyl)-imidazol-1-yl]-heptanoate

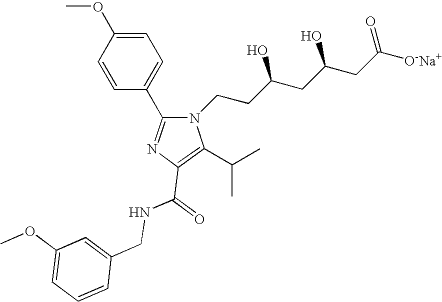

Starting from (Benzhydrylidene-amino)-acetic acid benzyl ester, this compound was prepared in a similar manner as described for Example 113 (Steps B, C and D). MS (APCI) m/z 540 [M+H]⁺; Anal. Calcd. for C₂₉H₃₆N₃Na₁O₇/1.35 H₂O: C, 59.45; H, 6.66; N, 7.17. Found: C, 59.37; H, 6.72; N, 7.16.

### Example 117

### Sodium :(3R,5R)-3,5-dihydroxy-7-[5-isopropyl-4-(4-methoxy-benzylcarbamoyl)-2-(4-methoxy-phenyl)-imidazol-1-yl]-heptanoate

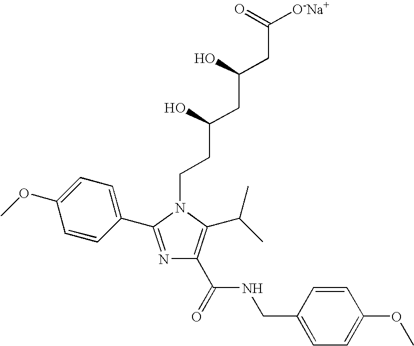

Starting from (Benzhydrylidene-amino)-acetic acid methyl ester, this compound was prepared in a similar manner as described for Example 113 (Steps B, C and D). MS (APCI) m/z 540 [M+H]⁺; Anal. Calcd. for C₂₉H₃₆N₃Na₁O₇/1.30 H₂O: C, 59.54; H, 6.65; N, 7.18. Found: C, 59.60; H, 6.74; N, 7.14.

### Example 118

### Sodium ;(3R5R)-7-[4-[2-(3-chloro-phenyl)-ethylcarbamoyl]-2-(4-fluoro-phenyl)-5-isopropyl-imidazol-1-yl]-3,5-dihydroxy-heptanoate

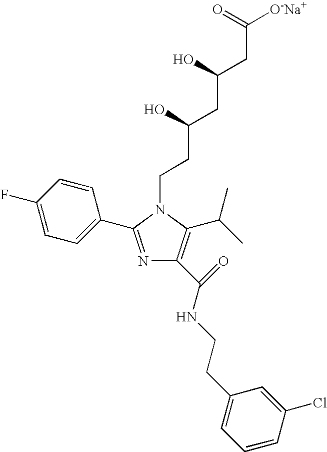

A solution of 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid (300 mg, 0.59 mmol), EDCI (170 mg, 0.89 mmol), and HOBt-monohydrate (140 mg, 0.89 mmol), in dichloromethane (2 ml) was stirred at rt for 30 minutes. 2-(3-Chloro-phenyl)-ethylamine (102 mg, 0.66 mmol) was added and the resultant mixture was stirred overnight. The reaction mixture was concentrated under reduced pressure, and the residue was partitioned between ethyl acetate and water. The organic layer was separated, washed with saturated sodium bicarbonate and brine, dried (Na₂SO₄), filtered and concentrated to a yellow glass. The crude glass was dissolved in a 30% trifluoroacetic acid/CH₂Cl₂ solution (4 ml) and stirred 1 hour. The reaction mixture was chilled (ice bath) diluted with water, made basic by the addition of 1 M NaOH, and concentrated under reduced pressure to a minimum volume. Purification by column chromatography (C18, CH3CN/water, 0 to 80% (3% n-propanol)) and lyophilization gave the desired product as an off-white powder: Yield 233 mg; MS (APCI) m/z 546 [M+H]⁺; Anal. Calcd. for C₂₈H₃₂Cl₁F₁N₃Na₁O₅/1.0 H₂O: C, 57.30; H, 5.71; N, 7.22. Found: C, 57.39; H, 5.85; N, 7.17.

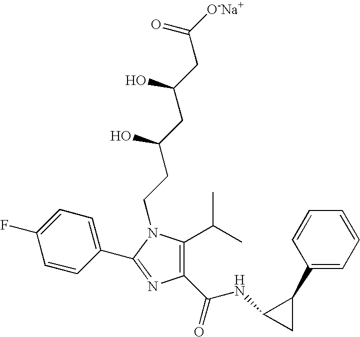

### Example 119

### Sodium; (3R,5R)-7-[2-(4-fluoro-phenyl)-5-isopropyl-4-((1S, 2R)-2-phenyl-cyclopropylcarbamoyl)-imidazol-1-yl]-3,5-dihydroxy-heptanoate

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 524 [M+H]⁺; Anal. Calcd. for C₂₉H₃₃F₁N₃Na₁O₅/1.2 H₂O: C, 61.41; H, 6.29; N, 7.41. Found: C, 61.20; H, 5.92; N, 7.44.

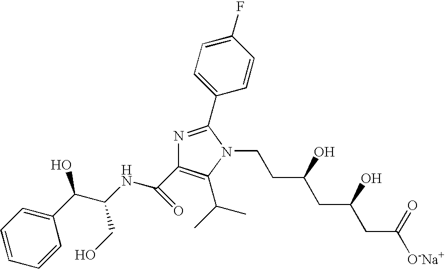

### Example 120

### Sodium; (3R,5R)-7-[2-(4-fluoro-phenyl)-4-((1R,2R)-2-hydroxy-1-hydroxymethyl-2-phenyl-ethylcarbamoyl)-5-isopropyl-imidazol-1-yl-3,5-dihydroxy-heptanoate

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 558 [M+H]⁺; ¹ H NMR (400 MHz, Methanol-D4) δ ppm 1.26 (d, J=7.1 Hz, 3H), 1.35 (d, J=7.1 Hz, 3H), 1.40 (dt,partially obscured, J=9.4, 4.9 Hz, 1H), 1.51 (dt, J=13.9, 8.1 Hz, 1H), 1.62 (m, 1H), 1.73 (m, 1H), 2.16 (dd, J=151.1, 7.3 Hz, 1H), 2.22 h(dd, J=14.9, 5.4 Hz, 1H), 3.30 (septet, partially obscured, J=7.1 Hz, 1H), 3.50 (dd, J=11.0, 5.6 Hz, 1H), 3.66 ( m, 2H), 3.93 (m, 2H), 4.13 (m, 2H), 4.93 (d, J=4.2 Hz, 1H), 7.12 (m, 1H), 7.19 (m, 4H), 7.34 (m, 2H), 7.56 (m, 2H).

### Example 121

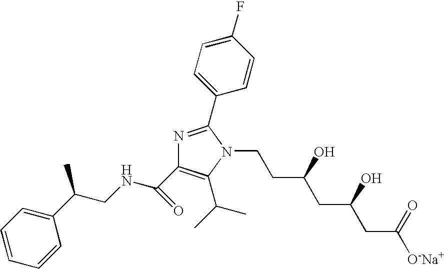

### Sodium; (3R,5R)-7-[2-(4-fluoro-phenyl)-5-isopropyl-4-((R)-2-phenyl-propylcarbamoyl)-imidazol-1-yl]-3,5-dihydroxy-heptanoate

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 526 [M+H]⁺; Anal. Calcd. for C₂₉H₃₅F₁N₃Na₁O₅/1.70 H₂O: C, 60.24; H, 6.69; N, 7.27. Found: C, 60.00H, 6.38; N, 7.15.

### Example 122

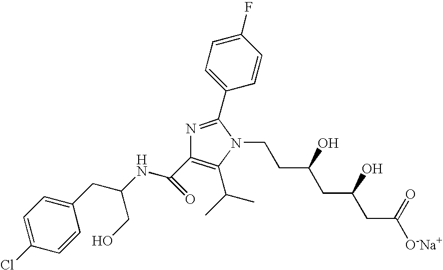

### Sodium: (3R,5R)-7-[4-[2-(4-chloro-phenyl)-1-hydroxymethyl-ethylcarbamoyl]-2-(4-fluoro-phenyl)-5-isopropyl-imidazol-1-yl]-3,5-dihydroxy-heptanoate

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 576 [M+H]⁺; Anal. Calcd. for C₂₉H₃₄Cl₁F₁N₃Na₁O₆/1.34 H₂O: C, 55.98; H, 5.94; N, 6.75. Found: C, 55.59; H, 5.94; N, 6.68.

### Example 123

### Sodium: (3R,5R)-7-[2-(4-fluoro-phenyl)-5-isopropyl-4-((S)-1-methyl-3-phenyl-propylcarbamoyl)-imidazol-1-yl]-3,5-dihydroxy-heptanoate

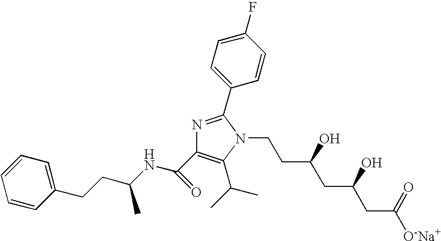

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 540 [M+H]⁺; ¹ H NMR (400 MHz, Methanol-D4 δ ppm 1.15 (d, J=6.6 Hz, 3H), 1.39 (t, J=4.9 Hz, 1H), 1.43 (dd, J=6.8, 2.0 Hz, 6H), 1.51 (dt, J=13.9, 8.2 Hz, 1H), 1.63 (m, 1H), 1.75 (m, 3H), 2.16 (dd, J=15.2, 7.3 Hz, 1H), 2.22 (dd, J=14.9, 5.1 Hz, 1H), 2.60 (m, 2H), 3.39 (septet, J=7.1 Hz, 1H), 3.66 (m, 1H), 3.93 (m, 3H), 4.14 (ddd, J=14.7, 11.1, 5.3 Hz, 1H), 7.01-7.23 (m, 7H), 7.55 (m, 2H).

### Example 124

### Sodium: (3R,5R)-7-{2-(4-fluoro-phenyl)-4-[2-(3-fluoro-phenyl)-ethylcarbamoyl]-5-isopropyl-imidazol-1-yl]-3,5-dihydroxy-heptanoate

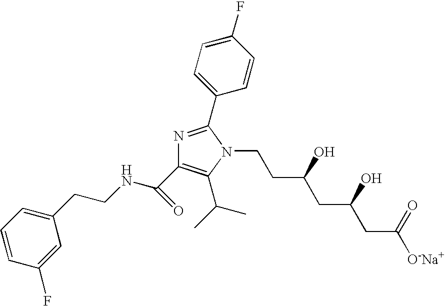

Starting from 1-[2-((4R, 6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118 MS (APCI) r/z 530 [M+H]⁺; ¹H NMR (400 MHz, Methanol-D4) δ ppm 1.39 (d, J=7.1, Hz, 3H), 1.40 (d, J=7.1, Hz, 3H), 1.42 (t, partially obscured, J=4.9 Hz,1H), 1.51 (dt, J=13.9, 8.3 Hz, 1H), 1.62 (m, 1H), 1.74 (m, 1H), 2.16 (dd, J=15.1, 7.3 Hz, 1H), 2.22 (dd, J=15.1, 5.4 Hz, 1H), 2.81 (t, J=7.3 Hz, 2H), 3.35 (septet, J=6.8 Hz, 1H), 3,48 (t, J=7.3 Hz, 2H), 3.66 (m, 1H), 3.93 (m, 2H), 4.13 (ddd, J=14.8, 11.1, 5.1 Hz, 1H), 6.83 (td, J=8.6, 1.8 Hz, 1H), 6.94 (dt, J=10.1, 1.9 Hz, 1H), 7.00 (d, J=7.6 Hz,1H), 7.13-7.23 (m, 3H), 7.49-7.57 (m, 2H).

### Example 125

### Sodium; (3R,5R)-7-[2-(4-fluoro-phenyl)-4-((1S,2S)-2-hydroxy-1-methoxymethyl-2phenyl-ethylcarbamoyl)-5-isopropyl-imidazol-1-yl]-3,5-dihydroxy-heptanoate

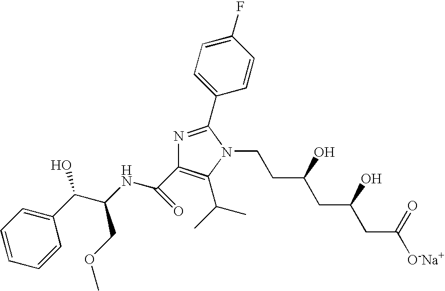

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118 MS (APCI) m/z 572 [M+H]⁺; ¹ H NMR (400 MHz, Methanol-D4) δ ppm 1.30 (d, J=6.8 Hz, 3H), 1.38 (d, J=6.8 Hz, 3H), 1.43 (dt, J=13.9, 4.8 Hz, 1H), 1.53 (dt, J=13.9, 8.1 Hz, 1H), 1.64 (m, 1H), 1.76 (m, 1H), 2.18 (dd, J=15.1, 7.3 Hz, 1H), 2.24 (dd, J=15.1, 5.4 Hz, 1H), 3.28 (m, 4H), 3.32 (septet, partially obscured, J=6.8 Hz, 1H), 3.53 (dd, J=9.5, 6.6 Hz, 1H), 3.68 (m, 1H), 3.89-4.02 (m, 2H), 4.15 (m, 1H), 4.26 (td, J=6.0, 5.1, 4.8 Hz, 1H), 4.90 (d, J=4.4 Hz, 1H), 7.12-7.25 (m, 5H), 7.34 (apparent d, J=7.3 Hz, 2H), 7.58 (m, 2H).

### Example 126

### Sodium: (3R,5R)-7-{2-(4-fluoro-phenyl)-5-isopropyl-4-[2-(4-methoxy-phenyl)-ethylcarbarbamoyl]-imidazol-1-yl]-3,5-dihydroxy-heptanoate

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 542 [M+H]⁺; ¹ H NMR (400 MHz, Methanol-D4) δ ppm 1.40 (d, J=7.1 Hz, 6H), 1.14 (m, partially obscured,1 H), 1.51 (dt, J=13.9, 8.1 Hz, 1H), 1.64 (m, 1H), 1.73 (m, 1H), 2.16 (dd, J=15.1, 7.3 Hz, 1H), 2.22 (dd, J=14.9, 5.1 Hz, 1H), 2.72 (t, J=7.3 Hz, 2H), 3.36 (septet, partially obscured, J=6.8 Hz, 1H), 3,43 (t, J=7.4 Hz, 2H), 3.66 (m, 4H), 3.91 (m, partially obscured, 1H), 3.98 (dd, J=10.7, 5.1 Hz, 1H), 4.13 (ddd, J=16.1, 11.5, 5.1 Hz, 1H), 6.75 (m, 2H), 7.1 (m, 2H), 7.17 (m, 2H), 7.52 (m, 2H).

### Example 127

### Sodium; (3R,5R)-7-{2-(4-fluoro-phenyl)-4-[(S)-2-hydroxy-1-hydroxymethyl-2-(4-methylsulfanyl-phenyl)-ethylcarbamoyl]-5-isopropyl-imidazol-1-yl-3,5-

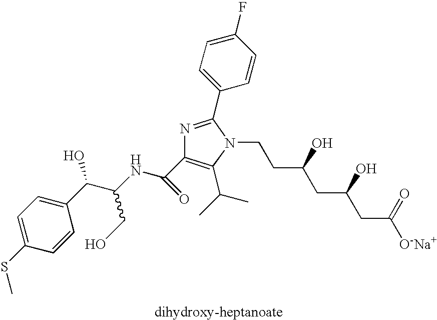

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 604 [M+H]⁺; ¹ H NMR (400 MHz, Methanol-D4) δ ppm 1.25 (dd, J=6.8, 4.9 Hz, 3H), 1.36 (dd, J=7.1, 2.7 Hz, 3H), 1.41 (m, 1H), 1.51 (m, 1H), 1.62 (m, 1H), 1.74 (m, 1H), 2.16 (ddd, J=15.1, 7.5, 1.6 Hz, 1H), 2.22 (ddd, J=15.2, 5.4, 2.1 Hz, 1H), 2.35 (d, J=2.9 Hz, 3H), 3.29 (m, partially obscured, 1H), 3.52 (dd, J=11.1, 5.5 Hz, 0.66H), 3.60 (dd, J=11.5, 4.2 Hz, 0.33H), 3.68 (dd, partially obscured, J=11.2, 6.6 Hz, 0.66H), 3.65 (m, obscured, 1H), 3.77 (dd, J=11.5, 5.9 Hz, 0.33H), 3.87-4.01 (m, 2H), 4.07-4.21 (m, 2H), 4.91 (d, J=3.7 Hz, 1H), 7.12 (m, 2H), 7.19 (m, 2H), 7.28 (m, 2H), 7.55 (m, 2H).

### Example 128

### Sodium; (3R,5R)-7-[2-(4-fluoro-phenyl)-5-isopropyl-4-((S)-2-phenyl-propylcarbamoyl)-imidazol-1-yl]-3,5-dihydroxy-heptanoate

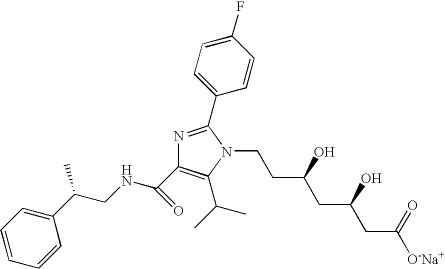

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for PF-02309081-02. MS (APCI) m/z 526 [M+H]⁺; ¹ H NMR (400 MHz, Methanol-D4) δ ppm 1.22 (d, J=6.8 Hz, 3H), 1.36-1.44 (m, 7H), 1.51 (dt, J=13.9, 8.1 Hz, 1H), 1.61 (m, 1H), 1.72 (m, 1H), 2.16 (dd, J=14.9, 7.3 Hz, 1H), 2.22 (dd, J=15.1, 5.4 Hz, 1 H), 2.95 (sextet, J=7.1 Hz, 1H), 3.28-3,50 (m, 3H), 3.65 (m, 1H), 3.91 (m, 1H), 3.97 (dd, J=10.9, 5.3 Hz, 1H), 4.12 (ddd, J=14.8, 11.1, 5.1 Hz, 1H), 7.06-7.25 (m, 7H), 7.50 (m, 2H), 7.63 (t, J=6.0 Hz, partially exchanged amide H).

### Example 129

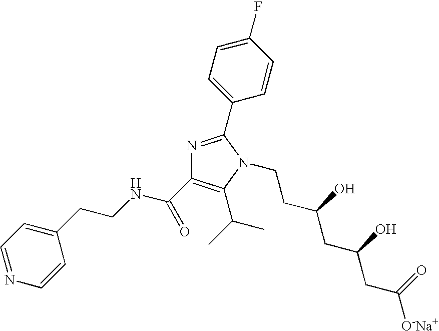

### Sodium: (3R,5R)-7-[2-(4-fluoro-phenyl)-5-isopropyl-4-(2-pyridin-4-yl-ethylcarbamoyl)-imidazol-1-yl]-3,5-dihydroxy-heptanoate

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 513 [M+H]⁺; Anal. Calcd. for C₂₇H₃₂F₁N₄Na₁O₅/1.6 H₂O:, 57.56; H, 6.30; N, 9.94. Found: C, 57.49; H, 6.00; N, 9.84.

### Example 130

### Sodium; (3R,5R)-7-[2-(4-fluoro-phenyl)-5-isopropyl-4-[2-(4-sulfamoyl-phenyl)-ethylcarbamoyl]-imidazol-1-yl}-3,5-dihydroxy-heptanoate

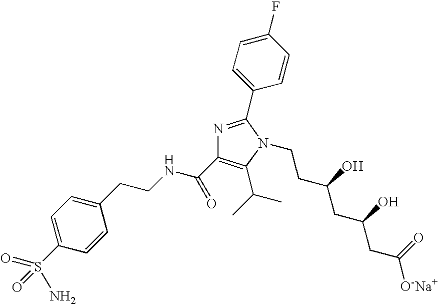

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI)+m/z 591 [M+H]⁺; ¹ H NMR (400 MHz, Methanol-D4) δ ppm 1.40 (d, J=7.1 Hz, 3H), 1.40 (d, J=7.1 Hz, 3H), 1.41 (m, partially obscured, 1H), 1.51 (dt, J=13.8, 8.1 Hz, 1H), 1.62 (m, 1H), 1.74 (m, 1H), 2.16 (dd, J=15.0, 7.3 Hz, 1 H), 2.22 (dd, J=15.0, 5.4 Hz, 1H), 2.88 (t, J=7.3 Hz, 2H), 3.36 (septet J=7.1 Hz, 1H), 3,51 (t, J=7.3 Hz, 2H), 3.66 (m, 1H), 3.91 (m, 1H), 3.98 (apparent dd, J=10.9, 5.0 Hz, 1H), 4.13 (apparent ddd, J=15.1, 11.2, 5.1 Hz, 1H), 7.17 (apparent t, J=8.7 Hz, 2H), 7.34 (d, J=8.3 Hz, 2H), 7.53 (m 2H), 7.74 (apparent d, J=8.3 Hz, 2H).

### Example 131

### Sodium: (3R,5R)-7-[4-((R)-1-carbamoyl-2-phenyl-ethylcarbamoyl)-2-(4-fluoro-phenyl)-5-isopropyl-imidazol-1-yl]-3,5-dihydroxy-heptanoate

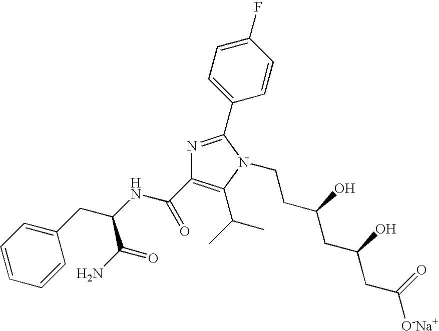

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 555 [M+H]⁺; Anal. Calcd. for C₂₉H₃₄F₁N₄Na₁O₆/2.8 H₂O: C, 55.55; H, 6.37; N, 8.94. Found: C, 55.20; H, 6.29; N, 8.77.

### Example 132

### Sodium: (3R,5R)-7-[2-(4-fluoro-phenyl)-5-isopropyl-4-(2-pyridin-3-yl-ethylcarbamoyl)-imidazol-1-yl]-3,5-dihydroxy-heptanoate

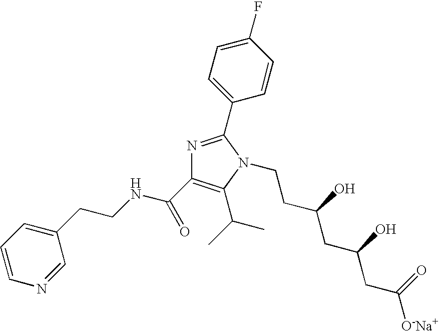

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 513 [M+H]⁺; Anal. Calcd. for C₂₇H₃₂F₁N₄Na₁O₅/1.0 H₂O: C, 58.69; H, 6.20; N, 10.14. Found: C, 58.46; H, 6.28; N, 10.00.

### Example 133

### Sodium: (3R,5R)-7-{2-(4-fluoro-phenyl)-4-[2-(4-fluoro-phenyl)-ethylcarbamoyl]-5-isopropyl-imidazol-1-yl]-3,5-dihydroxy-heptanoate

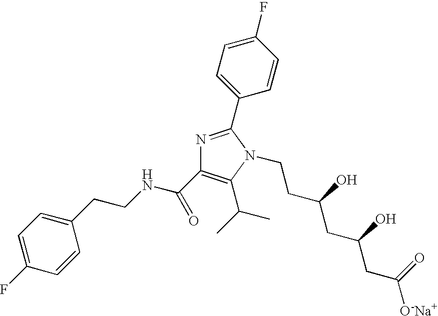

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 530 [M+H]⁺; Anal. Calcd. for C₂₈H₃₂F₂N₃Na₁O₅/0.95 H₂O: C, 59.14; H, 6.01; N, 7.39. Found: C, 58.97; H, 5.90; N, 7.30.

### Example 134

### Sodium; (3R,5R)-7-[2-(4-fluoro-phenyl)-5-isopropyl-4-(1-methyl-3-phenyl-propylcarbamoyl)-imidazol-1-yl]-3,5-dihydroxy-heptanoate

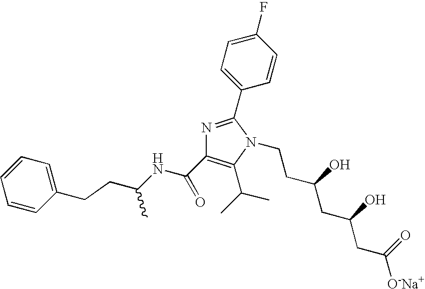

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 540 [M+H]⁺; Anal. Calcd. for C₃₀H₃₇F₁N₃Na₁O₅/1.85 H₂O: C, 60.56; H, 6.90; N, 7.06. Found: C, 60.43; H, 6.97; N, 7.00

### Example 135

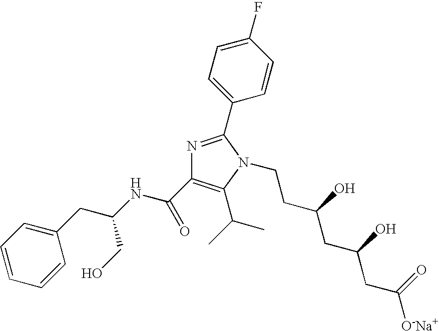

### Sodium: (3R,5R)-7-[4-((S)-1-benzyl-2-hydroxy-ethylcarbamoyl)-2-(4-fluoro-phenyl)-5-isopropyl-imidazol-1-yl]-3,5-dihydroxy-heptanoate

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 542 [M+H]⁺; ¹ H NMR (400 MHz, Methanol-D4) δ ppm 1.32 (d, J=7.1 Hz, 3H), 1.38 (d, J=7.1 Hz, 3H), 1.40 (m, partially obscured, 1H), 1.51 (dt, J=13.9, 8.2 Hz, 1H), 1.61 (m, 1H), 1.72 (m, 1H), 2.16 (dd, J=15.1, 7.3 Hz, 1H), 2.22 (dd, J=15.1, 5.1 Hz, 1H), 2.78 (dd, J=13.7, 7.6 Hz, 1H), 2.88 (dd, 13.4, 6.8 Hz,1 H), 3.33 (septet, J=7.1 Hz,1 H), 3,51 (d, J=4.9 Hz, 2H), 3.65 (m, 1H), 3.92 (m, 1H), 3.98 (dd, J=10.7, 5.4 Hz, 1H), 4.11 (dd, J=11.0, 4.9 Hz, 1H), 4.17 (m, 1H), 7.10 (m, 1H), 7.19 (m, 6H), 7.56 (m, 2H).

### Example 136

### Sodium: (3R,5R)-7-(2-(4-fluoro-phenyl)-5-isopropyl-4-[2-(3-methoxy-phenyl)-ethylcarbamoyl]-imidazol-1-yl-3,5-dihydroxy-heptanoate

Starting from 1-[2-((4R,6R)-6-tert-Butoxycarbonylmethyl-2,2-dimethyl-[1,3]dioxan-4-yl)-ethyl]-2-(4-fluoro-phenyl)-5-isopropyl-1H-imidazole-4-carboxylic acid, this compound was prepared in a similar fashion as described for Example 118. MS (APCI) m/z 542 [M+H]⁺; ¹ H NMR (400 MHz, Methanol-D4) δ ppm 1.39 (d, J=7.1 Hz, 3H), 1.39 (d, J=7.1 Hz, 3H), 1.40 (m, partially obscured, 1H), 1.51 (dt, J=14.0, 8.2 Hz, 1H), 1.61 (m, 1H), 1.73 (m, 1H), 2.15 (dd, J=15.1, 7.8,1 H), 2.22 (dd, J=15.1, 5.1, 1 H), 2.75 (t, apparent, J=7.6 Hz, 2H), 3.35 (septet, J=7.1 Hz, 1H), 3,46 (dd, J=8.1, 6.8 Hz, 2H), 3.64 (s, 3H), 3.66 (m, 1H), 3.91 (m, 1H), 3.97 (dd, J=11.0, 5.4 Hz, 1H), 4.12 (ddd, J=14.6, 11.0, 4.9 Hz, 1H), 6.65 (ddd, J=8.3, 2.7, 1.0 Hz, 1H), 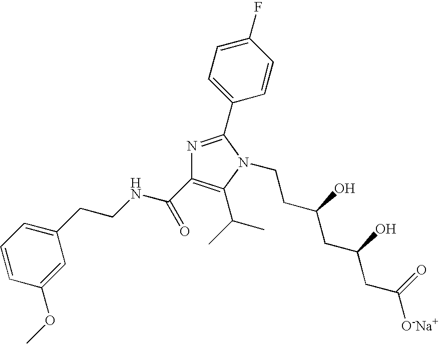
6.74 (m, 2H), 7.09 (m, 1H), 7.16 (m, 2H), 7.51 (m, 2H).

### Example 137

### Sodium: (3R,5R)-7-[4-benzyloxycarbonylamino-2-(4-fluoro-phenyl)-5-isopropyl-imidazol-1-yl]-3,5-dihydroxy-heptanoate sodium salt

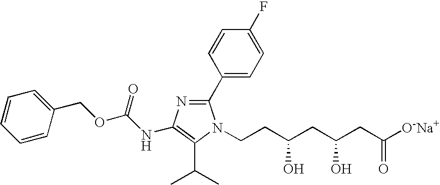

Step A

### (4R,6R)-(6-[2-r4-benzyloxycarbonylamino-2-(4-fluoro-phenyl)-5-isopropyl-imidazol-1-yl]-ethyl]-2,2-dimethyl-[1,3]dioxan-4-yl)-acetic acid tert-butyl ester

To a solution of (4R,6R)-1-[-2-(6-tert-butoxycarbonylmethyl-2,2-dimethyl-[1,3]dioxan-4-yl)-ethyl]-2-(4-fluoro-phenyl)-5-isopropyl-1H-imidazol-4-carboxylic acid (5.0 g, 9.9 mmoles) (Example 2) in 125 mL of toluene was added diphenylphosphoryl azide (DPPA) (2.4 mL, 3.0 g, 11 mmoles), followed by triethyl amine (2.2 mL, 1.6 g, 7.2 mmoles). The reaction mixture was refluxed for 3 hrs and then cooled to room temperature. Benzyl alcohol (1.5 mL, 1.6 g, 15 mmoles) was added and then the reaction mixture was stirred for 3 days. The reaction mixture was evaporated to give a brown oil, which was purified by flash chromatography (silica gel, 60% ethyl acetate in hexane, gradient elution) to provide 0.78 g (32% chr) of the desired product as a tan tacky solid: MS(APCI⁺) m/z 610; H¹ NMR (400 MHz DMSO-d₆) δ 8.60, 7.10-7.70, 5.05, 3.75-4.10, 2.90, 2.10-2.30, 0.95-1.70.

Step B

### (4,6)-{2-(4-Fluoro-phenyl)-1-[2-(4-hydroxy-6-oxo-tetrahydro-pyran-2-yl)-ethyl]-5-isopropyl-1H-imidazol-4-yl]-carbamic acid benzyl ester

To a solution of (4R,6R)-(6-{2-[4-benyloxycarbonylamino-2-(4-fluoro-phenyl)-5-isopropyl-imidazol-1-yl]-ethyl}-2,2-dimethyl-[1,3]dioxan-4-yl)-acetic acid tert-butyl ester (0.49 g, 0.80 mmoles) in 20 mL of dichloromethane was added 5 mL of trifluoroacetic acid (7.5 g, 65 mmoles). The reaction mixture was stirred at room temperature for 1.5 hrs. The reaction mixture was diluted with 200 mL of dichloromethane and 100 mL of saturated sodium bicarbonate solution. Solid sodium bicarbonate was added to pH=9. The organic layer was separated, dried (sodium sulfate), filtered, and then the filtrate was evaporated to afford a light-yellow foamy solid. Purification by flash chromatography (silica gel, 95% ethyl acetate in methanol) gave 269 mg (68%) of the desired product as a light yellow foamy solid: mp 86-90° C.; MS(APCI⁺) m/z 496.

Step C

### (3R,5R)-7-[4-benzyloxycarbonylamino-2-(4-fluoro-phenyl)-5-isopropyl-imidazol-1-yl]-3,5-dihydroxy-heptanoate sodium salt

To a solution of (4R,6R)-{2-(4-fluoro-phenyl)-1-[2-(4-hydroxy-6-oxo-tetrahydro-pyran-2-yl)-ethyl]-5-isopropyl-1H-imidazol-4-yl)-carbamic acid benyl ester (0.24 g, 0.47 mmoles) in 6 mL of methanol was added 0.51 mL of a 1.028 N aqueous solution of NaOH (0.02 g, 0.52 mmoles). The reaction mixture was stirred at room temperature for 3 hrs and then evaporated in vacuo to give a yellow oil, which was triturated in 50 mL of anhydrous diethyl ether at room temperature for 18 hrs. The mixture was filtered to collect a solid, which was rinsed with anhydrous diethyl ether and then dried to provide 198 mg (78%) of the desired product as an off-white solid: MS(APCI⁺) m/z 514; H¹ NMR (400 MHz DMSO-d₆) δ 8.65, 7.20-7.60, 5.05, 4.90, 3.80-4.10, 3,50-3.70, 2.90,1.1-1.95

Examples 138-423 are tabulated in the following Table I, (Lactones) and Table II (salts). The NMR data for each of the compounds of the following examples is consistant with its molecular structure.

TABLE I

| | | Lactone LC-MS |
|---|---|---|
| Example # | Lactone (IUPAC) | (APCI) [M + H]+ |
| 138 | 2-(4-Fluoro-phenyl)-1-[2-((2R,4R)-4-hydroxy-6-oxo- | 480 |
| | tetrahydro-pyran-2-yl)-ethyl]-5-isopropyl-1H-imidazole-4- | |
| | carboxylic acid benzylamide | |
| 139 | 2-(4-Fluoro-phenyl)-1-[2-((2R,4R)-4-hydroxy-6-oxo- | 481 |
| | tetrahydro-pyran-2-yl)-ethyl]-5-isopropyl-1H-imidazole-4- | |
| | carboxylic acid (pyridin-3-ylmethyl)-amide | |
| 140 | 2-(4-Fluoro-phenyl)-1-[2-(4-hydroxy-6-oxo-tetrahydro- | 494 |
| | pyran-2-yl)-ethyl]- | |
| | 5-isopropyl-1H-imidazole-4-carboxylic acid benzyl- | |
| | methyl-amide | |
| 141 | 2-(4-Fluoro-phenyl)-1-[2-((2R,4R)-4-hydroxy-6-oxo- | 550 |
| | tetrahydro-pyran-2-yl)-ethyl]-5-isopropyl-1H-imidazole-4- | |
| | carboxylic acid 2,3-dichloro-benzylamide | |
| 142 | 2-(4-Fluoro-phenyl)-1-[2-((2R,4R)-4-hydroxy-6-oxo- | 510 |
| | tetrahydro-pyran-2-yl)-ethyl]-5-isopropyl-1H-imidazole-4- | |
| | carboxylic acid 3-methoxy-benzylamide | |
| 143 | 2-(4-Fluoro-phenyl)-1-[2-((2R,4R)-4-hydroxy-6-oxo- | 574.2 |
| | tetrahydro-pyran-2-yl)-ethyl]-5-isopropyl-1H-imidazole-4- | |
| | carboxylic acid (2′-fluoro-biphenyl-3-ylmethyl)-amide | |
| 144 | 2-(4-Fluoro-phenyl)-1-[2-(4-hydroxy-6-oxo-tetrahydro- | 523 |
| | pyran-2-yl)-ethyl]-5-isopropyl-1H-imidazole-4-carboxylic | |
| | acid benzyl-isopropyl-amide | |
| 145 | 2-(4-Fluoro-phenyl)-1-[2-((2R,4R)-4-hydroxy-6-oxo- | 557 |
| | tetrahydro-pyran-2-yl)-ethyl]-5-isopropyl-1H-imidazole-4- | |
| | carboxylic acid (6-phenyl-pyridin-3-ylmethyl)-amide | |
| 146 | 2-(4-Fluoro-phenyl)-1-[2-(4-hydroxy-6-oxo-tetrahydro- | 522 |
| | pyran-2-yl)-ethyl]-5-isopropyl-1H-imidazole-4-carboxylic | |
| | acid benzyl-propyl-amide | |
| 147 | 2-(4-Fluoro-phenyl)-1-[2-(4-hydroxy-6-oxo-tetrahydro- | 498 |
| | pyran-2-yl)-ethyl]-5-isopropyl-1H-imidazole-4-carboxylic | |
| | acid (1,5-dimethyl-1H-pyrazol-3-ylmethyl)-amide | |
| 148 | 2-(4-Fluoro-phenyl)-1-[2-((2R,4R)-4-hydroxy-6-oxo- | 586 |
| | tetrahydro-pyran-2-yl)-ethyl]-5-isopropyl-1H-imidazole-4- | |
| | carboxylic acid (3′-hydroxymethyl-biphenyl-3-ylmethyl)- | |
| | amide | |
| 149 | 2-(4-Fluoro-phenyl)-1-[2-((2R,4R)-4-hydroxy-6-oxo- | 557 |
| | tetrahydro-pyran-2-yl)-ethyl]-5-isopropyl-1H-imidazole-4- | |
| | carboxylic acid 3-pyridin-3-yl-benzylamide | |
| 150 | 2-(4-Fluoro-phenyl)-1-[2-((2R,4R)-4-hydroxy-6-oxo- | 571 |
| | tetrahydro-pyran-2-yl)-ethyl]-5-isopropyl-1H-imidazole-4- | |
| | carboxylic acid (6-o-tolyl-pyridin-3-ylmethyl)-amide | |
| 151 | 2-(4-Fluoro-phenyl)-1-[2-((2R,4R)-4-hydroxy-6-oxo- | 572 |
| | tetrahydro-pyran-2-yl)-ethyl]-5-isopropyl-1H-imidazole-4- | |
| | carboxylic acid [(S)-1-(4-bromo-phenyl)-ethyl]-amide | |
| 152 | 2-(4-Fluoro-phenyl)-1-[2-((2R,4R)-4-hydroxy-6-oxo- | 572 |
| | tetrahydro-pyran-2-yl)-ethyl]-5-isopropyl-1H-imidazole-4- | |
| | carboxylic acid [(R)-1-(4-bromo-phenyl)-ethyl]-amide | |
| 153 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid ((R)-1-p-tolyl-ethyl)-amide | |
| 154 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid ((S)-1-p-tolyl-ethyl)-amide | |
| 155 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid [(R)-1-(4-methoxy-phenyl)-ethyl]-amide | |
| 156 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid [(S)-1-(4-methoxy-phenyl)-ethyl]-amide | |
| 157 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid [(R)-1-(3-methoxy-phenyl)-ethyl]-amide | |
| 158 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid [(S)-1-(3-methoxy-phenyl)-ethyl]-amide | |
| 159 | 2-(4-Fluoro-phenyl)-1-[2-((2R,4R)-4-hydroxy-6-oxo- | 570 |
| | tetrahydro-pyran-2-yl)-ethyl]-5-isopropyl-1H-imidazole-4- | |
| | carboxylic acid (2′-methyl-biphenyl-3-ylmethyl)-amide | |
| 160 | 3′-[({2-(4-Fluoro-phenyl)-1-[2-((2R,4R)-4-hydroxy-6-oxo- | 614 |
| | tetrahydro-pyran-2-yl)-ethyl]-5-isopropyl-1H-imidazole-4- | |
| | carbonyl}-amino)-methyl]-biphenyl-3-carboxylic acid | |
| | methyl ester | |
| 161 | 2-(4-Fluoro-phenyl)-1-[2-(4-hydroxy-6-oxo-tetrahydro- | 495 |
| | pyran-2-yl)-ethyl]-5-isopropyl-1H-imidazole-4-carboxylic | |
| | acid methyl-pyridin-2-ylmethyl-amide | |
| 162 | 2-(4-Fluoro-phenyl)-1-[2-(4-hydroxy-6-oxo-tetrahydro- | 495 |
| | pyran-2-yl)-ethyl]-5-isopropyl-1H-imidazole-4-carboxylic | |
| | acid methyl-pyridin-3-ylmethyl-amide | |
| 163 | 2-(4-Fluoro-phenyl)-1-[2-(4-hydroxy-6-oxo-tetrahydro- | 495 |
| | pyran-2-yl)-ethyl]-5-isopropyl-1H-imidazole-4-carboxylic | |
| | acid methyl-pyridin-4-ylmethyl-amide | |
| 164 | 3-{5-[({2-(4-Fluoro-phenyl)-1-[2-((2R,4R)-4-hydroxy-6- | 629 |
| | oxo-tetrahydro-pyran-2-yl)-ethyl]-5-isopropyl-1H- | |
| | imidazole-4-carbonyl}-amino)-methyl]-pyridin-2-yl}- | |
| | benzoic acid ethyl ester | |
| 165 | 2-(4-Fluoro-phenyl)-1-[2-((2R,4R)-4-hydroxy-6-oxo- | 557 |
| | tetrahydro-pyran-2-yl)-ethyl]-5-isopropyl-1H-imidazole-4- | |
| | carboxylic acid (2′-methoxy-biphenyl-3-ylmethyl)-amide | |
| 166 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid ((S)-1-phenyl-propyl)-amide | |
| 167 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid ((R)-1-phenyl-propyl)-amide | |
| 168 | 2-(4-Fluoro-phenyl)-1-[2-((2R,4R)-4-hydroxy-6-oxo- | 557 |
| | tetrahydro-pyran-2-yl)-ethyl]-5-isopropyl-1H-imidazole-4- | |
| | carboxylic acid (2-phenyl-pyridin-4-ylmethyl)-amide | |
| 169 | 2-(4-Fluoro-phenyl)-1-[2-((2R,4R)-4-hydroxy-6-oxo- | 510 |
| | tetrahydro-pyran-2-yl)-ethyl]-5-isopropyl-1H-imidazole-4- | |
| | carboxylic acid ((S)-2-hydroxy-1-phenyl-ethyl)-amide | |
| 170 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid methyl-((R)-1-phenyl-ethyl)-amide | |
| 171 | 2-(4-Fluoro-phenyl)-1-[2-((2R,4R)-4- | 528 |
| | hydroxy-6-oxo-tetrahydro-pyran-2-yl)- | |
| | ethyl]-5-isopropyl-1H-imidazole- | |
| | 4-carboxylic acid (4-chloro-benzyl)- | |
| | methyl-amide | |
| 172 | 2-(4-Fluoro-phenyl)-1-[2-((2R,4R)-4-hydroxy-6-oxo- | 510 |
| | tetrahydro-pyran-2-yl)-ethyl]-5-isopropyl-1H-imidazole-4- | |
| | carboxylic acid ((R)-2-hydroxy-1-phenyl-ethyl)-amide | |
| 173 | 2-(4-Fluoro-phenyl)-1-[2-((2R,4R)-4- | 528 |
| | hydroxy-6-oxo-tetrahydro-pyran-2-yl)- | |
| | ethyl]-5-isopropyl-1H-imidazole- | |
| | 4-carboxylic acid (3-chloro-benzyl)- | |
| | methyl-amide | |
| 174 | 2-(4-Fluoro-phenyl)-1-[2-((2R,4R)-4- | 528 |
| | hydroxy-6-oxo-tetrahydro-pyran-2-yl)- | |
| | ethyl]-5-isopropyl-1H-imidazole- | |
| | 4-carboxylic acid (2-chloro-benzyl)- | |
| | methyl-amide | |
| 175 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid methyl-((S)-1-phenyl-ethyl)-amide | |
| 176 | (4R,6R)-6-{2-[4-(3,4-Dihydro-2H-quinoline- | 506 |
| | 1-carbonyl)-2-(4-fluoro-phenyl)- | |
| | 5-isopropyl-imidazol-1-yl]-ethyl}- | |
| | 4-hydroxy-tetrahydro-pyran-2-one | |
| 177 | 2-(4-Fluoro-phenyl)-1-[2-((2R,4R)-4-hydroxy-6-oxo- | 516 |
| | tetrahydro-pyran-2-yl)-ethyl]-5-isopropyl-1H-imidazole-4- | |
| | carboxylic acid 2,4-difluoro-benzylamide | |
| 178 | 2-(4-Fluoro-phenyl)-1-[2-((2R,4R)-4-hydroxy-6-oxo- | 528 |
| | tetrahydro-pyran-2-yl)-ethyl]-5-isopropyl-1H-imidazole-4- | |
| | carboxylic acid 2-chloro-6-methyl-benzylamide | |
| 179 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid (1-methyl-1-phenyl-ethyl)-amide | |
| 180 | (4R,6R)-6-{2-[4-(3,4-Dihydro-1H-isoquinoline-2-carbonyl)- | 506 |
| | 2-(4-fluoro-phenyl)-5-isopropyl-imidazol-1-yl]-ethyl}-4- | |
| | hydroxy-tetrahydro-pyran-2-one | |
| 181 | 2-(4-Fluoro-phenyl)-1-[2-((2R,4R)-4-hydroxy-6-oxo- | 575 |
| | tetrahydro-pyran-2-yl)-ethyl]-5-isopropyl-1H-imidazole-4- | |
| | carboxylic acid [2-(2-fluoro-phenyl)-pyridin-4-ylmethyl]- | |
| | amide | |
| 182 | 2-(4-Fluoro-phenyl)-1-[2-((2R,4R)-4-hydroxy-6-oxo- | 516 |
| | tetrahydro-pyran-2-yl)-ethyl]-5-isopropyl-1H-imidazole-4- | |
| | carboxylic acid 3,4-difluoro-benzylamide | |
| 183 | 2-(4-Fluoro-phenyl)-1-[2-((2R,4R)-4-hydroxy-6-oxo- | 586 |
| | tetrahydro-pyran-2-yl)-ethyl]-5-isopropyl-1H-imidazole-4- | |
| | carboxylic acid (2′-methoxy-biphenyl-4-ylmethyl)-amide | |
| 184 | 2-(4-Fluoro-phenyl)-1-[2-((2R,4R)-4-hydroxy-6-oxo- | 548 |
| | tetrahydro-pyran-2-yl)-ethyl]-5-isopropyl-1H-imidazole-4- | |
| | carboxylic acid 2-trifluoromethyl-benzylamide | |
| 185 | 2-(4-Fluoro-phenyl)-1-[2-((2R,4R)-4-hydroxy-6-oxo- | 498 |
| | tetrahydro-pyran-2-yl)-ethyl]-5-isopropyl-1H-imidazole-4- | |
| | carboxylic acid 2-fluoro-benzylamide | |
| 186 | 2-(4-Fluoro-phenyl)-1-[2-((2R,4R)-4-hydroxy-6-oxo- | 570 |
| | tetrahydro-pyran-2-yl)-ethyl]-5-isopropyl-1H-imidazole-4- | |
| | carboxylic acid (2′-methyl-biphenyl-4-ylmethyl)-amide | |
| 187 | 2-(4-Fluoro-phenyl)-1-[2-(4-hydroxy-6-oxo-tetrahydro- | 496 |
| | pyran-2-yl)-ethyl]-5-isopropyl-1H-imidazole-4-carboxylic | |
| | acid (5-methyl-pyrazin-2-ylmethyl)-amide | |
| 188 | 2-(4-Fluoro-phenyl)-1-[2-(4-hydroxy-6-oxo-tetrahydro- | 520 |
| | pyran-2-yl)-ethyl]-5-isopropyl-1H-imidazole-4-carboxylic | |
| | acid (1H-benzoimidazol-2-ylmethyl)-amide | |
| 189 | 2-(4-Fluoro-phenyl)-1-[2-((2R,4R)-4- | 570 |
| | hydroxy-6-oxo-tetrahydro-pyran-2-yl)- | |
| | ethyl]-5-isopropyl-1H-imidazole- | |
| | 4-carboxylic acid benzhydryl-methyl- | |
| | amide | |
| 190 | 2-(4-Fluoro-phenyl)-1-[2-((2R,4R)-4- | 512 |
| | hydroxy-6-oxo-tetrahydro-pyran-2-yl)- | |
| | ethyl]-5-isopropyl-1H-imidazole- | |
| | 4-carboxylic acid (4-fluoro-benzyl)- | |
| | methyl-amide | |
| 191 | 2-(4-Fluoro-phenyl)-1-[2-((2R,4R)-4-hydroxy-6-oxo- | 587 |
| | tetrahydro-pyran-2-yl)-ethyl]-5-isopropyl-1H-imidazole-4- | |
| | carboxylic acid [6-(4-methoxy-phenyl)-pyridin-3-ylmethyl]- | |
| | amide | |
| 192 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid ((R)-2-phenyl-propyl)-amide | |
| 193 | 2-(4-Fluoro-phenyl)-1-[2-((2R,4R)-4-hydroxy-6-oxo- | 558 |
| | tetrahydro-pyran-2-yl)-ethyl]-5-isopropyl-1H-imidazole-4- | |
| | carboxylic acid [2-(4-chloro-phenyl)-1-hydroxymethyl- | |
| | ethyl]-amide | |
| 194 | 2-(4-Fluoro-phenyl)-1-[2-((2R,4R)-4-hydroxy-6-oxo- | 522 |
| | tetrahydro-pyran-2-yl)-ethyl]-5-isopropyl-1H-imidazole-4- | |
| | carboxylic acid ((S)-1-methyl-3-phenyl-propyl)-amide | |
| 195 | 2-(4-Fluoro-phenyl)-1-[2-((2R,4R)-4-hydroxy-6-oxo- | 481 |
| | tetrahydro-pyran-2-yl)-ethyl]-5-isopropyl-1H-imidazole-4- | |
| | carboxylic acid (pyridin-2-ylmethyl)-amide | |
| 196 | 2-(4-Fluoro-phenyl)-1-[2-((2R,4R)-4-hydroxy-6-oxo- | 549 |
| | tetrahydro-pyran-2-yl)-ethyl]-5-isopropyl-1H-imidazole-4- | |
| | carboxylic acid (4-trifluoromethyl-pyridin-2-ylmethyl)- | |
| | amide | |
| 197 | 2-(4-Fluoro-phenyl)-1-[2-((2R,4R)-4-hydroxy-6-oxo- | 495 |
| | tetrahydro-pyran-2-yl)-ethyl]-5-isopropyl-1H-imidazole-4- | |
| | carboxylic acid (1-pyridin-3-yl-ethyl)-amide | |
| 198 | 2-(4-Fluoro-phenyl)-1-[2-((2R,4R)-4-hydroxy-6-oxo- | 494 |
| | tetrahydro-pyran-2-yl)-ethyl]-5-isopropyl-1H-imidazole-4- | |
| | carboxylic acid 4-methyl-benzylamide | |
| 199 | 2-(4-Fluoro-phenyl)-1-[2-((2R,4R)-4-hydroxy-6-oxo- | 514 |
| | tetrahydro-pyran-2-yl)-ethyl]-5-isopropyl-1H-imidazole-4- | |
| | carboxylic acid 4-chloro-benzylamide | |
| 200 | 2-(4-Fluoro-phenyl)-1-[2-((2R,4R)-4-hydroxy-6-oxo- | 556 |
| | tetrahydro-pyran-2-yl)-ethyl]-5-isopropyl-1H-imidazole-4- | |
| | carboxylic acid (biphenyl-2-ylmethyl)-amide | |
| 201 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid [2-(4-methoxy-phenyl)-ethyl]-amide | |
| 202 | 2-(4-Fluoro-phenyl)-1-[2-(4-hydroxy-6-oxo-tetrahydro- | 509 |
| | pyran-2-yl)-ethyl]-5-isopropyl-1H-imidazole-4-carboxylic | |
| | acid (2-amino-2-phenyl-ethyl)-amide | |
| 203 | 2-(4-Fluoro-phenyl)-1-[2-((2R,4R)-4-hydroxy-6-oxo- | 574 |
| | tetrahydro-pyran-2-yl)-ethyl]-5-isopropyl-1H-imidazole-4- | |
| | carboxylic acid (2′-fluoro-biphenyl-4-ylmethyl)-amide | |
| 204 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid (benzo[1,3]dioxol-5-ylmethyl)-amide | |
| 205 | 2-(4-Fluoro-phenyl)-1-[2-((2R,4R)-4-hydroxy-6-oxo- | 536 |
| | tetrahydro-pyran-2-yl)-ethyl]-5-isopropyl-1H-imidazole-4- | |
| | carboxylic acid 4-tert-butyl-benzylamide | |
| 206 | 2-(4-Fluoro-phenyl)-1-[2-((2R,4R)-4-hydroxy-6-oxo- | 523 |
| | tetrahydro-pyran-2-yl)-ethyl]-5-isopropyl-1H-imidazole-4- | |
| | carboxylic acid 3-carbamoyl-benzylamide | |
| 207 | 2-(4-Fluoro-phenyl)-1-[2-((2R,4R)-4-hydroxy-6-oxo- | 558 |
| | tetrahydro-pyran-2-yl)-ethyl]-5-isopropyl-1H-imidazole-4- | |
| | carboxylic acid 3-methanesulfonyl-benzylamide | |
| 208 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid ((S)-2-phenyl-propyl)-amide | |
| 209 | 2-(4-Fluoro-phenyl)-1-[2-((2R,4R)-4-hydroxy-6-oxo- | 599 |
| | tetrahydro-pyran-2-yl)-ethyl]-5-isopropyl-1H-imidazole-4- | |
| | carboxylic acid [6-(3-acetyl-phenyl)-pyridin-3-ylmethyl]- | |
| | amide | |
| 210 | 2-(4-Fluoro-phenyl)-1-[2-((2R,4R)-4-hydroxy-6-oxo- | 494 |
| | tetrahydro-pyran-2-yl)-ethyl]-5-isopropyl-1H-imidazole-4- | |
| | carboxylic acid 2-methyl-benzylamide | |
| 211 | 2-(4-Fluoro-phenyl)-1-[2-((2R,4R)-4- | 510 |
| | hydroxy-6-oxo-tetrahydro-pyran-2-yl)- | |
| | ethyl]-5-isopropyl-1H-imidazole- | |
| | 4-carboxylic acid (2-hydroxy-benzyl)- | |
| | methyl-amide | |
| 212 | 2-(4-Fluoro-phenyl)-1-[2-((2R,4R)-4- | 512 |
| | hydroxy-6-oxo-tetrahydro-pyran-2-yl)- | |
| | ethyl]-5-isopropyl-1H-imidazole- | |
| | 4-carboxylic acid (2-fluoro-benzyl)- | |
| | methyl-amide | |
| 213 | 2-(4-Fluoro-phenyl)-1-[2-((2R,4R)-4- | 544 |
| | hydroxy-6-oxo-tetrahydro-pyran-2-yl)- | |
| | ethyl]-5-isopropyl-1H-imidazole- | |
| | 4-carboxylic acid methyl-naphthalen- | |
| | 1-ylmethyl-amide | |
| 214 | 2-(4-Fluoro-phenyl)-1-[2-((2R,4R)-4- | 510 |
| | hydroxy-6-oxo-tetrahydro-pyran-2-yl)- | |
| | ethyl]-5-isopropyl-1H-imidazole- | |
| | 4-carboxylic acid 2-methoxy-benzylamide | |
| 215 | 2-(4-Fluoro-phenyl)-1-[2-((2R,4R)-4-hydroxy-6-oxo- | 512 |
| | tetrahydro-pyran-2-yl)-ethyl]-5-isopropyl-1H-imidazole-4- | |
| | carboxylic acid [2-(3-fluoro-phenyl)-ethyl]-amide | |
| 216 | 2-(4-Fluoro-phenyl)-1-[2-((2R,4R)-4-hydroxy-6-oxo- | 554 |
| | tetrahydro-pyran-2-yl)-ethyl]-5-isopropyl-1H-imidazole-4- | |
| | carboxylic acid (1S,2S)-2-hydroxy-1-methoxymethyl-2- | |
| | phenyl-ethyl)-amide | |
| 217 | 2-(4-Fluoro-phenyl)-1-[2-((2R,4R)-4-hydroxy-6-oxo- | 579 |
| | tetrahydro-pyran-2-yl)-ethyl]-5-isopropyl-1H-imidazole-4- | |
| | carboxylic acid 4-morpholin-4-ylmethyl-benzylamide | |
| 218 | 2-(4-Fluoro-phenyl)-1-[2-((2R,4R)-4-hydroxy-6-oxo- | 586 |
| | tetrahydro-pyran-2-yl)-ethyl]-5-isopropyl-1H-imidazole-4- | |
| | carboxylic acid (6-methoxy-biphenyl-3-ylmethyl)-amide | |
| 219 | 2-(4-Fluoro-phenyl)-1-[2-((2R,4R)-4-hydroxy-6-oxo- | 592 |
| | tetrahydro-pyran-2-yl)-ethyl]-5-isopropyl-1H-imidazole-4- | |
| | carboxylic acid (3,2′-difluoro-biphenyl-4-ylmethyl)-amide | |
| 220 | 2-(4-Fluoro-phenyl)-1-[2-((2R,4R)-4-hydroxy-6-oxo- | 576 |
| | tetrahydro-pyran-2-yl)-ethyl]-5-isopropyl-1H-imidazole-4- | |
| | carboxylic acid 4-bromo-2-fluoro-benzylamide | |
| 221 | 2-(4-Fluoro-phenyl)-1-[2-((2R,4R)-4-hydroxy-6-oxo- | 574 |
| | tetrahydro-pyran-2-yl)-ethyl]-5-isopropyl-1H-imidazole-4- | |
| | carboxylic acid (3-fluoro-2′-methyl-biphenyl-4-yl)-amide | |
| 222 | 2-(4-Fluoro-phenyl)-1-[2-((2R,4R)-4-hydroxy-6-oxo- | 548 |
| | tetrahydro-pyran-2-yl)-ethyl]-5-isopropyl-1H-imidazole-4- | |
| | carboxylic acid 4-trifluoromethyl-benzylamide | |
| 223 | 2-(4-Fluoro-phenyl)-1-[2-((2R,4R)-4- | 562 |
| | hydroxy-6-oxo-tetrahydro-pyran-2-yl)- | |
| | ethyl]-5-isopropyl-1H-imidazole- | |
| | 4-carboxylic acid (3,4-dichloro-benzyl)- | |
| | methyl-amide | |
| 224 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid [2-(3-methoxy-phenyl)-ethyl]-amide | |
| 225 | 2-(4-Fluoro-phenyl)-1-[2-((2R,4R)-4-hydroxy-6-oxo- | 604 |
| | tetrahydro-pyran-2-yl)-ethyl]-5-isopropyl-1H-imidazole-4- | |
| | carboxylic acid (3-fluoro-3′-methoxy-biphenyl-4-ylmethyl)- | |
| | amide | |
| 226 | 2-(4-Fluoro-phenyl)-1-[2-((2R,4R)-4-hydroxy-6-oxo- | 514 |
| | tetrahydro-pyran-2-yl)-ethyl]-5-isopropyl-1H-imidazole-4- | |
| | carboxylic acid 2-chloro-benzylamide | |
| 227 | 4-[({2-(4-Fluoro-phenyl)-1-[2-((2R,4R)-4-hydroxy-6-oxo- | 538 |
| | tetrahydro-pyran-2-yl)-ethyl]-5-isopropyl-1H-imidazole-4- | |
| | carbonyl}-amino)-methyl]-benzoic acid methyl ester | |
| 228 | 2-(4-Fluoro-phenyl)-1-[2-((2R,4R)-4-hydroxy-6-oxo- | 586 |
| | tetrahydro-pyran-2-yl)-ethyl]-5-isopropyl-1H-imidazole-4- | |
| | carboxylic acid [(1S,2S)-2-hydroxy-1-hydroxymethyl-2-(4- | |
| | methylsulfanyl-phenyl)-ethyl]-amide | |
| 229 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid ((S)-1-benzyl-2-hydroxy-ethyl)-amide | |
| 230 | 2-(4-Fluoro-phenyl)-1-[2-((2R,4R)-4-hydroxy-6-oxo- | 495 |
| | tetrahydro-pyran-2-yl)-ethyl]-5-isopropyl-1H-imidazole-4- | |
| | carboxylic acid (2-pyridin-3-yl-ethyl)-amide | |
| 231 | 2-(4-Fluoro-phenyl)-1-[2-((2R,4R)-4- | 530 |
| | hydroxy-6-oxo-tetrahydro-pyran-2-yl)- | |
| | ethyl]-5-isopropyl-1H-imidazole- | |
| | 4-carboxylic acid (2,4-difluoro-benzyl)- | |
| | methyl-amide | |
| 232 | 2-(4-Fluoro-phenyl)-1-[2-((2R,4R)-4-hydroxy-6-oxo- | 494 |
| | tetrahydro-pyran-2-yl)-ethyl]-5-isopropyl-1H-imidazole-4- | |
| | carboxylic acid 3-methyl-benzylamide. | |
| 233 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid 3-methoxy-4-methyl-benzylamide | |
| 234 | 2-(4-Fluoro-phenyl)-1-[2-((2R,4R)-4-hydroxy-6-oxo- | 508 |
| | tetrahydro-pyran-2-yl)-ethyl]-5-isopropyl-1H-imidazole-4- | |
| | carboxylic acid methyl-(4-methyl-benzyl)-amide | |
| 235 | 2-(4-Fluoro-phenyl)-1-[2-((2R,4R)-4-hydroxy-6-oxo- | 551 |
| | tetrahydro-pyran-2-yl)-ethyl]-5-isopropyl-1H-imidazole-4- | |
| | carboxylic acid 4-dimethylcarbamoyl-benzylamide | |
| 236 | 2-(4-Fluoro-phenyl)-1-[2-((2R,4R)-4-hydroxy-6-oxo- | |
| | tetrahydro-pyran-2-yl)-ethyl]-5-isopropyl-1H-imidazole-4- | |
| | carboxylic acid 3-chloro-4-methyl-benzylamide | |
| 237 | 4-[({2-(4-Fluoro-3-trifluoromethyl-phenyl)-1-[2-((2R,4R)-4- | 606 |
| | hydroxy-6-oxo-tetrahydro-pyran-2-yl)-ethyl]-5-isopropyl- | |
| | 1H-imidazole-4-carbonyl}-amino)-methyl]-benzoic acid | |
| | methyl ester | |
| 238 | 2-(4-Fluoro-phenyl)-1-[2-((2R,4R)-4-hydroxy-6-oxo- | 557 |
| | tetrahydro-pyran-2-yl)-ethyl]-5-isopropyl-1H-imidazole-4- | |
| | carboxylic acid 4-pyridin-2-yl-benzylamide | |
| 239 | 2-(4-Fluoro-phenyl)-1-[2-((2R,4R)-4-hydroxy-6-oxo- | 557 |
| | tetrahydro-pyran-2-yl)-ethyl]-5-isopropyl-1H-imidazole-4- | |
| | carboxylic acid (2-phenyl-pyridin-3-ylmethyl)-amide | |
| 240 | (4R,6R)-6-{2-[2-(4-Fluoro-phenyl)-5-isopropyl-4-(2- | 520 |
| | phenyl-pyrrolidine-1-carbonyl)-imidazol-1-yl]-ethyl}-4- | |
| | hydroxy-tetrahydro-pyran-2-one | |
| 241 | 2-(4-Fluoro-phenyl)-1-[2-((2R,4R)-4- | 524 |
| | hydroxy-6-oxo-tetrahydro-pyran-2-yl)- | |
| | ethyl]-5-isopropyl-1H-imidazole- | |
| | 4-carboxylic acid (4-methoxy-benzyl)- | |
| | methyl-amide | |
| 242 | 2-(4-Fluoro-phenyl)-1-[2-((2R,4R)-4- | 512 |
| | hydroxy-6-oxo-tetrahydro-pyran-2-yl)- | |
| | ethyl]-5-isopropyl-1H-imidazole- | |
| | 4-carboxylic acid (3-fluoro-benzyl)- | |
| | methyl-amide | |
| 243 | 2-(4-Fluoro-phenyl)-1-[2-((2R,4R)-4- | 524 |
| | hydroxy-6-oxo-tetrahydro-pyran-2-yl)- | |
| | ethyl]-5-isopropyl-1H-imidazole- | |
| | 4-carboxylic acid (3-methoxy-benzyl)- | |
| | methyl-amide | |
| 244 | 2-(4-Fluoro-phenyl)-1-[2-((2R,4R)-4-hydroxy-6-oxo- | 538 |
| | tetrahydro-pyran-2-yl)-ethyl]-5-isopropyl-1H-imidazole-4- | |
| | carboxylic acid [(S)-1-(3-methoxy-phenyl)-ethyl]-methyl- | |
| | amide | |
| 245 | 2-(4-Fluoro-3-trifluoromethyl-phenyl)-1-[2-((2R,4R)-4- | 578 |
| | hydroxy-6-oxo-tetrahydro-pyran-2-yl)-ethyl]-5-isopropyl- | |
| | 1H-imidazole-4-carboxylic acid 4-methoxy-benzylamide | |
| 246 | 2-(4-Fluoro-phenyl)-1-[2-((2R,4R)-4-hydroxy-6-oxo- | 524 |
| | tetrahydro-pyran-2-yl)-ethyl]-5-isopropyl-1H-imidazole-4- | |
| | carboxylic acid ((R)-2-hydroxy-1-phenyl-ethyl)-methyl- | |
| | amide | |
| 247 | 2-(4-Fluoro-phenyl)-1-[2-((2R,4R)-4-hydroxy-6-oxo- | 557 |
| | tetrahydro-pyran-2-yl)-ethyl]-5-isopropyl-1H-imidazole-4- | |
| | carboxylic acid 2-pyridin-2-yl-benzylamide | |
| 248 | 3-[({2-(4-Fluoro-phenyl)-1-[2-((2R, | 552 |
| | 4R)-4-hydroxy-6-oxo-tetrahydro-pyran- | |
| | 2-yl)-ethyl]-5-isopropyl-1H-imidazole- | |
| | 4-carbonyl}-methyl-amino)-methyl]- | |
| | benzoic acid methyl ester | |
| 249 | 2-(4-Fluoro-phenyl)-1-[2-((2R,4R)-4- | 562 |
| | hydroxy-6-oxo-tetrahydro-pyran-2-yl)- | |
| | ethyl]-5-isopropyl-1H-imidazole- | |
| | 4-carboxylic acid methyl-(2-trifluoromethyl- | |
| | benzyl)-amide | |
| 250 | 2-(4-Fluoro-phenyl)-1-[2-((2R,4R)-4- | 530 |
| | hydroxy-6-oxo-tetrahydro-pyran-2-yl)- | |
| | ethyl]-5-isopropyl-1H-imidazole- | |
| | 4-carboxylic acid (3,4-difluoro-benzyl)- | |
| | methyl-amide | |
| 251 | 4-[({2-(4-Fluoro-phenyl)-1-[2-((2R, | 552 |
| | 4R)-4-hydroxy-6-oxo-tetrahydro-pyran- | |
| | 2-yl)-ethyl]-5-isopropyl-1H-imidazole- | |
| | 4-carbonyl}-methyl-amino)-methyl]- | |
| | benzoic acid methyl ester | |
| 252 | 2-(4-Fluoro-phenyl)-1-[2-((2R,4R)-4-hydroxy-6-oxo- | 522 |
| | tetrahydro-pyran-2-yl)-ethyl]-5-isopropyl-1H-imidazole-4- | |
| | carboxylic acid methyl-((R)-1-p-tolyl-ethyl)-amide | |
| 253 | 2-(4-Fluoro-phenyl)-1-[2-((2R,4R)-4-hydroxy-6-oxo- | 486 |
| | tetrahydro-pyran-2-yl)-ethyl]-5-isopropyl-1H-imidazole-4- | |
| | carboxylic acid cyclohexylmethyl-amide | |
| 254 | 3-[({2-(4-Fluoro-phenyl)-1-[2-((2R, | 552 |
| | 4R)-4-hydroxy-6-oxo-tetrahydro-pyran- | |
| | 2-yl)-ethyl]-5-isopropyl-1H-imidazole- | |
| | 4-carbonyl}-methyl-amino)-methyl]- | |
| | benzoic acid methyl ester | |
| 255 | (4R,6R)-6-{2-[2-(4-Fluoro-phenyl)-5- | 458 |
| | isopropyl-4-(piperidine-1-carbonyl)- | |
| | imidazol-1-yl]-ethyl}-4-hydroxy-tetrahydro- | |
| | pyran-2-one | |
| 256 | (4R,6R)-6-{2-[2-(4-Fluoro-phenyl)-5- | 534 |
| | isopropyl-4-(4-phenyl-piperidine-1- | |
| | carbonyl)-imidazol-1-yl]-ethyl}-4- | |
| | hydroxy-tetrahydro-pyran-2-one | |
| 257 | 2-(4-Fluoro-phenyl)-1-[2-((2R,4R)-4-hydroxy-6-oxo- | 549 |
| | tetrahydro-pyran-2-yl)-ethyl]-5-isopropyl-1H-imidazole-4- | |
| | carboxylic acid (6-trifluoromethyl-pyridin-3-ylmethyl)- | |
| | amide | |
| 258 | 2-(4-Fluoro-phenyl)-1-[2-((2R,4R)-4-hydroxy-6-oxo- | 591 |
| | tetrahydro-pyran-2-yl)-ethyl]-5-isopropyl-1H-imidazole-4- | |
| | carboxylic acid 3-(4-methyl-piperidin-1-ylmethyl)- | |
| | benzylamide | |
| 259 | (4R,6R)-6-{2-[2-(4-Fluoro-phenyl)-5- | 534 |
| | isopropyl-4-(3-phenyl-piperidine-1- | |
| | carbonyl)-imidazol-1-yl]-ethyl}-4- | |
| | hydroxy-tetrahydro-pyran-2-one | |
| 260 | 2-(4-Fluoro-phenyl)-1-[2-((2R,4R)-4-hydroxy-6-oxo- | 577 |
| | tetrahydro-pyran-2-yl)-ethyl]-5-isopropyl-1H-imidazole-4- | |
| | carboxylic acid 3-piperidin-1-ylmethyl-benzylamide | |
| 261 | 2-(4-Fluoro-phenyl)-1-[2-((2R,4R)-4-hydroxy-6-oxo- | 418 |
| | tetrahydro-pyran-2-yl)-ethyl]-5-isopropyl-1H-imidazole-4- | |
| | carboxylic acid dimethylamide | |
| 262 | N-{2-(4-Fluoro-phenyl)-1-[2-((2R,4R)- | 480 |
| | 4-hydroxy-6-oxo-tetrahydro-pyran- | |
| | 2-yl)-ethyl]-5-isopropyl-1H-imidazol- | |
| | 4-yl}-2-phenyl-acetamide | |
| 263 | 4-[({2-(4-Fluoro-phenyl)-1-[2-((2R,4R)-4-hydroxy-6-oxo- | 552 |
| | tetrahydro-pyran-2-yl)-ethyl]-5-isopropyl-1H-imidazole-4- | |
| | carbonyl}-methyl-amino)-methyl]-benzoic acid methyl | |
| | ester | |
| 264 | 1-{2-(4-Fluoro-phenyl)-1-[2-((2R,4R)-4-hydroxy-6-oxo- | 530 |
| | tetrahydro-pyran-2-yl)-ethyl]-5-isopropyl-1H-imidazole-4- | |
| | carbonyl}-piperidine-4-carboxylic acid ethyl ester | |
| 265 | 2-(4-Fluoro-phenyl)-1-[2-((2R,4R)-4-hydroxy-6-oxo- | 444 |
| | tetrahydro-pyran-2-yl)-ethyl]-5-isopropyl-1H-imidazole-4- | |
| | carboxylic acid cyclopropylmethyl-amide | |
| 266 | 2-(4-Fluoro-phenyl)-1-[2-((2R,4R)-4-hydroxy-6-oxo- | 490 |
| | tetrahydro-pyran-2-yl)-ethyl]-5-isopropyl-1H-imidazole-4- | |
| | carboxylic acid (3-isopropoxy-propyl)-amide | |
| 267 | 2-(4-Fluoro-phenyl)-1-[2-((2R,4R)-4-hydroxy-6-oxo- | 446 |
| | tetrahydro-pyran-2-yl)-ethyl]-5-isopropyl-1H-imidazole-4- | |
| | carboxylic acid butylamide | |
| 268 | 2-(4-Fluoro-phenyl)-1-[2-((2R,4R)-4-hydroxy-6-oxo- | 500 |
| | tetrahydro-pyran-2-yl)-ethyl]-5-isopropyl-1H-imidazole-4- | |
| | carboxylic acid ((R)-1-cyclohexyl-ethyl)-amide | |
| 269 | 4-Fluoro-N-{2-(4-fluoro-phenyl)-1- | 484 |
| | [2-((2R,4R)-4-hydroxy-6-oxo-tetrahydro- | |
| | pyran-2-yl)-ethyl]-5-isopropyl- | |
| | 1H-imidazol-4-yl}-benzamide | |
| 270 | N-{2-(4-Fluoro-phenyl)-1-[2-((2R,4R)- | 496 |
| | 4-hydroxy-6-oxo-tetrahydro-pyran- | |
| | 2-yl)-ethyl]-5-isopropyl-1H-imidazol- | |
| | 4-yl}-4-methoxy-benzamide | |
| 271 | N-{2-(4-Fluoro-phenyl)-1-[2-((2R,4R)- | 466 |
| | 4-hydroxy-6-oxo-tetrahydro-pyran- | |
| | 2-yl)-ethyl]-5-isopropyl-1H-imidazol- | |
| | 4-yl}-benzamide | |
| 272 | 2-(4-Fluoro-phenyl)-1-[2-((2R,4R)-4-hydroxy-6-oxo- | 495 |
| | tetrahydro-pyran-2-yl)-ethyl]-5-isopropyl-1H-imidazole-4- | |
| | carboxylic acid (2-pyridin-4-yl-ethyl)-amide | |
| 273 | 2-(4-Fluoro-phenyl)-1-[2-((2R,4R)-4-hydroxy-6-oxo- | 573 |
| | tetrahydro-pyran-2-yl)-ethyl]-5-isopropyl-1H-imidazole-4- | |
| | carboxylic acid [2-(4-sulfamoyl-phenyl)-ethyl]-amide | |
| 274 | 2-(4-Fluoro-phenyl)-1-[2-((2R,4R)-4-hydroxy-6-oxo- | 522 |
| | tetrahydro-pyran-2-yl)-ethyl]-5-isopropyl-1H-imidazole-4- | |
| | carboxylic acid (1-methyl-3-phenyl-propyl)-amide | |
| 275 | N-{2-(4-Fluoro-phenyl)-1-[2-((2R,4R)-4-hydroxy-6-oxo- | 454 |
| | tetrahydro-pyran-2-yl)-ethyl]-5-isopropyl-1H-imidazol-4- | |
| | ylmethyl}-methanesulfonamide. | |
| 276 | 2-(4-Fluoro-phenyl)-1-[2-((2R,4R)-4-hydroxy-6-oxo- | 418 |
| | tetrahydro-pyran-2-yl)-ethyl]-5-isopropyl-1H-imidazole-4- | |
| | carboxylic acid ethyl amide | |

TABLE II

| | | Mass Spectra |
|---|---|---|
| Example | Sodium Salt (IUPAC) | (APCI) [M + H]⁺ |
| 277 | Sodium; (3R,5R)-7-[2-(4-Fluoro-phenyl)-5-isopropyl-4- | 499 |
| | phenylcarbamoyl-imidazol-1-yl]-3,5-dihydroxy-heptanoate | |
| 278 | Sodium; (3R,5R)-7-[4-(benzylsulfonyl)-2-(4-fluorophenyl)- | 518 |
| | 5-isopropyl-1H-imidazol-1-yl]-3,5-dihydroxyheptanoate | |
| 279 | Sodium; (3R,5R)-7-[2-(4-fluorophenyl)-5-isopropyl-4- | 442 |
| | (methylsulfonyl)-1H-imidazol-1-yl]-3,5- | |
| | dihydroxyheptanoate | |
| 280 | Sodium; (3R,5R)-7-[4-{[benzyl(methyl)amino]carbonyl}-2- | 512 |
| | (4-fluorophenyl)-5-isopropyl-1H-imidazol-1-yl]-3,5- | |
| | dihydroxyheptanoate | |
| 281 | Sodium; (3R,5R)-7-[4-{[(2,3- | 566 |
| | dichlorobenzyl)amino]carbonyl}-2-(4-fluorophenyl)-5- | |
| | isopropyl-1H-imidazol-1-yl]-3,5-dihydroxyheptanoate | |
| 282 | Sodium; (3R,5R)-7-(2-(4-fluorophenyl)-5-isopropyl-4-{[(3- | 528 |
| | methoxybenzyl)amino]carbonyl}-1H-imidazol-1-yl)-3,5- | |
| | dihydroxyheptanoate | |
| 283 | Sodium; (3R,5R)-7-[4-({[(2′-fluorobiphenyl-3- | 592 |
| | yl)methyl]amino}carbonyl)-2-(4-fluorophenyl)-5-isopropyl- | |
| | 1H-imidazol-1-yl]-3,5-dihydroxyheptanoate | |
| 284 | Sodium; (3R,5R)-7-[4-{[benzyl(isopropyl)amino]carbonyl}- | 540 |
| | 2-(4-fluorophenyl)-5-isopropyl-1H-imidazol-1-yl]-3,5- | |
| | dihydroxyheptanoate | |
| 285 | Sodium; (3R,5R)-7-[2-(4-fluorophenyl)-5-isopropyl-4-({[(6- | 575 |
| | phenylpyridin-3-yl)methyl]amino}carbonyl)-1H-imidazol-1- | |
| | yl]-3,5-dihydroxyheptanoate | |
| 286 | Sodium; (3R,5R)-7-[4-{[benzyl(propyl)amino]carbonyl}-2- | 540 |
| | (4-fluorophenyl)-5-isopropyl-1H-imidazol-1-yl]-3,5- | |
| | dihydroxyheptanoate | |
| 287 | Sodium; (3R,5R)-7-[4-({[(1,5-dimethyl-1H-pyrazol-3- | 538 |
| | yl)methyl]amino}carbonyl)-2-(4-fluorophenyl)-5-isopropyl- | |
| | 1H-imidazol-1-yl]-3,5-dihydroxyheptanoate | |
| 288 | Sodium; (3R,5R)-7-{2-(4-fluorophenyl)-4-[({[3′- | 604 |
| | (hydroxymethyl)biphenyl-3-yl]methyl}amino)carbonyl]-5- | |
| | isopropyl-1H-imidazol-1-yl}-3,5-dihydroxyheptanoate | |
| 289 | Sodium; (3R,5R)-7-(2-(4-fluorophenyl)-5-isopropyl-4-{[(3- | 575 |
| | pyridin-3-ylbenzyl)amino]carbonyl}-1H-imidazol-1-yl)-3,5- | |
| | dihydroxyheptanoate | |
| 290 | Sodium; 7-{2-(4-Fluoro-phenyl)-5-isopropyl-4-[(6-o-tolyl- | 589 |
| | pyridin-3-ylmethyl)-carbamoyl]-imidazol-1-yl}-3,5- | |
| | dihydroxyheptanoate | |
| 291 | Sodium; (3R,5R)-7-[4-({[(1S)-1-(4- | 590 |
| | bromophenyl)ethyl]amino}carbonyl)-2-(4-fluorophenyl)-5- | |
| | isopropyl-1H-imidazol-1-yl]-3,5-dihydroxyheptanoate | |
| 292 | Sodium; (3R,5R)-7-[4-({[(1R)-1-(4- | 590 |
| | bromophenyl)ethyl]amino}carbonyl)-2-(4-fluorophenyl)-5- | |
| | isopropyl-1H-imidazol-1-yl]-3,5-dihydroxyheptanoate | |
| 293 | Sodium; (3R,5R)-7-[2-(4-fluorophenyl)-5-isopropyl-4- | 526 |
| | ({[(1R)-1-(4-methylphenyl)ethyl]amino}carbonyl)-1H- | |
| | imidazol-1-yl]-3,5-dihydroxyheptanoate | |
| 294 | Sodium; (3R,5R)-7-[2-(4-fluorophenyl)-5-isopropyl-4- | 526 |
| | ({[(1S)-1-(4-methylphenyl)ethyl]amino}carbonyl)-1H- | |
| | imidazol-1-yl]-3,5-dihydroxyheptanoate | |
| 295 | Sodium; (3R,5R)-7-[2-(4-fluorophenyl)-5-isopropyl-4- | 542 |
| | ({[(1R)-1-(4-methoxyphenyl)ethyl]amino}carbonyl)-1H- | |
| | imidazol-1-yl]-3,5-dihydroxyheptanoate | |
| 296 | Sodium; (3R,5R)-7-[2-(4-fluorophenyl)-5-isopropyl-4- | 542 |
| | ({[(1S)-1-(4-methoxyphenyl)ethyl]amino}carbonyl)-1H- | |
| | imidazol-1-yl]-3,5-dihydroxyheptanoate | |
| 297 | Sodium; (3R,5R)-7-[2-(4-fluorophenyl)-5-isopropyl-4- | 542 |
| | ({[(1R)-1-(3-methoxyphenyl)ethyl]amino}carbonyl)-1H- | |
| | imidazol-1-yl]-3,5-dihydroxyheptanoate | |
| 298 | Sodium; (3R,5R)-7-[2-(4-fluorophenyl)-5-isopropyl-4- | 542 |
| | ({[(1S)-1-(3-methoxyphenyl)ethyl]amino}carbonyl)-1H- | |
| | imidazol-1-yl]-3,5-dihydroxyheptanoate | |
| 299 | Sodium; (3R,5R)-7-[2-(4-fluorophenyl)-5-isopropyl-4-({[(2′- | 588 |
| | methylbiphenyl-3-yl)methyl]amino}carbonyl)-1H-imidazol- | |
| | 1-yl]-3,5-dihydroxyheptanoate | |
| 300 | Disodium; 3′-[({[1-[(3R,5R)-6-carboxy-3,5-dihydroxyhexyl]- | 618 |
| | 2-(4-fluorophenyl)-5-isopropyl-1H-imidazol-4- | |
| | yl]carbonyl}amino)methyl]biphenyl-3-carboxylate | |
| 301 | Sodium; (3R,5R)-7-(2-(4-fluorophenyl)-5-isopropyl-4- | 513 |
| | {[methyl(pyridin-2-ylmethyl)amino]carbonyl}-1H-imidazol-1- | |
| | yl)-3,5-dihydroxyheptanoate | |
| 302 | Sodium; (3R,5R)-7-(2-(4-fluorophenyl)-5-isopropyl-4- | 513 |
| | {[methyl(pyridin-3-ylmethyl)amino]carbonyl}-1H-imidazol-1- | |
| | yl)-3,5-dihydroxyheptanoate | |
| 303 | Sodium; (3R,5R)-7-(2-(4-fluorophenyl)-5-isopropyl-4- | 513 |
| | {[methyl(pyridin-4-ylmethyl)amino]carbonyl}-1H-imidazol-1- | |
| | yl)-3,5-dihydroxyheptanoate | |
| 304 | Sodium; (3R,5R)-7-[2-(4-fluorophenyl)-5-isopropyl-4-({[4- | 556 |
| | (methoxycarbonyl)benzyl]amino}carbonyl)-1H-imidazol-1- | |
| | yl]-3,5-dihydroxyheptanoate | |
| 305 | Sodium; (3R,5R)-7-[4-[({4- | 605 |
| | [(dimethylamino)sulfonyl]benzyl}amino)carbonyl]-2-(4- | |
| | fluorophenyl)-5-isopropyl-1H-imidazol-1-yl]-3,5- | |
| | dihydroxyheptanoate | |
| 306 | Sodium; (3R,5R)-7-[4-[({3- | 605 |
| | [(dimethylamino)sulfonyl]benzyl}amino)carbonyl]-2-(4- | |
| | fluorophenyl)-5-isopropyl-1H-imidazol-1-yl]-3,5- | |
| | dihydroxyheptanoate | |
| 307 | Sodium; (3R,5R)-7-[4-[({3- | 569 |
| | [(dimethylamino)carbonyl]benzyl}amino)carbonyl]-2-(4- | |
| | fluorophenyl)-5-isopropyl-1H-imidazol-1-yl]-3,5- | |
| | dihydroxyheptanoate | |
| 308 | Sodium; (3R,5R)-7-[2-(4-fluorophenyl)-5-isopropyl-4-({[3- | 609 |
| | (piperidin-1-ylcarbonyl)benzyl]amino}carbonyl)-1H- | |
| | imidazol-1-yl]-3,5-dihydroxyheptanoate | |
| 309 | Sodium; (3R,5R)-7-[2-(4-fluorophenyl)-5-isopropyl-4-({[3- | 611 |
| | (morpholin-4-ylcarbonyl)benzyl]amino}carbonyl)-1H- | |
| | imidazol-1-yl]-3,5-dihydroxyheptanoate | |
| 310 | Sodium; (3R,5R)-7-[4-{[({6-[3- | 647 |
| | (ethoxycarbonyl)phenyl]pyridin-3- | |
| | yl}methyl)amino]carbonyl}-2-(4-fluorophenyl)-5-isopropyl- | |
| | 1H-imidazol-1-yl]-3,5-dihydroxyheptanoate | |
| 311 | Sodium; (3R,5R)-7-[2-(4-fluorophenyl)-5-isopropyl-4-({[(2′- | 604 |
| | methoxybiphenyl-3-yl)methyl]amino}carbonyl)-1H-imidazol- | |
| | 1-yl]-3,5-dihydroxyheptanoate | |
| 312 | Sodium; (3R,5R)-7-[2-(4-fluorophenyl)-5-isopropyl-4- | 526 |
| | ({[(1S)-1-phenylpropyl]amino}carbonyl)-1H-imidazol-1-yl]- | |
| | 3,5-dihydroxyheptanoate | |
| 313 | Sodium; (3R,5R)-7-[2-(4-fluorophenyl)-5-isopropyl-4- | 526 |
| | ({[(1R)-1-phenylpropyl]amino}carbonyl)-1H-imidazol-1-yl]- | |
| | 3,5-dihydroxyheptanoate | |
| 314 | Sodium; (3R,5R)-7-[2-(4-fluorophenyl)-5-isopropyl-4-({[(2- | 575 |
| | phenylpyridin-4-yl)methyl]amino}carbonyl)-1H-imidazol-1- | |
| | yl]-3,5-dihydroxyheptanoate | |
| 315 | Sodium; (3R,5R)-7-[2-(4-fluorophenyl)-4-({[(1S)-2-hydroxy- | 528 |
| | 1-phenylethyl]amino}carbonyl)-5-isopropyl-1H-imidazol-1- | |
| | yl]-3,5-dihydroxyheptanoate | |
| 316 | Sodium; (3R,5R)-7-[2-(4-fluorophenyl)-5-isopropyl-4- | 526 |
| | ({methyl[(1R)-1-phenylethyl]amino}carbonyl)-1H-imidazol- | |
| | 1-yl]-3,5-dihydroxyheptanoate | |
| 317 | Sodium; (3R,5R)-7-[4-{[(4- | 546 |
| | chlorobenzyl)(methyl)amino]carbonyl}-2-(4-fluorophenyl)-5- | |
| | isopropyl-1H-imidazol-1-yl]-3,5-dihydroxyheptanoate | |
| 318 | Sodium; (3R,5R)-7-[2-(4-fluorophenyl)-4-({[(1R)-2-hydroxy- | 528 |
| | 1-phenylethyl]amino}carbonyl)-5-isopropyl-1H-imidazol-1- | |
| | yl]-3,5-dihydroxyheptanoate | |
| 319 | Sodium; (3R,5R)-7-[4-{[(3- | 546 |
| | chlorobenzyl)(methyl)amino]carbonyl}-2-(4-fluorophenyl)-5- | |
| | isopropyl-1H-imidazol-1-yl]-3,5-dihydroxyheptanoate | |
| 320 | Sodium; (3R,5R)-7-[4-{[(2- | 546 |
| | chlorobenzyl)(methyl)amino]carbonyl}-2-(4-fluorophenyl)-5- | |
| | isopropyl-1H-imidazol-1-yl]-3,5-dihydroxyheptanoate | |
| 321 | Sodium; (3R,5R)-7-[2-(4-fluorophenyl)-5-isopropyl-4- | 526 |
| | ({methyl[(1S)-1-phenylethyl]amino}carbonyl)-1H-imidazol- | |
| | 1-yl]-3,5-dihydroxyheptanoate | |
| 322 | Sodium; (3R,5R)-7-[4-(3,4-dihydroquinolin-1(2H)- | 524 |
| | ylcarbonyl)-2-(4-fluorophenyl)-5-isopropyl-1H-imidazol-1- | |
| | yl]-3,5-dihydroxyheptanoate | |
| 323 | Sodium; (3R,5R)-7-[4-{[(2,4- | 534 |
| | difluorobenzyl)amino]carbonyl}-2-(4-fluorophenyl)-5- | |
| | isopropyl-1H-imidazol-1-yl]-3,5-dihydroxyheptanoate | |
| 324 | Sodium; (3R,5R)-7-[4-{[(2-chloro-6- | 546 |
| | methylbenzyl)amino]carbonyl}-2-(4-fluorophenyl)-5- | |
| | isopropyl-1H-imidazol-1-yl]-3,5-dihydroxyheptanoate | |
| 325 | Sodium; (3R,5R)-7-(2-(4-fluorophenyl)-5-isopropyl-4-{[(1- | 526 |
| | methyl-1-phenylethyl)amino]carbonyl}-1H-imidazol-1-yl)- | |
| | 3,5-dihydroxyheptanoate | |
| 326 | Sodium; (3R,5R)-7-[4-(3,4-dihydroisoquinolin-2(1H)- | 524 |
| | ylcarbonyl)-2-(4-fluorophenyl)-5-isopropyl-1H-imidazol-1- | |
| | yl]-3,5-dihydroxyheptanoate | |
| 327 | Sodium; (3R,5R)-7-{2-(4-fluorophenyl)-4-[({[2-(2- | 593 |
| | fluorophenyl)pyridin-4-yl]methyl}amino)carbonyl]-5- | |
| | isopropyl-1H-imidazol-1-yl}-3,5 dihydroxyheptanoate | |
| 328 | Sodium; (3R,5R)-7-[4-{[(3,4- | 534 |
| | difluorobenzyl)amino]carbonyl}-2-(4-fluorophenyl)-5- | |
| | isopropyl-1H-imidazol-1-yl]-3,5-dihydroxyheptanoate | |
| 329 | Sodium; (3R,5R)-7-[2-(4-fluorophenyl)-5-isopropyl-4-({[(2′- | 604 |
| | methoxybiphenyl-4-yl)methyl]amino}carbonyl)-1H-imidazol- | |
| | 1-yl]-3,5-dihydroxyheptanoate | |
| 330 | Sodium; (3R,5R)-7-[2-(4-fluorophenyl)-5-isopropyl-4-({[2- | 566 |
| | (trifluoromethyl)benzyl]amino}carbonyl)-1H-imidazol-1-yl]- | |
| | 3,5-dihydroxyheptanoate | |
| 331 | Sodium; (3R,5R)-7-[4-{[(2-fluorobenzyl)amino]carbonyl}-2- | 516 |
| | (4-fluorophenyl)-5-isopropyl-1H-imidazol-1-yl]-3,5- | |
| | dihydroxyheptanoate | |
| 332 | Sodium; (3R,5R)-7-[2-(4-fluorophenyl)-5-isopropyl-4-({[(2′- | 588 |
| | methylbiphenyl-4-yl)methyl]amino}carbonyl)-1H-imidazol- | |
| | 1-yl]-3,5-dihydroxyheptanoate | |
| 333 | Sodium; (3R,5R)-7-[2-(4-fluorophenyl)-5-isopropyl-4-({[(5- | 514 |
| | methylpyrazin-2-yl)methyl]amino}carbonyl)-1H-imidazol-1- | |
| | yl]-3,5-dihydroxyheptanoate | |
| 334 | Sodium; (3R,5R)-7-[4-{[(1H-benzimidazol-2- | 538 |
| | ylmethyl)amino]carbonyl}-2-(4-fluorophenyl)-5-isopropyl- | |
| | 1H-imidazol-1-yl]-3,5-dihydroxyheptanoate | |
| 335 | Sodium; (3R,5R)-7-[4- | 588 |
| | {[(diphenylmethyl)(methyl)amino]carbonyl}-2-(4- | |
| | fluorophenyl)-5-isopropyl-1H-imidazol-1-yl]-3,5- | |
| | dihydroxyheptanoate | |
| 336 | Sodium; (3R,5R)-7-[4-{[(4- | 530 |
| | fluorobenzyl)(methyl)amino]carbonyl}-2-(4-fluorophenyl)-5- | |
| | isopropyl-1H-imidazol-1-yl]-3,5-dihydroxyheptanoate | |
| 337 | Sodium; (3R,5R)-7-{2-(4-fluorophenyl)-5-isopropyl-4-[({[6- | 605 |
| | (4-methoxyphenyl)pyridin-3-yl]methyl}amino)carbonyl]-1H- | |
| | imidazol-1-yl}-3,5-dihydroxyheptanoate | |
| 338 | Sodium; (3R,5R)-7-(2-(4-fluorophenyl)-5-isopropyl-4- | 499 |
| | {[(pyridin-2-ylmethyl)amino]carbonyl}-1H-imidazol-1-yl)- | |
| | 3,5-dihydroxyheptanoate | |
| 339 | Sodium; (3R,5R)-7-{2-(4-fluorophenyl)-5-isopropyl-4-[({[4- | 567 |
| | (trifluoromethyl)pyridin-2-yl]methyl}amino)carbonyl]-1H- | |
| | imidazol-1-yl}-3,5-dihydroxyheptanoate | |
| 340 | Sodium; (3R,5R)-7-(2-(4-fluorophenyl)-5-isopropyl-4-{[(1- | 513 |
| | pyridin-3-ylethyl)amino]carbonyl}-1H-imidazol-1-yl)-3,5- | |
| | dihydroxyheptanoate | |
| 341 | Sodium; (3R,5R)-7-(2-(4-fluorophenyl)-5-isopropyl-4-{[(4- | 512 |
| | methylbenzyl)amino]carbonyl}-1H-imidazol-1-yl)-3,5- | |
| | dihydroxyheptanoate | |
| 342 | Sodium; (3R,5R)-7-[4-{[(4-chlorobenzyl)amino]carbonyl}-2- | 532 |
| | (4-fluorophenyl)-5-isopropyl-1H-imidazol-1-yl]-3,5- | |
| | dihydroxyheptanoate | |
| 343 | Sodium; (3R,5R)-7-[4-{[(biphenyl-2- | 574 |
| | ylmethyl)amino]carbonyl}-2-(4-fluorophenyl)-5-isopropyl- | |
| | 1H-imidazol-1-yl]-3,5-dihydroxyheptanoate | |
| 344 | Sodium; (3R,5R)-7-[4-({[(2R)-2-amino-2- | 527 |
| | phenylethyl]amino}carbonyl)-2-(4-fluorophenyl)-5- | |
| | isopropyl-1H-imidazol-1-yl]-3,5-dihydroxyheptanoate | |
| 345 | Sodium; (3R,5R)-7-[4-({[(2′-fluorobiphenyl-4- | 615 |
| | yl)methyl]amino}carbonyl)-2-(4-fluorophenyl)-5-isopropyl- | |
| | 1H-imidazol-1-yl]-3,5-dihydroxyheptanoate | |
| 346 | Sodium; (3R,5R)-7-[4-{[(1,3-benzodioxol-5- | 542 |
| | ylmethyl)amino]carbonyl}-2-(4-fluorophenyl)-5-isopropyl- | |
| | 1H-imidazol-1-yl]-3,5-dihydroxyheptanoate | |
| 347 | Sodium; (3R,5R)-7-[4-{[(4-tert- | 554 |
| | butylbenzyl)amino]carbonyl}-2-(4-fluorophenyl)-5- | |
| | isopropyl-1H-imidazol-1-yl]-3,5-dihydroxyheptanoate | |
| 348 | Sodium; (3R,5R)-7-[4-({[3- | 541 |
| | (aminocarbonyl)benzyl]amino}carbonyl)-2-(4-fluorophenyl)- | |
| | 5-isopropyl-1H-imidazol-1-yl]-3,5-dihydroxyheptanoate | |
| 349 | Sodium; (3R,5R)-7-[2-(4-fluorophenyl)-5-isopropyl-4-({[3- | 576 |
| | (methylsulfonyl)benzyl]amino}carbonyl)-1H-imidazol-1-yl]- | |
| | 3,5-dihydroxyheptanoate | |
| 350 | Sodium; (3R,5R)-7-[4-[({[6-(3-acetylphenyl)pyridin-3- | 617 |
| | yl]methyl}amino)carbonyl]-2-(4-fluorophenyl)-5-isopropyl- | |
| | 1H-imidazol-1-yl]-3,5-dihydroxyheptanoate | |
| 351 | Sodium; (3R,5R)-7-(2-(4-fluorophenyl)-5-isopropyl-4-{[(2- | 512 |
| | methylbenzyl)amino]carbonyl}-1H-imidazol-1-yl)-3,5- | |
| | dihydroxyheptanoate | |
| 352 | Sodium; (3R,5R)-7-(2-(4-fluorophenyl)-4-{[(2- | 528 |
| | hydroxybenzyl)(methyl)amino]carbonyl}-5-isopropyl-1H- | |
| | imidazol-1-yl)-3,5-dihydroxyheptanoate | |
| 353 | Sodium; (3R,5R)-7-[4-{[(2- | 530 |
| | fluorobenzyl)(methyl)amino]carbonyl}-2-(4-fluorophenyl)-5- | |
| | isopropyl-1H-imidazol-1-yl]-3,5-dihydroxyheptanoate | |
| 354 | Sodium; (3R,5R)-7-(2-(4-fluorophenyl)-5-isopropyl-4- | 562 |
| | {[methyl(1-naphthylmethyl)amino]carbonyl}-1H-imidazol-1- | |
| | yl)-3,5-dihydroxyheptanoate | |
| 355 | Sodium; (3R,5R)-7-(2-(4-fluorophenyl)-5-isopropyl-4-{[(2- | 528 |
| | methoxybenzyl)amino]carbonyl}-1H-imidazol-1-yl)-3,5- | |
| | dihydroxyheptanoate | |
| 356 | Sodium; (3R,5R)-7-[2-(4-fluorophenyl)-5-isopropyl-4-({[3- | 556 |
| | (methoxycarbonyl)benzyl]amino}carbonyl)-1H-imidazol-1- | |
| | yl]-3,5-dihydroxyheptanoate | |
| 357 | Sodium; (3R,5R)-7-[2-(4-fluorophenyl)-5-isopropyl-4-({[4- | 597 |
| | (morpholin-4-ylmethyl)benzyl]amino}carbonyl)-1H- | |
| | imidazol-1-yl]-3,5-dihydroxyheptanoate | |
| 358 | Sodium; (3R,5R)-7-[2-(4-fluorophenyl)-5-isopropyl-4-({[(6- | 604 |
| | methoxybiphenyl-3-yl)methyl]amino}carbonyl)-1H-imidazol- | |
| | 1-yl]-3,5-dihydroxyheptanoate | |
| 359 | Sodium; (3R,5R)-7-[4-({[(2′,3-difluorobiphenyl-4- | 610 |
| | yl)methyl]amino}carbonyl)-2-(4-fluorophenyl)-5-isopropyl- | |
| | 1H-imidazol-1-yl]-3,5-dihydroxyheptanoate | |
| 360 | Sodium; (3R,5R)-7-[4-{[(4-bromo-2- | 594 |
| | fluorobenzyl)amino]carbonyl}-2-(4-fluorophenyl)-5- | |
| | isopropyl-1H-imidazol-1-yl]-3,5-dihydroxyheptanoate | |
| 361 | Sodium; (3R,5R)-7-[4-({[(3-fluoro-2′-methylbiphenyl-4- | 606 |
| | yl)methyl]amino}carbonyl)-2-(4-fluorophenyl)-5-isopropyl- | |
| | 1H-imidazol-1-yl]-3,5-dihydroxyheptanoate | |
| 362 | Sodium; (3R,5R)-7-[2-(4-fluorophenyl)-5-isopropyl-4-({[4- | 566 |
| | (trifluoromethyl)benzyl]amino}carbonyl)-1H-imidazol-1-yl]- | |
| | 3,5-dihydroxyheptanoate | |
| 363 | Sodium; (3R,5R)-7-[4-{[(3,4- | 580 |
| | dichlorobenzyl)(methyl)amino]carbonyl}-2-(4-fluorophenyl)- | |
| | 5-isopropyl-1H-imidazol-1-yl]-3,5-dihydroxyheptanoate | |
| 364 | Sodium; (3R,5R)-7-{2-(4-fluorophenyl)-5-isopropyl-4-[({4- | 591 |
| | [(methylsulfonyl)amino]benzyl}amino)carbonyl]-1H- | |
| | imidazol-1-yl}-3,5-dihydroxyheptanoate | |
| 365 | Sodium; (3R,5R)-7-[4-({[(3-fluoro-3′-methoxybiphenyl-4- | 622 |
| | yl)methyl]amino}carbonyl)-2-(4-fluorophenyl)-5-isopropyl- | |
| | 1H-imidazol-1-yl]-3,5-dihydroxyheptanoate | |
| 366 | Sodium; (3R,5R)-7-[4-{[(2-chlorobenzyl)amino]carbonyl}-2- | 532 |
| | (4-fluorophenyl)-5-isopropyl-1H-imidazol-1-yl]-3,5- | |
| | dihydroxyheptanoate | |
| 367 | Sodium; (3R,5R)-7-[4-[(benzylamino)carbonyl]-2-(3,4- | 516 |
| | difluorophenyl)-5-isopropyl-1H-imidazol-1-yl]-3,5- | |
| | dihydroxyheptanoate | |
| 368 | Sodium; 4-[({[1-[(3R,5R)-6-carboxy-3,5-dihydroxyhexyl]-2- | 542 |
| | (4-fluorophenyl)-5-isopropyl-1H-imidazol-4- | |
| | yl]carbonyl}amino)methyl]benzoate | |
| 369 | Sodium; (3R,5R)-7-[2-(3,4-difluorophenyl)-5-isopropyl-4- | 574 |
| | ({[4-(methoxycarbonyl)benzyl]amino}carbonyl)-1H- | |
| | imidazol-1-yl]-3,5-dihydroxyheptanoate | |
| 370 | Sodium; (3R,5R)-7-(2-(3,4-difluorophenyl)-5-isopropyl-4- | 546 |
| | ({[4-methoxybenzyl)amino]carbonyl}-1H-imidazol-1-yl)-3,5- | |
| | dihydroxyheptanoate | |
| 371 | Sodium; (3R,5R)-7-[4-{[(2,4- | 548 |
| | difluorobenzyl)(methyl)amino]carbonyl}-2-(4-fluorophenyl)- | |
| | 5-isopropyl-1H-imidazol-1-yl]-3,5-dihydroxyheptanoate | |
| 372 | Sodium; (3R,5R)-7-(2-(4-fluorophenyl)-5-isopropyl-4-{[(3- | 512 |
| | methylbenzyl)amino]carbonyl}-1H-imidazol-1-yl)-3,5- | |
| | dihydroxyheptanoate | |
| 373 | Sodium; (3R,5R)-7-(2-(4-fluorophenyl)-5-isopropyl-4-{[(4- | 528 |
| | methoxybenzyl)amino]carbonyl}-1H-imidazol-1-yl)-3,5- | |
| | dihydroxyheptanoate | |
| 374 | Sodium; (3R,5R)-7-(2-(4-fluorophenyl)-5-isopropyl-4-{[(3- | 542 |
| | methoxy-4-methylbenzyl)amino]carbonyl}-1H-imidazol-1- | |
| | yl)-3,5-dihydroxyheptanoate | |
| 375 | Sodium; (3R,5R)-7-(2-(4-fluorophenyl)-5-isopropyl-4- | 526 |
| | {[methyl(4-methylbenzyl)amino]carbonyl}-1H-imidazol-1- | |
| | yl)-3,5-dihydroxyheptanoate | |
| 376 | Sodium; (3R,5R)-7-[4-[({4- | 569 |
| | [(dimethylamino)carbonyl]benzyl}amino)carbonyl]-2-(4- | |
| | fluorophenyl)-5-isopropyl-1H-imidazol-1-yl]-3,5- | |
| | dihydroxyheptanoate | |
| 377 | Sodium; (3R,5R)-7-[4-{[(3-chloro-4- | 546 |
| | methylbenzyl)amino]carbonyl}-2-(4-fluorophenyl)-5- | |
| | isopropyl-1H-imidazol-1-yl]-3,5-dihydroxyheptanoate | |
| 378 | Sodium; (3R,5R)-7-{4-[(benzylamino)carbonyl]-2-[4-fluoro- | 566 |
| | 3-(trifluoromethyl)phenyl]-5-isopropyl-1H-imidazol-1-yl}- | |
| | 3,5-dihydroxyheptanoate | |
| 379 | Sodium; (3R,5R)-7-[2-[4-fluoro-3-(trifluoromethyl)phenyl]-5- | 624 |
| | isopropyl-4-({[4-(methoxycarbonyl)benzyl]amino}carbonyl)- | |
| | 1H-imidazol-1-yl]-3,5-dihydroxyheptanoate | |
| 380 | Sodium; (3R,5R)-7-(2-(4-fluorophenyl)-5-isopropyl-4-{[(4- | 575 |
| | pyridin-2-ylbenzyl)amino]carbonyl}-1H-imidazol-1-yl)-3,5- | |
| | dihydroxyheptanoate | |
| 381 | Sodium; (3R,5R)-7-[2-(4-fluorophenyl)-5-isopropyl-4-({[(2- | 575 |
| | phenylpyridin-3-yl)methyl]amino}carbonyl)-1H-imidazol-1- | |
| | yl]-3,5-dihydroxyheptanoate | |
| 382 | Sodium; (3R,5R)-7-{2-(4-fluorophenyl)-5-isopropyl-4-[(2- | 538 |
| | phenylpyrrolidin-1-yl)carbonyl]-1H-imidazol-1-yl}-3,5- | |
| | dihydroxyheptanoate | |
| 383 | Sodium; (3R,5R)-7-[4-[(benzylamino)carbonyl]-5-isopropyl- | 510 |
| | 2-(4-methoxyphenyl)-1H-imidazol-1-yl]-3,5- | |
| | dihydroxyheptanoate | |
| 384 | Sodium; (3R,5R)-7-(2-(4-fluorophenyl)-5-isopropyl-4-{[(4- | 542 |
| | methoxybenzyl)(methyl)amino]carbonyl}-1H-imidazol-1-yl)- | |
| | 3,5-dihydroxyheptanoate | |
| 385 | Sodium; (3R,5R)-7-[4-{[(3- | 530 |
| | fluorobenzyl)(methyl)amino]carbonyl}-2-(4-fluorophenyl)-5- | |
| | isopropyl-1H-imidazol-1-yl]-3,5-dihydroxyheptanoate | |
| 386 | Sodium; (3R,5R)-7-(2-(4-fluorophenyl)-5-isopropyl-4-{[(3- | 542 |
| | methoxybenzyl)(methyl)amino]carbonyl}-1H-imidazol-1-yl)- | |
| | 3,5-dihydroxyheptanoate | |
| 387 | Sodium; (3R,5R)-7-{2-(4-fluorophenyl)-5-isopropyl-4- | 422 |
| | [(methylamino)carbonyl]-1H-imidazol-1-yl}-3,5- | |
| | dihydroxyheptanoate | |
| 388 | Sodium; (3R,5R)-7-(2-(4-fluorophenyl)-5-isopropyl-4- | 556 |
| | {[[(1S)-1-(3-methoxyphenyl)ethyl](methyl)amino]carbonyl}- | |
| | 1H-imidazol-1-yl)-3,5-dihydroxyheptanoate | |
| 389 | Sodium; (3R,5R)-7-(2-[4-fluoro-3-(trifluoromethyl)phenyl]- | 596 |
| | 5-isopropyl-4-{[(4-methoxybenzyl)amino]carbonyl}-1H- | |
| | imidazol-1-yl)-3,5-dihydroxyheptanoate | |
| 390 | Sodium; (3R,5R)-7-(2-(4-fluorophenyl)-4-{[[(1R)-2-hydroxy- | 542 |
| | 1-phenylethyl](methyl)amino]carbonyl}-5-isopropyl-1H- | |
| | imidazol-1-yl)-3,5-dihydroxyheptanoate | |
| 391 | Sodium; (3R,5R)-7-(2-(4-fluorophenyl)-5-isopropyl-4-{[(2- | 575 |
| | pyridin-2-ylbenzyl)amino]carbonyl}-1H-imidazol-1-yl)-3,5- | |
| | dihydroxyheptanoate | |
| 392 | Sodium; (3R,5R)-7-[4-[(benzylamino)carbonyl]-2-(2,4- | 516 |
| | difluorophenyl)-5-isopropyl-1H-imidazol-1-yl]-3,5- | |
| | dihydroxyheptanoate | |
| 393 | Sodium; (3R,5R)-7-(2-(4-fluorophenyl)-5-isopropyl-4-{[[3- | 570 |
| | (methoxycarbonyl)benzyl](methyl)amino]carbonyl}-1H- | |
| | imidazol-1-yl)-3,5-dihydroxyheptanoate | |
| 394 | Sodium; (3R,5R)-7-[2-(4-fluorophenyl)-5-isopropyl-4- | 580 |
| | ({methyl[2-(trifluoromethyl)benzyl]amino}carbonyl)-1H- | |
| | imidazol-1-yl]-3,5-dihydroxyheptanoate | |
| 395 | Sodium; (3R,5R)-7-[4-{[(3,4- | 548 |
| | difluorobenzyl)(methyl)amino]carbonyl}-2-(4-fluorophenyl)- | |
| | 5-isopropyl-1H-imidazol-1-yl]-3,5-dihydroxyheptanoate | |
| 396 | Sodium; (3R,5R)-7-(2-(4-fluorophenyl)-5-isopropyl-4-{[[4- | 570 |
| | (methoxycarbonyl)benzyl](methyl)amino]carbonyl}-1H- | |
| | imidazol-1-yl)-3,5-dihydroxyheptanoate | |
| 397 | Sodium; (3R,5R)-7-[2-(4-fluorophenyl)-5-isopropyl-4- | 540 |
| | ({methyl[(1R)-1-(4-methylphenyl)ethyl]amino}carbonyl)-1H- | |
| | imidazol-1-yl]-3,5-dihydroxyheptanoate | |
| 398 | Sodium; (3R,5R)-7-[4-{[(cyclohexylmethyl)amino]carbonyl}- | 504 |
| | 2-(4-fluorophenyl)-5-isopropyl-1H-imidazol-1-yl]-3,5- | |
| | dihydroxyheptanoate | |
| 399 | Sodium; 3-{[{[1-[(3R,5R)-6-carboxy-3,5-dihydroxyhexyl]-2- | 556 |
| | (4-fluorophenyl)-5-isopropyl-1H-imidazol-4- | |
| | yl]carbonyl}(methyl)amino]methyl}benzoate | |
| 400 | Sodium; (3R,5R)-7-[2-(4-fluorophenyl)-5-isopropyl-4- | 476 |
| | (piperidin-1-ylcarbonyl)-1H-imidazol-1-yl]-3,5- | |
| | dihydroxyheptanoate | |
| 401 | Sodium; (3R,5R)-7-{2-(4-fluorophenyl)-5-isopropyl-4-[(4- | 552 |
| | phenylpiperidin-1-yl)carbonyl]-1H-imidazol-1-yl}-3,5- | |
| | dihydroxyheptanoate | |
| 402 | Sodium; (3R,5R)-7-{2-(4-fluorophenyl)-5-isopropyl-4-[({[6- | 567 |
| | (trifluoromethyl)pyridin-3-yl]methyl}amino)carbonyl]-1H- | |
| | imidazol-1-yl}-3,5-dihydroxyheptanoate | |
| 403 | Sodium; (3R,5R)-7-{2-(4-fluorophenyl)-5-isopropyl-4-[({3- | 609 |
| | [(4-methylpiperidin-1-yl)methyl]benzyl}amino)carbonyl]-1H- | |
| | imidazol-1-yl}-3,5-dihydroxyheptanoate | |
| 404 | Sodium; (3R,5R)-7-(2-(4-fluorophenyl)-5-isopropyl-4-{[(4- | 542 |
| | methoxy-3-methylbenzyl)amino]carbonyl}-1H-imidazol-1- | |
| | yl)-3,5-dihydroxyheptanoate | |
| 405 | Sodium; (3R,5R)-7-{2-(4-fluorophenyl)-5-isopropyl-4-[(3- | 552 |
| | phenylpiperidin-1-yl)carbonyl]-1H-imidazol-1-yl}-3,5- | |
| | dihydroxyheptanoate | |
| 406 | Sodium; (3R,5R)-7-[2-(4-fluorophenyl)-5-isopropyl-4-({[3- | 595 |
| | (piperidin-1-ylmethyl)benzyl]amino}carbonyl)-1H-imidazol- | |
| | 1-yl]-3,5-dihydroxyheptanoate | |
| 407 | Sodium; (3R,5R)-7-[4-[(dimethylamino)carbonyl]-2-(4- | 436 |
| | fluorophenyl)-5-isopropyl-1H-imidazol-1-yl]-3,5- | |
| | dihydroxyheptanoate | |
| 408 | Sodium; (3R,5R)-7-{2-(4-fluorophenyl)-5-isopropyl-4- | 498 |
| | [(phenylacetyl)amino]-1H-imidazol-1-yl}-3,5- | |
| | dihydroxyheptanoate | |
| 409 | Sodium; 4-{[{[1-[(3R,5R)-6-carboxy-3,5-dihydroxyhexyl]-2- | 556 |
| | (4-fluorophenyl)-5-isopropyl-1H-imidazol-4- | |
| | yl]carbonyl}(methyl)amino]methyl}benzoate | |
| 410 | Sodium; (3R,5R)-7-[4-{[4-(ethoxycarbonyl)piperidin-1- | 548 |
| | yl]carbonyl}-2-(4-fluorophenyl)-5-isopropyl-1H-imidazol-1- | |
| | yl]-3,5-dihydroxyheptanoate | |
| 411 | Sodium; (3R,5R)-7-[4- | 462 |
| | {[(cyclopropylmethyl)amino]carbonyl}-2-(4-fluorophenyl)-5- | |
| | isopropyl-1H-imidazol-1-yl]-3,5-dihydroxyheptanoate | |
| 412 | Sodium; (3R,5R)-7-(2-(4-fluorophenyl)-4-{[(3- | 508 |
| | isopropoxypropyl)amino]carbonyl}-5-isopropyl-1H- | |
| | imidazol-1-yl)-3,5-dihydroxyheptanoate | |
| 413 | Sodium; (3R,5R)-7-[4-[(butylamino)carbonyl]-2-(4- | 464 |
| | fluorophenyl)-5-isopropyl-1H-imidazol-1-yl]-3,5- | |
| | dihydroxyheptanoate | |
| 414 | Sodium; (3R,5R)-7-[4-({[(1R)-1- | 518 |
| | cyclohexylethyl]amino}carbonyl)-2-(4-fluorophenyl)-5- | |
| | isopropyl-1H-imidazol-1-yl]-3,5-dihydroxyheptanoate | |
| 415 | Sodium; (3R,5R)-7-[4-[(4-fluorobenzoyl)amino]-2-(4- | 502 |
| | fluorophenyl)-5-isopropyl-1H-imidazol-1-yl]-3,5- | |
| | dihydroxyheptanoate | |
| 416 | Sodium; (3R,5R)-7-{2-(4-fluorophenyl)-5-isopropyl-4-[(4- | 514 |
| | methoxybenzoyl)amino]-1H-imidazol-1-yl}-3,5- | |
| | dihydroxyheptanoate | |
| 417 | Sodium; (3R,5R)-7-[4-(benzoylamino)-2-(4-fluorophenyl)-5- | 484 |
| | isopropyl-1H-imidazol-1-yl]-3,5-dihydroxyheptanoate | |
| 418 | Sodium; (3R,5R)-7-[4-{[(4-chlorobenzoyl)amino]methyl}-2- | 532 |
| | (4-fluorophenyl)-5-isopropyl-1H-imidazol-1-yl]-3,5- | |
| | dihydroxyheptanoate | |
| 419 | Sodium; (3R,5R)-7-[4-[(benzoylamino)methyl]-2-(4- | 498 |
| | fluorophenyl)-5-isopropyl-1H-imidazol-1-yl]-3,5- | |
| | dihydroxyheptanoate | |
| 420 | Sodium; (3R,5R)-7-(2-(4-fluorophenyl)-5-isopropyl-4- | 472 |
| | {[(methylsulfonyl)amino]methyl}-1H-imidazol-1-yl)-3,5- | |
| | dihydroxyheptanoate | |
| 421 | Sodium; (3R,5R)-7-[2-(4-fluorophenyl)-4-({[(4- | 530 |
| | fluorophenyl)acetyl]amino}methyl)-5-isopropyl-1H- | |
| | imidazol-1-yl]-3,5-dihydroxyheptanoate | |
| 422 | Sodium; 7-[4-Ethylcarbamoyl-2-(4-fluoro-phenyl)-5- | 436 |
| | isopropyl-imidazol-1-yl]-3,5-dihydroxyheptanoate | |
| 423 | Sodium; 7-[4-(4-Chloro-benzoylamino)-2-(4-fluoro-phenyl)- | 518 |
| | 5-isopropyl-imidazol-1-yl]-3,5-dihydroxyheptanoate | |

### Example 424

### Sodium: (3R,5R)-7-[4-Benzylcarbamoyl-2-(4-fluorophenyl)-5-isopropyl-imidazol-1-yl]-3.5-dihydroxy-heptanoate

Step A

### (Benzhydrylideneamino)-acetic acid benzyl ester

A 3-necked, 5 L round-bottomed flask was equipped with a mechanical stirrer, a J-KEM temperature probe, and a N₂ inlet adapter connected to a bubbler. The round-bottomed flask was charged with glycine benzyl ester hydrochloride ( 505.2 g, 2.51 mol, 1.0 equiv.) and CH₂Cl₂ (3.0 L). The milky, white reaction mixture was treated with benzophenone imine (471.1 g, 97%, 2.6 mol, 1.00 equiv.) and an exotherm (+4.5 ° C.) was observed. The reaction mixture stirred at 20° C. for 3h and TLC (50% ethyl acetate/heptane) showed a trace of starting material . Additional benzophenone imine (25.0 g, 0.14 mol) was added to the reaction mixture and the mixture was stirred for 15 h at 20° C. TLC confirmed reaction completion. This mixture was filtered through a short pad of Celite to remove ammonium chloride, and the filter cake was rinsed with CH₂Cl₂ (1.5 L). The filtrates were concentrated in vacuo to produce a white solid that was dried in vacuo to give the desired crude product: 878.7 g (106%); ¹H-NMR(DMSO-d₆): 7.53-7.25 (m, 13H), 7.12 (dd, 2H), 5.10 (s, 2H), and 4.17 (s, 2H). HPLC Purity: >95%.

Step B

### 2-Amino-4-methyl-3-oxo-pentanoic acid benzyl ester hydrochloride

A 3-necked, 3 L round-bottomed flask was equipped with a magnetic stir bar, a J-KEM temperature probe, an addition funnel, and a N₂ inlet adapter connected to a bubbler. The flask was charged with potassium tert-butoxide (112.0 g, 998 mmol, 1.53 equiv) and THF (750 mL). The white suspension was cooled to −70 ° C. and was treated with (Benzhydrylideneamino)-acetic acid benzyl ester (215.0 g, 658 mmol, 1.00 equiv.) as a solution in THF (700 mL). The orange solution stirred for 30 min at −70° C. and was then transferred via cannula into a solution of isobutyryl chloride (100.0 mL, 101 g, 947 mmol, 1.45 equiv.) in THF (200 mL) at −70 ° C. The addition rate was such that the reaction temperature did not warm past −50° C. After complete addition, the reaction mixture was held at −50 ° C. for 1 h, and was then warmed to −30° C. At this temperature, the reaction was quenched with 3 M HCl (670 mL, 2.0 mol, 3.1 equiv.). The cold bath was removed, and the reaction mixture stirred at 20 ° C. for 15 h. The reaction mixture was concentrated in vacuo to produce a yellow residue that was re-dissolved in water (400 mL). The benzophenone side-product was removed by extraction with diethyl ether (2×400 mL), and the aqueous layer was concentrated in vacuo to produce a light yellow residue that was concentrated twice on the rotary evaporator from methanol (2×500 mL) to azeotropically remove water. The resulting residue was then re-dissolved in anhydrous methanol (500 mL) and potassium chloride (KCl, −82.0 g) was removed by vacuum filtration. The light yellow filtrate was concentrated in vacuoto produce a light yellow residue (16, 143.1 g, 81%). ¹H-NMR (DMSO-d₆): 9.08 (s, 3H, NH₃Cl), 7.41-7.31 (m, 5H), 5.48 (s, 1H), 5.26 (s, 2H), 3.05 (sept, 1H), 1.08 (d, 3H, CH₃), and 0.90 (d, 3H, CH₃). HPLC purity: 88.2%. MS: (M−HCl)=235. This crude residue 16 can be recrystallized from a 1:1 wt/wt ratio of crude 16 to water to provide 16 >99% HPLC purity.

Step C

### 2-(4-Fluorobenzoylamino)-4-methyl-3-oxo-pentanoic acid benzyl ester

A 4-necked, 5 L round-bottomed flask was equipped with a J-KEM temperature probe and a mechanical stirrer. The flask was charged with 2-Amino-4-methyl-3-oxo-pentanoic acid benzyl ester hydrochloride (427.8 g, 99.6% HPLC purity, 1.57 mol) and CH₂Cl₂ (1.0 L). The resultant solution was cooled to 0° C. and was treated with a solution of potassium carbonate (546 g, 3.95 mol, 2.51 equiv.) in deionized water (1.5 L) to produce a creamy reaction mixture. The pot temperature was kept below 5° C. during the potassium carbonate addition. Then, the mixture was treated with a solution of 4-fluorobenzoyl chloride (209 mL, 276 g, 1.74 mol, 1.11 equiv.) in CH₂Cl₂ (500 mL) at 0° C. at a rate such that the pot temperature was kept below 5° C. TLC (50% ethyl acetate/50% hexanes) showed reaction completion after 20 min and a phase cut gave the product-containing bottom yellow organic layer. The aqueous layer was extracted with CH₂Cl₂ (1×750 mL) and discarded. The combined organic layers were washed with 0.2 M HCl (1×90 mL), washed with water (1×2 L, deionized), dried over MgSO₄, and filtered. The yellow filtrate was concentrated in vacuo to produce a light yellow solid (583,5 g, 104%) which was recrystallized from into a refluxing mixture of MTBE (1 L) and heptane (2.5 L) to give an solid, which was collected by filtration and washed with heptane (2×0.5 L). This material was dried in vacuo (35° C.) for 12 h to give the desired product as an off white solid: 504.0 g, (90%); ¹H-NMR (CDCl₃): 7.86 (m, 2H), 7.41-7.10 (m, 7H), 5.59 (d, 1H), 5.27 (dd, 2H), 3.05 (m, 1H), 1.21 (d, 3H), and 1.19 (d, 3H); ¹⁹F-NMR (CDCl₃): −107.54; Low resolution mass spectroscopy (APCI) m/z 358 [M+H]⁺.

Step D

### N-(1-Benzylcarbamoyl-3-methyl-2-oxo-butyl)-4-fluorobenzamide

A 4-necked, 3 L round-bottomed flask was equipped with a J-KEM temperature probe, a magnetic stirrer, a condenser connected to a bubbler via a N₂ inlet adapter, and an addition funnel. The flask was charged with 2-(4-Fluorobenzoylamino)-4-methyl-3-oxo-pentanoic acid benzyl ester (200.0 g, 0.56 mol, 1.00 equiv.) and NMP (850 mL). The resultant solution was heated to 160° C. and treated in one portion with neat benzylamine (65.0 mL, 31.48 g, 0.29 mol, 1.05 equiv.). The reaction mixture was maintained at 160° C. for 3 h, TLC and HPLC (50:50 ethyl acetate/hexanes) showed desired product and very little starting material. The reaction mixture was cooled to 75° C. and NMP (˜600 mL) was removed by vacuum distillation. The concentrated reaction mixture was poured portionwise onto a cold brine solution (1.5 L; approximately 1:2 in ice/water) and was diluted with ethyl acetate (1 L). The organic layer was collected and the aqueous layer was extracted with ethyl acetate (1×500 mL). The combined ethyl acetate filtrate was concentrated in vacuo to produce a beige solid (˜284 g). ¹H-NMR still showed NMP in solid residue. The solid residue was re-dissolved in ethyl acetate (1.5 L) and washed with Y2 saturated brine solution (2×2 L; 1 L saturated brine). The organic layer was collected and concentrated in vacuo to produce a light yellow solid (˜254 g). ¹H-NMR showed very little NMP in crude solid. Using a mechanical stirrer, crude solid (˜254 g) was recrystallized with absolute EtOH (700 mL) and deionized water (700 mL) to produce an off-white solid. The off-white solid was collected by filtration and air-dried in the hood over 15 h. The off-white solid (-400 g, wet) was re-slurried in a solution of absolute ethanol (600 mL) and deionized water (600 mL), collected by filtration, and dried in vacuo 75° C. (16 h) to give the desired product as an off-white solid: (112.3 g, 56% yleld, 90% HPLC purity); ¹H-NMR (CDCl₃): 7.83 (m, 2H), 7.78, (d, 1H), 7.41-7.10 (m, 6H), 5.33 (d, 1H), 4.42 (m, 2H), 3.15 (m, 1H), and 1.10 (m, 6H); ¹⁹F-NMR (CDCl₃): −106.95; Low resolution mass spectroscopy (APCI) m/z 357 [M+H]⁺.

Step E

### [(4R,6R)-6-(2-Amino-ethyl)-2,2-dimethyl-[1,3dioxan-4-yl]-acetic acid tert-butyl ester

A 5-gallon stainless steel reactor was charged with 250 g of Ra—Ni, ((4R,6R)-6-Cyanomethyl-2,2-dimethyl-[1,3]dioxan-4-yl)-acetic acid tert-butyl ester (1.0 kg, 3.71 mol), toluene (6 L), methanol (675 mL), and with 6.5M NH₃/MeOH (800 mL). The reactor was sealed, pressure tested to 3,5 bar with N₂, and purged 3 times with 3,5 bar of N₂. The reactor was purged with H₂ to 3,5 bar three times without any agitation. After the reactor was pressurized to 3,5 bar with H₂, the reaction stirred for 2-6 h, and a small exotherm to 30 to 40° C. was observed. Stirring was continued until H₂ uptake ceased, then the reaction mixture was stirred at 30 to 40° C. for a further 30 min. The mixture was cooled to 20 to 25° C., the H₂ source and the agitator were switched off, and the H₂ was vented from the reactor. The agitator was switched on and the stainless steel reactor was purged with N₂ to 3,5 bar 3 times. Spent Ni catalyst was filtered under a bed of nitrogen, and the stainless steel reactor and spent catalyst bed were washed with toluene (250 mL). The combined filtrates were concentrated to an approximate volume of 500 mL at a maximum temperature of 55° C. under vacuum. [Note: the vacuum was broken with nitrogen]. A saturated sodium chloride solution was added and stirred for 10 minutes under nitrogen. The agitation was stopped and the phases were separated. The lower aqueous layer was discarded, and the organic layer was concentrated to produce the desired product as a yellow oil: (1.054 kg, 104%, -7% residual toluene); ¹H-NMR (400 MHz, CDCl₃): 4.23-4.19 (m, 1H), 3.99-3.95 (m, 1H), 2.74 (t, J=7.1 Hz, 2H), 2.40-2.36 (m, 1 H), 2.27-2.22 (m, 1H), 1.58-1.41 (m, 2H), 1.40 (s, 9H), 1.31 (s, 6H), 0.89 (s, 9H); Low resolution mass spectroscopy (APCI) m/z 273 [M+H]⁺.

Step F

### 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid benzylamide

To a 2-L 3-necked, round-bottomed flask outfitted with a mechanical stirrer, a J-KEM/heating mantle setup, and a Dean-Stark trap (with condenser) was charged a mixture of N-(1-Benzylcarbamoyl-3-methyl-2-oxo-butyl)-4-fluorobenzamide (123.0 g, 345.1 mmol), benzoic acid (63.0 g, 517.5 mmol, 1.5 equiv.), and heptane (700 mL). This slurry was treated with [(4R,6R)-6-(2-Amino-ethyl)-2,2-dimethyl-[1,3]dioxan-4-yl]-acetic acid tert-butyl ester (119.4 g, 414.0 mmol, 1.2 equiv.). The reactor was purged with nitrogen, then heated to reflux (approximately 99° C.) over 14 h in order to azeotropically remove the water formed during the reaction. After 14 h, a small amount of starting material remained by TLC (1:1 heptane:ethyl acetate). A small portion of TBIA (5.0 g, 18.0 mmol, 0.06 equiv) was added to the reactor, and the mixture was stirred at reflux for another 2 h, after which time TLC showed no more starting material remaining. The reactor was cooled to 30° C., and the contents were fully dissolved with ethyl acetate (600 mL), washed with saturated sodium bicarbonate solution (2×400 mL), washed with 10% aqueous sodium chloride, then concentrated in vacuo to provide 400.1 g of a very thick orange oily solid. This solid was taken up into MeOH (600 mL) while heating to 40° C. (difficult to dissolve). The solution was charged with a premixed solution of concentrated HCl (136 g) in water (400 mL), and the remaining solution was heated back to 40° C. and held at this temperature for over 2 h. The walls of the reactor were washed down with MeOH (20 mL) and TLC after an additional 1 h showed mainly diol tert-butyl ester. To the reaction mixture was added MTBE (500 mL), followed by slow addition (-10 min) of a pre-mixed solution of NaOH (110 g) in water (200 mL). The pH of the mixture at this point was 13.0, and the pot temperature rose to almost 50° C. The reaction was stirred and slowly cooled to 23° C. over 2 h, after which time TLC (6:1 ethyl acetate:heptane) showed that all tert-butyl ester was consumed (only baseline remaining). The mixture was diluted with more MTBE (1 L) and water (500 mL), and was phase separated. The bottom aqueous product-containing layer was extracted again with MTBE (500 mL) and set aside. The combined MTBE layers were vigorously washed with 5% NaOH solution (200 mL), then discarded. The combined aqueous extracts were combined and distilled down to approximately 1/2 volume on the rotary evaporator using full vacuum at 70° C. (CAUTION! Severe bumping was possible; use large round-bottom flask and a bump-trap for this concentration). The mixture was then stirred at 23° C. and treated with 6N HCl (200 mL, added over 1 min), at which point the mixture turned cloudy. The pH of this suspension was 7.0 (pH was measured with pH meter). To this mixture was added ethyl acetate (800 mL), and the mixture was stirred vigorously. The mixture was then treated with 6N HCl until pH of the aqueous layer (phase-cut; lower layer) was 5.5. In total, additional 6N HCl (75 mL) was added to achieve this pH. The layers were separated and the top organic layer was set aside. The aqueous layer was extracted with ethyl acetate (200 mL) and then discarded. The combined organics were washed with water and then concentrated in vacuo to give 175 g of an orange oil that foamed slightly under vacuum. To this mixture was added 1% HCl (1 mL) and toluene (900 mL), and the reaction mixture was heated to reflux under a Dean-Stark trap for 2.5 h [Note: Not completely in solution until near reflux]. TLC showed clean conversion to lactone. The reaction mixture was cooled to 30° C., and toluene was removed by rotary evaporator to give 171 g of a brown oil that solidified while under vacuum for 2 h. This solid was taken up in dichloromethane (60 mL) and the solution was added to the top of a 900 g silica gel column that was pre-packed in 4:1 ethyl acetate/heptane. A solution of 4:1 ethyl acetate/heptane (4 L) eluted initially a purple impurity of high R_{f} (0.8), followed by elution of lactone cleanly by ramping eventually to neat ethyl acetate over another 12 L. Additional ethyl acetate (6 L) was charged until the product was completely eluted as indicated by TLC (5:1 ethyl acetate/heptane). Fractions 3-6 (500 mL each) contained the purple impurity, and fractions 10-22 were combined and concentrated to afford 103.5 g of a dark grey oil that formed a tan foamy residue while drying under vacuum. NMR of this residue showed contamination with benzoic acid, so this crude product was re-dissolved in ethyl acetate (500 mL), washed with saturated sodium bicarbonate solution (2×200 mL), followed by washing with 100 mL water. The organic solvent was concentrated in vacuo to yield the desired product as a pale tan foamy amorphous solid: (88.4 g 53% over 4 combined steps); ¹H-NMR (CDCl₃): 7.61 (m, 2H), 7.34-7.22 (m, 7H), 4.57 (m,1H), 4.51 (s, 2H), 4.31 (m,1H), 4.20 (m, 2H), 3.29 (p, 1H), 2.62 (dd, 1 H), 2.44 (dd, 1H), 1.90 (m, 2H), 1.71 (m, 2H), and 1.43 (d, 6H); ¹⁹F-NMR (CDCl₃): −113.66; Low resolution mass spectroscopy (APCI) m/z 480 [M+H]⁺

Step G

A 3-necked, 3-L round-bottomed flask was outfitted with a large (400 mL) Dean-Stark trap (with a condenser) and a J-KEM temperature probe was charged with 2-(4-Fluoro-phenyl)-1-[2-((2R,4R)-4-hydroxy-6-oxo-tetrahydro-pyran-2-yl)-ethyl]-5-isopropyl-1H-imidazole-4-carboxylic acid benzylamide_(88.4 g, 184 mmol) and 1M NaOH (180.3 mL, 180.3 mmol, 0.98 equiv, based on HPLC purity of lactone 23, 98% purity in this case). The resulting mixture was diluted with water (750 mL) and warmed to 60° C. for 2h to aid in dissolution/conversion of lactone to sodium salt. After 2 h, TLC (100% ethyl acetate) showed nearly complete consumption of lactone (R_{f}=0.5). The biphasic solution was heated to reflux (˜95° C.) to azeotrope off water (-700 mL, some water loss through top of condenser) over 3 h. The remaining white slurry was diluted with toluene (500 mL) and concentrated in vacuo to produce a beige residue (p110 g). The crude residue was transferred to the vacuum oven at 80° C. for 12 h under a nitrogen sweep to afford a white solid (92.2 g). In a wide-mouth 2-L Erlenmeyer flask with a gentle nitrogen sweep, this solid was dissolved in refluxing MeOH (900 mL) with vigorous stirring. The solution was concentrated down by boiling off methanol until approximately 800 mL of total volume remained. While refluxing, 2-propanol (500 mL) was added dropwise over 60 min (so that the total volume remains -800 mL; i.e. as methanol continued to boil off, 2-propanol was added at the same rate to keep a constant reaction mixture volume), during which time the refluxing solution began to precipitate sodium salt. After full addition, the mixture was refluxed until the total volume reached 700 mL, after which heating was discontinued (stirring continued), and the slurry was cooled to 23° C (uncontrolled, no temperature ramp was used). The bright, white fluffy solid was filtered on a glass fitted filter funnel, and the cake was rinsed with 2-propanol (100 mL). The cake was sucked dry under a nitrogen sweep for 0.5 h to provide 135 g of wet cake that was placed in the vacuum oven at 75° C. for 12 h under a slight nitrogen purge to afford 67.7 g of a white, fluffy solid. ¹H-NMR (CD₃OD): □ ppm 1.48 (m, 7 H), 1.58 (m, 1 H), 1.70 (m, 1 H), 1.81 (m, 1 H), 2.23 (dd, J=15.04, 7.42 Hz, 1 H), 2.29 (dd, J=15.24, 5.47 Hz, 1 H), 3,46 (m, 1 H), 3.73 (m, 1 H), 4.11-3.92 (m, 2 H), 4.21 (ddd, J=14.85, 11.33, 5.08 Hz, 1 H), 4.51 (s, 2 H), 7.33-7.19 (m, 7 H), 7.62 (m, 2 H); ¹⁹F-NMR (CD₃OD): −113.83; Low resolution mass spectroscopy (APCI) m/z 498 [M+H]⁺; Anal. calculated for C₂₇H₃₁F₁N₃Na₁O₅: C, 62.42; H, 6.01; N, 8.09; Na, 4.40. Found: C, 62.32; H, 5.93; N, 8.05; Na, 4.39; IR(neat) vₘₐₓ=1657, 1574, 1512, 1411, 1223, 846, and 700 cm⁻¹.

### EXAMPLES

### Pyrroles

Example 1P shows the preparation of a compound of Formula I wherein R² and R³ are each para-fluorophenyl, R³ is isopropyl and R⁴ is SO₂NR⁹R¹⁰. In Example 1P, one of R⁹ and R¹⁰ is H and the other one of R⁹ and R¹⁰ is phenyl. Compounds with variations on R⁹ and R¹⁰ were made using a similar reaction scheme and are shown, along with characterizing data, in TABLE I which follows Example 1P.

### Example 1P

### (3R,5R)-7-[2,3-bis-(4-fluoro-phenyl)-5-isopropyl-4-phenylsulfamoyl-pyrrol-1-yl]-3,5-dihydroxy-heptanoic acid monosodium salt

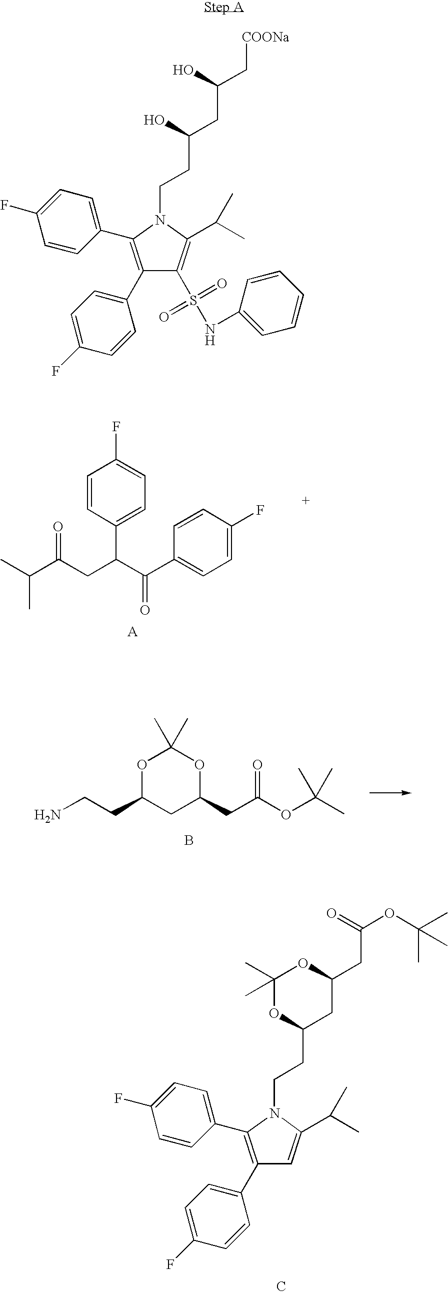

Preparation:

To a solution of the above starting material-A and the above starting material-B in heptane/toluene (9/1, v/v) was added trimethylacetic acid. The resulting reaction mixture was refluxed under N₂ for 16 hrs. The water formed was removed with a Dean-Stark trap. The total volume of the water removed was 1.85 mL (1.91 mL by theory). The reaction mixture was cooled to RT, washed successively with 1 N HCl, 1N NaOH, sat. NaHCO₃ and brine, and concentrated in vacuo to give a brown syrup. The brown syrup was dissolved in 80 mL of MeOH, chilled in an ice-bath, yellow solid precipitated and the desired product was isolated via filtration (24.65 g), MP 88-91° C.

Combustion Analysis for (C₃₃H₄₁F₂NO₄.0.15CH₃OH):

| | Carbon | Hydrogen | Nitrogen | F |
|---|---|---|---|---|
| Theory | 71.29 | 7.51 | 2.51 | 6.80 |
| Found | 71.61 | 7.90 | 2.60 | 7.00 |

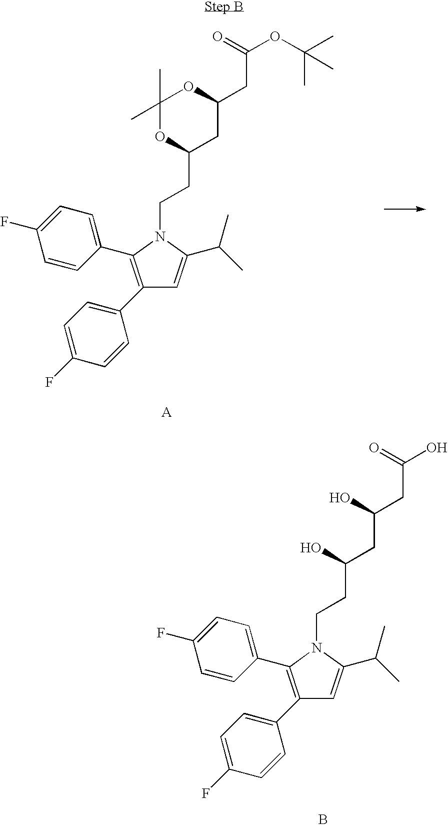

Preparation:

To a suspension of the above starting material-A from Step A in MeOH (8.88 mmol, 308 mL) was added 1 N HCl (20.8 mL). The resulting mixture was stirred using a mechanical stirrer for 24 hours. An aqueous NaOH solution (1N, 55.5 mL) was added. The reaction mixture was stirred for another 16 hours. The reaction mixture was diluted with 100 mL of water, washed with hexane (2×200 mL), and acidified with con. HCl to pH=2. White precipitate formed, the mixture was extracted with EtOAc (3×200 mL), and the combined organic solution was dried over Na₂SO₄. The mixture was filtered and the filtrate was concentrated in vacuo, two phases formed, water was separated out, and the organic phase was concentrated affording a beige solid (14.5 g). MP 195-196° C.; MS, APCI+440.2 (M−18+H).

Combustion Analysis for [C₂₆H₂₉F₂NO₄]:

| | Carbon | Hydrogen | Nitrogen | F |
|---|---|---|---|---|
| Theory | 68.26 | 6.39 | 3.06 | 8.30 |
| Found | 69.46 | 6.17 | 3.05 | 8.64 |

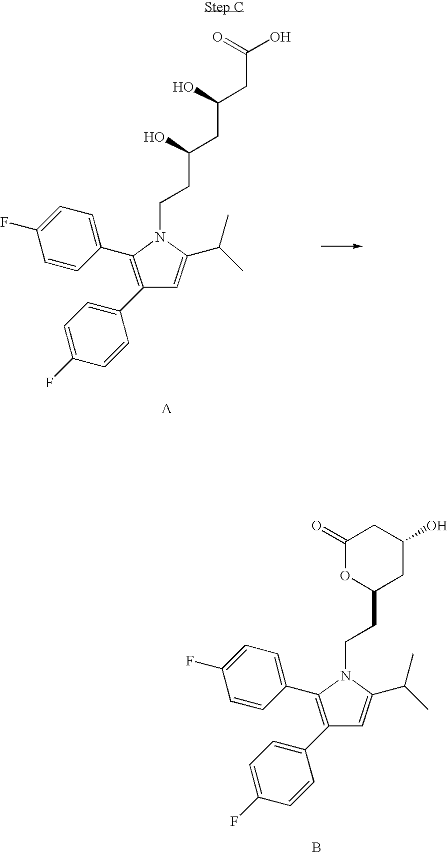

Preparation:

To a suspension of the above starting material-A from Step B in toluene (200 mL) was added conc. HCl (3 drops). The resulting mixture was refluxed for 5 hrs. Water formed was continuously removed from the system with a Dean-Stark trap. The residual solid was removed via a hot filtration, the filtrate was cooled to RT, white crystals formed and were isolated via filtration (12.2970 g). MS, APCI+440.2 (M+H);

Combustion Analysis for: C₂₆H₂₇F₂NO3

| | Carbon | Hydrogen | Nitrogen | F |
|---|---|---|---|---|
| Theory | 71.05 | 6.19 | 3.19 | 8.65 |
| Found | 71.10 | 6.45 | 3.40 | 8.56 |

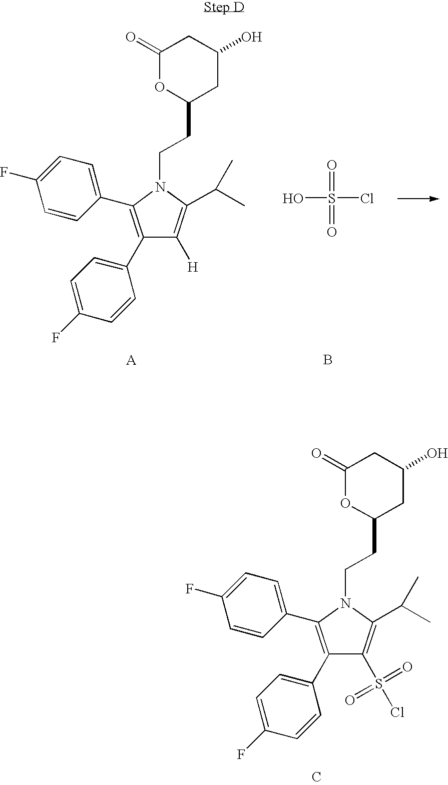

Preparation:

To a suspension of the starting material A from Step C in dichloromethane (9.0 mL) was added 12.0 mL of chlorosulfonic acid. A brown reaction solution was obtained. The reaction mixture was stirred at RT for 3 hours and 35 minutes. The reaction mixture was cooled to −60° C. , diluted with 100 mL of ethyl acetate and cooled at −60° C. 200 mL of ethyl acetate at RT was added. The solution was warmed to 22° C., then poured onto ice. When the ice was, two phases were separated (aqueous layer was about 25 mL). The aqueous phase was extracted with EtOAc (1×20 mL), and the combined organic phase was poured onto ice again. The temperature of the mixture was kept at around 22° C. with a hot water bath. When the ice was melted, two phases were separated (aqueous layer was about 30 mL). The organic phase was washed again with brine (60 mL), the mixture was allowed to stand for 20 minutes for good phase separation, and then the two phases were separated. The organic phase was dried over Na₂SO₄ first, then solid NaHCO₃ was added to neutralize the residual acid in the solution. After 5 minutes, the mixture was filtered. The filtrate was concentrated in vacuo. When the volume was reduced to about 150 mL, the solution became cloudy. The mixture was filtered and the filtrate was concentrated to give the desired product (3.7247 g) as a light yellow foam which was used in the next step without further purifications.

Step E

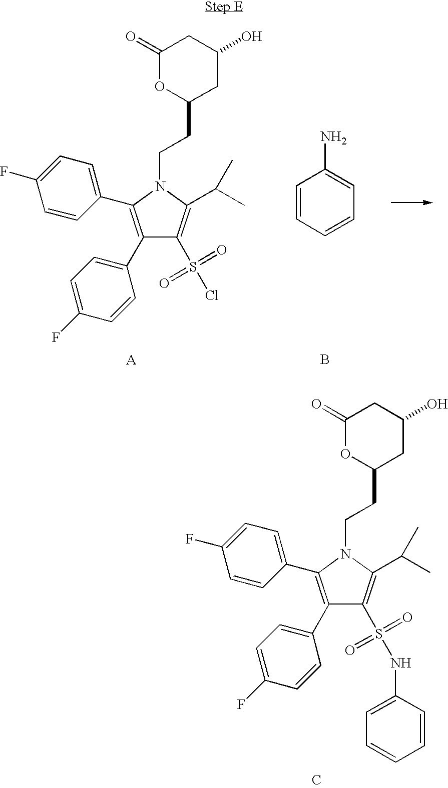

Preparation:

To a solution of the starting material A from Step D, in DMF (2.0 mL), was added 0.666 mL of aniline. The reaction mixture was stirred at RT under nitrogen for 3 hours. After 3 hours, the reaction mixture was diluted with 50 mL of ethyl acetate, washed with 1 N HCl (2×30 mL) and brine, and dried over Na₂SO₄. The crude product was purified by chromatography and the desired product was isolated as a white foam (0.2685 g). MP, 93-101° C. Combustion Analysis for [C₃₂H₃₂F₂N₂O₅S.0.5C₄H₈O₂ (ethyl acetate)]:

| | Carbon | Hydrogen | Nitrogen | F |
|---|---|---|---|---|
| Theory | 63.93 | 5.68 | 4.39 | 5.95 |
| Found | 63.56 | 5.76 | 4.35 | 6.11 |

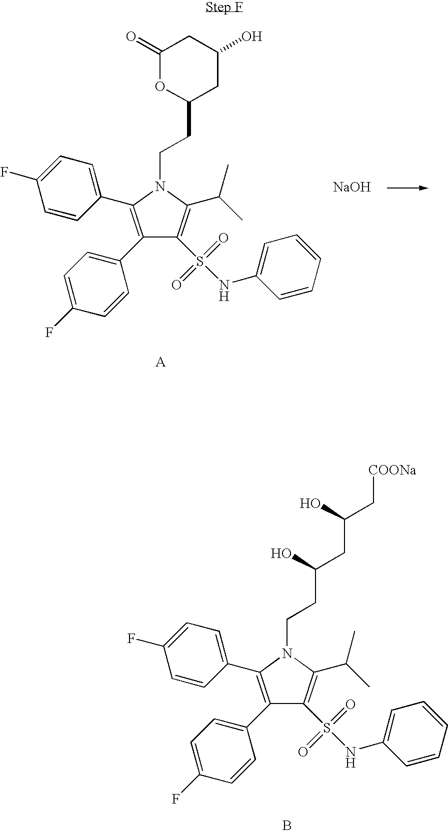

Preparation:

To a solution of the above starting material-A from Step E in MeOH was added 1N NaOH. The resulting reaction solution was stirred at RT for 40 minutes, and then concentrated in vacuo. 2 mL of MeOH was added to dissolve the residue, 10 mL of toluene was added, and then evaporated to azeotropically remove water. This process was repeated (twice) until a white solid was obtained. The white solid was dissolved in a very small amount of MeOH, then diluted with 20 mL of 5% MeOH in methylene chloride. A cloudy solution was obtained. After standing for 0.5 hour, the mixture was filtered to remove the solid (excess of NaOH, the sodium salt is soluble in 5% MeOH in methylene chloride). The filtrate was concentrated in vacuo to afford a solid, which was triturated with ether to form a white precipitate. Filtration gave the desired product as a beige solid (0.1885 g), MS (APCI+) 613.2; MP 130-134° C. (decomposed).

Combustion Analysis for (C₃₂H₃₃F₂N₂NaO₆S.C₄H₁₀O.1.5H₂O):

| | Carbon | Hydrogen | Nitrogen | F |
|---|---|---|---|---|
| Theory | 58.23 | 5.66 | 4.14 | 5.62 |
| Found | 58.04 | 5.29 | 4.00 | 5.62 |

Compounds (sodium salt thereof) with variations on R⁹ and R¹⁰ were made using a similar reaction scheme to Example 1P and are shown, along with characterizing data, in TABLE I below.

TABLE I
Variations On Example 1P

| R⁹ | R¹⁰ | MP | MS |
|---|---|---|---|
| H, | 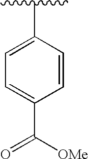 | >240° C. | 671.1 (APCl+) |
| H, | 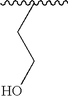 | 147-150° C. | 581.1, acid + H (APCl+) |
| H, | 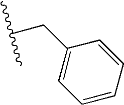 | 115-119° C. | |
| H, | 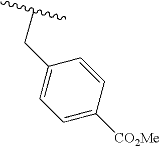 | 131-135° C. | 685.2, acid + H (APCl+) |
| H, | 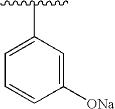 | 166-169° C. | 629.1, acid + H (APCl+) |
| H, | 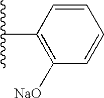 | 175-178° C. | 629.2, acid + H (APCl+) |
| H, | CH₃ | 140-143° C. | |
| H, | 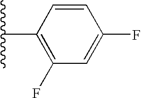 | 80-91° C. | 631.1 (M + H) (APCl+) |
| H, | 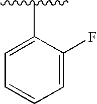 | 84-90° C. | 613.1 (M + H) (ASPCl+) |
| H, | 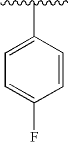 | 161-166° C. | 631.1 (APCl+) |
| H, | 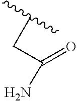 | 108-110° C. | 594.2 (acid + H) (APCl+) |
| NR⁹R¹⁰ = | 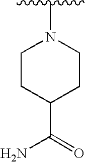 | 161-165° C. | 648.2 (acid + H) (APCl+) |
| H, | 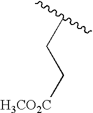 | 120-124° C. | 623.2 (acid + H) (APCl+) |
| H, | 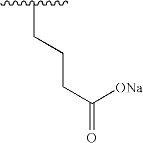 | 155-157° C. | 623.1 (acid + H) (APCl+) |
| NR⁹R¹⁰ = | 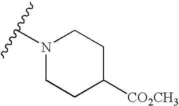 | 150-155° C. | 663.2 (acid + H) (APCl+) |
| H, | 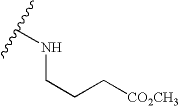 | 105-108° C. | 637.2 (acid + H) (APCl+) |
| H, | 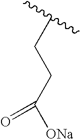 | >240° C. | 609.1 (acid + H) (APCl+) |
| H, | 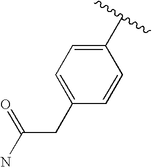 | 125-127° C. | 670.3 (APCl+, acid + H) |
| H, | 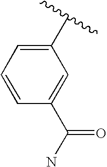 | 99-101° C. | 654.3 (APCl−, acid − H) |
| H, | 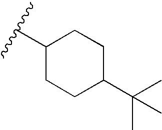 | 99-101° C. | 654.3 (APCl−, acid − H) |
| NR⁹R¹⁰ = | 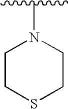 | 92-94° C. | 623.1 (APCl+, acid + H) |
| NR⁹R¹⁰ = | 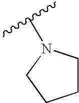 | 138-140° C. | 591.2 (APCl+, acid + H) |
| H, | 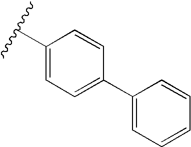 | 150-152° C. | 689.2 (APCl+, acid + H) |
| NR⁹R¹⁰ = | 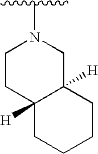 | 123-126° C. | 657 (APCl−, acid − H) |
| NR⁹R¹⁰ = | 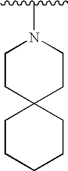 | 151-153° C. | 673.1 (APCl+, acid + H) |

In Examples 2P-17P, the numbers refer to compounds shown in Schemes 15,15a, 15b, 15c, 16,16a, 17,17a or 18.

### Example: 2P

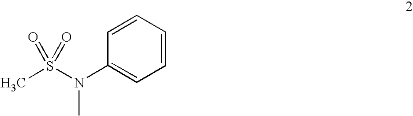

To a DCM solution (125 mL) containing methanesulfonyl chloride (10 g, 0.087 mole) at 0° C. was added N-methyl aniline (1.25 equiv), followed by dropwise addition of triethylamine (1.25 equiv). The reaction mixture was stirred at 0 OC for one hour and slowly warmed to room temperature. The TLC result showed a spot to spot transformation of the methanesulfonamide 2 (Rf=0.02 to 0.3. in 30% EtOAc/Hex). Work-up: The reaction mixture was evaporated under reduced pressure, and 1N aqueous HCl was added until the pH of the solution became acidic. The desired compound was extracted using EtOAc (25 mL×2), and the organic phase was washed with water (20 mL×2), brine (10 mL), dried over Na₂SO₄, and filtered. The filtrate was then evaporated under reduced pressure to give a pale yellow solid (crude 14.2 g, 88% recrystallized with MeOH or 12.0 g. MS M+H=186 found: 186, ¹H NMR structure confirmed).

### Example 3P

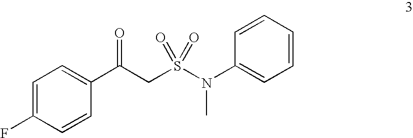

To a THF solution (20 mL) containing the methanesulfonamide 2 (2.5 g) at −78° C. was dropwise added n-butyllithium (10.3 mL of 2.5 M in Hexane). The reaction mixture was then warmed to 0° C. and cooled back to −78° C. before methyl p-fluorobenzoate (2.5 g in THF (5 mL)) was added. The reaction mixture was stirred for 1 h after the dry ice bath was removed. Work-up: The reaction mixture was concentrated under reduced pressure, and the resultant suspension was treated with 1N aqueous HCl solution. Once acidified the reaction mixture was extracted with DCM (10 mL ×2). The organic phase was then washed with water (10 mL ×2), dried over Na₂SO₄, and filtered. The filtrate then was evaporated under reduced pressure to give a white solid (4.47 g MS M+H=308 found: 308, ¹H NMR structure confirmed).

### Example 4P

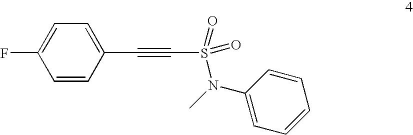

Dry triethylamine (3 mL) was slowly added to a DCM solution (5 mL) containing the □-ketosulfonamide 3 (460 mg, 1.5 mmole) and 2-chloro N-methylpyridinium iodide (511 mg, 2.0 mmole) at ambient temperature. The suspension was stirred at room temperature for 2 days. To take a TLC a small aliquot of the sample was treated with 1 N NaOH, extracted with DCM. The TLC spot was taken from the organic phase. Work-Up: After 2 days, the suspension was treated with 1N NaOH (5 mL) for 5 min. Then it was extracted with DCM (20 mL×2). This organic phase was successively washed with 1N NaOH, 1N HCl, water, dried over Na₂SO₄, and filtered. The filtrate was then passed through a short column of basic alumina. The resultant DCM solution was then evaporated under reduced pressure to give a yellow solid (282 mg, 0.975 mmole, 65%).

### Example 5P

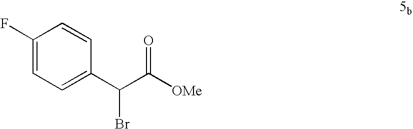

To a dry MeOH solution (50 mL) containing 4-fluorophenylacetic acid 5a (5 g, 0.0324 mole) was added a catalytic amount of 4-toluene sulfonicacid (0.324 mmole, 61 mg). The solution was refluxed for 4 h. The resultant solution was concentrated under reduced pressure to give pale-yellow syrup. The material was diluted with EtOAc (100 mL), and neutralized with NaHCO₃ (1M, 5 mL). The organic layer was then washed with H₂O (10 mL×2), followed by brine (10 mL), dried over MgSO₄ and filtered. The filtrate was concentrated to give a pale-yellow liquid. (5.33 g, 31.75 mmole, 98%, MS M+H=169 found: 169, ¹H NMR structure confirmed).

The methyl ester (2.0 g, 11.9 mmole) was then added to a CCl₄ solution (100 mL) containing NBS (2.33 g, 13.09 mmole). The reaction mixture was refluxed at 80° C. for 3 h to yield the brominated methyl ester 5_{b}. The cooled solution was filtered through a pad of silica gel to remove excess succinimide, the filtrate was evaporated under reduced pressure, and the resultant material was transferred to the next reaction without further purification.

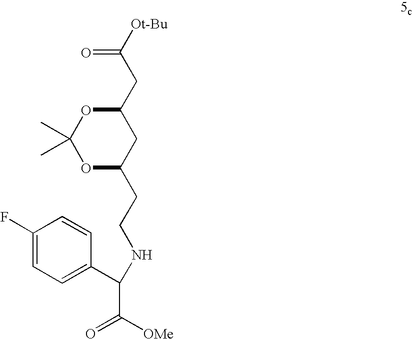

To an acetonitrile solution containing the amine (TBIA, 2.44 g (8.94 mmole)/15 mL ACN) was added the compound 5_{b} (ca. 2 g). While the reaction mixture was stirred triethylamine was added dropwise (1.70 mL, 12.2 mmole 1.5 equiv.). The reaction mixture was stirred at ambient temperature for 16 h. After completion of the reaction, the reaction mixture was concentrated under reduced pressure, and diluted with EtOAc (25 mL). The organic layer was treated with H₂O, dried over MgSO₄, and filtered. The filtrate was then concentrated under reduced pressure to give the compound 5_{c}, 3.29 g.

Isobutyryl chloride (0.53 mL, 4.99 mmole in 5 mL DCM) was added dropwise to a chilled DCM solution (10 mL) containing the compound 5_{c} (2.0 g, 4.54 mmole). While the reaction mixture was stirred, a triethylamine solution (1.27 mL, 2 equiv. in 5 mL DCM) was added dropwise. The reaction mixture was agitated as it was warmed to room temperature for 2 h. After completion of the reaction, the reaction mixture was treated with 1 N HCl (20 mL), followed by sat. NaHCO₃(3 mL). The organic layer was then washed with water and brine, dried over MgSO₄, and filtered. The filtrate was concentrated under reduced pressure to give pale-yellow syrup. This was purified by a column chromatography using a gradient of EtOAc-Hexane mixture (from 0 to 25% of EtOAc). The isolated yield of the methyl ester was 2.10 g, 4.13 mmole, 90.9%.

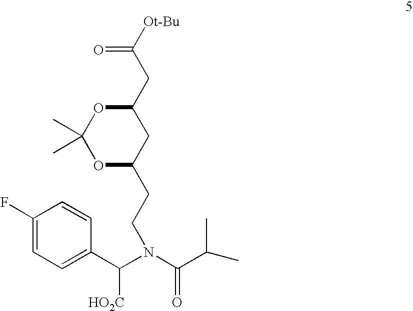

The methyl ester (250 mg, 0.50 mmole) was dissolved in a LiOH solution (1 M, THF:water (5:1) mixture), and vigorously stirred for 3 h. The reaction mixture was neutralized to pH 7 by titrating it with 1 N HCl solution. The desired product was then extracted with EtOAc (20 mL). The organic layer was washed with H₂O and brine, dried over MgSO4, and filtered. The filtrate was then evaporated under reduced pressure to give a white amorphous material 5 (200 mg, 0.40 mmole, 80%, MS M+H=496 found: 496, ¹H NMR structure confirmed).

### Example 6P

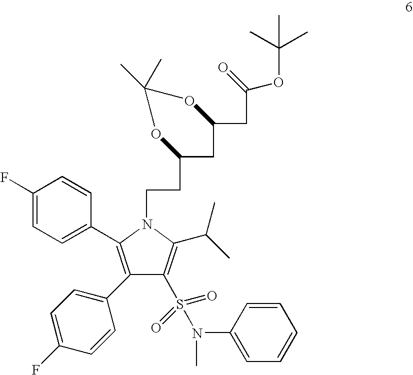

To a toluene solution (5 mL) containing the compounds 4 (0.38 g, 1.30 mmole) and 5 (0.450 g, 0.91 mmole) was added acetic anhydride (0.30 mL). The reaction mixture was heated to 50° C. and stirred at that temperature for 2 h. After the reaction was complete, the reaction mixture was evaporated under reduced pressure to give a dark amorphous material from which desired product (compound 6, 499 mg, 0.690 mmole, 76%)was isolated through a column chromatography using a gradient of EtOAc-Hexane mixture (from Q to 20% of EtOAc).

### Example 7P

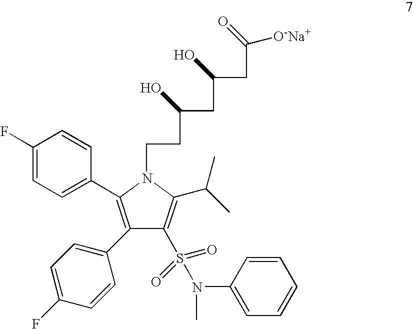

To a DCM solution (5 mL) containing the pyrrolesulfonamide 6, was added 10 (v/v)% TFA in DCM (5 mL) at once at room temperature. The reaction mixture was stirred for 1 h to be complete, and was evaporated under reduced pressure to give a pale yellow amorphous material (yield: 0.25 g, 99%, MS M+H=609 found: 609, ¹H NMR structure confirmed).

The lactone (250 mg, 0.411 mmole) was dissolved in THF (5 mL), and to this solution was added 1 N NaOH solution (400□L). After 2 h most of the lactone disappeared in TLC (R_{f}=0.11 in a 7:3 mix hex:EtOAc) to give a baseline spot. The additional NaOH solution (11 □L) was added dropwise. The solution was stirred for an additional 1 h, and was evaporated under reduced pressure. The resultant solid was then re-dissolved in water and frozen, and was lyophilized overnight to yield a white solid 7 (0.211 g, 0.325 mmole, 79%, MS M+H=649 found: 649, ¹H NMR structure confirmed).

### Example 8P

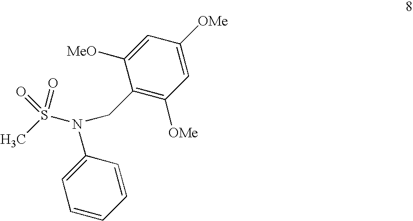

To a DCM solution (50 mL) containing methanesulfonyl chloride 1 (1.614 g, 14.1 mmole) at 0° C. was added 2,4,6-trimethoxybenzylaniline (3,50 g, 13 mmole), followed by dropwise addition of triethylamine (2.68 mL, 19.2 mmole). The reaction mixture was stirred at 0° C. for 1 h and slowly warmed to room temperature. Work-up: The reaction mixture was evaporated under reduced pressure, and 1N HCl solution was added until the pH of the solution was neutral. The desired compound was extracted using EtOAc (25 mL×2), and the organic phase was washed with water (20 mL×2), and brine (10 mL), dried over Na₂SO₄, and filtered. The filtrate was then evaporated under reduced pressure to give a pale yellow solid. The product was recrystallized using hot MeOH. Yield: crude: 3.80 g, 84%, recrystallization: 3.11 g.

### Example 9P

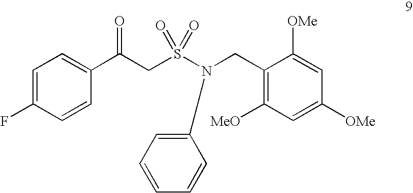

To a THF solution (7.5 mL) containing 8 (1.5 g) at −78° C. was added dropwise n-butyllithium (0.41 mL of 2.5 M in Hexane). The reaction mixture was then warmed to 0° C. and cooled back to −78° C. before methyl p-fluorobenzoate (0.158 g in THF (2.5 mL) ) was added. The reaction mixture was stirred for 1 h after the dry ice bath was removed. Work-up: The reaction mixture was concentrated under reduced pressure, and the resultant suspension was treated with 1N HCl solution. Once acidified, the reaction mixture was extracted with DCM (10 mL×2). The organic phase was then washed with water (5 mL×2), dried over Na₂SO₄, and filtered. The filtrate then was evaporated under reduced pressure to give a white solid (1.527 g, 71.9%).

### Example 10P

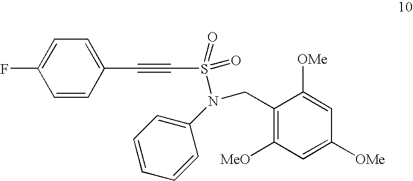

Dry triethylamine (5 mL) was slowly added to a DCM solution (5 mL) containing the □-ketosulfonamide 9 (721 mg, 1.52 mmole) and 2-chloro N-methylpyridinium iodide (580 mg, 2.3 mmole) at ambient temperature. The suspension was stirred at room temperature for 2 days. To take a TLC, a small aliquot of the sample was treated with 1 N NaOH, extracted with DCM. The TLC spot was taken from the organic phase. Work-Up: After 2 days, the suspension was treated with 1N NaOH (5 mL) for 5 min. Then it was extracted with DCM (20 mL×2). This organic phase was successively washed with 1N NaOH, 1N HCl, water, dried over Na₂SO₄, and filtered. The filtrate was then passed through a short column of basic alumina. The resultant DCM solution was then evaporated under reduced pressure to give a yellow solid (520 mg, 75%).

### Example 11P

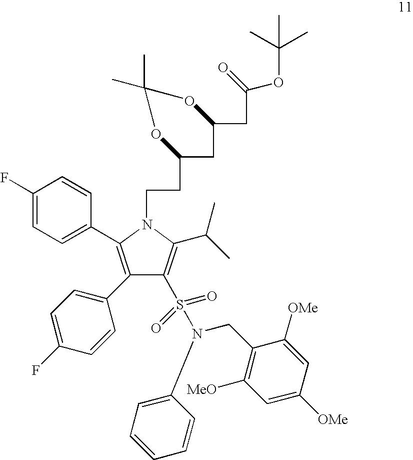

To a toluene solution (5 mL) containing the compounds 10 (0.342 g, 1.10 mmole) and 5 (0.560 g, 0.751 mmole) was added acetic anhydride (0.30 mL). The reaction mixture was heated to 50° C. and stirred at the temperature for 2 h. After the reaction was complete, the reaction mixture was evaporated under reduced pressure to give a dark amorphous material from which the desired product (compound 11, 540 mg, 0.607 mmole, 80.9%)was isolated through column chromatography using a gradient of EtOAc-Hexane mixture (from 0 to 20% of EtOAc).

Example 12P

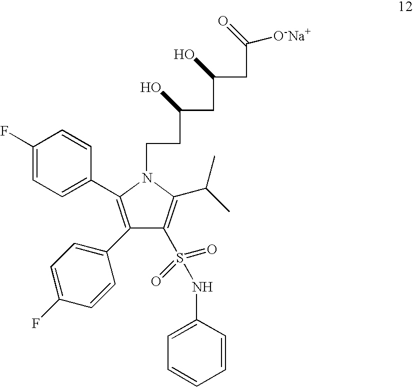

To a DCM solution (5 mL) containing the pyrrolesulfonamide 11 (0.350 g, 0.394 mmole), was added 10 (v/v)% TFA in DCM ( 5 mL) at once at room temperature. The reaction mixture was stirred for 1 h to be complete, and was evaporated under reduced pressure to give a pale yellow amorphous material (yield: 0.199g, 85%, MS M+H=595 found: 595, ¹H NMR structure confirmed).

The lactone (90777×048, 69.7 mg, 0.117 mmole) was dissolved in THF (5 mL), and to this solution was added 1 N NaOH solution (100□L). After 2 h most of the lactone disappeared in TLC in a 7:3 mix hex:EtOAc to give a baseline spot. The additional NaOH solution (17 □L) was added dropwise. The solution was stirred for an additional 1 h, and was evaporated under reduced pressure. The resultant solid was then re-dissolved in water and frozen, and was lyophilized overnight to yield a white solid 12 70 mg, 0.114 mmole, 97%, MS M+H−Na⁺=612 found: 612, ¹H NMR structure confirmed).

### Example 13P

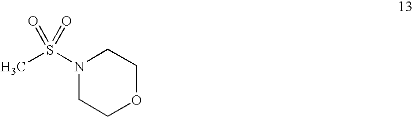

To a DCM solution (50 mL) containing methanesulfonyl chloride at 0° C. was added morpholine, followed by dropwise addition of triethylamine. The reaction mixture was stirred at 0° C. for one hour and slowly warmed to room temperature. The TLC result showed a spot to spot transformation of the morpholine (R_{f}=0.02 to 0.20 in 30% EtOAc/Hex, iodine chamber). Work-up: The reaction mixture was evaporated under reduced pressure, and 1N HCl was added until the pH of the solution was acidic. The desired compound was extracted using EtOAc (25 mL×2), and the organic phase was washed with water (20 mL×2), and brine (10 mL), dried over Na₂SO₄ and filtered. The filtrate was then evaporated under reduced pressure to give a pale yellow solid (8.06 g, 56%).

### Example 14P

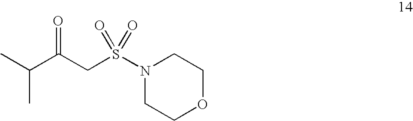

To a THF solution (30 mL) containing morpholino methanesulfonamide (2.0 g) at −78° C. was dropwise added n-butyllithium (6.4 mL of 2.5 M in Hexane). The reaction mixture was then warmed to 0° C. and cooled to −78° C. before methyl isobutyrate (1.081 g in THF (5 mL)) was added. The reaction mixture was stirred for 1 h after the dry ice bath was removed. Work-up: The reaction mixture was acidified with 1 N HCl (5 mL) and then concentrated under reduced pressure. The resultant material was extracted with EtOAc, and the organic phase was washed with water, brine, dried over Na₂SO₄ and filtered. The filtrate was then evaporated under reduced pressure to yield a pale yellow liquid (1.87 g, crude). The crude material was then purified by column chromatography (a 4:1 mixture of Hex. and EtOAc as eluent) to give a transparent liquid (1.16 g).

### Example 15P

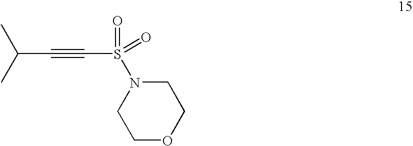

To a DCM solution containing the □-ketosulfonamide 14 (150 mg, 0.638 mmol in 3 mL DCM) was added Hunig's base (333 uL) at 0° C., followed by trifluoromethanesulfonic anhydride (133 uL). The reaction mixture was stirred at the temperature for 24 h. Work-up: The reaction mixture was treated with 1 N aqueous NH₄Cl solution, and the aqueous phase was extracted with DCM (3×10 mL). The combined extracts were washed with sat. aqueous NH₄Cl solution (2×10 mL), water (2×10 mL), dried over MgSO4 and concentrated under reduced pressure. Flash column chromatography of the resulting crude product on silica gel (a gradient up to 30% EtOAc in Hexane) gave of the desired alkynesulfonamide 15 (75 mg, 0.343 mmole, 54%, MS M+H=217 found: 217, ¹H NMR structure confirmed, IR=2193 cm⁻1)

### Example 16P

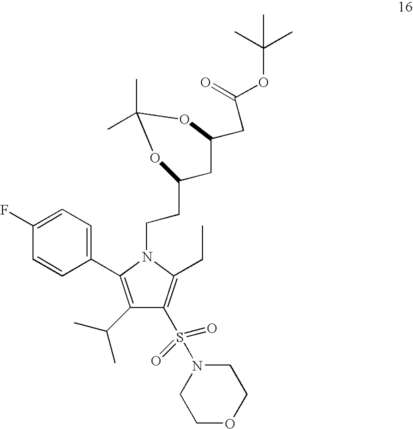

A trifluorotoluene solution (5 mL) containing compound 5′* (0.554 g, 2.5 equiv.) and compound 15 (0.100 g, 0.46 mmole) and acetic anhydride (100 □L) was treated under microwave conditions (180° C., 10 min). After the reaction was complete, the reaction mixture was concentrated under reduced pressure to give a dark brown amorphous material. The compound was submitted for purification and structure analysis. The MS analysis gave the desired mass of the product (M+H 637 found 637).

### Example 17P

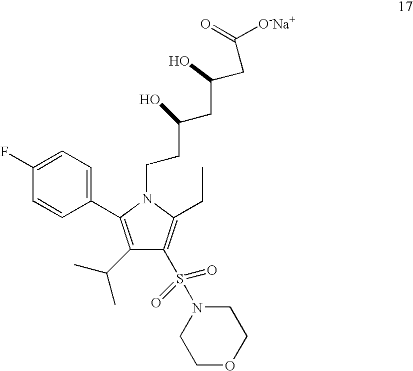

To a DCM solution (10 mL) containing 16 (320 mg, 0.5025 mmole) was added TFA ((2.5 mL) at 0° C. The ice bath was removed after 30 minutes. After 2 h the reaction was complete, the resultant solution was evaporated under reduced pressure to give a pale yellow amorphous material. Work-up: The amorphous material dissolved in 25 mL of DCM and treated with 5 mL of 1N NaHCO₃ solution followed by washing with water (2 mL). The organic layer was then dried over MgSO₄, filtered. The filtrate was evaporated under reduced pressure to give a pale-yellow amorphous material from which the desired material was isolated by column chromatography (50% EtOAc in hex). Isolated yield: 0.190 g, 72.3%. The lactone (120 mg, 0.223 mmole) was dissolved in THF (5 mL), and to this solution was added 1 N NaOH solution (100 uM). After 2 h most of the lactone disappeared in TLC (R_{f}=0.11 in a 2:8 mixture of hexane:EtOAc) to give a baseline spot. The additional NaOH solution (20 uL) was added a drop-wise manner. The solution was stirred for an additional 1 h, and was evaporated under reduced pressure. The resultant solid was then re-dissolved in water and the solution was frozen, and lyophilized overnight to yield a white solid (129 mg, 0.223 mmole: Yield, 99.8%).

### Example 18P

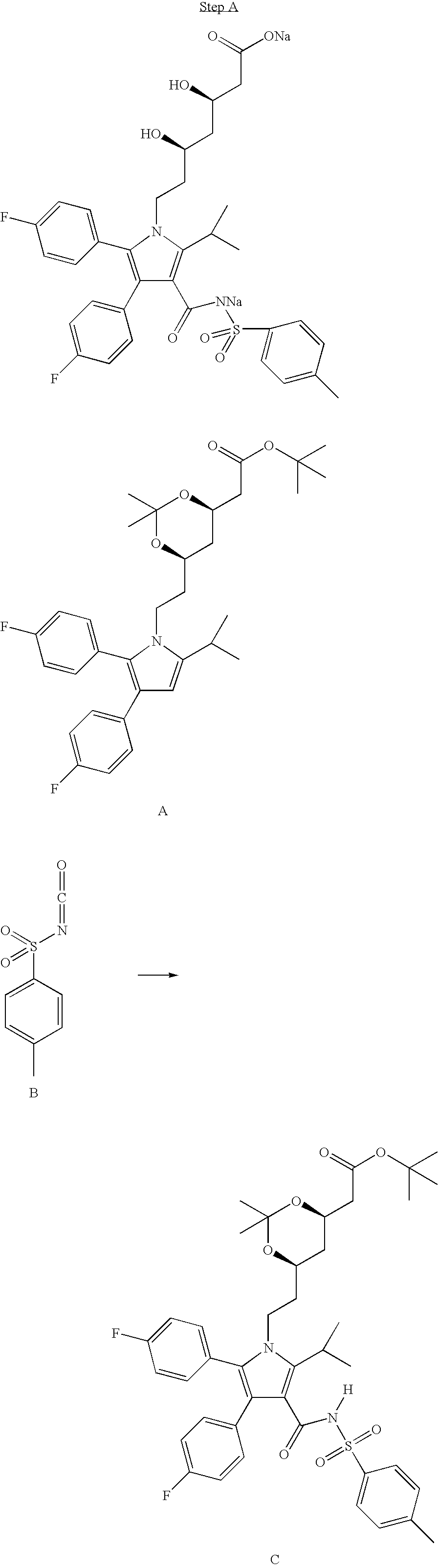

Preparation:

A solution of the above starting material-B in Et₂O (2 mL) was added to a solution of A from Example 1, Step A in Et₂O (3 mL) under N₂ over 2 minutes. The reaction mixture was stirred for another 1 hour, TLC (20% EtOAc in hexanes) indicated that A was not completely consumed. More B was added (2×0.083 mL). After stirring for another 3 hours, the reaction was concentrated in vacuo, and the residue was diluted with EtOAc. The solution was washed with 1 N HCl (2×30 mL) and brine (2×30 mL), and dried over Na₂SO₄. The mixture was filtered and the filtrate was concentrated. A white solid was formed, which was removed via filtration. The filtrate was concentrated, and further purified by chromatography (5-40% EtOAc in hexanes) to give the desired compound as a white foam, 0.1164 g. MS APCI-749.3 (M−H); MP 79-88° C.

Combustion Analysis for [C₄₁H₄₈F₂N₂O₇S.0.1H₂O]:

| | Carbon | Hydrogen | Nitrogen | F |
|---|---|---|---|---|
| Theory | 65.42 | 6.45 | 3.72 | 5.05 |
| Found | 65.16 | 6.50 | 3.66 | 5.29 |

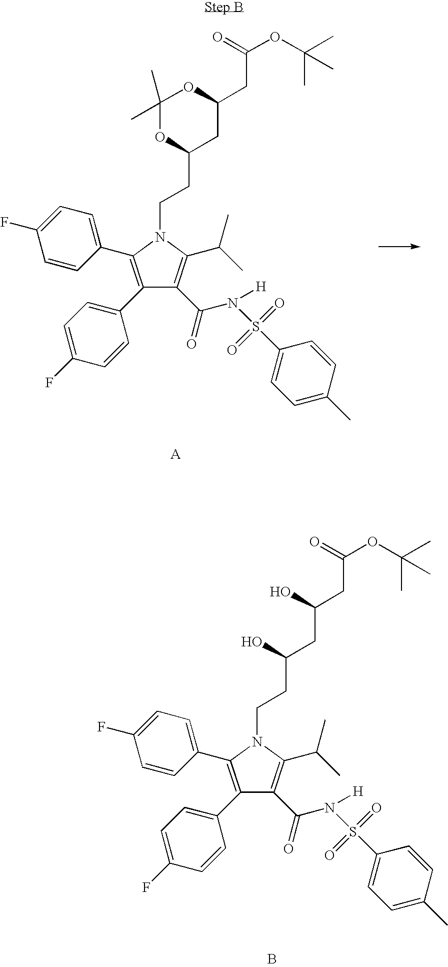

Preparation:

To a suspension of the above starting material-A from Step A in MeOH (8.88 mmol, 2 mL) was added 1 N HCl (0.100 mL). The resulting mixture was stirred for 5 hours. The reaction mixture was diluted with 30 mL of EtOAc, and then washed with 1 N HCl (2×20 mL) and brine (2×20 mL), dried over Na₂SO₄. The mixture was filtered and the filtrate was concentrated in vacuo to give an oil, which was further purified by chromatography (10-60% EtOAc in hexanes). The desired product was isolated. (74 mg.) MS (APCI+, 711.2 M+H) MP 80-91° C.

Combustion Analysis for (C38H₄₄F₂N₂O₇S):

| | Carbon | Hydrogen | Nitrogen |
|---|---|---|---|
| Theory | 64.21 | 6.24 | 3.94 |
| Found | 63.95 | 6.31 | 3.84 |

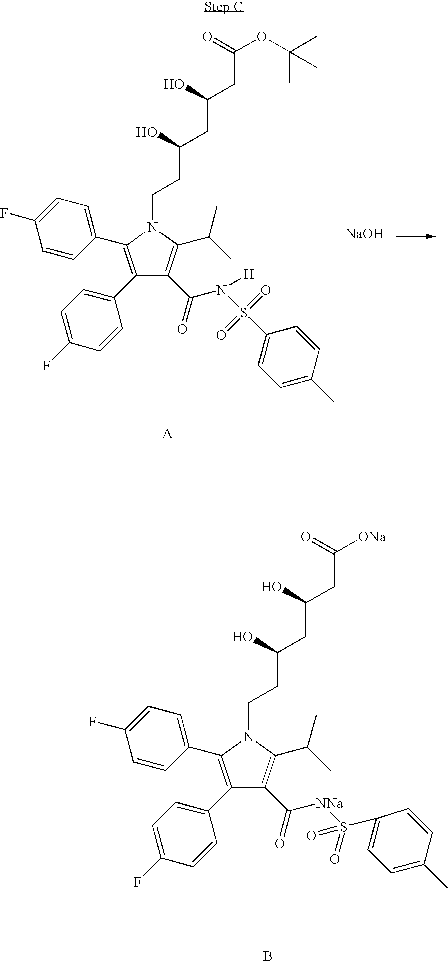

Preparation:

To a suspension of the above starting material-A from Step B in MeOH (2 mL) was added 1N NaOH. The resulting reaction solution was stirred at RT for 2.5 hours. MS showed that A was consumed and product was formed (655.1, acid+H). The reaction mixture was then concentrated in vacuo. 2 mL of MeOH was added to dissolve the residue and 5 mL of toluene was added, and then evaporated to azeotropically remove water. This process was repeated (twice) until a white solid was obtained. The white solid was dissolved in 4 mL of MeOH, then methylene chloride was added dropwise until a cloudy solution was obtained (the final solution was approximately 15-20% MeOH in methylene chloride). After standing for 0.5 hour, the mixture was filtered to remove the solid (excess of NaOH, the di-sodium salt is soluble in 15% MeOH in methylene chloride). The filtrate was concentrated in vacuo to afford a solid, which was triturated with ether to afford a white precipitate. Filtration gave a white solid, (0.0267 g), desired product. NMR and MS showed the acid-ester peak APCI+(655.2, acid+H). MP>240° C. (decomposed).

Combustion Analysis for (C₃₄H₃₄F₂N₂Na₂O₇S.2.25NaOH.2.1H₂O):

| | Carbon | Hydrogen | Nitrogen |
|---|---|---|---|
| Theory | 49.41 | 4.93 | 3.39 |
| Found | 49.27 | 4.54 | 2.99 |

### Example 19P

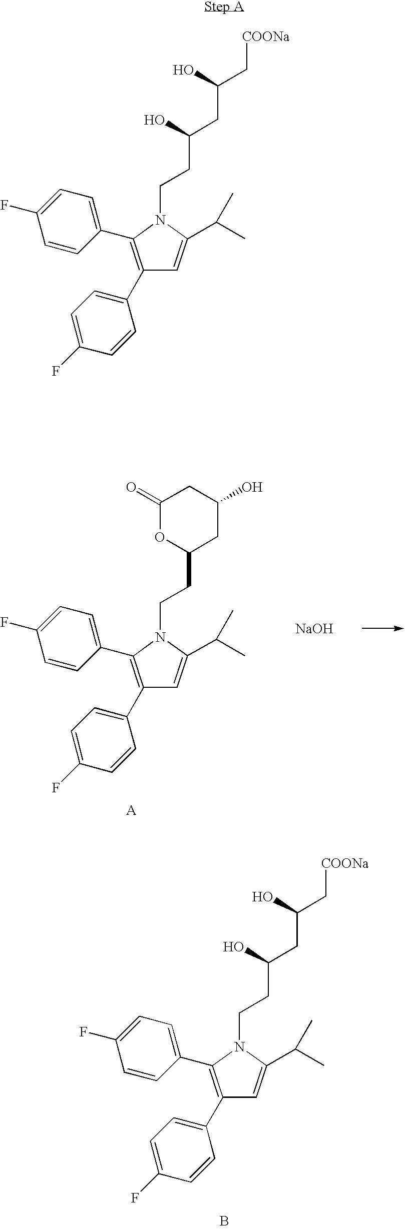

Preparation:

To a solution of the above starting material-A from Example 1, Step E in MeOH (20 mL) and THF (15 mL) was added 1 N NaOH (2.9 mL). The resulting reaction solution was stirred at RT for 2 hours, and then concentrated in vacuo. 5 mL of MeOH was added to dissolve the residue and 20 mL of toluene was added, and then evaporated to azeotropically remove water. This process was repeated (twice) until a white solid was obtained. The white solid was dissolved in 10 mL of MeOH, then diluted with 40 mL of methylene chloride. A cloudy solution was obtained. After standing for 0.5 hour, the mixture was filtered to remove the solid (excess of NaOH, the sodium salt is soluble in 20% MeOH in methylene chloride), the filtrate was concentrated in vacuo to afford a solid, which was triturated with ether to afford a white precipitate. Filtration gave a white solid. The solid was dissolved in 2 mL of MeOH again, and then diluted with 28 mL of dichloromethane (6% MeOH in dichloromethane solution). The solution became cloudy and was allowed to stand at RT for 10 minutes, then filtered. The filtrate was concentrated in vacuo to give a solid which was triturated with ether. A yellow gel was obtained. Ether was stripped off and a yellow foam was obtained. (0.31 g, desired product) NMR (product+H₂O+Et₂O) and MS showed the acid peak APCI+(458, acid+H). MP 215-220° C. (decomposed).

Combustion Analysis for (C₃₂H₃₃F₂N₂NaO₆S.C₄H₁₀O.1.5H₂O):

| | Carbon | Hydrogen | Nitrogen | F |
|---|---|---|---|---|
| Theory | 63.34 | 6.57 | 2.64 | 7.16 |
| Found | 63.13 | 6.48 | 2.52 | 7.33 |

### Example 20P

### (3R,5R)-7-[2,3-Bis-(4-fluoro-phenyl)-5-isopropyl-4-(4-sulfamoyl-phenylsulfamoyl)-pyrrol-1-yl]-3,5-dihydroxy-heptanoic acid disodium salt

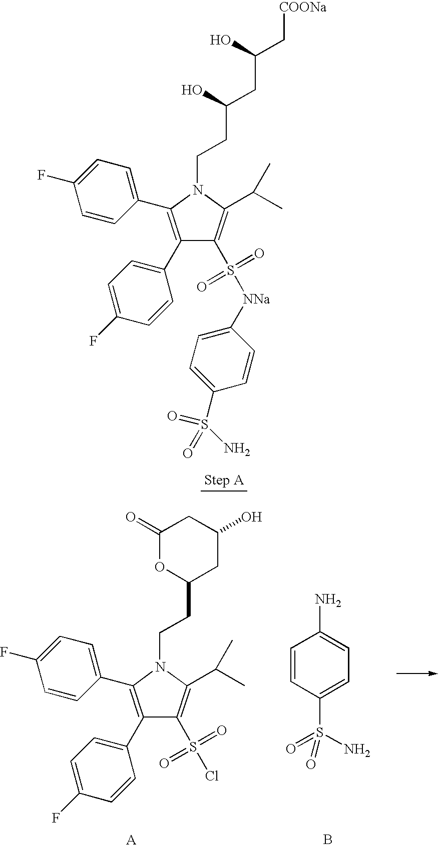

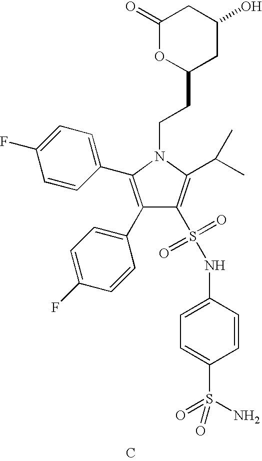

Preparation:

To a solution of the above starting material A from Example 1, Step D in DMF (4.0 mL) was added sulfanilamide. The reaction mixture was stirred at RT under nitrogen for 3,5 hours. The reaction mixture was diluted with 50 mL of ethyl acetate and then washed with 1 N HCl (3×30 mL) and brine, and dried over Na₂SO₄. The mixture was filtered and the filtrate was concentrated. The crude product was purified by chromatography and the desired product was isolated as a beige foam, (0.1615 g). MS APCI+674.1 (M+H), MP, 111-115° C. Combustion Analysis for [C₃₂H₃₃F₂N₃O₇S₂.1.0C₄H₈O₂ (ethyl acetate)]:

| | Carbon | Hydrogen | Nitrogen |
|---|---|---|---|
| Theory | 56.75 | 5.42 | 5.52 |
| Found | 56.38 | 5.09 | 5.50 |

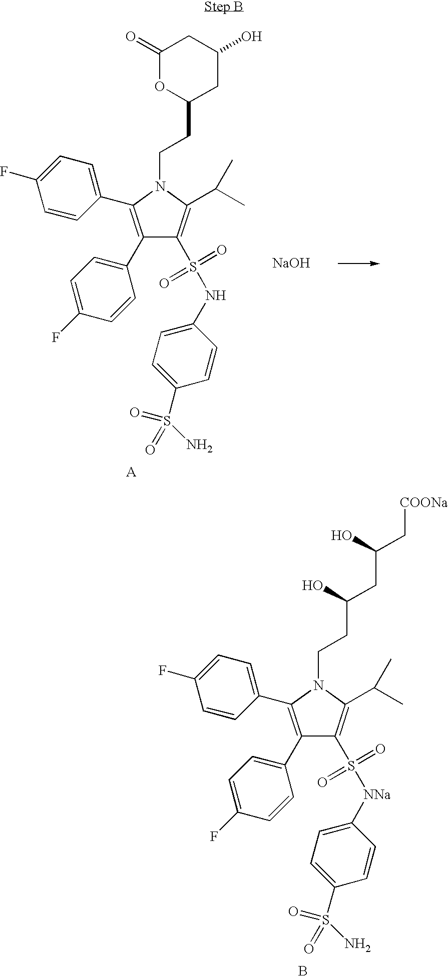

Preparation:

To a solution of the above starting material-A from Step A in MeOH (2 mL) was added 1N NaOH. The resulting reaction solution was stirred at RT for 2.0 hours. MS showed peak 692.2 (acid+H). The reaction mixture was then concentrated in vacuo. 2 mL of MeOH was added to dissolve the residue and 5 mL of toluene was added, and then evaporated to azeotropically remove water. This process was repeated (twice) until a white solid was obtained. The white solid was dissolved in 4 mL of MeOH, then diluted with 16 mL of methylene chloride (overall solution would be 20% MeOH in methylene chloride). A cloudy solution was obtained. After standing for 0.5 hour, the mixture was filtered to remove the solid (excess of NaOH, the sodium salt is soluble in 20% MeOH in methylene chloride), the filtrate was concentrated in vacuo to afford a solid, which was triturated with ether to get a white solid. Filtration gave a white solid, (0.1248 g, desired product). NMR and MS showed the acid peak APCI+(692.3, acid+H). MP 205-207° C. (decomposed).

Combustion Analysis for (C₃₂H₃₃F₂N₃Na₂O₈S₂.2.5H₂O):

| | Carbon | Hydrogen | Nitrogen |
|---|---|---|---|
| Theory | 49.23 | 4.91 | 5.38 |
| Found | 49.45 | 4.75 | 5.00 |

### Example 21P

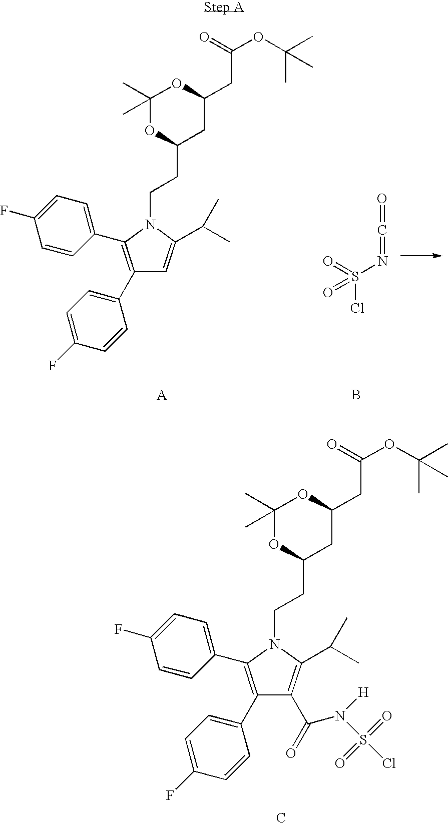

Preparation:

A solution of the above starting material-B in Et₂O (5 mL) was added to a solution of starting material A from Example 1, Step A in Et₂O (5 mL) under N₂ over 5 minutes. The reaction mixture was stirred for another 10 minutes, concentrated in vacuo. A yellow foam was obtained. The crude product was used in the next step without further purifications.

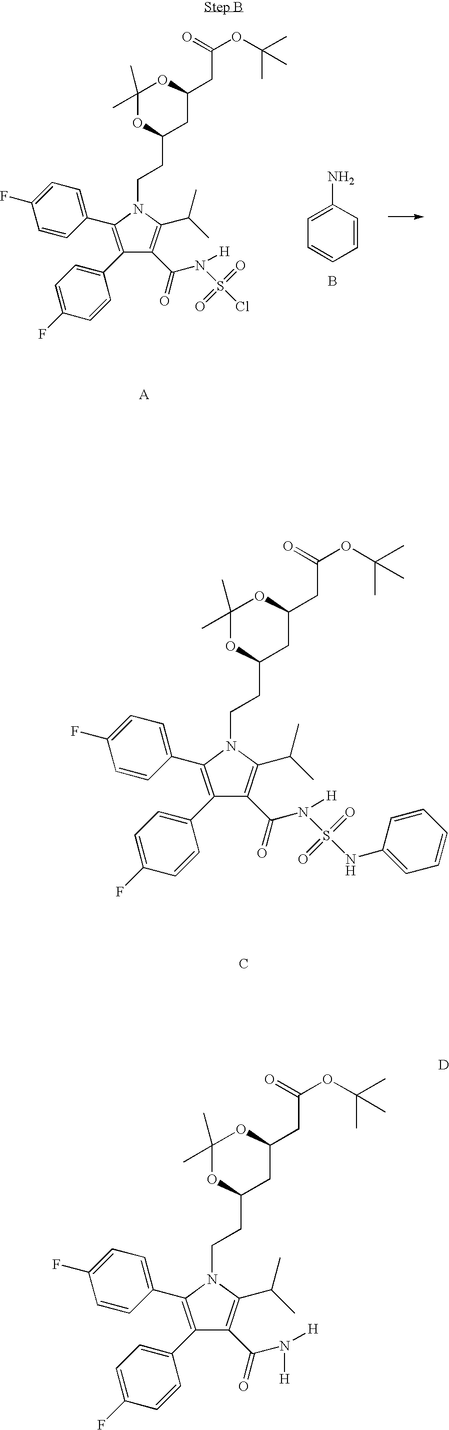

Preparation:

To a solution of the above starting material-A from Step A in THF (5 mL) was added aniline (0.189 mL) under N₂. White precipitate formed instantly. The reaction mixture was stirred for another 16 hours. The reaction mixture was diluted with EtOAc, washed with 1 N HCl (2×30 mL), and brine, and dried over Na₂SO₄. The mixture was concentrated in vacuo. The crude product was purified by chromatography (5-50% EtOAc in hexanes). The desired product was isolated as a yellow foam and characterized by NMR and MS. (0.37 g, 71% over two steps) MP 79-85° C.; MS, APCI- 750.4 (M−H). Combustion Analysis for [C40H47F2N₃O₇S.0.2 H₂O]:

| | Carbon | Hydrogen | Nitrogen | F |
|---|---|---|---|---|
| Theory | 63.59 | 6.32 | 5.56 | 5.03 |
| Found | 63.25 | 6.53 | 5.21 | 4.99 |

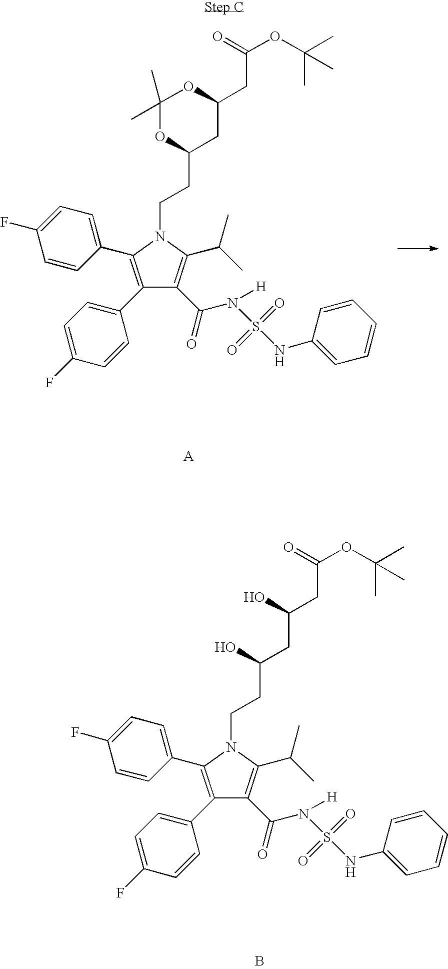

Preparation:

To a suspension of the above starting material-A from Step B in MeOH (8.88 mmol, 4 mL) was added 1 N HCl (0.1517 mL). The resulting mixture was stirred for 6 hours. The reaction mixture was diluted with 30 mL of EtOAc, washed with 1 N HCl (2×20 mL) and brine (2×20 mL), and dried over Na₂SO₄. The mixture was filtered and the filtrate was concentrated in vacuo to afford a white foam. Fairly pure desired product B based NMR and MS. (1229 g.) was used in the next step without further purifications.

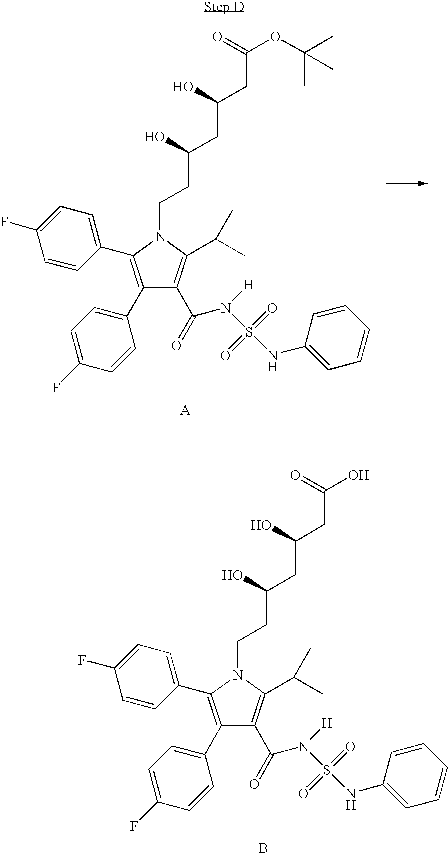

Preparation:

To a solution of the above starting material-A from Step C in MeOH (8.88 mmol, 4 mL) was added 1 N NaOH (0.3596 mL). The resulting mixture was stirred for 6 hours. The reaction mixture was diluted with 30 mL of EtOAc, washed with 1 N HCl (2×20 mL) and brine (2×20 mL), and dried over Na₂SO₄. The mixture was filtered and the filtrate was concentrated in vacuo. The residue was purified by chromatography (10-30% MeOH in DCM). The compound with an Rf value of 0.1 was isolated as a white solid, (41.1 mg).

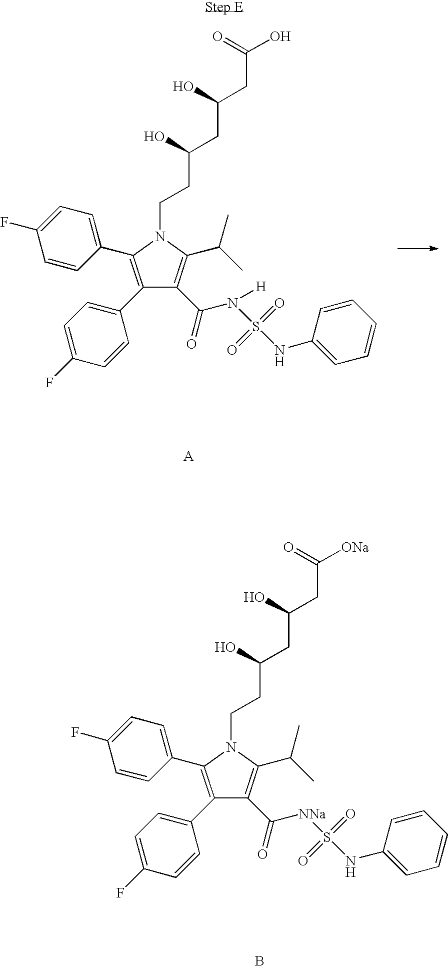

Preparation:

To a solution of the above starting material-A from Step D in MeOH (3 mL) was added 1 N NaOH (0.1254 mL). The resulting mixture was stirred for 10 minutes. The reaction mixture was concentrated in vacuo. The residue was mixed with toluene and concentrated. The residue was mixed with 5 mL of MeOH and filtered to remove insoluble material. The filtrate was concentrated, and triturated with Et₂O to afford a white solid which was isolated. (43.3 mg. desired product) MS APCI+656.1 (M+H for the parent). MP >240° C.

Combustion Analysis for [C₃₃H₃₃F₂N₃Na₂O₇S.0.10C₄H₈O₂ (ethyl acetate).0.30H₂O.1.85NaOH]:

| | Carbon | Hydrogen | Nitrogen |
|---|---|---|---|
| Theory | 51.63 | 5.07 | 5.10 |
| Found | 51.38 | 4.69 | 4.71 |

### Example 22P

### (3R,5R)-7-[3-Carbamoyl-4,5-bis-(4-fluoro-phenyl)-2-isopropyl-pyrrol- 1-yl]-3,5-dihydroxy-heptanoic acid monosodium salt

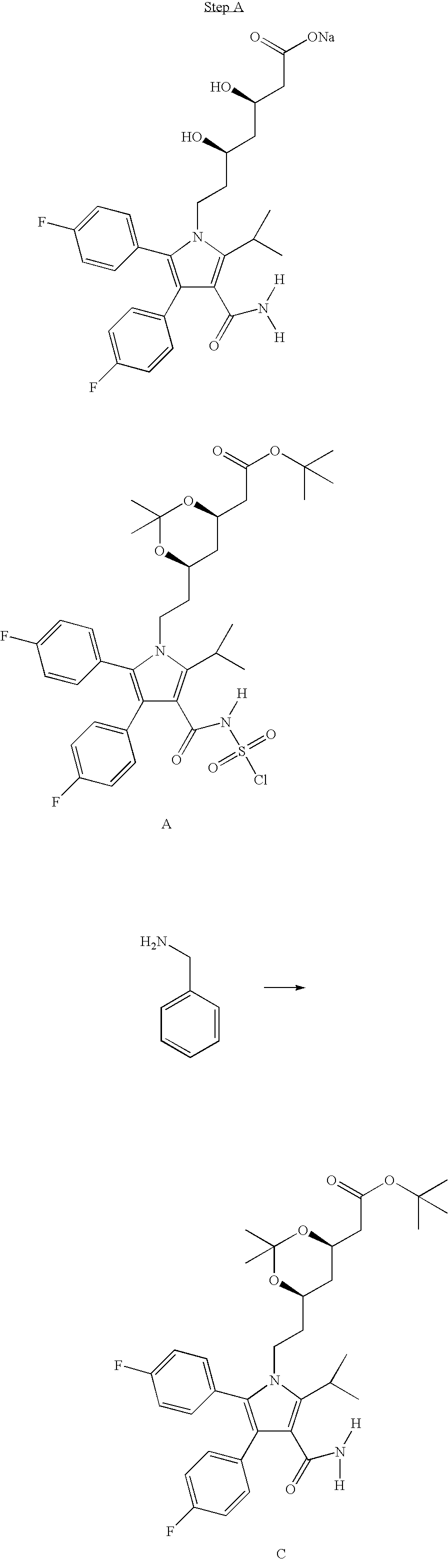

Preparation:

A solution of the above starting material-B in THF (5 mL) was added to a solution of the above starting material-A from Example 29, Step A, in THF (5 mL) under N₂ over 1 minute. White precipitate formed instantly. The reaction mixture was stirred for another 15 minutes. The reaction mixture was diluted with EtOAc, washed with 1 N HCl (2×30 mL), and brine, and dried over Na₂SO₄. The mixture was filtered and the filtrate was concentrated in vacuo. The crude product was purified by chromatography (10-50% EtOAc in hexanes) to give the desired product. The product was purified again (10-50% EtOAc in hexanes) to give 170 mg. pure product D based on MS and NMR. MP: 76-84° C.; MS, APCI+597.2 (M+H).

Combustion Analysis for [C₃₄H₄₂F₂N₂O₅.1.0 H₂O]:

| | Carbon | Hydrogen | Nitrogen | F |
|---|---|---|---|---|
| Theory | 66.43 | 7.21 | 4.56 | 6.18 |
| Found | 66.61 | 7.07 | 4.58 | 6.15 |

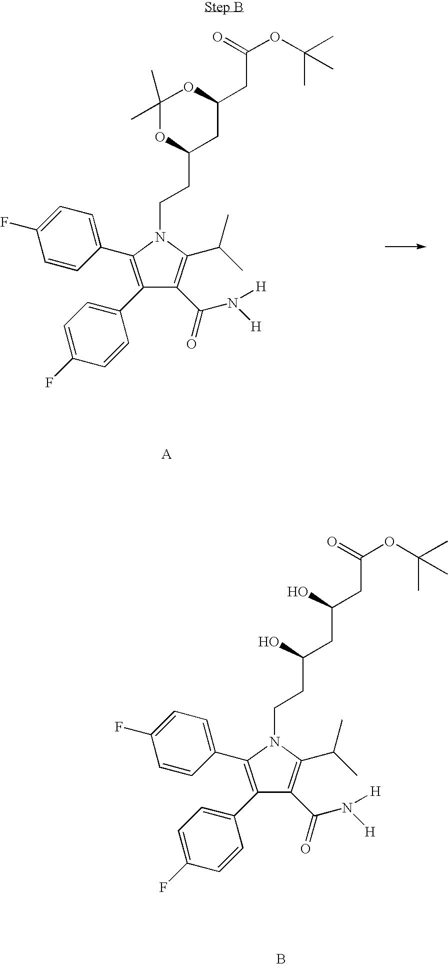

Preparation:

To a suspension of the above starting material-A from Step A in MeOH (8.88 mmol, 2 mL) was added 1 N HCl (0.133 mL). The resulting mixture was stirred for 5 hours. The reaction mixture was diluted with 30 mL of EtOAc, washed with 1 N HCl (2×20 mL) and brine (2×20 mL), and dried over Na₂SO₄. The mixture was filtered and the filtrate was concentrated in vacuo to afford a white foam pure desired product (0.077 g. )based NMR and MS (APCI+, 557.2 M+H). MP 73-78° C. Combustion Analysis for (C₃₁H₃₈F₂N₂O₅):

| | Carbon | Hydrogen | Nitrogen |
|---|---|---|---|
| Theory | 66.89 | 6.88 | 5.03 |
| Found | 66.62 | 7.03 | 4.86 |

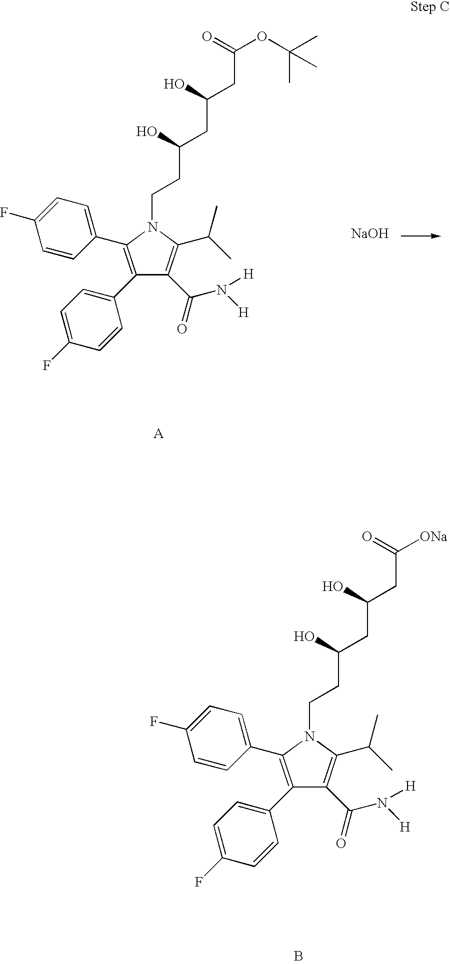

Preparation:

To a suspension of the above starting material-A from Step B in MeOH (2 mL) was added 1N NaOH. The resulting reaction solution was stirred at RT for 2 hours. MS showed that A was consumed and product was formed (501.2, acid+H). The reaction mixture was then concentrated in vacuo. 2 mL of MeOH was added to dissolve the residue and 5 mL of toluene was added, and then evaporated to azeotropically remove water. This process was repeated (twice) until a white solid was obtained. The white solid was dissolved in 0.5 mL of MeOH, then diluted with 9.5 mL of methylene chloride (overall solution would be 5% MeOH in methylene chloride). A cloudy solution was obtained. After standing for 0.5 hour, the mixture was filtered to remove the solid (excess of NaOH, the sodium salt is soluble in 5% MeOH in methylene chloride). The filtrate was concentrated in vacuo to afford a solid, which was triturated with ether to afford a white precipitate. Filtration gave a white solid, 0.0303 g, desired product based on NMR. MS showed the acid-ester peak APCI+(501.2, acid+H). MP 195-198° C. (decomposed).

Combustion Analysis for (C₂₇H₂₉F₂N₂NaO₅.0.3NaOH.2.40H₂O):

| | Carbon | Hydrogen | Nitrogen |
|---|---|---|---|
| Theory | 56.13 | 5.95 | 4.85 |
| Found | 56.22 | 5.56 | 4.46 |

### Example 23P

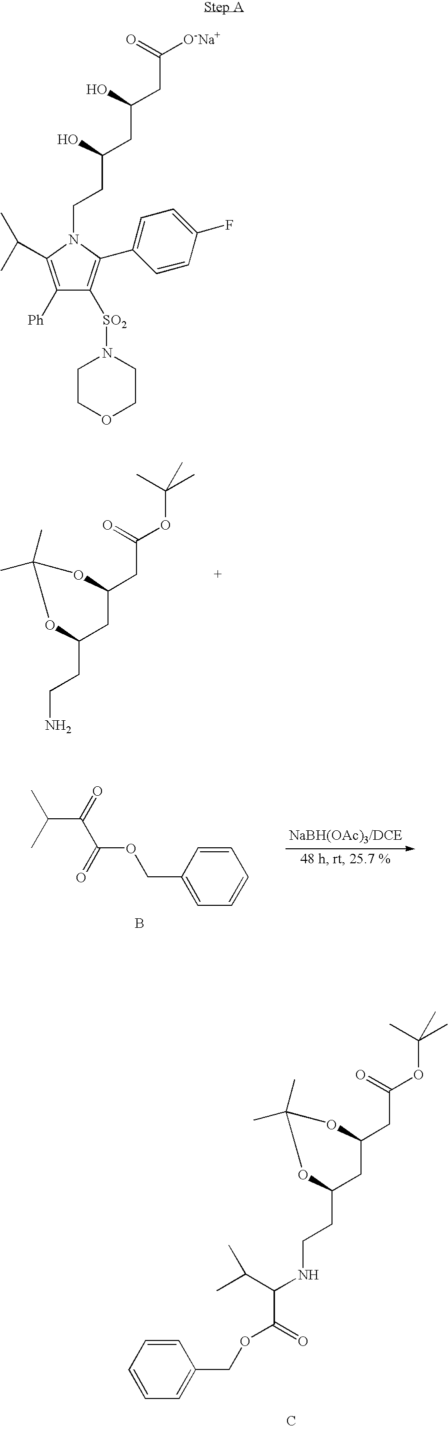

The chiral amine, starting material A, and the α-ketoester, starting material B, were combined in 150 ml of DCE. After stirring @ RT for 1 h, solid sodium triacetoxyborohydride (“NaBH(OAc)₃”) was added and the resulting mixture was allowed to stir © RT for 48 h. The reaction mixture was quenched with sat. aqueous NH₄Cl (10 mL) and water (200 mL). The aqueous layer was adjusted to pH>10 with KOH. The organic layer was diluted with dichloromethane, removed, washed with brine, dried (Na₂SO₄), and concentrated to a crude yellow oil. TLC indicates several major components including starting amine and starting ketone as well as the desired product [R_{f}=0.48, Hexanes/ethyl acetate (1:1), KmnO₄ )], and reduced ketoester [R_{f}=0.67, Hexanes/ethyl acetate (1:1), KmnO₄)]

This material was purified by silica gel chromatography eluting with a gradient of hexanes/ethyl acetate mixture [Hexanes/ethyl acetate (95:5 to 70:30)] to give 3.85 g of the desired product, C, as a light oil.
Loop LC-MS [M+H]⁺=464
¹H NMR is consistent with expected product that appears to be contaminated with benzyl alcohol ˜1 equiv
The resulting material, C will be used in next reaction without additional purification.

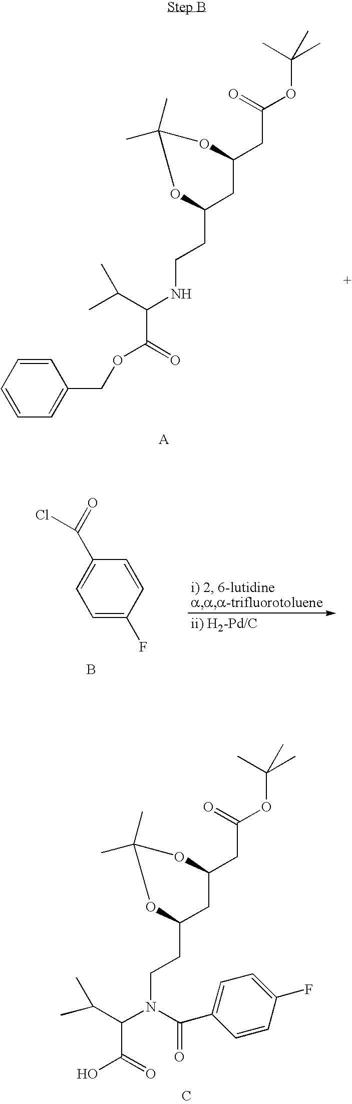

P-fluorobenzoyl chloride was added dropwise to a RT solution of amine, (product C from Step A), and 2,6-lutidine in a,a,a-trifluorotoluene (10 mL). After sonication, (3 min) a TLC shows only traces of starting material, a new less polar component, and acid chloride. The reaction was treated with N,N,2,2-tetramethyl-1,3-propanediamine. After 15 minutes, the reaction mixture was diluted with CH₂Cl₂ (10 mL), and washed sequentially with dilute HCl (pH<1), 1 N NaHCO₃, and brine. The organic layer was dried (Na₂SO4), and concentrated to an oil.
TLC indicates baseline material and one major component : [Hexanes/Ethyl Acetate (3:1; R_{f}=0.27; UV, KMnO₄)].
Purification by flash SiO₂-gel chromatography [Hexanes/Ethyl Acetate 90:10 to 50:50] provides product as a transparent solid.
LC-MS [M+H]⁺=586, (base peak=528).

Hydrogenation
The benzyl ester was submitted to High Pressure Lab for hydrogenation
The returned sample was concentrated and dried under high vacuum to give a colorless solid.
Loop LC-MS [M−H]⁻=494
¹H MNR (CD₃CN) appears to be consistent with the desired product.
The resulting material, C, is used in the subsequent reaction without additional purification.

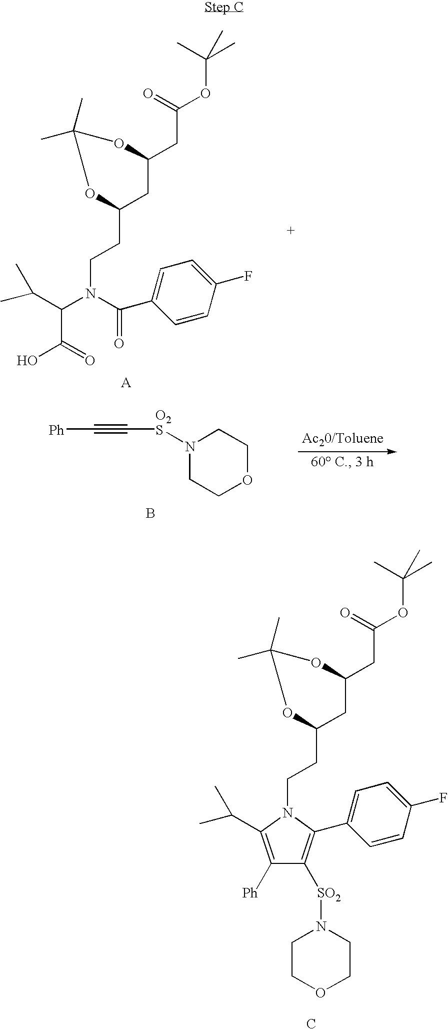

To a toluene solution (10 mL) containing the Munchnone precursor (176 mg, 0.355 mmole), (compound C from Step B), and starting material B (89.3 mg, 0.355 mmole), was added acetic anhydride (200 uL). The reaction mixture was then heated to 60° C. for 3 h. The reaction mixture was cooled and evaporated under reduced pressure to give a pale-yellow syrup. Using a silica plug, most of the polar contaminants were removed by using 30% EtOAc in hexane as eluent. Liquid Chromatography coupled with mass spectrometer, (“LCMS”) results showed the presence of both regioisomers: Sought 684.2, observed 685.2. Retention time at 2.872 minutes and 3.040 minutes was 8% and 33%, respectively (% =UV ratio at 214 nm). The peak with the retention time of 3.040 min. was identified as desired compound C. ¹H NMR structure confirmed.

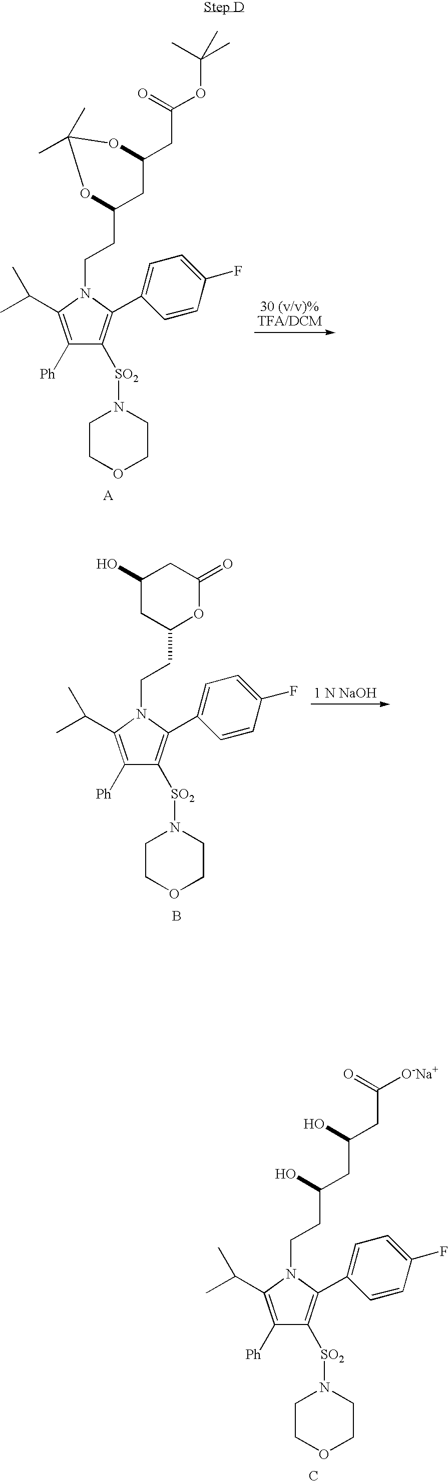

To a DCM solution (10 mL) containing compound C from Step C (125 mg, 0.5025 mmole) was added TFA (2.5 mL) at 0° C. The ice-bath was removed after the reaction mixture was well mixed. After 3 h, the reaction was complete. The resultant solution was evaporated under reduced pressure to give a pale yellow amorphous material. Work-up: The amorphous material was dissolved in 25 mL of DCM and treated with 5 mL of 1 N NaHCO₃ solution followed by washing with water (2 mL). The organic layer was then dried over MgSO₄ and filtered. The filtrate was evaporated under reduced pressure to give a pale-yellow amorphous material from which the desired material was isolated by column chromatography (50% EtOAc in hexane). Isolated yield of desired lactone compound B: 94.4 mg, 90.27%: LCMS result (retention time: 2.065 min (1:1 ACN:H₂O), surface area=100% at 214 nm; Sought 570, observed M+H=571); ¹H NMR structure confirmed.

The lactone B was dissolved in THF (5 mL), and to this solution was added 1 N NaOH solution (450 uM). After 2 h, most of the lactone disappeared to give a baseline spot in TLC. The additional NaOH solution (20 uL) was added dropwise. The solution was stirred for an additional 1 h, and was evaporated under reduced pressure. The resultant solid was then re-dissolved in water and frozen. This was lyophilized overnight to yield a white solid of C: 60.55 mg, 99.9%; LCMS result (retention time: 1.597 min (1:1 ACN:H₂O), surface area=100% at 214 nm; Sought 587, observed M+H=588); ¹H NMR structure confirmed.

### Example 24P

### (3R,5R)-7-[3-(Azetidine-1-sulfonyl)-5-(4-fluoro-phenyl)-2-isopropyl-4-phenyl-pyrrol-1-yl]-3,5-dihydroxy-heptanoic acid sodium salt

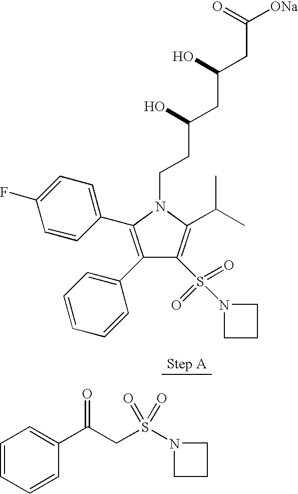

### 2-(Azetidine-1-sulfonyl)-1-phenyl-ethanone

To a THF solution (30 mL) containing Azetidine methanesulfonamide (2.0 g) at −78° C. was dropwise added n-butyllithium (6.4 mL of 2.5 M in Hexane). The reaction mixture was then warmed to 0° C. and cooled back to −78° C. before methyl benzoate (2.01 g in THF (5 mL) )was dropwise added. The reaction mixture was stirred for 1 h after the dry ice bath was removed. Work-up: The reaction mixture was acidified with 1N HCl (5 mL) and then concentrated under reduced pressure. The resultant material was extracted with EtOAc, and the org. phase was washed with water, brine and dried over Na₂SO₄, filtered. The filtrate was then evaporated under reduced pressure to yield a pale yellow liquid (2.95 g, crude). The crude material was then purified by column chromatography (a 4:1 mixture of Hex. and EtOAc as eluent) to give a transparent liquid (2.30 g). MS, APCI+240.0 (M+H); ¹H NMR spectrum (400 MHz, CDCl₃) δ 7.90 (d, J=7.6 Hz, 2H), 7.65 (m, 3H), 4.13 (dd, J=7.9 Hz, J=7.80 Hz, 4H), 2.75 (s, 2H), 1.730 (m, 2H).

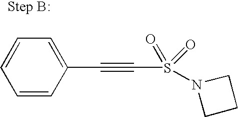

### 1-(2-Phenyl-ethynesulfonyl)-azetidine

Dry triethylamine (7.5 mL) was slowly added to a DCM solution (10 mL) containing the ketosulfonamide from Step A and N-methylpyridinium Iodide at ambient temperature. The suspension was stirred at room temperature for 2 days. To take a TLC a small aliquot of the sample was treated with 1 N NaOH, extracted with DCM. The TLC spot was taken from the org. phase. Work-Up: After 2 days, the suspension was treated with 1 N NaOH (5 mL) for 5 min. Then it was extracted with DCM (20 mL×2). This organic phase was successively washed with 1N NaOH, 1N HCl, water, and dried over Na2SO4, and filtered. The filtrate was then passed through a short column of basic alumina. The resultant DCM solution was then evaporated under reduced pressure to give a yellow solid. MS, APCI+222.1 (M+H); ¹H NMR spectrum (400 MHz, CDCl₃) δ 7.65 (d, J=7.6 Hz, 2H), 7.50 (m, 1H), 7.43 (m, 2H), 4.13 (dd, J=7.9 Hz, J=6.80 Hz, 4H), 2.30 (q, J=6.8 Hz, 2H).

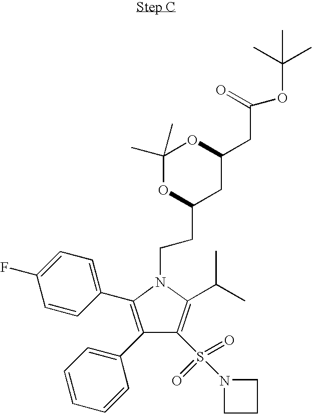

### ((4R,6R)-6-{2-[3-(Azetidine-1-sulfonyl)-5-(4-fluoro-phenyl)-2-isopropyl-4-phenyl-pyrrol-1-yl]-ethyl}-2,2-dimethyl-[1,3]dioxan-4-yl)-acetic acid tert-butyl ester

A solution of the Munchnone acid (1.23 g), an alkynyl azetidine sulfonamide from Step B (0.50 g, 2.26 mmole) and acetic anhydride (2.3 mmole, 300 uL) in toluene (10 mL) was heated to 60° C. for 3 h. After the reaction was complete the mixture was cooled to r.t., and evaporated under reduced pressure to yield a dark-yellow amorphous material. The desired product was isolated by a column chromatography using a gradient from 0 to 30%(v/v) of EtOAC and Hex, respectively. MS, APCI+655.3 (M+H); ¹H NMR spectrum (400 MHz, CDCl₃) δ 7.24-7.05 (m, 7H), 6.90 (dd, J=8.5 Hz, 8.6 Hz, 2H), 4.20-4.01 (m Hz, 4H), 3.88 (m,1 H), 3,48 (m, 4H), 2.33 (dd, J=7.1, 6.8 Hz 1H), 2.18 (dd, 6.1, 6.4 Hz, 1H), 1.90 (m, 2H), 1.45 (m, 6H), 1.40 (s, 9H), 1.29 (d, 6H).

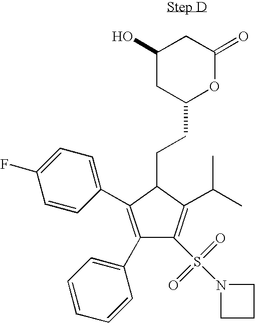

### (4R,6R)-6-[2-[3-(Azetidine-1-sulfonyl)-5-(4-fluoro-phenyl)-2-isopropyl-4-phenyl-pyrrol-1-yl]-ethyl}-4-hydroxy-tetrahydro-pyran-2-one

A TFA solution (30% (v/v), 15 mL) was added to the protected pyrrole from Step C (1.509 mmole) at r.t.. The reaction was complete within 30 min., indicated by a new spot in TLC (Rf=0.11 in 7:3 hex:EtOAc mix). The reaction mixture was evaporated under reduced pressure, and the resultant yellow amorphous material was diluted with EtOAc (20 mL), and treated with NaHCO₃ (1.0 mL), washed with water (5 mL) and brine. The org. layer was then dried over Na₂SO₄, and filtered. The filtrate was subsequently evaporated under reduced pressure to give a pale yellow mat. (0.722 g, crude). The desired lactone was obtained by a column chromatography by using a gradient of 6:4 to 8:2 EtOAc: hex mix, respectively: 0.503 g. MS, APCI+541.2 (M+H); ¹H NMR spectrum (400 MHz, CD₃OD) δ 7.24-7.05 (m, 7H), 6.98 (dd, J=8.5 Hz, 8.6 Hz, 2H), 4.45(m, 1H), 4.15 (m, 2H), 3,57(m, 4H), 2.63-2.44 (m, 2H), 1.95 (m, 2H), 1.90 (m, 2H), 1.45 (2s, 6H).

Step E

### (3R,5R)-7-[3-(Azetidine-1-sulfonyl)-5-(4-fluoro-phenyl)-2-isopropyl-4-phenyl-pyrrol-1-yl]-3,5-dihydroxy-heptanoic acid sodium salt

To a solution containing the lactone from Step D (0.670 mmole in 10 mL THF) was dropwise added 1 N NaOH (669.75 uL, 1 equiv.) at room temperature. The reaction mixture was stirred until all lactone disappeared. The reaction mixture was evaporated under reduced pressure and redissolved in water (2 mL). This was freeze-dried to give a white solid (377.9 mg).

MS, APCI+559.2 (M+H);

Combustion Analysis for (C₂₉H₃₄F₁N₂O₆S₁Na₁0.74 H₂O):

| | Carbon | Hydrogen | Nitrogen |
|---|---|---|---|
| Theory | 58.64 | 6.02 | 4.72 |
| Found | 58.25 | 6.01 | 4.41 |

Following a similar reaction scheme as described in the previous Example, the following compounds are a representative sample of additional final compounds synthesized.

### Example 25P

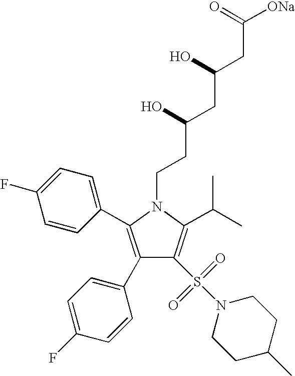

### (3R,5R)-7-[2,3-Bis-(4-fluoro-phenyl)-5-isopropyl-4-(4-methyl-piperidine-1-sulfonyl)-pyrrol-1-yl]-3,5-dihydroxy-heptanoic acid sodium salt

MS, APCI+619.0 (M+H);

Combustion Analysis for (C₃₂H₃₉F₂N₂O₆S₁Na₁ 2.05 H₂O):

| | Carbon | Hydrogen | Nitrogen |
|---|---|---|---|
| Theory | 56.72 | 6.41 | 4.13 |
| Found | 56.32 | 6.33 | 3.74 |

### Example 26P

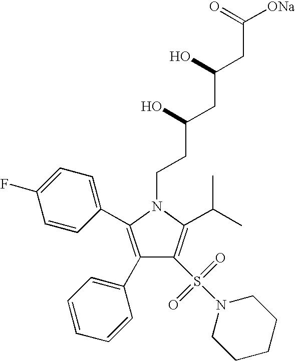

### (3R,5R)-7-[2-(4-Fluoro-phenyl)-5-isopropyl-3-phenyl-4-(piperidine-1-sulfonyl)-pyrrol-1-yl]-3,5-dihydroxy-heptanoic acid sodium salt

MS, APCI+587.2 (M+H);

Combustion Analysis for (C₃₁H₃₈F₁N₂O₆S₁Na₁ 1.81 H₂O):

| | Carbon | Hydrogen | Nitrogen |
|---|---|---|---|
| Theory | 58.06 | 6.54 | 4.37 |
| Found | 57.67 | 6.35 | 4.04 |

### Example 27P

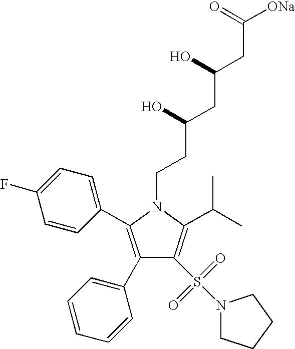

### (3R,5R)-7-[2-(4-Fluoro-phenyl)-5-isopropyl-3-phenyl-4-(pyrrolidine-1-sulfonyl)-pyrrol-1-yl]-3,5-dihydroxy-heptanoic acid sodium salt

MS, APCI+573.2 (M+H);

Combustion Analysis for (C₃₀H₃₆F₁N₂O₆S₁Na₁ 1.90 H₂O):

| | Carbon | Hydrogen | Nitrogen |
|---|---|---|---|
| Theory | 57.29 | 6.38 | 4.45 |
| Found | 56.90 | 6.23 | 4.09 |

### Example 28P

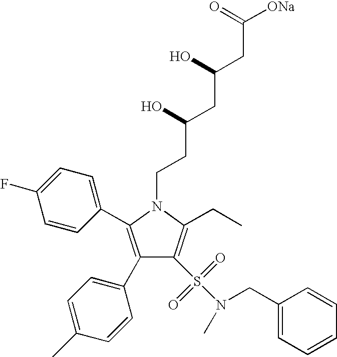

### (3R,5R)-7-[3-(Benzyl-methyl-sulfamoyl-2-ethyl-5-(4-fluoro-phenyl)-4-p-tolyl-pyrrol-1-yl]-3,5-dihydroxy-heptanoic acid sodium salt

MS, APCI+623.3 (M+H);

Combustion Analysis for (C₃₄H₃₈F₁N₂O₆S₁Na₁ 2.68 H₂O):

| | Carbon | Hydrogen | Nitrogen |
|---|---|---|---|
| Theory | 58.93 | 6.31 | 4.04 |
| Found | 58.54 | 6.11 | 3.70 |

### Example 29P

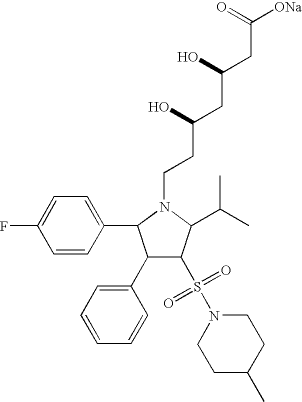

### (3R,5R)-7-[2-(4-Fluoro-phenyl)-5-isopropyl-4-(4-methyl-piperidine-1-sulfonyl)-3- phenyl-pyrrol-1-yl]-3,5-dihydroxy-heptanoic acid sodium salt

MS, APCI+601.3 (M+H);

Combustion Analysis for (C₃₂H₄₀F₁N₂O₆S₁Na₁ 2.29 H₂O):

| | Carbon | Hydrogen | Nitrogen |
|---|---|---|---|
| Theory | 57.88 | 6.77 | 4.22 |
| Found | 57.72 | 6.72 | 3.82 |

### Example 30P

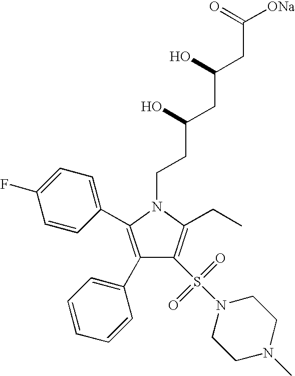

### (3R,5R)-7-[2-Ethyl-5-(4-fluoro-phenyl)-3-(4-methyl-piperazine-1-sulfonyl)-4-phenyl-pyrrol-1-yl]-3,5-dihydroxy-heptanoic acid sodium salt

MS, APCI+588.1 (M+H);

Combustion Analysis for (C₃₀H₃₇F₁N₃O₆S₁Na₁ 2.75 H₂O):

| | Carbon | Hydrogen | Nitrogen |
|---|---|---|---|
| Theory | 54.66 | 6.50 | 6.37 |
| Found | 54.27 | 6.28 | 6.04 |

### Example 31P

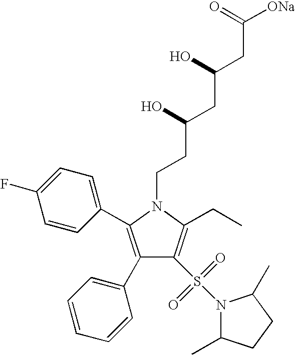

### (3R,5R)-7-[3-(2,5-Dimethyl-pyrrolidine-1-sulfonyl)-2-ethyl-5-(4-fluoro-phenyl)-4-phenyl-pyrrol-1-yl]-3,5-dihydroxy-heptanoic acid sodium salt

MS, APCI+587.1 (M+H);

Combustion Analysis for (C₃₁H₃₈F₁N₂O₆S₁Na₁ 2.08 H₂O):

| | Carbon | Hydrogen | Nitrogen |
|---|---|---|---|
| Theory | 57.62 | 6.58 | 4.34 |
| Found | 57.23 | 6.35 | 4.08 |

### Example 32

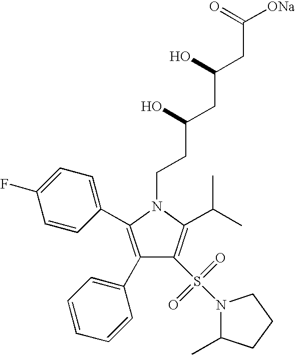

### (3R,5R)-7-[2-(4-Fluoro-phenyl)-5-isopropyl-4-(2-methyl-pyrrolidine-1-sulfonyl)-3-phenyl-pyrrol-1-yl]-3,5-dihydroxy-heptanoic acid sodium salt

MS, APCI+587.3 (M+H);

Combustion Analysis for (C₃₁H₃₈F₁N₂O₆S₁Na₁ 1.13 H₂O):

| | Carbon | Hydrogen | Nitrogen |
|---|---|---|---|
| Theory | 59.19 | 6.45 | 4.45 |
| Found | 58.80 | 6.46 | 4.22 |

### Example 33P

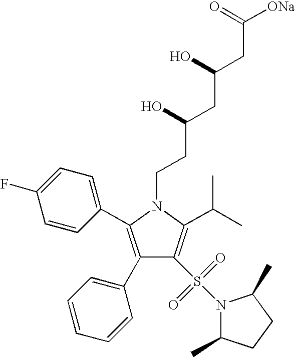

### (3R,5R)-7-[3-((2S,5R)-2,5-Dimethyl-pyrrolidine-1-sulfonyl)-5-(4-fluoro-phenyl)-2-isopropyl-4-phenyl-pyrrol-1-yl]-3,5-dihydroxy-heptanoic acid sodium salt

MS, APCI+601.3 (M+H);

Combustion Analysis for (C₃₂H₄₀F₁N₂O₆S₁Na₁ 1.27 H₂O):

| | Carbon | Hydrogen | Nitrogen |
|---|---|---|---|
| Theory | 59.53 | 6.64 | 4.34 |
| Found | 59.14 | 6.66 | 4.16 |

### Example 34P

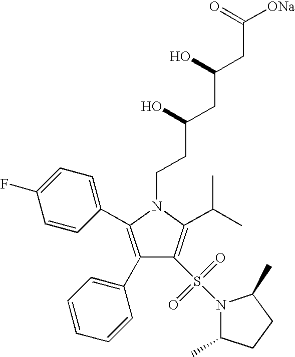

### (3R,5R)-7-[3-((2S,5S)-2,5-Dimethyl-pyrrolidine-1-sulfonyl)-5-(4-fluoro-phenyl)-2-isopropyl-4-phenyl-pyrrol-1-yl]-3,5-dihydroxy-heptanoic acid sodium salt

MS, APCI+601.3 (M+H);

Combustion Analysis for (C₃₂H₄₀F₁N₂O₆S₁Na₁ 1.75 H₂O):

| | Carbon | Hydrogen | Nitrogen |
|---|---|---|---|
| Theory | 58.75 | 6.70 | 4.28 |
| Found | 58.36 | 6.44 | 3.98 |

### Example 35P

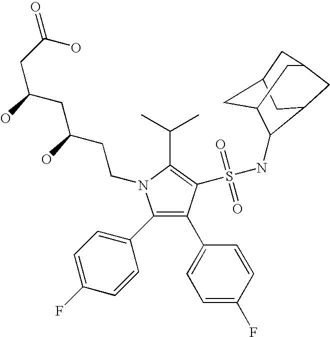

### 7-[3-(Adamantan-2-ylsulfamoyl)-4,5-bis-(4-fluoro-phenyl)-2-isopropyl-pyrrol-1-yl]-3,5-dihydroxy-heptanoic acid Sodium Salt

MS APCI+671.2 (acid+1)

Analyzed for: C36H43F2N2Na1O6S1.2.06H2O1

| | C | H | N |
|---|---|---|---|
| Theory | 59.24 | 6.51 | 3.84 |
| Found | 58.84 | 6.11 | 3.79 |

### Example 36P

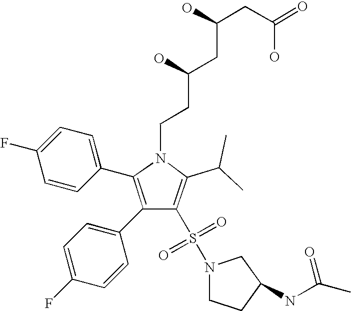

### 7-[3-(3-Acetylamino-pyrrolidine-1-sulfonyl)-4,5-bis-(4-fluoro-phenyl)-2-isopropyl-pyrrol-1-yl]-3,5-dihydroxy-heptanoic acid Sodium Salt

MS APCI+647.1 (acid+1)

Analyzed for: C32H38F2N3Na1O7S1.040H2O1

| | C | H | N |
|---|---|---|---|
| Theory | 56.78 | 5.78 | 6.21 |
| Found | 56.39 | 5.86 | 5.81 |

### Example 37P

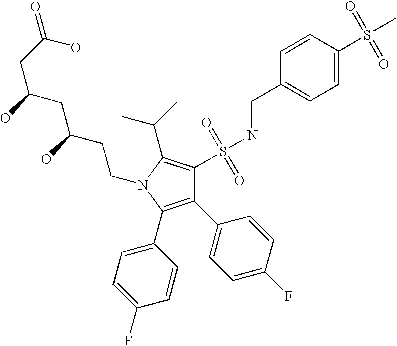

### 7-[2,3-Bis-(4-fluoro-phenyl)-5-isopropyl-4-(4-methanesulfonyl-benzylsulfamoyl)-pyrrol-1-yl]-3,5-dihydroxy-heptanoic acid Sodium Salt

| | C | H | N |
|---|---|---|---|
| Theory | 55.37 | 5.08 | 3.78 |
| Found | 54.98 | 5.24 | 3.65 |

### Example 38P

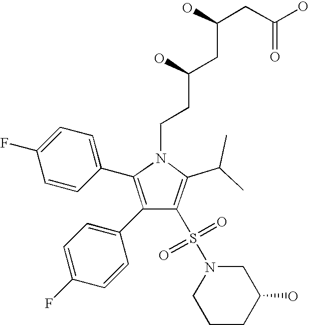

### 7-[2,3-Bis-(4-fluoro-phenyl)-4-(3-hydroxy-piperidine-1-sulfonyl)-5-isopropyl-pyrrol-1-yl]-3,5-dihydroxy-heptanoic acid Sodium Salt

MS APCI+621.2 (acid+1)

Analyzed for: C31 H37F2N2Na1O7S1.2.35H2O1

| | C | H | N |
|---|---|---|---|
| Theory | 54.35 | 6.14 | 4.09 |
| Found | 54.61 | 5.74 | 3.69 |

### Example 39P

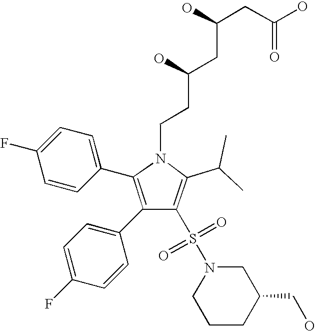

### 7-[2,3-Bis-(4-fluoro-phenyl)-4-(3-hydroxymethyl-piperidine-1-sulfonyl)-5-isopropyl-pyrrol-1-yl]-3,5-dihydroxy-heptanoic acid Sodium Salt

MS APCI+635.2 (acid+1)

Analyzed for: C32H39F2N2Na1O7S1.1.29H2O1

| | C | H | N |
|---|---|---|---|
| Theory | 56.52 | 6.16 | 4.12 |
| Found | 56.13 | 6.07 | 3.89 |

### Example 40P

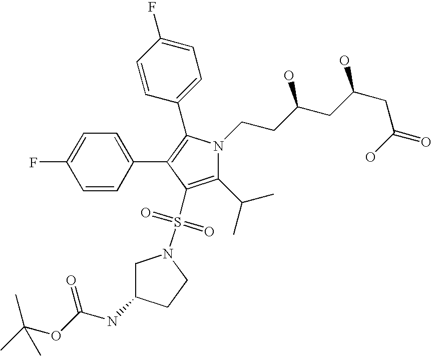

### 7-[3-(3-tert-Butoxycarbonylamino-pyrrolidine-1-sulfonyl)-4,5-bis-(4-fluoro-phenyl)-2-isopropyl-pyrrol-1-yl]-3,5-dihydroxy-heptanoic acid Sodium Salt

MS APCI+706.2 (acid+1)

Analyzed for: C35H44F2N3Na1O8S1.2.07H2O1

| | C | H | N |
|---|---|---|---|
| Theory | 54.94 | 6.34 | 5.49 |
| Found | 54.55 | 6.03 | 5.25 |

### Example 41P

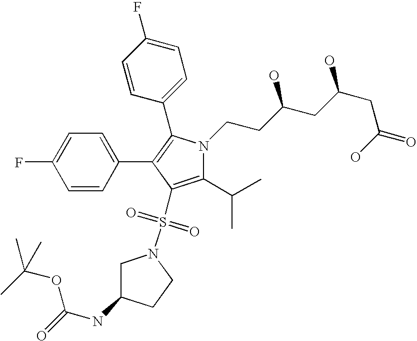

### 7-[3-(3-tert-Butoxycarbonylamino-pyrrolidine-1-sulfonyl)-4,5-bis-(4-fluoro-phenyl)-2-isopropyl-pyrrol-1-yl]-3,5-dihydroxy-heptanoic acid Sodium Salt

MS APCI+706.1 (acid+1)

Analyzed for: C35H44F2N3Na1O8S1.0.96H2O1

| | C | H | N |
|---|---|---|---|
| Theory | 56.42 | 6.21 | 5.64 |
| Found | 56.03 | 6.12 | 5.30 |

### Example 42P

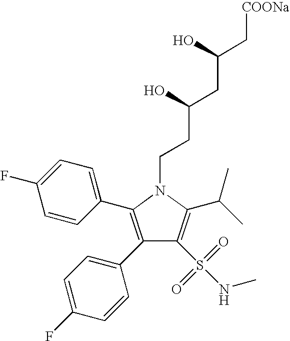

MP: 140-143° C.

Combustion Analysis: (C27H31F2N2Na1O6S1.0.35C4H10O1 (ethyl ether).1.7H2O1):

| | Carbon | Hydrogen | Nitrogen | F |
|---|---|---|---|---|
| Theory | 54.22 | 6.07 | 4.45 | 6.04 |
| Found | 54.33 | 5.71 | 4.06 | 6.10 |

### Example 43P

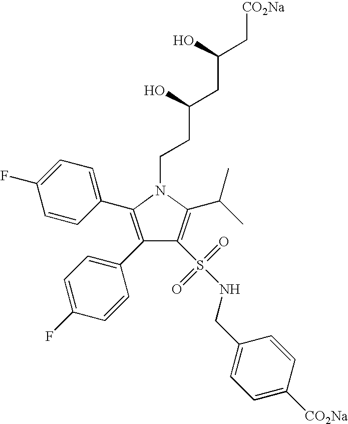

MS showed the di-acid peak APCI+(671.2, acid+H). MP >250° C.

Combustion Analysis for (C34H34F2N2Na2O8S1.4.0H2O1.1.60NaOH):

| | Carbon | Hydrogen | Nitrogen |
|---|---|---|---|
| Theory | 48.00 | 5.17 | 3.29 |
| Found | 47.76 | 4.78 | 2.92 |

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

### Formulations

The compounds of the present invention including those exemplified herein and all compounds of Formula I, hereafter referred to as “compound(s)” can be administered alone or in combination with one or more therapeutic agents. These include, for example, other agents for treating, preventing or controlling dyslipidemia, non-insulin dependent diabetes mellitus, obesity, hyperglycemia, hypercholesteremia, hyperlipidemia, atherosclerosis, hypertriglyceridemia, or hyperinsulinemia.

The compounds are thus well suited to formulation for convenient administration to mammals for the prevention and treatment of such disorders.

The following examples further illustrate typical formulations of the compounds provided by the invention.

Formulation 1

| Ingredient | Amount | |
|---|---|---|
| compound | 0.5 to 800 | mg |
| sodium benzoate | 5 | mg |
| isotonic saline | 1000 | mL |

The above ingredients are mixed and dissolved in the saline for IV administration to a patient.

Formulation 2

| Ingredient | Amount | |
|---|---|---|
| compound | 0.5 to 800 | mg |
| cellulose, microcrystalline | 400 | mg |
| stearic acid | 5 | mg |
| silicon dioxide | 10 | mg |
| sugar, confectionery | 50 | mg |

The ingredients are blended to uniformity and pressed into a tablet that is well suited for oral administration to a patient.

Formulation 3

| Ingredient | Amount | |
|---|---|---|
| compound | 0.5 to 800 | mg |
| starch, dried | 250 | mg |
| magnesium stearate | 10 | mg |

The ingredients are combined and milled to afford material suitable for filling hard gelatin capsules administered to a patient.

Formulation 4

| Ingredient | Amount % wt./(total wt.) |
|---|---|
| compound | 1 to 50 |
| Polyethylene glycol 1000 | 32 to 75 |
| Polyethylene glycol 4000 | 16 to 25 |

The ingredients are combined via melting and then poured into molds containing 2.5 g total weight.

### Biological Assays

The compounds of the invention have demonstrated HMG Co-A reductase inhibition in standard assays commonly employed by those skilled in the art. (See, e.g., J. of Lipid Research 1998; 39:75-84; Analytical Biochemistry, 1991; 196:211-214; RR 740-01077 Pharmacology 8-Nov.-82) Accordingly, such compounds and formulations comprising such compounds are useful for treating, controlling or preventing inter alia hypercholesterolemia, hyperlipidemia, hypertriglyceridemia or atherosclerosis.

A.) In Vitro assay

Rat Liver Microsomal Isolation Procedure:

Male Charles River Sprague-Dawley rats were fed with 2.5% cholestyramine in rat chow diets for 5 days before sacrificing. Livers were minced and homogenized in a sucrose homogenizing solution in an ice bath 10 times. Homogenates were diluted into a final volume of 200 mL, and centrifuged 15 min. with a Sorvall Centrifuge at 5° C., 10,000 rpm (12,000×G). The upper fat layer was removed and the supernatant decanted into fresh tubes. This step was repeated one more time before transferring the supernatant into ultracentrifuge tubes and centrifuged at 36,000 rpm (105,000×G) for an hour at 5° C. The resulting supernatant was discarded and the pellet was added to total of 15 mL 0.2 M KH₂PO₄. Pellets were homogenized gently by hand about 10 times. Samples were pooled and diluted into total of 60 mL buffer. The protein concentration of the homogenate was determined by the Lowry Method using a BCA (Bicinchoninic acid), kit from Pierce Chemical Company. 1 mL aliquots of microsomes were kept frozen in liquid nitrogen.

### HMGCoA (3-Hydroxy-3-methylglutaryl CoA) Reductase Assay:

Materials and Methods:

[3-¹⁴C]-HMGCoA (57.0 mCi/mmol) was purchased from Amersham Biosciences, UK. HMGCoA, mevalonolactone, P-NADPH (P-Nicotinamide Adenine Dinucleotide Phosphate, Reduced form) were purchased from Sigma Chemical Co. AG 1-8×resin was purchased from Bio-Rad Laboratory.
1. One μL of dimethyl sulfoxide (DMSO) or 1 μL of DMSO containing a test compound at a concentration sufficient to give a final assay concentration of between 0.1 nM to 1 mM was placed into each well of a Corning 96 well plate. A Volume of 34 □L of buffer (100 mM NaH₂PO₄, 10 mM Imidazole and 10 mM EDTA), (Ethylenediaminetetra acetic acid) containing with 50 g/mL rat liver microsomes was added into each well. After incubation for 30 min. on ice, 15 □L of ¹⁴C-HMGCoA (0.024 μCi) with 15 mM NADPH, 25 mM DTT, (Dithiothreitol) was added and incubated at 37° C. for an additional 45 min.

The reaction was terminated by the addition of 10 μL of HCl followed by 5 μL of mevalonolactone. Plates were incubated at room temperature overnight to allow lactonization of mevalonate to mevalonolactone. The incubated samples were applied to columns containing 300 μL of AG1-X8 anion exchange resin in a Corning filter plate.

The eluates were collected into Corning 96 well capture plates. Scintillation cocktail (Ultima-Flo-M) was added into each well and plates counted on a Trilux Microbeta Counter. The IC₅₀ values were calculated with GraphPad software (Prism). Procedure:
2. Add 1 μL DMSO or compounds into the wells according to the protocol
3. Add 35 μL incubation buffer with the rat microsomes into each well. Incubate 30 min. at 4° C.
4. Add 15 μL ¹⁴C-HMGCoA. Incubate 45 min. at 37° C.
5. Add 10 μL HCl stop reagent
6. Add 5 μL mevelonolactone. Incubate overnight at room temperature
7. Apply the containing into the AG 1-X8 anion exchange resin in Corning filter plate
8. Collect the eluate into Corning capture plate
9. Add scintillation cocktail Ultima-Flo-M
10. Count on a Trilux Microbeta Counter μ
11. Calculate IC₅₀ values

Compounds of the invention exhibit a range of IC₅₀ values of less than about 500 nM in the aforementioned in vitro assay. Preferred compounds of the invention exhibit a range of IC₅₀ values of less than about 100 nM. More preferred compounds of the invention exhibit a range of IC₅₀ values of less than about 20 nM. See, for example, the compounds of: Example 4, which has an IC₅₀ of 7.9 nM, Example 62, which has an IC₅₀ of 7.2 nM, Example 69, which has an IC₅₀ of 2.2 nM, Example 103, which has an IC₅₀ of 50.4 nM, Example 104, which has an IC₅₀of 75.8 nM, Example 110, which has an IC₅₀ of 1.38 nM, Example 111, which has an IC₅₀ of 1.17 nM, and Example 112, which has an IC₅₀ of 8.39 nM.

B.) Cell Assay

Protocol for Sterol Biosynthesis in Rat Hepatocytes:

Cell culture, compounds treatment and cell labeling:

Frozen rat hepatocytes purchased from XenoTech (cat# N400572) were seeded on 6-well collagen I coated plates at a density of 10⁵ cells/per well. The cells were grown in DMEM, (Dulbecco's Modified Eagle Medium) (Gibco, #11054-020) containing 10% FBS (Fetal Bovine Serum) and 10 mM HEPES, (N-2-hydroxyethyl-piperazine-N¹-2-ethane sulfonic acid) (Gibco # 15630-080) for 24 hrs. The cells were pre-incubated with compounds for 4 hrs and then labeled by incubating in medium containing 1 uCVper mL of ¹⁴C acetic acid for an additional 4 hrs. After labeling, the cells were washed twice with 5 mM MOPS, (3-[N-morpholino] propane sulfonic acid) solution containing 150 mM NaCl and 1 mM EDTA and collected in the lysis buffer containing 10% KOH and 80% (vol.) ethanol. Cholesterol extraction and data analysis:

In order to separate labeled cholesterol from labeled non-cholesterol lipids, the cells lysates were subject to saponification at 60° C. for 2 hrs. The lysates were then combined with 0.5 volume of H₂O and 2 volumes of hexane, followed by 30 minutes of vigorous shaking. After the separation of two phases, the upper-phase solution was collected and combined with 5 volumes of scintillation cocktail. The amount of ¹⁴C cholesterol was quantified by liquid scintillation counting. The IC₅₀ values were calculated with GraphPad software (Prism 3.03).

Compounds of the invention exhibit a range of IC₅₀ values of less than about 1000 nM in the aforementioned cell assay. Preferred compounds of the invention exhibit a range of IC₅₀values of less than about 100 nM. See, for example, the compounds of: Example 4, which has an IC₅₀ of 0.42 nM, Example 62, which has an IC₅₀ of 0.58 nM, Example 69, which has an IC₅₀ of 0.18 nM, Example 103, which has an IC₅₀ of 0.0880 nM, Example 110, which has an IC₅₀ of 0.218 nM, Example 111, which has an IC₅₀ of 0.146 nM, and Example 112, which has an IC₅₀ of 1.15 nM.

C.) Protocol for Sterol Biosynthesis in L6 Rat Myoblast:

Cell culture, compounds treatment and cell labeling:

L6 rat myoblast purchased from ATCC (CRL-1458) were grown in T-150 vented culture flasks and seeded on 12-well culture plates at a density of 60,000 cells per well. The cells were grown in DMEM, (Dulbecco's Modified Eagle Medium) (Gibco, #10567-014) containing 10% heat inactivated FBS (Fetal Bovine Serum) (Gibco # 10082-139) for 72 hours until reaching confluence. The cells were pre-incubated in media with compound and 0.2% DMSO (dimethyl sulfoxide) for 3 hours and then labeled by incubating in medium containing compound, 0.2% DMSO and 1 □]Ci/per mL of ¹⁴C acetic acid for an additional 3 hours. After labeling, the cells were washed once with 1×PBS (Gibco #14190-144) then lysed overnight at 4° C. in buffer containing 10% KOH and 78%(vol.) ethanol. Cholesterol extraction and data analysis:

Lipid ester bonds were hydrolyzed by saponification of the lysates at 60° C. for 2 hours. Sterols (including cholesterol) were extracted from saponified lysates by combining with 3 volumes of hexane and mixing by pipette 6 times. The upper organic phase solution was collected and combined with an equal volume of 1 N KOH in 50% methanol and mixed by pipette 6 times. The upper organic phase was collected in a scintilant-coated plate (Wallac #1450-501) and hexanes removed by evaporation at room temperature for 3 hours. The amount of ¹⁴C cholesterol was quantified by scintillation counting in a Trilux 1450 plate reader (Wallac). The IC₅₀ values were calculated from % inhibitions relative to negative controls vs. compound concentration on Microsoft excel 2000 data analysis wizard using a sigmoid inhibition curve model with formula:
y=Bmax (1−(xⁿ/Kⁿ+xⁿ))+y2

Where K is the IC₅₀ for the inhibition curve, X is inhibitor concentration, Y is the response being inhibited and Bmax+Y2 is the limiting response as X approaches zero. Compounds of the invention have a L6 IC₅₀ value greater than about 100 nM in the aforementioned L6 Rat Myoblast. See, for example, the compounds of: Example 4, which has an L6 IC₅₀ of 3069 nM, Example 62, which has an L6 IC₅₀ of 703 nM, Example 69, which has an L6 IC₅₀ of 159 nM, Example 110, which has an L6 IC₅₀ of 632 nM, Example 111, which has an L6 IC₅₀ of 6400 nM, and Example 112, which has an L6 IC₅₀ of 73,500 nM. Preferred compounds of the invention exhibit a hepatocyte selectivity greater than about 1000 ((L6 IC₅₀/Rat hepatocyte IC₅₀) >1000), and have a L6 IC₅₀ value greater than about 1000 nM.

What is claimed is:

1. A compound having a Formula I, 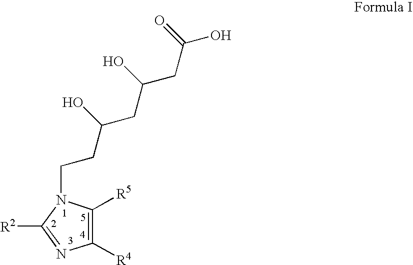
or a pharmaceutically acceptable salt, ester, amide or stereoisomer thereof wherein:
R² and R⁵ are each independently H; halogen; C₁-C₆ alkyl, C₃-C₈ cycloalkyl, aryl, aralkyl, heteroaryl or heteroaralkyl; optionally substituted;
R⁴ is halogen; H; C₁-C₆ alkyl, C₃-C₈ cycloalkyl, aryl, aralkyl, heteroaryl or heteroaralkyl; optionally substituted; —(CH₂)ₙC(O)NR⁶R⁷; R⁸S(O)ₙ—;
—(CH₂)ₙNR⁶R⁷; —(CH₂)ₙCOOR′; or —(CH₂)ₙCOR′;
R⁶ and R⁷ are each independently H; C₁-C₁₀ alkyl, C₃-C₈ cycloalkyl, aryl, aralkyl, heteroaryl or heteroaralkyl; optionally substituted with aryl, heteroaryl, lower alkyl, halogen, OR′, —(CH₂)ₙCOOR′, —(CH₂)ₙCONR′R″, (CH₂)ₙSO₂R′, SO₂NR′R″ or CN;
—(CH₂)ₙCOR′, —(CH₂)ₙCOOR′, —(CH₂)ₙCONR′R″ or —(CH₂)ₙSO₂R′; or
N, R⁶ and R⁷ taken together form a 4-11 member ring optionally containing up to two heteroatoms selected from O, N and S, said ring being optionally substituted with aryl, aralkyl, heteroaryl, heteroaralkyl, C₁-C₁₀ alkyl, C₃-C₈ cycloalkyl, halogen, OR′, —(CH₂)ₙCOOR′, —(CH₂)ₙCONR′R″, —(CH₂)ₙSO₂R′, SO₂NR′R″ or CN;
R⁸ is aryl, aralkyl, alkyl, heteroaryl, or heteroaralkyl; optionally substituted;
R and R″ are each independently H; C₁-C₁₂ alkyl, aryl or aralkyl; optionally substituted; and n is 0-2.

2. The compound of the Formula I of claim 1, a pharmaceutically acceptable salt, ester, amide or stereoisomer thereof wherein R² is aryl, aralkyl, heteroaryl or heteroaralkyl; optionally substituted.

3. The compound of claim 1 or claim 2, a pharmaceutically acceptable salt, ester, amide or stereoisomer thereof wherein R⁴ is —(CH₂)ₙC(O)NR⁶R⁷.

4. The compound of claim 2, a pharmaceutically acceptable salt, ester, amide or stereoisomer thereof wherein R² is phenyl, optionally substituted with one or more halogen.

5. The compound of claim 1, or a pharmaceutically acceptable salt, ester, amide or stereoisomer thereof wherein one of R⁶ and R⁷is aralkyl, optionally substituted; and the other one of R⁶ and R⁷ is H.

6. The compound of claim 5, a pharmaceutically acceptable salt, ester, amide or stereoisomer thereof wherein one of R⁶ and R⁷is benzyl, optionally substituted.

7. The compound of the Formula I of claim 1, a pharmaceutically acceptable salt, ester, amide or stereoisomer thereof wherein R⁵ is isopropyl or cyclopropyl.

8. A pharmaceutically acceptable salt of a compound of the Formula I of claim 1 wherein the salt is a sodium salt.

9. A lactone form of a compound of claim 1 having a Formula C: 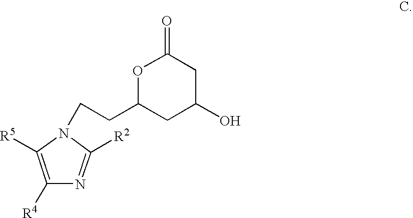
wherein R², R⁴ and R⁵ are as defined in claim 1.

10. The lactone form of claim 9, wherein R²is phenyl optionally substituted with one or more halogen, R⁴ is —(CH₂)ₙC(O)NR⁶R⁷, one of R⁶ and R⁷ is aralkyl, optionally substituted, and the other one of R⁶ and R⁷ is H; and R⁵ is C₁-C₆ alkyl or C₃-C₈ cycloalkyl.

11. A process for preparing a compound having a Formula b. 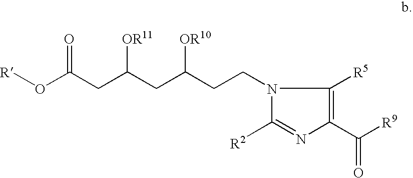
from a compound having a Formula a. 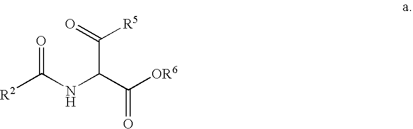
comprising the following steps:
1.) Reacting the compound a. with a compound having a formula c., 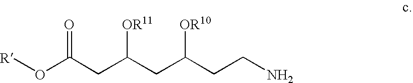
in a solvent; and
optionally reacting the compound a. with a compound NHR⁶R⁷, in a solvent, prior to the first step;
wherein R² and R⁵ are each independently H; halogen; C₁-C₆ alkyl, C₃-C₈ cycloalkyl, aryl, aralkyl, heteroaryl or heteroaralkyl; optionally substituted;
R⁹ is —OR⁶ or —NR⁶R⁷;
R⁶ is H; C₁-C₁₀ alkyl, C₃-C₈ cycloalkyl, aryl, aralkyl, heteroaryl or heteroaralkyl;
optionally substituted with aryl, heteroaryl, lower alkyl, halogen, OR′, —(CH₂)ₙCOOR′, —(CH₂)ₙCONR′R″, (CH₂)ₙSO₂R′, SO₂NR′R″ or CN;
R⁷ is H; C₁-C₁₀ alkyl, C₃-C₈ cycloalkyl, aryl, aralkyl, heteroaryl or heteroaralkyl; optionally substituted with aryl, heteroaryl, lower alkyl, halogen, OR′,
—(CH₂)ₙCOOR′, —(CH₂)ₙCONR′R″, (CH₂)ₙSO₂R′, SO₂NR′R″ or CN; —(CH₂)ₙCOR′, —(CH₂)ₙCOOR′, —(CH₂)ₙCONR′R″ or —(CH₂)ₙSO₂R′; or
N, R⁶ and R⁷ taken together form a 4-11 member ring optionally containing up to two heteroatoms selected from O, N and S, said ring being optionally substituted with aryl, aralkyl, heteroaryl, heteroaralkyl, C₁-C₁₀ alkyl, C₃-C₈ cycloalkyl, halogen, OR′, —(CH₂)ₙCOOR′, —(CH₂)ₙCONR′R″, —(CH₂)ₙSO₂R′, SO₂NR′R″ or CN;
R′ and R″ are each independently H; C₁-C₁₂ alkyl, aryl or aralkyl; optionally substituted; n is 0-2;
R¹⁰ and R¹¹ are each independently C₁-C₁₀alkyl, C(O)R⁷, —SiR¹²R¹³R¹⁴or R¹⁰ and R¹¹ taken together from isopropyl; and R¹², R¹³ and R¹⁴ are each independently C₁-C₆ alkyl.

12. A compound of the Formula I of claim 1 selected from the group consisting of:
(3R,5R)-7-[4-Benzylcarbamoyl-2-(4-fluoro-phenyl)-5-isopropyl-imidazol-1-yl]-3,5-dihydroxy-heptanoic acid;
(3R,5R)-7-[2-(4-Fluoro-phenyl)-5-isopropyl-4-(2-methoxy-ethylcarbamoyl)-imidazol-1-yl]-3,5-dihydroxy-heptanoic acid;
(3R,5R)-7-[2-(4-Fluoro-phenyl)-5-isopropyl-4-phenylcarbamoyl-imidazol-1-yl]-3,5-dihydroxy-heptanoic acid;
(3R,5R)-7-[4-(1,3-Dihydro-isoindole-2-carbonyl)-2-(4-fluoro-phenyl)-5-isopropyl-imidazol-1-yl]-3,5-dihydroxy-heptanoic acid;
(3R,5R)-7-[4-(Benzyl-ethyl-carbamoyl)-2-(4-fluoro-phenyl)-5-isopropyl-imidazol-1-yl]-3,5-dihydroxy-heptanoic acid;
(3R,5R)-7-{2-(4-Fluoro-phenyl)-5-isopropyl-4-[(pyridin-3-ylmethyl)-carbamoyl]-imidazol-1-yl}-3,5-dihydroxy-heptanoic acid;
(3R,5R)-7-[2-(4-Fluoro-phenyl)-5-isopropyl-4-(2-pyridin-3-yl-ethylcarbamoyl)-imidazol-1-yl]-3,5-dihydroxy-heptanoic acid;
(3R,5R)-7-[2-(4-Fluoro-phenyl)-5-isopropyl-4-((R)-2-phenyl-propylcarbamoyl)-imidazol-1-yl]-3,5-dihydroxy-heptanoic acid;
(3R,5R)-7-[4-[2-(4-Chloro-phenyl)-3-hydroxy-propylcarbamoyl]-2-(4-fluoro-phenyl)-5-isopropyl-imidazol-1-yl]-3,5-dihydroxy-heptanoic acid;
(3R,5R)-7-{2-(4-Fluoro-phenyl)-5-isopropyl-4-[2-(4-sulfamoyl-phenyl)-ethylcarbamoyl]-imidazol-1-yl}-3,5-dihydroxy-heptanoic acid;
(3R,5R)-7-[2-(4-fluoro-phenyl)-5-isopropyl-4-((S)-1-methyl-3-phenyl-propylcarbamoyl)-imidazol-1-yl]-3,5-dihydroxy-heptanoic acid;
(3R,5R)-7-{2-(4-fluoro-phenyl)-4-[2-(3-fluoro-phenyl)-ethylcarbamoyl]-5-isopropyl-imidazol-1-yl}-3,5-dihydroxy-heptanoic acid;
(3R,5R)-7-[2-(4-fluoro-phenyl)-4-((1 S,2S)-2-hydroxy-1-methoxymethyl-2-phenyl-ethylcarbamoyl)-5-isopropyl-imidazol-1-yl]-3,5-dihydroxy-heptanoic acid; (3R,5R)-7-{2-(4-fluoro-phenyl)-5-isopropyl-4-[2-(4-methoxy-phenyl)-ethylcarbamoyl]-imidazol-1-yl}-3,5-dihydroxy-heptanoic acid;
(3R,5R)-7-[2-(4-fluoro-phenyl)-4-((S)-1-hydroxymethyl-2-phenyl-ethylcarbamoyl)-5isopropyl-imidazol-1-yl]-3,5-dihydroxy-heptanoic acid;
(3R ,5R)-7-{2-(4-fluoro-phenyl)-4-[(1 S,2S)-2-hydroxy-1-hydroxymethyl-2-(4-methylsulfanyl-phenyl)-ethylcarbamoyl]-5-isopropyl-imidazol-1-yl}-3,5-dihydroxy-heptanoic acid;
(3R,5R)-7-[4-[2-(4-chloro-phenyl)-ethylcarbamoyl]-2-(4-fluoro-phenyl)-5-isopropyl-imidazol-1 -yl]-3,5-dihydroxy-heptanoic acid;
(3R,5R)-7-[2-(4-fluoro-phenyl)-5-isopropyl-4-((S)-2-phenyl-propylcarbamoyl)-imidazol-1-yl]-3,5-dihydroxy-heptanoic acid;
(3R,5R)-7-(2-(4-fluoro-phenyl)-5-isopropyl-4-[2-(3-methoxy-phenyl)-ethylcarbamoyl]-imidazol-1-yl}-3,5-dihydroxy-heptanoic acid;
(3R,5R)-7-(2-(4-fluoro-phenyl)-4-[2-(4-fluoro-phenyl)-ethylcarbamoyl]-5-isopropyl-imidazol-1-yl}-3,5-dihydroxy-heptanoic acid;
(3R,5R)-7-[4-[2-(3-chloro-phenyl)-ethylcarbamoyl]-2-(4-fluoro-phenyl)-5-isopropyl-imidazol-1-yl]-3,5-dihydroxy-heptanoic acid;
(3R,5R)-7-[2-(4-fluoro-phenyl)-5-isopropyl-4-(2-pyridin-4-yl-ethylcarbamoyl)-imidazol-1-yl]-3,5-dihydroxy-heptanoic acid;
(3R,5R)-7-[2-(4-fluoro-phenyl)-4-((1 R,2R)-2-hydroxy-1-hydroxymethyl-2-phenyl-thylcarbamoyl)-5-isopropyl-imidazol-1-yl]-3,5-dihydroxy-heptanoic acid;
(3R,5R)-7-[2-(4-Fluoro-phenyl)-5-isopropyl-4-benzylcarbamoyl-imidazol-1-yl]-3,5-dihydroxy-heptanoic acid;
(3R,5R)-7-[2-(4-Fluoro-phenyl)-5-isopropyl-4-phenylcarbamoyl-imidazol-1-yl]-3,5-dihydroxy-heptanoic acid;
(3S,5R)-7-[2-(4-fluoro-phenyl)-5-isopropyl-4-(toluene-4-sulfonyl)-imidazol-1-yl]-3,5-dihydroxy-heptanoic acid; (3R,5R)-7-[2-(4-Fluoro-phenyl)-5-ethyl-4-(4-fluorophenylcarbamoyl)-imidazol-1-yl]-3,5-dihydroxy-heptanoic acid;
(3R,5R)-7-[2-(4-Fluoro-phenyl)-5-propyl-4-phenylcarbamoyl-imidazol- 1-yl]-3,5-dihydroxy-heptanoic acid;
(3R,5R)-7-[2-(4-Fluoro-phenyl)-5-propyl-4-benzylcarbamoyl-imidazol-1-yl]-3,5-dihydroxy-heptanoic acid;
(3R,5R)-7-[2-(4-Fluoro-phenyl)-5-propyl-4-phenethyl-carbamoyl-imidazol-1-yl]-3,5-dihydroxy-heptanoic acid;
(3R,5R)-7-[2-(4-Fluoro-phenyl)-5-propyl-4-(4-fluorophenylcarbamoyl)-imidazol-l-yl]-3,5-dihydroxy-heptanoic acid;
(3R,5R)-7-[2-(4-Fluoro-phenyl)-5-methyl-4-phenylcarbamoyl-imidazol-1-yl]-3,5-dihydroxy-heptanoic acid;
(3R,5R)-7-[2-(4-Fluoro-phenyl)-5-methyl-4-benzylcarbamoyl-imidazol-1-yl]-3,5-dihydroxy-heptanoic acid;
(3R ,5R)-7-[2-(4-Fluoro-phenyl)-5-methyl-4-phenethylcarbamoyl-imidazol- 1-yl]-3,5-dihydroxy-heptanoic acid;
(3R,5R)-7-[4-[(Biphenyl-3-ylmethyl)-carbamoyl]-2-(4-fluoro-phenyl)-5-isopropyl-imidazol-1-yl]-3,5-dihydroxy-heptanoic acid;
(3R,5R)-7-[2-(4-Fluoro-phenyl)-5-isopropyl-4-phenethylcarbamoyl-imidazol-1-yl]-3,5-dihydroxy-heptanoic acid;
(3R,5R)-7-[2-(4-Fluoro-phenyl)-5-methyl-4-(4-sulfamoyl-benzylcarbamoyl)-imidazol-1-yl]-3,5-dihydroxy-heptanoic acid;
(3R,5R)-7-[4-benzylcarbamoyl-2-phenyl-5-isopropyl-imidazol-1-yl]-3,5-dihydroxy-heptanoic acid;
(3R,5R)-7-[4-(3-Chloro-benzylcarbamoyl)-2-(4-fluoro-phenyl)-5-isopropyl-imidazol-1-yl]-3,5-dihydroxy-heptanoic acid;
(3R,5R)-7-[2-(4-Fluoro-phenyl)-4-(indan-1-ylcarbamoyl)-5-isopropyl-imidazol-1-yl]-3,5-dihydroxy-heptanoic acid;
(3R,5R)-7-[4-Benzylcarbamoyl-5-cyclopropyl-2-(4-fluoro-phenyl)-imidazol-1-yl]-3,5-dihydroxy-heptanoic acid;
(3R,5R)-7-[5-Cyclopropyl-2-(4-fluoro-phenyl)-4-(4-methoxy-benzylcarbamoyl)-imidazol-1-yl]-3,5-dihydroxy-heptanoic acid;
and pharmaceutically acceptable salts and lactone forms thereof.

13. The compound of claim 1 selected from the group consisting of (3R,5R)-7-[4-Benzylcarbamoyl-2-(4-fluoro-phenyl)-5-isopropyl-imidazol-1-yl]-3,5-dihydroxy-heptanoic acid; pharmaceutically acceptable salts and lactone forms thereof.

14. A combination of the compound of claim 1 and one or more additional pharmaceutically active agent.

15. A pharmaceutical composition comprising the compound of claim 1 and a pharmaceutically acceptable carrier, diluent or vehicle.

16. A compound having a Formula I, 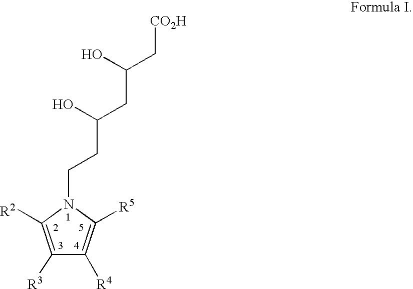
or a pharmaceutically acceptable salt, ester, amide, stereoisomer or prodrug thereof, or a pharmaceutically acceptable salt of the prodrug, wherein:
R² is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms;
One of R³ and R⁴ is H; aryl, aralkyl, heteroaryl, heteroaralkyl, optionally substituted;
C₁-C₈ alkyl straight chain or branched; or C₃-C₈ cycloalkyl; and the other one of R³ and R⁴ is I, COOR′, R⁶R⁷NC(O)— or SO₂NR⁹R¹⁰;
One of R⁶ and R⁷ is SO₂NHR⁸ or SO₂R⁸; and the other one of R⁶ and R⁷is H or C₁-C₄ alkyl;
R⁸ is aryl or heteroaryl, optionally substituted;
R⁹ and R¹⁰ are each independently H; aryl, aralkyl, heteroaryl or heteroaralkyl optionally substituted with halogen, OR′, (CH₂)ₙCOOR′, (CH₂)ₙCONR′R″, (CH₂)ₙSO₂NR′R″, (CH₂)ₙSO₂R′ or CN; C₁-C₁₀ alkyl unsubstituted or substituted with OH, CO₂R′ or CONR′R″;
or N, R⁹ and R¹⁰ taken together form a 4-11 member ring optionally containing up to 2 heteroatoms selected from O, N and S, said ring optionally substituted with ═O, OH, benzyl, phenyl, CO₂R′, R′OR″, (CH₂)ₙSO₂R′ or CONR′R″;
R⁵ is alkyl of from one to four carbon atoms, optionally substituted with a halogen;
R′ and R″ are each independently H, lower alkyl or taken together form a 4-7 member ring;
and n is 0-2.

17. A compound of the Formula I of claim 16 selected from the group consisting of:
(3R,5R)-7-[2,3-Bis-(4-fluoro-phenyl)-5-isopropyl-4-methylsulfamoyl-pyrrol-1-yl]-3,5-dihydroxy-heptanoic acid; (3R,5R)-7-[2,3-Bis-(4-fluoro-phenyl)-5-isopropyl-pyrrol-1-yl]-3,5-dihydroxy-heptanoic acid; (3R,5R)-7-[2,3-Bis-(4-fluoro-phenyl)-4-(2-hydroxy-phenysulfamoyl)-5-isopropyl-pyrrol-1-yl]-3,5-dihydroxy-heptanoic acid; (3R,5R)-7-[2,3-Bis-(4-fluoro-phenyl)-5-isopropyl-4-phenylsulfamoyl-pyrrol-1-yl]-3,5-dihydroxy-heptanoic acid; 4-[1-((3R,5R)-6-Carboxy-3,5-dihydroxy-hexyl)-4,5-bis-(4-fluoro-phenyl)-2-isopropyl-1H-pyrrole-3-sulfonylamino]-benzoic acid; 1-[1-((3R,5R)-6-Carboxy-3,5-dihydroxy-hexyl)-4,5-bis-(4-fluoro-phenyl)-2-isopropyl-1H-pyrrole-3-sulfonyl]-piperidine-4-carboxylic acid; (3R,5R)-7-[2,3-Bis-(4-fluoro-phenyl)-5-isopropyl-4-(2-methoxycarbonyl-ethylsulfamoyl)-pyrrol-1-yl]-3,5-dihydroxy-heptanoic acid; (3R,5R)-7-[2,3-Bis-(4-fluoro-phenyl)-5-isopropyl-4-(3-methoxycarbonyl-propylsulfamoyl)-pyrrol-1-yl]-3,5-dihydroxy-heptanoic acid; (3R,5R)-7-[3-(2,4-Difluoro-phenylsulfamoyl)-4,5-bis-(4-fluoro-phenyl)-2-isopropyl-pyrrol-1-yl]-3,5-dihydroxy-heptanoic acid; (3R,5R)-7-[3-Carbamoyl-4,5-bis-(4-fluoro-phenyl)-2-isopropyl-pyrrol-1-yl]-3,5-dihydroxy-heptanoic acid; (3R,5R)-7-[2,3-Bis-(4-fluoro-phenyl)-5-isopropyl-4-(toluene-4-sulfonylaminocarbonyl)-pyrrol-1-yl]-3,5-dihydroxy-heptanoic acid; (3R,5R)-7-[2,3-Bis-(4-fluoro-phenyl)-4-(2-hydroxy-ethylsulfamoyl)-5-isopropyl-pyrrol-1-yl]-3,5-dihydroxy-heptanoic acid; and pharmaceutically acceptable salts, esters, and amides thereof.

18. A compound of the Formula I of claim 16 selected from the group consisting of: 4-{[1-((3R,5R)-6-Carboxy-3,5-dihydroxy-hexyl)-5-(4-fluoro-phenyl)-2-isopropyl-4-phenyl-1H-pyrrole-3-carbonyl]-amino}-benzoic acid; (3R,5R)-7-[3-(4-Cyano-phenyl)-2-(4-fluoro-phenyl)-5-isopropyl-4-phenylcarbamoyl-pyrrol-1-yl]-3,5-dihydroxy-heptanoic acid; (3R ,5R)-7-[3-(4-Bromo-phenyl)-2-(4-fluoro-phenyl)-5-isopropyl-4-phenylcarbamoyl-pyrrol-1-yl]-3,5-dihydroxy-heptanoic acid; (3R,5R)-7-[3-(3,4-Difluoro-phenyl)-2-(4-fluoro-phenyl)-5-isopropyl-4-phenylcarbamoyl-pyrrol-1-yl]-3,5-dihydroxy-heptanoic acid; 4-{[1-((3R,5R)-6-Carboxy-3,5-dihydroxy-hexyl)-5-(4-fluoro-phenyl)-2-isopropyl-4-phenyl- 1H-pyrrole-3-carbonyl]-amino}-benzoic acid; (3R,5R)-7-[2,3-Bis-(4-fluoro-phenyl)-4-(2-hydroxy-phenylsulfamoyl)-5-isopropyl-pyrrol- 1-yl]-3,5-dihydroxy-heptanoic acid; (3R,5R)-7-[2-(4-Fluoro-phenyl)-5-isopropyl-3-naphthalen-2-yl-4-phenylcarbamoyl-pyrrol-1-yl]-3,5-dihydroxy-heptanoic acid; (3R,5R)-7-[3-Cyclopropyl-2-(4-fluoro-phenyl)-5-isopropyl-4-phenylcarbamoyl-pyrrol-1-yl]-3,5-5 dihydroxy-heptanoic acid; (3R,5R)-7-[3-(4-Dimethylcarbamoyl-phenylcarbamoyl)-5-(4-fluoro-phenyl)-2-isopropyl-4-phenyl-pyrrol-1-yl]-3,5-dihydroxy-heptanoic acid; and
pharmaceutically acceptable salts, esters and amides thereof.

19. A pharmaceutical composition comprising the compound of claim 16, the pharmaceutically acceptable salt, ester, amide or prodrug thereof, or the pharmaceutically acceptable salt of the prodrug; and a pharmaceutically acceptable carrier, diluent, or vehicle.

20. A compound having a Formula 15 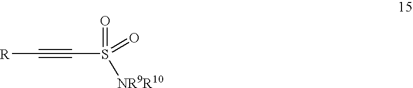
wherein R is C₁-C₈ alkyl straight chain or branched or C₃-C₈ cycloalkyl;
R⁹ and R¹⁰ are each independently H; aryl, aralkyl, heteroaryl, or heteroaralkyl;
optionally substituted; C₁-C₁₀alkyl unsubstituted or substituted with OH, CO₂R′ or CONR′R″; or N, R⁹ and R¹⁰ taken together form a 4-7 member ring, optionally containing up to 2 heteroatoms selected from O, N and S, said ring optionally substituted with OH, benzyl, phenyl, CO₂R′ or CONR′R″; and R and R are each independently H, lower alkyl or taken together form a 4-7 member ring.

21. A compound of the Formula I of claim 16 selected from the group consisting of:
(3R,5R)-7-[2-(4-fluoro-phenyl)-4-iodo-5-isopropyl-3-phenyl-pyrrol-1-yl]-3,5-dihydroxy-heptanoic acid; (3R,5R)-7-[3-(4-Carbamoyl-phenylsulfamoyl)-4,5-bis-(4-fluoro-phenyl)-2-isopropyl-pyrrol-1-yl]-3,5-dihydroxy-heptanoic acid; (3R,5R)-7-[2,3-Bis-(4-fluoro-phenyl)-5-isopropyl-4-(4-sulfamoyl-phenylsulfamoyl)-pyrrol-1-yl]-3,5-dihydroxy-heptanoic acid; (3R,5R)-7-[2-(4-Fluoro-phenyl)-5-isopropyl-4-(morpholine-4-sulfonyl)-3-phenyl-pyrrol-1-yl]-3,5-dihydroxy-heptanoic acid; (3R,5R)-7-[3-(Benzyl-methyl-sulfamoyl)-5-(4-fluoro-phenyl)-2-isopropyl-4-naphthalen-2-yl-pyrrol-1-yl]-3,5-dihydroxy-heptanoic acid; (3R,5R)-7-[3-(4-Benzyl-piperidine-1-sulfonyl)-5-(4-fluoro-phenyl)-2-isopropyl-4-phenyl-pyrrol-1-yl]-3,5-dihydroxy-heptanoic acid; and pharmaceutically acceptable salts, esters and amides thereof.

22. A compound having a formula C, 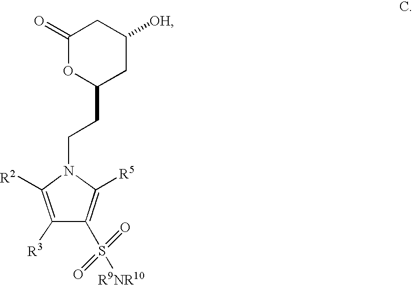
Wherein R²is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms;
R³ is H; aryl, aralkyl, heteroaryl, heteroaralkyl, optionally substituted; Cl-C₈ alkyl straight chain or branched; or C₃-C₈ cycloalkyl;
R⁵ is alkyl of from one to four carbon atoms, optionally substituted with a halogen; and
R⁹ and R¹⁰ are each independently H; aryl, aralkyl, heteroaryl or heteroaralkyl optionally substituted with halogen, OR′, (CH₂)ₙCOOR′, (CH₂)ₙCONR′R″, (CH₂)ₙSO₂NR′R″, (CH₂)ₙSO₂R′ or CN; C₁-C₁₀ alkyl unsubstituted or substituted with OH, CO₂R′ or CONR′R″;
or N, R⁹ and R¹⁰ taken together form a 4-7 member ring optionally containing up to 2 heteroatoms selected from O, N and S, said ring optionally substituted with OH, benzyl, phenyl, CO₂R″ or CONR′R″;
R⁵ is alkyl of from one to four carbon atoms, optionally substituted with a halogen;
R′ and R″ are each independently H, lower alkyl or taken together form a 4-7 member ring; and n is 0-2.

23. A compound having a Formula, 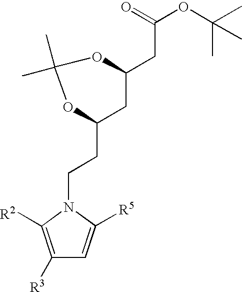
or a pharmaceutically acceptable salt, ester, amide, stereoisomer or prodrug thereof or a pharmaceutically acceptable salt of the prodrug
Wherein R² is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms;
R³ is H; aryl, aralkyl, heteroaryl, heteroaralkyl, optionally substituted; C₁-C₈ alkyl straight chain or branched; or C₃-C₈ cycloalkyl; and
R⁵ is alkyl of from one to four carbon atoms, optionally substituted with a halogen.

24. A compound having a formula 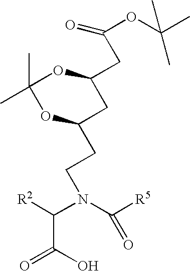
wherein R² is benzyl, naphthyl or cyclohexyl, optionally substituted; or phenyl optionally substituted with fluorine, chlorine, bromine, hydroxyl or trifluoromethyl; pyridinyl or pyridinyl substituted with fluorine, chlorine, bromine, hydroxyl or trifluoromethyl; or alkyl of from one to seven carbon atoms; and R⁵ is alkyl of from one to four carbon atoms, optionally substituted with a halogen.
